(19) United States
(12) Patent Application Publication
(10) Pub. No.: US 2023/0404466 A1
(43) Pub. Date: Dec. 21, 2023

(54) APPARATUS AND METHOD FOR "TRANSPLANTING" BRAIN STATES VIA BRAIN ENTRAINMENT

(71) Applicant: Neuroenhancement Lab, LLC, Pomona, NY (US)

(72) Inventor: Alexander I. Poltorak, Pomona, NY (US)

(21) Appl. No.: 18/211,230

(22) Filed: Jun. 16, 2023

Related U.S. Application Data
(60) Provisional application No. 63/354,213, filed on Jun. 21, 2022.

(51) Int. Cl. A61B 5/00; A61B 5/37

(52) U.S. Cl. CPC A61B 5/4064; A61B 5/37; A61N 1/36025

(57) ABSTRACT

Brain states, which correlate with specific motor, cognitive, and emotional states, are non-invasively monitored, representing macroscopic cortical activity manifested as oscillatory network dynamics. Sensory and/or transcranial stimulation, entraining brain rhythms, effectively induce desired brain states correlated with a state of sleep or state of attention. Brain waves are recorded from a donor which are then inverted by processing and used to entrain the brain of a “recipient”. Brain states may thus be transferred between people by acquiring an associated cortical signature from a donor, which, following processing, is applied to a recipient through sensory or transcranial stimulation. This technique provides an effective neuromodulation approach to the noninvasive, non-pharmacological treatment of a variety of psychiatric and neurological disorders for which current treatments are mostly limited to pharmacotherapeutic interventions.

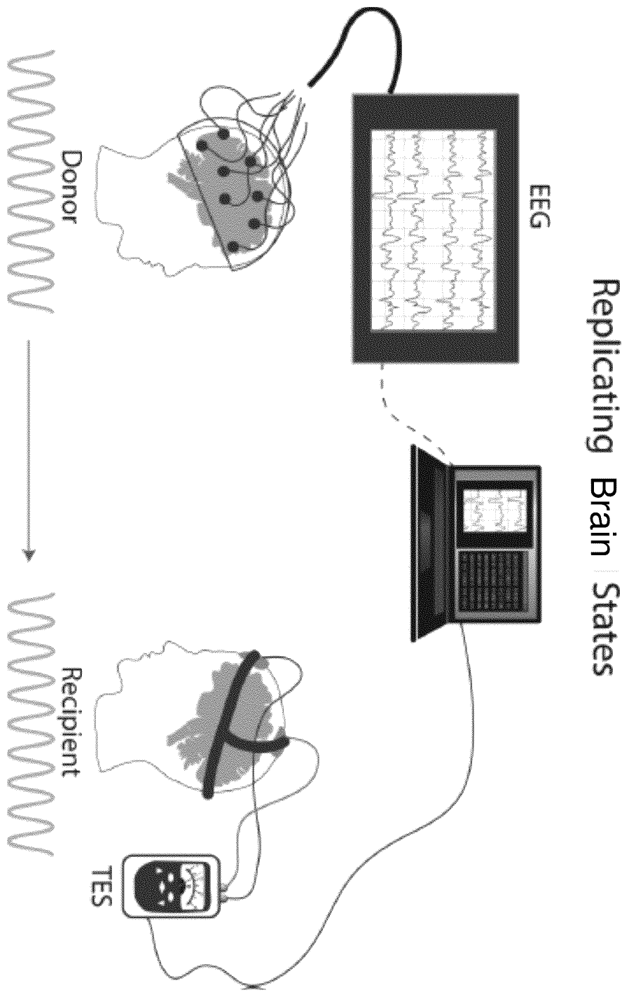

### CROSS REFERENCE TO RELATED APPLICATIONS

The present application is a non-provisional of, and claims benefit of priority under 35 U.S.C. § 119(e) from U.S. Provisional Patent Application No. 63/354,213, filed Jun. 21, 2023, the entirety of which is expressly incorporated herein by reference.

### FIELD OF THE INVENTION

The present invention relates to the field of neuromodulation, and more particularly to replicating brain states through transcranial and/or sensory brain stimulation.

### BACKGROUND OF THE INVENTION

Each reference and document cited herein is expressly incorporated herein by reference in its entirety, for all purposes.

It has been suggested (Crick and Koch, 1990) (see also (Rees et al., 20 Crick and Koch, 2006)) that every mental state is expressed through unique neural signals, such as neuronal oscillations, that are correlated with mood, cognition, and motor functions. Crick and Koch (1990) thus proposed that neuronal oscillations are the neural correlates of mental states. However, individual neuronal oscillations are difficult to observe, since doing so usually requires inserting electrodes into the brain. The oscillations of neurons and groups of neurons produce macroscopic activity signatures through the summation of synchronized electromagnetic signals from individual neurons (often referred to as brain rhythms or brain waves), which can be observed using noninvasive techniques such as electroencephalography (EEG) or magnetoencephalography (MEG). EEG measures electrical activity with respect to a reference point and does not provide strong spatial localization of the signal. MEG measures the magnetic fields generated by electrical activity and provides better spatial location. However, MEG is biased toward measuring activity that originates in the sulci and activity close to the surface, since magnetic field activity degrades more quickly than electrical activity does. These brain waves can be considered neural correlates of brain states.

Brainwaves have been widely studied in neural activity generated by large groups of neurons, mostly by EEG. In general, EEG signals reveal oscillatory activity (groups of neurons periodically firing in synchrony), in specific frequency bands: alpha (7.5-12.5 Hz) that can be detected from the occipital lobe during relaxed wakefulness and which increases when the eyes are closed; delta (1-4 Hz), theta (4-8 Hz), beta (13-30 Hz), low gamma (30-70 Hz), and high gamma (70-150 Hz) frequency bands, where faster rhythms such as gamma activity have been linked to cognitive processing. Higher frequencies imply multiple groups of neurons firing in coordination, either in parallel or in series, or both, since individual neurons do not fire at rates of 100 Hz. Neural oscillations of specific characteristics have been linked to cognitive states, such as awareness and consciousness and different sleep stages. See, Chang-Hwan Im, Computational EEG Analysis: Methods and Applications (Biological and Medical Physics, Biomedical Engineering), Sep. 11, 2019.

Alpha is the frequency range from 7 Hz to 14 Hz. This was the “posterior basic rhythm” (also called the “posterior dominant rhythm” or the “posterior alpha rhythm”), seen in the posterior regions of the head on both sides, higher in amplitude on the dominant side. It emerges with the closing of the eyes and with relaxation and attenuates with eye opening or mental exertion. The posterior basic rhythm is slower than 8 Hz in young children (therefore technically in the theta range). In addition to the posterior basic rhythm, there are other normal alpha rhythms such as the sensorimotor, or mu rhythm (alpha activity in the contralateral sensory and motor cortical areas) that emerges when the hands and arms are idle; and the “third rhythm” (alpha activity in the temporal or frontal lobes). Alpha can be abnormal; for example, an EEG that has diffuse alpha occurring in a coma and is not responsive to external stimuli is referred to as “alpha coma.”

Beta is the frequency range from 15 Hz to about 30 Hz. It is usually seen on both sides in symmetrical distribution and is most evident frontally. Beta activity is closely linked to motor behavior and is generally attenuated during active movements. Low-amplitude beta with multiple and varying frequencies is often associated with active, busy, or anxious thinking and active concentration. Rhythmic beta with a dominant set of frequencies is associated with various pathologies, such as Dup15q syndrome, and drug effects, especially benzodiazepines. It may be absent or reduced in areas of cortical damage. It is the dominant rhythm in patients who are alert or anxious or who have their eyes open.

Gamma is the frequency range of approximately 30-100 Hz. Gamma rhythms are thought to represent binding of different populations of neurons together into a network to carry out a certain cognitive or motor function.

Delta waves are in the frequency range up to 4 Hz. It tends to be the highest in amplitude and the slowest waves. It is normally seen in adults in NREM (en.wikipedia.org/wiki/NREM). It is also seen normally in babies. It may occur focally with subcortical lesions and in general distribution with diffuse lesions, metabolic encephalopathy hydrocephalus or deep midline lesions. It is, usually, most prominent frontally in adults (e.g., FIRDA-frontal intermittent rhythmic delta) and posteriorly in children (e.g., DIRDA-occipital intermittent rhythmic delta).

Theta is the frequency range from 4 Hz to 7 Hz. Theta is normally seen in young children. It may be seen in drowsiness or arousal in older children and adults; it can also be seen in meditation. Excess theta for age represents abnormal activity. It can be seen as a focal disturbance in focal subcortical lesions; it can be seen in the generalized distribution in diffuse disorder or metabolic encephalopathy or deep midline disorders or some instances of hydrocephalus. On the contrary, this range has been associated with reports of relaxed, meditative, and creative states.

Mu range is 8-13 Hz and partly overlaps with other frequencies. It reflects the synchronous firing of motor neurons in a rest state. Mu suppression is thought to reflect motor mirror neuron systems because when an action is observed, the pattern extinguishes, possibly because of the normal neuronal system and the mirror neuron system “go out of sync” and interfere with each other. (en.wikipedia.org/wiki/Electroencephalography).

Brainwaves may be acquired in various ways. Traditional signal acquisition by neurologists and encephalography/EEG technicians involves pasted-on electrodes or caps with arrays of electrodes, e.g., 20-256 electrodes positioned on the scalp. However, in some cases, especially where high spatial resolution is not required, and dominant brainwave patterns are sought, simpler and less controlled EEG acquisition systems may be employed, including through commercially available device intended to interface with smartphones. See, kokoonio, www.thinkmindset.com/; www.choosemuse.com (Muse, Muse2); Neurosky; getvi.com (Vi Sense); Strickland, Eliza, “In-Ear EEG Makes Unobtrusive Brain-Hacking Gadgets a Real Possibility”, IEEE Spectrum Jul. 7, 2018; Strickland, Eliza, “Wireless Earbuds Will Record Your EEG, Send Brainwave Data To Your Phone”, IEEE Spectrum May 17, 2018. The Unicorn “Hybrid Black” wearable EEG headset provides a headset with eight electrode channels and digital data acquisition electronics (24 bit, 250 Hz), intended to provide a brain-computer interface for artistic, control and other tasks. See, www.unicorn-bi.com/. Starkey Laboratories, Inc.

US 20190188434 discloses an ear-worn electronic device having a plurality of sensors for EEG signals from a wearer's ear, as a brain-computer interface. A number of designs provide in-ear headphones which integrate EEG electrodes that pick up signals from the ear canal.

Another recently released application pertains to virtual reality (VR) technology. On Sep. 18, 2017 Looxid Labs launched a technology that harnesses EEG from a subject waring a VR headset. Looxid Labs intention is to factor in brainwaves into VR applications in order to accurately infer emotions. Other products such as MindMaze and even Samsung have tried creating similar applications through facial muscles recognition. (scottamyx.com/2017/10/13/looxid-labs-vr-brain-waves-human-emotions/). According to its website (looxidlabs.com/device-2/), the Looxid Labs Development Kit provides a VR headset embedded with miniaturized eye and brain sensors. It uses 6 EEG channels: Fp1, Fp2, AF7, AF8, AF3, AF4 in the international 10-20 system.

A number of virtual reality (VR) headsets are available, which provide headphones, display or retinal projection technology, head tracking, and other sensors or outputs. See, www.apple.com/apple-vision-pro, Meta (Oculus) Quest 2, Meta Quest Pro, Sony PlayStation VR, Valve Index VR Kit, HTE Vive Pro 2, HP Reverb G2, etc. en.wikipedia.org/wiki/Virtual_reality_headset

Principal component analysis (PCA) is a statistical procedure that uses an orthogonal transformation to convert a set of observations of possibly correlated variables into a set of values of linearly uncorrelated variables called principal components. If there are n observations with p variables, then the number of distinct principal components is min(n−1,p). This transformation is defined in such a way that the first principal component has the largest possible variance (that is, accounts for as much of the variability in the data as possible), and each succeeding component in turn has the highest variance possible under the constraint that it is orthogonal to the preceding components. The resulting vectors are an uncorrelated orthogonal basis set. PCA is sensitive to the relative scaling of the original variables. PCA is the simplest of the true eigenvector-based multivariate analyses. Often, its operation can be thought of as revealing the internal structure of the data in a way that best explains the variance in the data. If a multivariate dataset is visualized as a set of coordinates in high-dimensional data space (1 axis per variable), PCA can supply the user with a lower-dimensional picture, a projection of this object when viewed from its most informative viewpoint. This is done by using only the first few principal components so that the dimensionality of the transformed data is reduced. PCA is closely related to factor analysis. Factor analysis typically incorporates more domain specific assumptions about the underlying structure and solves eigenvectors of a slightly different matrix. PCA is also related to canonical correlation analysis (CCA). CCA defines coordinate systems that optimally describe the cross-covariance between two datasets while PCA defines a new orthogonal coordinate system that optimally describes variance in a single dataset. See, en.wikipedia.org/wiki/Principal_component_analysis.

The present technology, in an EEG feedback embodiment, may employ an event-correlated EEG time and/or frequency analysis performed on neuronal activity patterns. In a time-analysis, the signal is analyzed temporally and spatially, generally looking for changes with respect to time and space. In a frequency analysis, over an epoch of analysis, the data, which is typically a time-sequence of samples, is transformed, using e.g., a Fourier transform (FT, or one implementation, the Fast Fourier Transform, FFT), into a frequency domain representation, and the frequencies present during the epoch are analyzed. The window of analysis may be rolling, and so the frequency analysis may be continuous. In a hybrid time-frequency analysis, for example, a wavelet analysis, the data during the epoch is transformed using a “wavelet transform”, e.g., the Discrete Wavelet Transform (DWT) or continuous wavelet transform (CWT), which has the ability to construct a time-frequency representation of a signal that offers very good time and frequency localization. Changes in transformed data over time and space may be analyzed. In general, the spatial aspect of the brainwave analysis is anatomically modeled. In most cases, anatomy is considered universal, but in some cases, there are significant differences. For example, brain injury, psychiatric disease, age, race, native language, training, sex, handedness, and other factors may lead to distinct spatial arrangement of brain function, and therefore when transferring mood from one individual to another, it is preferred to normalize the brain anatomy of both individuals by experiencing roughly the same experiences, and measuring spatial parameters of the EEG or MEG. Note that spatial organization of the brain is highly persistent, absent injury or disease, and therefore, this need only be performed infrequently. However, since electrode placement may be inexact, a spatial calibration may be performed after electrode placement.

Different aspects of EEG magnitude and phase relationships may be captured, to reveal details of the neuronal activity. The “time-frequency analysis” reveals the brain's parallel processing of information, with oscillations at various frequencies within various regions of the brain reflecting multiple neural processes co-occurring and interacting. See, Lisman J, Buzsaki G. A neural coding scheme formed by the combined function of gamma and theta oscillations. Schizophr Bull. Jun. 16, 2008; doi:10:1093/schbul/sbn060. Such a time-frequency analysis may take the form of a wavelet transform analysis. This may be used to assist in integrative and dynamically adaptive information processing. Of course, the transform may be essentially lossless and may be performed in any convenient information domain representation. These EEG-based data analyses reveal the frequency-specific neuronal oscillations and their synchronization in brain functions ranging from sensory processing to higher-order cognition. Therefore, these patterns may be selectively analyzed, for transfer to or induction in, a subject.

A statistical clustering analysis may be performed in high dimension space to isolate or segment regions (e.g., spatial regions, or pseudo-spatial regions) which act or appear to act as signal sources, and to characterize the coupling between various regions. This analysis may also be used to establish signal types within each brain region and decision boundaries characterizing transitions between different signal types. These transitions may be state dependent, and therefore the transitions may be detected based on a temporal analysis, rather than merely a concurrent oscillator state. Thus, the present invention is not limited to whole brain entrainment, and may target brain regions. This may permit sequential recruitment of regions to the desired state, which may be easier that concurrent recruitment.

The various measures make use of the magnitude and/or phase angle information derived from the complex data extracted from the EEG during spectral decomposition and/or temporal/spatial/spectral analysis. Some measures estimate the magnitude or phase consistency of the EEG within one channel across trials, whereas others estimate the consistency of the magnitude or phase differences between channels across trials. Beyond these two families of calculations, there are also measures that examine the coupling between frequencies, within trials and recording sites. Of course, in the realm of time-frequency analysis, many types of relationships can be examined beyond those already mentioned.

It is believed that brainwaves represent, or are formed as a result of a resonance, where spatially dispersed ensembles of neurons interact over time in a coordinated fashion. The frequency of the wave is related to neural responsiveness to neurotransmitters, distances along neural pathways, diffusion limitations, etc. That is, the same sleep stage may be represented by slightly different frequencies in two different individuals, based on differences in the size of their brains, neuromodulators present, other anatomical, morphological and physiological differences, etc. These differences may be measured in microseconds or less, resulting in small changes in frequency. However, in a normal individual under non-pathologic circumstances, the low frequency brainwaves tend to encompass the entire brain, and the resonance is global. Note that a single nucleus may serve as a pacemaker for the brain, so that the resonance does not necessarily encompass the while brain. Note also that the resonance may be related to an ion leakage rate with corresponding membrane potential to generate periodic action potentials in a subset of neurons, similar to the operation of the sinoatrial node of the heart.

Under any theory of etiology, it is clear that the resonance is responsive to brain activity, representing at some level a modulation of the resonance, leading to periods of chaotic activity and changes in the rhythm. The modulation signal would precede the change in rhythm, and may be largely hidden from external brainwave monitoring.

Therefore, a model component of a controller can determine the parameters of neural transmission and ensemble characteristics, vis-à-vis stimulation, and synthesize a stimulus signal to match the correct frequency and phase of the subject's brainwave, with the optimization of the waveform adaptively determined. This may not be as simple as speeding up or slowing down playback of a signal, as different elements of the various brainwaves representing neural correlates of a sleep stage may have different relative differences between subjects. A non-feedback or minimal feedback (e.g., respiratory monitoring through use of a microphone to analyze breath sounds) embodiment does not benefit from the luxury of reading brainwave patterns directly, and therefore must use open loop control paradigms and inferential feedback to achieve the results.

The binaural sounds may be harmonic, for example major chords, using tremolo/isochronic tones, binaural beats, etc. to achieve brain entrainment a desired EEG pattern corresponding to a sleep stage. In order to initially induce sleep, the system may generate a lullaby, or other somnolescent music, with semantic or abstract content. Since the target is awake, higher level brain functions may be accessed, and sounds without semantic or abstract content may actually prevent sleep progression.

Artificial intelligence (AI) and machine learning methods, such as artificial neural networks, deep neural networks, etc., may be implemented to extract the signals of interest. Neural networks act as an optimized statistical classifier and may have arbitrary complexity. A so-called deep neural network having multiple hidden layers may be employed. The processing is typically dependent on labeled training data, such as EEG data, or various processed, transformed, or classified representations of the EEG data. The label represents the sleep stage of the subject during acquisition. In order to handle the continuous stream of data represented by the EEG, a recurrent neural network architecture may be implemented. Depending on the preprocessing before the neural network, formal implementations of recurrence may be avoided. A four or more dimensional data matrix may be derived from the traditional spatial-temporal processing of the EEG and fed to a neural network. Since the time parameter is represented in the input data, a neural network temporal memory is not required, though this architecture may require a larger number of inputs. Principal component analysis (PCA, en.wikipedia.org/wiki/Principal_component_analysis), spatial PCA (arxiv.org/pdf/1501.03221v3.pdf, adegenet.r-forge.r-project.org/files/tutorial-spca.pdf, www.ncbi.nlm.nih.gov/pubmed/1510870); and clustering analysis may also be employed (en.wikipedia.org/wiki/Eluster_analysis, see U.S. Pat. Nos. 9,338,302, 9,807,023).

The technology may be embodied in apparatuses for acquiring the brain activity information from the source, processing the brain activity information to reveal a target brain activity state and a set of stimuli, which seek to achieve that state in a recipient, and generating stimuli for the recipient to achieve and maintain the target brain activity state over a period of time and potential state transitions. A general-purpose computer may be used for the processing of the information, a microprocessor, an FPGA, an ASIC, a system-on-a-chip, or a specialized system, which employs a customized configuration to efficiently achieve the information transformations required. Typically, the source and recipient act asynchronously, with the brain activity of the source recorded and later processed. However, real-time processing and brain activity transfer are also possible. In the case of a general purpose programmable processor implementation or portions of the technology, computer instructions may be stored on a nontransitory computer readable medium. Typically, the system will have special-purpose components, such as a sensory stimulator, or a modified audio and/or display system, and therefore the system will not be a general purpose system. Further, even in a general purpose system, the operation per se is enhanced according to the present technology.

The source brain wave pattern may be acquired through multichannel EEG or MEG, from a human in the desired brain state. A computational model of the brain state is difficult to create. However, such a model is not required according to the present technology. Rather, the signals may be processed by a statistical process (e.g., PCA or a related technology), or a statistically trained process (e.g., a neural network). The processed signals preferably retain information regarding signal source special location, frequency, and phase. In stimulating the recipient's brain, the source may be modified to account for brain size differences, electrode locations, etc. Therefore, the preserved characteristics are normalized spatial characteristics, frequency, phase, and modulation patterns.

While methods exist for using voxel blood flow dependent technologies, such as fMRI, for determining brain activity, these do not provide sufficient temporal resolution to track neuron-based communications within the brain.

The normalization may be based on feedback from the target subject, for example, based on a comparison of a present state of the target subject and a corresponding state of the source subject, or another comparison of known states between the target and source. Typically, the excitation electrodes in the target subject do not correspond to the feedback electrodes or the electrodes on the source subject. Therefore, an additional type of normalization is required, which may also be based on a statistical or statistically trained algorithm.

In some cases, the target has an abnormal or unexpected response to stimulation based on a model maintained within the system. In this case, when the deviance from the expected response is identified, the system may seek to implement a new model, such as from a model repository that may be online, e.g., through the Internet. If the models are predictable, a translation may be provided between an applicable model of a source or trainer, and the applicable model of the target, to account for differences. In some cases, the desired mental state is relatively universal, such as sleep and awake. In this case, the brain response model may be a statistical model, rather than a neural network or deep neural network type implementation.

A hybrid approach may be provided, with the use of donor-derived brainwaves, on the one hand, which may be extracted from the brain activity readings (e.g., EEG or MEG) of a first subject (donor), preferably processed by principal component analysis, or spatial principal component analysis, autocorrelation, or other statistical processing technique (clustering, PCA, etc.) or statistically trained technique (backpropagation of errors, etc.) that separates components of brain activity, which can then be modified or modulated based on high-level parameters, e.g., abstractions. See, m14a.github.io/m14a/how_neural_networks_are_ trained/. Thus, the stimulator may be programmed to induce a series of brain states defined by name (e.g., sleep stage 1, sleep stage 2, etc.) or as a sequence of “abstract” semantic labels, icons, or other representations, each corresponding to a technical brain state or sequence of sub-states. The sequence may be automatically defined, based on biology and the system training, and thus relieve the programmer of low-level tasks. However, in a general case, the present technology maintains the use of components or subcomponents of the donor's brain activity readings, e.g., EEG or MEG, and does not seek to characterize or abstract them to a semantic level.

A neural network system or statistical classifier may be employed to characterize the brain wave activity and/or other data from a subject, e.g., respiratory or muscular response to stimulation. In addition to the classification or abstraction, a reliability parameter may be presented, which predicts the accuracy of the output. Where the accuracy is high, a model-based stimulator may be provided to select and/or parameterize the model and generate a stimulus for a target subject. Where the accuracy is low, a filtered representation of the signal may be used to control the stimulator, bypassing the model(s). The advantage of this hybrid scheme is that when the model-based stimulator is employed, many different parameters may be explicitly controlled independently of the source subject. On the other hand, where the data processing fails to yield a highly useful prediction of the correct model-based stimulator parameters, the model itself may be avoided, in favor of a direct stimulation type system.

In some cases, the base frequency, modulation, coupling, noise, phase jitter, or another characteristic of the signal may be substituted. For example, if the first subject is listening to music, there will be significant components of the neural correlates that are synchronized with the particular music. On the other hand, the music per se may not be part of the desired stimulation of the target subject. Therefore, through signal analysis and decomposition, the components of the signal from the first subject, which have a high temporal correlation with the music, may be extracted or suppressed from the resulting signal. Further, the target subject may be in a different acoustic environment, and it may be appropriate to modify the residual signal dependent on the acoustic environment of the target subject, so that the stimulation is appropriate for achieving the desired effect, and does not represent phantoms, distractions, or irrelevant or inappropriate content. In order to perform processing, it is convenient to store the signals or a partially processed representation, though a complete real-time signal processing chain may be implemented. Such a real-time signal processing chain is generally characterized in that the average size of a buffer remains constant, i.e., the lag between output and input is relatively constant, bearing in mind that there may be periodicity to the processing.

A self-learning or genetic algorithm may be used to tune the system, including both or either the signal processing at the donor system and the recipient system. In a genetic algorithm feedback-dependent self-learning system, the responsivity of a subject, e.g., the target, to various kinds of stimuli may be determined over a stimulus space. This stimulation may be in the context of use, with a specific target state provided, or unconstrained. The stimulator may operate using a library of stimulus patterns, or seek to generate synthetic patterns or modifications of patterns. Dyer some time, the system will learn to map the desired mental state to optimal context-dependent parameters of the stimulus pattern.

The technology may be used for both creation of a desired mental states in the recipient, elimination of existing (undesired) mental states in the recipient. In the latter case, a decision of what end state is to be achieved is less constrained, and therefore, the optimization is distinct. For example, in the former case, it may be hard to achieve a particular mental state that is desired, requiring a set of transitions to cause the brain of the recipient to be enabled/prepared to enter the target state. In the case of a system seeking to eliminate an undesired mental state, the issue is principally what path to take to most efficiently leave the current state, bearing in mind the various costs, such as the comfort/discomfort of the stimulation, the time value cost, etc. Therefore, the series of states may differ in the implementation of these distinct goals, even if the endpoints are identical, i.e., the optimal algorithm to achieve state B from state A, may be different from the optimal algorithm to exist state A, and end up at state B.

The waveform may be derived from an EEG recordings of brainwaves of at least one donor, processed using at least one of a principal component analysis (PCA), a correspondence analysis (EA), a factor analysis, a K-means clustering, a non-negative matrix factorization (NMF), a sparse PCA, a non-linear PCA, a robust PCA, an independent component analysis (ICA), a network component analysis, and a singular spectral analysis.

The user device may include a speaker and the stimulus may be a sound signal delivered through the speaker, e.g., an isochronic tone. The sound signal may be delivered to the subject through a pair of wireless earbuds, e.g., the modulated selected waveform may comprise binaural beats. Spatialized audio may be used, based on a head-related transfer function, to provide high source separation between ears without contacting headphones.

The user device may be configured to control an ambient light, which is selectively controllable to change at least one of brightness and color, and wherein the stimulus comprises a light signal which is presented to the subject through the ambient light. The light signal may be generated by at least one light emitting diode (LED). The LED may be disposed in proximity to the subject's eyes, e.g., in a mask or sleep mask. The user device may comprise at least one biometric sensor, further comprising the step of monitoring and collecting biometric data of the subject from said at least one biometric sensor.

A spatially distributed model of cortical computation is provided by Gepshtein et al. (2022). An excitatory-inhibitory circuit is used as a motif repeated across neural tissue and connected to form a chain. Responses of this model are always distributed among neurons, forming characteristic waveforms that termed neural waves. Waves elicited by different parts of the stimulus spread across the chain and interfere with one another. Patterns of interference created by this process have a number of characteristics that determine the circuit's preference for stimulation.

Theoretical studies have explored phenomena such as propagation of activity in spatially structured networks and formation of patterned activity in neural fields, by means of traveling waves. These investigations suggested that traveling waves can contribute to formation of stimulus selectivity in cortical mechanisms, such as directional selectivity, and that interaction between patterns of activity propagating across cortex can perform computations. Neural waves create a distributed spatiotemporal pattern. Study of interference of these waves allows prediction of perceptual and physiological properties of biological vision.

Brainwaves: At the root of all our thoughts, emotions and behaviors is the communication between neurons within our brains, a rhythmic or repetitive neural activity in the central nervous system. The oscillation can be produced by a single neuron or by synchronized electrical pulses from ensembles of neurons communicating with each other. The interaction between neurons can give rise to oscillations at a different frequency than the firing frequency of individual neurons. The synchronized activity of large numbers of neurons produces macroscopic oscillations, which can be observed in an electroencephalogram. They are divided into bandwidths to describe their purported functions or functional relationships. Oscillatory activity in the brain is widely observed at different levels of organization and is thought to play a key role in processing neural information. Numerous experimental studies support a functional role of neural oscillations. A unified interpretation, however, is still not determined. Neural oscillations and synchronization have been linked to many cognitive functions such as information transfer, perception, motor control and memory. Electroencephalographic (EEG) signals are relatively easy and safe to acquire, have a long history of analysis, and can have high dimensionality, e.g., up to 128 or 256 separate recording electrodes. While the information represented in each electrode is not independent of the others, and the noise in the signals high, there is much information available through such signals that has not been fully characterized to date.

Brain waves have been widely studied in neural activity generated by large groups of neurons, mostly by EEG. In general, EEG signals reveal oscillatory activity (groups of neurons periodically firing in synchrony), in specific frequency bands: alpha (7.5-12.5 Hz) that can be detected from the occipital lobe during relaxed wakefulness and which increases when the eyes are closed; delta (1-4 Hz), theta (4-8 Hz), beta (13-30 Hz), low gamma (30-70 Hz), and high gamma (70-150 Hz) frequency bands, where faster rhythms such as gamma activity have been linked to cognitive processing. Higher frequencies imply multiple groups of neurons firing in coordination, either in parallel or in series, or both, since individual neurons do not fire at rates of 100 Hz. Neural oscillations of specific characteristics have been linked to cognitive states, such as awareness and consciousness and different sleep stages.

Nyquist Theorem states that the highest frequency that can be accurately represented is one-half of the sampling rate. Practically, the sampling rate should be ten times higher than the highest frequency of the signal. (See, www.slideshare.net/ertvk/eeg-examples). While EEG signals are largely band limited, the superimposed noise may not be. Further, the EEG signals themselves represent components from a large number of neurons, which fire independently. Therefore, large bandwidth signal acquisition may have utility.

It is a useful analogy to think of brainwaves as musical notes. Like in symphony, the higher and lower frequencies link and cohere with each other through harmonics, especially when one considers that neurons may be coordinated not only based on transitions, but also on phase delay. Oscillatory activity is observed throughout the central nervous system at all levels of organization. The dominant neuro oscillation frequency is associated with a respective mental state.

The functions of brain waves are wide-ranging and vary for different types of oscillatory activity. Neural oscillations also play an important role in many neurological disorders.

In standard EEG recording practice, 19 recording electrodes are placed uniformly on the scalp (the International 10-20 System). In addition, one or two reference electrodes (often placed on earlobes) and a ground electrode (often placed on the nose to provide amplifiers with reference voltages) are required. However, additional electrodes may add minimal useful information unless supplemented by computer algorithms to reduce raw EEG data to a manageable form. When large numbers of electrodes are employed, the potential at each location may be measured with respect to the average of all potentials (the common average reference), which often provides a good estimate of potential at infinity. The common average reference is not appropriate when electrode coverage is sparse (perhaps less than 64 electrodes). See, Paul L. Nunez and Ramesh Srinivasan (2007) Electroencephalogram. Scholarpedia, 2(2):1348, scholarpedia.org/article/Electroencephalogram. Dipole localization algorithms may be useful to determine spatial emission patterns in EEG.

Scalp potential may be expressed as a volume integral of dipole moment per unit volume over the entire brain provided P(r,t) is defined generally rather than in columnar terms. For the important case of dominant cortical sources, scalp potential may be approximated by the following integral over the cortical volume Θ, VS(r,t)=∫∫∫ΘG(r,r′)·P(r′,t)dΘ(r′). If the volume element dΘ(r′) is defined in terms of cortical columns, the volume integral may be reduced to an integral over the folded cortical surface. The time-dependence of scalp potential is the weighted sum of all dipole time variations in the brain, although deep dipole volumes typically make negligible contributions. The vector Green's function G(r,r′) contains all geometric and conductive information about the head volume conductor and weights the integral accordingly. Thus, each scalar component of the Green's function is essentially an inverse electrical distance between each source component and scalp location. For the idealized case of sources in an infinite medium of constant conductivity, the electrical distance equals the geometric distance. The Green's function accounts for the tissue's finite spatial extent and its inhomogeneity and anisotropy. The forward problem in EEG consists of choosing a head model to provide G(r,r′) and carrying out the integral for some assumed source distribution. The inverse problem consists of using the recorded scalp potential distribution VS(r,t) plus some constraints (usual assumptions) on P(r,t) to find the best fit source distribution P(r,t). Since the inverse problem has no unique solution, any inverse solution depends critically on the chosen constraints, for example, only one or two isolated sources, distributed sources confined to the cortex, or spatial and temporal smoothness criteria. High-resolution EEG uses the experimental scalp potential VS(r,t) to predict the potential on the dura surface (the unfolded membrane surrounding the cerebral cortex) VD(r,t). This may be accomplished using a head model Green's function G(r,r′) or by estimating the surface Laplacian with either spherical or 3D splines. These two approaches typically provide very similar dura potentials VD(r,t); the estimates of dura potential distribution are unique subject to head model, electrode density, and noise issues.

In an EEG recording system, each electrode is connected to one input of a differential amplifier (one amplifier per pair of electrodes); a common system reference electrode (or synthesized reference) is connected to the other input of each differential amplifier. These amplifiers amplify the voltage between the active electrode and the reference (typically 1,000-100,000 times, or 60-100 dB of voltage gain). The amplified signal is digitized via an analog-to-digital converter, after being passed through an anti-aliasing filter. Analog-to-digital sampling typically occurs at 256-512 Hz in clinical scalp EEG; sampling rates of up to 20 kHz are used in some research applications. The EEG signals can be captured with open-source hardware such as OpenBCI, and the signal can be processed by freely available EEG software such as EEGLAB or the Neurophysiological Biomarker Toolbox. A typical adult human EEG signal is about 10 μV to 100 μV in amplitude when measured from the scalp and is about 10-20 mV when measured from subdural electrodes.

Delta wave (en.wikipedia.org/wiki/Delta_wave) is the frequency range up to 4 Hz. It tends to be the highest in amplitude and the slowest waves. It is normally seen in adults in NREM (en.wikipedia.org/wiki/NREM). It is also seen normally in babies. It may occur focally with subcortical lesions and in general distribution with diffuse lesions, metabolic encephalopathy hydrocephalus or deep midline lesions. It is usually most prominent frontally in adults (e.g., FIRDA-frontal intermittent rhythmic delta) and posteriorly in children (e.g., DIRDA-occipital intermittent rhythmic delta).

Theta is the frequency range from 4 Hz to 7 Hz. Theta is normally seen in young children. It may be seen in drowsiness or arousal in older children and adults; it can also be seen in meditation. Excess theta for age represents abnormal activity. It can be seen as a focal disturbance in focal subcortical lesions; it can be seen in generalized distribution in diffuse disorder or metabolic encephalopathy or deep midline disorders or some instances of hydrocephalus. On the contrary, this range has been associated with reports of relaxed, meditative, and creative states.

Alpha is the frequency range from 7 Hz to 14 Hz. This was the “posterior basic rhythm” (also called the “posterior dominant rhythm” or the “posterior alpha rhythm”), seen in the posterior regions of the head on both sides, higher in amplitude on the dominant side. It emerges with the closing of the eyes and with relaxation and attenuates with eye opening or mental exertion. The posterior basic rhythm is actually slower than 8 Hz in young children (therefore technically in the theta range). In addition to the posterior basic rhythm, there are other normal alpha rhythms such as the sensorimotor, or mu rhythm (alpha activity in the contralateral sensory and motor cortical areas) that emerges when the hands and arms are idle; and the “third rhythm” (alpha activity in the temporal or frontal lobes). Alpha can be abnormal; for example, an EEG that has diffuse alpha occurring in coma and is not responsive to external stimuli is referred to as “alpha coma.”

Beta is the frequency range from 15 Hz to about 30 Hz. It is usually seen on both sides in symmetrical distribution and is most evident frontally. Beta activity is closely linked to motor behavior and is generally attenuated during active movements. Low-amplitude beta with multiple and varying frequencies is often associated with active, busy or anxious thinking and active concentration. Rhythmic beta with a dominant set of frequencies is associated with various pathologies, such as Dup15q syndrome, and drug effects, especially benzodiazepines. It may be absent or reduced in areas of cortical damage. It is the dominant rhythm in patients who are alert or anxious or who have their eyes open.

Gamma is the frequency range approximately 30-100 Hz. Gamma rhythms are thought to represent binding of different populations of neurons together into a network to carry out a certain cognitive or motor function.

Mu range is 8-13 Hz and partly overlaps with other frequencies. It reflects the synchronous firing of motor neurons in a rest state. Mu suppression is thought to reflect motor mirror neuron systems, because when an action is observed, the pattern extinguishes, possibly because of the normal neuronal system and the mirror neuron system “go out of sync” and interfere with each other. (en.wikipedia.org/wiki/Electroencephalography). See:
Abeles M, Local Cortical Circuits (1982) New York: Springer-Verlag.
Braitenberg V and Schuz A (1991) Anatomy of the Cortex. Statistics and Geometry. New York: Springer-Verlag.
Ebersole J S (1997) Defining epileptogenic foci: past, present, future. J. Clin. Neurophysiology 14: 470-483.
Edelman G M and Tononi G (2000) A Universe of Consciousness, New York: Basic Books.
Freeman W J (1975) Mass Action in the Nervous System, New York: Academic Press.
Gevins A S and Cutillo B A (1995) Neuroelectric measures of mind. In: P L Nunez (Au), Neocortical Dynamics and Human EEG Rhythms. N Y: Oxford U. Press, pp. 304-338.
Gevins A S, Le J, Martin N, Brickett P, Desmond J, and Reutter B (1994) High resolution EEG: 124-channel recording, spatial enhancement, and MRI integration methods. Electroencephalography and Clin. Neurophysiology 90: 337-358.
Gevins A S, Smith M E, McEvoy L and Yu D (1997) High-resolution mapping of cortical activation related to working memory: effects of task difficulty, type of processing, and practice. Cerebral Cortex 7: 374-385.
Haken H (1983) Synergetics: An Introduction, 3rd Edition, Springer-Verlag.
Haken H (1999) What can synergetics contribute to the understanding of brain functioning? In: Analysis of Neurophysiological Brain Functioning, C Uhl (Ed), Berlin: Springer-Verlag, pp 7-40.
Ingber L (1995) Statistical mechanics of multiple scales of neocortical interactions. In: P L Nunez (Au), Neocortical Dynamics and Human EEG Rhythms. N Y: Oxford U. Press, 828-881.
Izhikevich E M (1999) Weakly connected quasi-periodic oscillators, FM interactions, and multiplexing in the brain, SIAM J. Applied Mathematics 59: 2193-2223.
Jirsa V K and Haken H (1997) A derivation of a macroscopic field theory of the brain from the quasi-microscopic neural dynamics. Physica D 99: 503-528.
Jirsa V K and Kelso J A S (2000) Spatiotemporal pattern formation in continuous systems with heterogeneous connection topologies. Physical Review E 82: 8482-8485.
Katznelson R D (1981) Normal modes of the brain: Neuroanatomical basis and a physiological theoretical model. In P L Nunez (Au), Electric Fields of the Brain: The Neurophysics of EEG, 1st Edition, NY: Oxford U. Press, pp 401-442.
Klimesch W (1998) Memory processes, brain oscillations and EEG synchronization. International J. Psychophysiology 24: 61-100.
Law S K, Nunez P L and Wijesinghe R S (1993) High resolution EEG using spline generated surface Laplacians on spherical and ellipsoidal surfaces. IEEE Transactions on Biomedical Engineering 40:145-153.
Liley D T J, Cadusch P J and Dafilis M P (2002) A spatially continuous mean field theory of electrocortical activity network. Computation in Neural Systems 13: 87-113.
Malmuvino J and Plonsey R (1995) Bioelectromagetism. NY: Oxford U. Press.
Niedermeyer E and Lopes da Silva F H (Eds) (2005) Electroencephalography. Basic Principals, Clin. Applications, and Related Fields. Fifth Edition. London: Williams and Wilkins.
Nunez P L (1989) Generation of human EEG by a combination of long and short range neocortical interactions. Brain Topography 1:199-215.
Nunez P L (1995) Neocortical Dynamics and Human EEG Rhythms. NY: Oxford U. Press.
Nunez P L (2000) Toward a large-scale quantitative description of neocortical dynamic function and EEG (Target article), Behavioral and Brain Sciences 23: 371-398.
Nunez P L (2000) Neocortical dynamic theory should be as simple as possible, but not simpler (Response to 18 commentaries on target article), Behavioral and Brain Sciences 23: 415-437.
Nunez P L (2002) EEG. In V S Ramachandran (Ed) Encyclopedia of the Human Brain, La Jolla: Academic Press, 189-179.
Nunez P L and Silberstein R B (2001) On the relationship of synaptic activity to macroscopic measurements: Does co-registration of EEG with fMRI make sense? Brain Topog. 13: 79-98.
Nunez P L and Srinivasan R (2006) Electric Fields of the Brain: The Neurophysics of EEG, 2nd Edition, NY: Oxford U. Press.
Nunez P L and Srinivasan R (2006) A theoretical basis for standing and traveling brain waves measured with human EEG with implications for an integrated consciousness. Clin. Neurophysiology 117: 2424-2435.
Nunez P L, Srinivasan R, Westdorp A F, Wijesinghe R S, Tucker D M, Silberstein R B, and Cadusch P J (1997) EEG coherency I: Statistics, reference electrode, volume conduction, Laplacians, cortical imaging, and interpretation at multiple scales. Electroencephalography and Clin. Neurophysiology 103: 516-527.
Nunez P L. Wingeier B M and Silberstein R B (2001) Spatial-temporal structures of human alpha rhythms: theory, micro-current sources, multiscale measurements, and global binding of local networks, Human Brain Mapping 13:125-184.
Nuwer M (1997) Assessment of digital EEG, quantitative EEG, and EEG brain mapping: report of the American Academy of Neurology and the American Clin. Neurophysiology Society. Neurology 49: 277-292.
Penfield W and Jasper H D (1954) Epilepsy and the Functional Anatomy of the Human Brain. London: Little, Brown and Co.
Robinson P A, Rennie C I Rowe D L and O'Conner S C (2004) Estimation of multiscale neurophysiologic parameters by electroencephalographic means. Human Brain Mapping 23: 53-72.
Scott A C (1995) Stairway to the Mind. New York: Springer-Verlag.
Silberstein R B, Danieli F and Nunez P L (2003) Fronto-parietal evoked potential synchronization is increased during mental rotation, NeuroReport 14: 67-71.
Silberstein R B, Song J, Nunez P L and Park W (2004) Dynamic sculpting of brain functional connectivity is correlated with performance, Brain Topography 16: 240-254.
Srinivasan R and Petrovic S (2006) MEG phase follows conscious perception during binocular rivalry induced by visual stream segregation. Cerebral Cortex, 16: 597-608.
Srinivasan R, Nunez P L and Silberstein R B (1998) Spatial filtering and neocortical dynamics: estimates of EEG coherence. IEEE Trans. on Biomedical Engineering, 45: 814-825.
Srinivasan R, Russell D P, Edelman G M, and Tononi G (1999) Frequency tagging competing stimuli in binocular rivalry reveals increased synchronization of neuromagnetic responses during conscious perception. J. Neuroscience 19: 5435-5448.
Uhl C (Ed) (1999) Analysis of Neurophysiological Brain Functioning. Berlin: Springer-Verlag,
Wingeier B M, Nunez P L and Silberstein R B (2001) Spherical harmonic decomposition applied to spatial-temporal analysis of human high-density electroencephalogram. Physical Review E 64: 051916-1 to 9.

TABLE 1
Comparison of EEG bands

| Band | Freq. (Hz) | Location | Normally | Pathologically |
|---|---|---|---|---|
| Delta | <4 | frontally in adults, | adult slow-wave sleep | subcortical lesions |
| | | posteriorly in children; | in babies | diffuse lesions |
| | | high-amplitude waves | Has been found during some | metabolic encephalopathy hydrocephalus |
| | | | continuous-attention tasks | deep midline lesions |
| Theta | 4-7 | Found in locations not | higher in young children | focal subcortical lesions |
| | | related to task at hand | drowsiness in adults and teens | metabolic encephalopathy |
| | | | idling | deep midline disorders |
| | | | Associated with inhibition of elicited | some instances of hydrocephalus |
| | | | responses (has been found to spike | |
| | | | in situations where a person is | |
| | | | actively trying to repress a | |
| | | | response or action). | |
| Alpha | 8-15 | posterior regions of head, | relaxed/reflecting | Coma |
| | | both sides, higher in | closing the eyes | |
| | | amplitude on dominant | Also associated with inhibition | |
| | | side. Central sites (c3-c4) | control, seemingly with the purpose | |
| | | at rest | of timing inhibitory activity in | |
| | | | different locations across the | |
| | | | brain. | |
| Beta | 16-31 | both sides, symmetrical | range span: active calm → intense | Benzodiazepines |
| | | distribution, most evident | → stressed → mild obsessive | (en.wikipedia.org/wiki/Benzodiazepines) |
| | | frontally; low-amplitude | active thinking, focus, high alert, | Dup15q syndrome |
| | | waves | anxious | |
| Gamma | >32 | Somatosensory cortex | Displays during cross-modal | A decrease in gamma-band activity may be |
| | | | sensory processing (perception | associated with cognitive decline, especially |
| | | | that combines two different senses, | when related to the theta band; however, this |
| | | | such as sound and sight) | has not been proven for use as a clinical |
| | | | Also is shown during short-term | diagnostic measurement |
| | | | memory matching of recognized | |
| | | | objects, sounds, or tactile | |
| | | | sensations | |
| Mu | 8-12 | Sensorimotor cortex | Shows rest-state motor neurons. | Mu suppression could indicate that motor |
| | | | | mirror neurons are working. Deficits in Mu |
| | | | | suppression, and thus in mirror neurons, |
| | | | | might play a role in autism. |

EEG AND qEEG: An EEG electrode will mainly detect the neuronal activity in the brain region just beneath it. However, the electrodes receive the activity from thousands of neurons. One square millimeter of cortex surface, for example, has more than 100,000 neurons. It is only when the input to a region is synchronized with electrical activity occurring at the same time that simple periodic waveforms in the EEG become distinguishable. The temporal pattern associated with specific brainwaves can be digitized and encoded a non-transient memory, and embodied in or referenced by, computer software.

EEG (electroencephalography) and MEG (magnetoencephalography) are available technologies to monitor brain electrical activity. Each generally has sufficient temporal resolution to follow dynamic changes in brain electrical activity. Electroencephalography (EEG) and quantitative electroencephalography (gEEG) are electrophysiological monitoring methods that analyze the electrical activity of the brain to measure and display patterns that correspond to cognitive states and/or diagnostic information. It is typically noninvasive, with the electrodes placed on the scalp, although invasive electrodes are also used in some cases. EEG signals may be captured and analyzed by a mobile device, often referred as “brain wearables”. There are a variety of “brain wearables” readily available on the market today. EEGs can be obtained with a non-invasive method where the aggregate oscillations of brain electric potentials are recorded with numerous electrodes attached to the scalp of a person. Most EEG signals originate in the brain's outer layer (the cerebral cortex), believed largely responsible for our thoughts, emotions, and behavior. Cortical synaptic action generates electrical signals that change in the 10 to 100-millisecond range. Transcutaneous EEG signals are limited by the relatively insulating nature of the skull surrounding the brain, the conductivity of the cerebrospinal fluid and brain tissue, relatively low amplitude of individual cellular electrical activity, and distances between the cellular current flows and the electrodes. EEG is characterized by: (1) Voltage; (2) Frequency; (3) Spatial location; (4) Inter-hemispheric symmetries; (5) Reactivity (reaction to state change); (6) Character of waveform occurrence (random, serial, continuous); and (7) Morphology of transient events. EEGs can be separated into two main categories. Spontaneous EEG which occur in the absence of specific sensory stimuli and evoked potentials (EPs) which are associated with sensory stimuli like repeated light flashes, auditory tones, finger pressure or mild electric shocks. The latter is recorded for example by time averaging to remove effects of spontaneous EEG. Non-sensory triggered potentials are also known. EP's typically are time synchronized with the trigger, and thus have an organization principle. Event-related potentials (ERPs) provide evidence of a direct link between cognitive events and brain electrical activity in a wide range of cognitive paradigms. It has generally been held that an ERP is the result of a set of discrete stimulus-evoked brain events. Event-related potentials (ERPs) are recorded in the same way as EPs, but occur at longer latencies from the stimuli and are more associated with an endogenous brain state.

Typically, a magnetic sensor with sufficient sensitivity to individual cell depolarization or small groups is a superconducting quantum interference device (SQUID), which requires cryogenic temperature operation, either at liquid nitrogen temperatures (high temperature superconductors, HTS) or at liquid helium temperatures (low temperature superconductors, LTS). However, current research shows possible feasibility of room temperature superconductors (20 C). Magnetic sensing has an advantage, due to the dipole nature of sources, of having better potential volumetric localization; however, due to this added information, complexity of signal analysis is increased.

In general, the electromagnetic signals detected represent action potentials, an automatic response of a nerve cell to depolarization beyond a threshold, which briefly opens conduction channels. The cells have ion pumps which seek to maintain a depolarized state. Once triggered, the action potential propagates along the membrane in two-dimensions, causing a brief high level of depolarizing ion flow. There is a quiescent period after depolarization that generally prevents oscillation within a single cell. Since the exon extends from the body of the neuron, the action potential will typically proceed along the length of the axon, which terminates in a synapse with another cell. While direct electrical connections between cells occur, often the axon releases a neurotransmitter compound into the synapse, which causes a depolarization or hyperpolarization of the target cell. Indeed, the result may also be release of a hormone or peptide, which may have a local or more distant effect.

The electrical fields detectable externally tend to not include signals which low frequency signals, such as static levels of polarization, or cumulative depolarizating or hyperpolarizing effects between action potentials. In myelinated tracts, the current flows at the segments tend to be small, and therefore the signals from individual cells are small. Therefore, the largest signal components are from the synapses and cell bodies. In the cerebrum and cerebellum, these structures are mainly in the cortex, which is largely near the skull, making electroencephalography useful, since it provides spatial discrimination based on electrode location. However, deep signals are attenuated, and poorly localized. Magnetoencephalography detects dipoles, which derive from current flow, rather than voltage changes. In the case of a radially or spherically symmetric current flow within a short distance, the dipoles will tend to cancel, while net current flows long axons will reinforce. Therefore, an electroencephalogram reads a different signal than a magnetoencephalogram.

EEG-based studies of emotional specificity at the single-electrode level demonstrated that asymmetric activity at the frontal site, especially in the alpha (8-12 Hz) band, is associated with emotion. Voluntary facial expressions of smiles of enjoyment produce higher left frontal activation. Decreased left frontal activity is observed during the voluntary facial expressions of fear. In addition to alpha band activity, theta band power at the frontal midline (Fm) has also been found to relate to emotional states. Pleasant (as opposed to unpleasant) emotions are associated with an increase in frontal midline theta power. Many studies have sought to utilize pattern classification, such as neural networks, statistical classifiers, clustering algorithms, etc., to differentiate between various emotional states reflected in EEG.

EEG-based studies of emotional specificity at the single-electrode level demonstrated that asymmetric activity at the frontal site, especially in the alpha (8-12 Hz) band, is associated with emotion. Ekman and Davidson found that voluntary facial expressions of smiles of enjoyment produced higher left frontal activation (Ekman P, Davidson R J (1993) Voluntary Smiling Changes Regional Brain Activity. Psychol Sci 4: 342-345). Another study by Coen et al. found decreased left frontal activity during the voluntary facial expressions of fear (Coen J A, Allen J J, Harmon-Jones E (2001) Voluntary facial expression and hemispheric asymmetry over the frontal cortex. Psychophysiology 38: 912-925). In addition to alpha band activity, theta band power at the frontal midline (Fm) has also been found to relate to emotional states. Sammler and colleagues, for example, showed that pleasant (as opposed to unpleasant) emotion is associated with an increase in frontal midline theta power (Sammler D, Grigutsch M, Fritz T, Koelsch S (2007) Music and emotion: Electrophysiological correlates of the processing of pleasant and unpleasant music. Psychophysiology 44: 293-304). To further demonstrate whether these emotion-specific EEG characteristics are strong enough to differentiate between various emotional states, some studies have utilized a pattern classification analysis approach. See, for example:
Dan N, Xiao-Wei W, Li-Chen S, Bao-Liang L. EEG-based emotion recognition during watching movies; 2011 Apr. 27 2011-May 1, 2011: 887-870;
Lin Y P, Wang H, Jung T P, Wu T L, Jeng S K, et al. (2010) EEG-Based Emotion Recognition in Music Listening. Ieee T Bio Med Eng 57: 1798-1808;
Murugappan M, Nagarajan R, Yaacob S (2010) Classification of human emotion from EEG using discrete wavelet transform. J Biomed Sci Eng 3: 390-396;
Murugappan M, Nagarajan R, Yaacob S (2011) Combining Spatial Filtering and Wavelet Transform for Classifying Human Emotions Using EEG Signals. J Med. Bio. Eng. 31: 45-51.

Detecting different emotional states by EEG may be more appropriate using EEG-based functional connectivity. There are various ways to estimate EEG-based functional brain connectivity: correlation, coherence and phase synchronization indices between each pair of EEG electrodes had been used. The assumption is that a higher correlation map indicates a stronger relationship between two signals. (Brazier M A, Casby J U (1952) Cross-correlation and autocorrelation studies of electroencephalographic potentials. Electroen clin neuro 4: 201-211). Coherence gives information similar to correlation, but also includes the covariation between two signals as a function of frequency. (Cantero J L, Atienza M, Sales R M, Gomez C M (1999) Alpha EEG coherence in different brain states: an electrophysiological index of the arousal level in human subjects. Neurosci lett 271: 167-70.) The assumption is that higher correlation indicates a stronger relationship between two signals. (Guevara M A, Corsi-Cabrera M (1998) EEG coherence or EEG correlation? Int J Psychophysiology 23:145-153; Cantero J L, Atienza M, Sales R M, Gomez C M (1999) Alpha EEG coherence in different brain states: an electrophysiological index of the arousal level in human subjects. Neurosci lett 271:187-70; Adler G, Brassen 5, Jajcevic A (2003) EEG coherence in Alzheimer's dementia. J Neural Transm 110:1051-1058; Deeny S P, Hillman H, Janelle C M, Hatfield B D (2003) Cortico-cortical communication and superior performance in skilled marksmen: An EEG coherence analysis. J Sport Exercise Psy 25:188-204.) Phase synchronization among the neuronal groups estimated based on the phase difference between two signals is another way to estimate the EEG-based functional connectivity among brain areas. It is. (Franaszczuk P J, Bergey G K (1999) An autoregressive method for the measurement of synchronization of interictal and ictal EEG signals. Biol Cybern 81: 3-9.)

A number of groups have examined emotional specificity using EEG-based functional brain connectivity. For example, Shin and Park showed that, when emotional states become more negative at high room temperatures, correlation coefficients between the channels in temporal and occipital sites increase (Shin J-H, Park D-H. (2011) Analysis for Characteristics of Electroencephalogram (EEG) and Influence of Environmental Factors According to Emotional Changes. In Lee G, Howard D, Ślȩzak D, editors. Convergence and Hybrid Information Technology. Springer Berlin Heidelberg, 488-500.) Hinrichs and Machleidt demonstrated that coherence decreases in the alpha band during sadness, compared to happiness (Hinrichs H, Machleidt W (1992) Basic emotions reflected in EEG-coherences. Int J Psychophysiol 13: 225-232). Miskovic and Schmidt found that EEG coherence between the prefrontal cortex and the posterior cortex increased while viewing highly emotionally arousing (i.e., threatening) images, compared to viewing neutral images (Miskovic V, Schmidt L A (2010) Cross-regional cortical synchronization during affective image viewing. Brain Res 1382:102-111). Costa and colleagues applied the synchronization index to detect interaction in different brain sites under different emotional states (Costa T, Rognoni E, Galati D (2008) EEG phase synchronization during emotional response to positive and negative film stimuli. Neurosci Lett 406: 159-164). Costa's results showed an overall increase in the synchronization index among frontal channels during emotional stimulation, particularly during negative emotion (i.e., sadness). Furthermore, phase synchronization patterns were found to differ between positive and negative emotions. Costa also found that sadness was more synchronized than happiness at each frequency band and was associated with a wider synchronization both between the right and left frontal sites and within the left hemisphere. In contrast, happiness was associated with a wider synchronization between the frontal and occipital sites.

Different connectivity indices are sensitive to different characteristics of EEG signals. Correlation is sensitive to phase and polarity, but is independent of amplitudes. Changes in both amplitude and phase lead to a change in coherence (Guevara M A, Corsi-Cabrera M (1999) EEG coherence or EEG correlation? Int J Psychophysiol 23:145-153). The phase synchronization index is only sensitive to a change in phase (Lachaux J P, Rodriguez E, Martinerie J, Varela F J (1999) Measuring phase synchrony in brain signals. Hum Brain Mapp 8:194-208).

A number of studies have tried to classify emotional states by means of recording and statistically analyzing EEG signals from the central nervous systems. See for example:
Lin Y P, Wang H, Jung T P, Wu T L, Jeng S K, et al. (2010) EEG-Based Emotion Recognition in Music Listening. IEEE T Bio Med Eng 57:1798-1808
Murugappan M, Nagarajan R, Yeacob S (2010) Classification of human emotion from EEG using discrete wavelet transform. J Biomed Sci Eng 3: 390-398.
Murugappan M, Nagarajan R, Yeacob S (2011) Combining Spatial Filtering and Wavelet Transform for Classifying Human Emotions Using EEG Signals. J Med. Bio. Eng. 31: 45-51.
Berkman E, Wong D K, Guimaraes M P, Uy E T, Gross J J, et al. (2004) Brain wave recognition of emotions in EEG. Psychophysiology 41: S71-S71.
Chanel G, Kronegg J, Grandjean D, Pun T (2009) Emotion assessment: Arousal evaluation using EEG's and peripheral physiological signals. Multimedia Content Representation, Classification and Security 4105: 530-537.
Hagiwara KlaM (2003) A Feeling Estimation System Using a Simple Electroencephalograph. IEEE International Conference on Systems, Man and Cybernetics. 4204-4209.
You-Yun Lee and Shulan Hsieh studied different emotional states by means of EEG-based functional connectivity patterns. They used emotional film clips to elicit three different emotional states.

The dimensional theory of emotion, which asserts that there are neutral, positive, and negative emotional states, may be used to classify emotional states, because numerous studies have suggested that the responses of the central nervous system correlate with emotional valence and arousal. (See for example, Davidson R J (1993) Cerebral Asymmetry and Emotion—Conceptual and Methodological Conundrums. Cognition Emotion 7: 115-138; Jones N A, Fox N A (1992) Electroencephalogram asymmetry during emotionally evocative films and its relation to positive and negative affectivity. Brain Cogn 20: 280-299; Schmidt L A, Trainor L J (2001) Frontal brain electrical activity (EEG) distinguishes valence and intensity of musical emotions. Cognition Emotion 15: 487-500; Tomarken A J, Davidson R J, Henriques J B (1990) Resting frontal brain asymmetry predicts affective responses to films. J Pers Soc Psychol 59: 791-801.) As suggested by Mauss and Robins (2009), “measures of emotional responding appear to be structured along dimensions (e.g., valence, arousal) rather than discrete emotional states (e.g., sadness, fear, anger)”.

EEG-based functional connectivity change was found to be significantly different among emotional states of neutral, positive, or negative. Lee Y-Y, Hsieh S (2014) Classifying Different Emotional States by Means of EEG-Based Functional Connectivity Patterns. PLoS ONE 9(4): e95415. doi.org/10.1371/journal.pone.0095415. A connectivity pattern may be detected by pattern classification analysis using Quadratic Discriminant Analysis. The results indicated that the classification rate was better than chance. They concluded that estimating EEG-based functional connectivity provides a useful tool for studying the relationship between brain activity and emotional states.

Emotions affects learning. Intelligent Tutoring Systems (ITS) learner model initially composed of a cognitive module was extended to include a psychological module and an emotional module. Alicia Heraz et al. introduced an emomental agent. It interacts with an ITS to communicate the emotional state of the learner based upon his mental state. The mental state was obtained from the learner's brainwaves. The agent learns to predict the learner's emotions by using machine learning techniques. (Alicia Heraz, Ryad Razaki; Claude Frasson, “Using machine learning to predict learner emotional state from brainwaves” Advanced Learning Technologies, 2007. ICALT 2007. Seventh IEEE International Conference on Advanced Learning Technologies (ICALT 2007)) See also:
Ella T. Mampusti, Jose S. Ng, Jarren James I. Quinto, Grizelda L. Teng, Merlin Teodosia C. Suarez, Rhia S. Trogo, “Measuring Academic Affective States of Students via Brainwave Signals”, Knowledge and Systems Engineering (KSE) 2011 Third International Conference on, pp. 228-231, 2011

Judith J. Azcarraga, John Francis Ibanez Jr., Ianne Robert Lim, Nestor Lumanas Jr., “Use of Personality Profile in Predicting Academic Emotion Based on Brainwaves Signals and Mouse Behavior”, Knowledge and Systems Engineering (KSE) 2011 Third International Conference on, pp. 239-244, 2011.
Yi-Hung Liu, Chien-Te Wu, Yung-Hwa Kao, Ya-Ting Chen, “Single-trial EEG-based emotion recognition using kernel Eigen-emotion pattern and adaptive support vector machine”, Engineering in Medicine and Biology Society (EMBC) 2013 35th Annual International Conference of the IEEE, pp. 4308-4309, 2013, ISSN 1557-170X.
Thong Tri Vo, Nam Phuong Nguyen, Toi Vo Van, IFMBE Proceedings, vol. 63, pp. 621, 2018, ISSN 1880-0737, ISBN 978-981-10-4360-4.
Adrian Rodriguez Aguiñaga, Miguel Angel Lopez Ramirez, Lecture Notes in Computer Science, vol. 9456, pp. 177, 2015, ISSN 0302-9743, ISBN 978-3-319-26507-0.
Judith Azcarraga, Merlin Teodosia Suarez, “Recognizing Student Emotions using Brainwaves and Mouse Behavior Data”, International Journal of Distance Education Technologies, vol. 11, pp. 1, 2013, ISSN 1539-3100.
Tri Thong Vo, Phuong Nam Nguyen, Van Toi Vo, IFMBE Proceedings, vol. 61, pp. 67, 2017, ISSN 1680-0737, ISBN 978-981-10-4219-5.
Alicia Heraz, Claude Frasson, Lecture Notes in Computer Science, vol. 5535, pp. 367, 2009, ISSN 0302-9743, ISBN 978-3-642-02248-3.
Hamwira Yaacob, Wahab Abdul, Norhaslinda Kamaruddin, “Classification of EEG signals using MLP based on categorical and dimensional perceptions of emotions”, Information and Communication Technology for the Muslim World (ICT4M) 2013 5th International Conference on, pp. 1-6, 2013.
Yuan-Pin Lin, Chi-Hong Wang, Tzyy-Ping Jung, Tien-Lin Wu, Shyh-Kang Jeng, Jeng-Ren Duann, Jyh-Horng Chen, “EEG-Based Emotion Recognition in Music Listening”, Biomedical Engineering IEEE Transactions on, vol. 57, pp. 1798-1806, 2010, ISSN 0018-9294.
Yi-Hung Liu, Wei-Teng Cheng, Yu-Tsung Hsiao, Chien-Te Wu, Mu-Der Jeng, “EEG-based emotion recognition based on kernel Fisher's discriminant analysis and spectral powers”, Systems Man and Cybernetics (SMC) 2014 IEEE International Conference on, pp. 2221-2225, 2014.

Using EEG to assess the emotional state has numerous practical applications. One of the first such applications was the development of a travel guide based on emotions by measuring brainwaves by the Singapore tourism group. “By studying the brainwaves of a family on vacation, the researchers drew up the Singapore Emotion Travel Guide, which advises future visitors of the emotions they can expect to experience at different attractions.” (www.lonelyplanet.com/news/2017/04/12/singapore-emotion-travel-guide) Joel Pearson at University of New South Wales and his group developed the protocol of measuring brainwaves of travelers using EEG and decoding specific emotional states.

Another recently released application pertains to virtual reality (VR) technology. On Sep. 18, 2017, Looxid Labs launched a technology that harnesses EEG from a subject waring a VR headset. Looxid Labs intention is to factor in brain waves into VR applications in order to accurately infer emotions. Other products such as MindMaze and even Samsung have tried creating similar applications through facial muscles recognition. (scottamyx.com/2017/10/13/looxid-labs-vr-brain-waves-human-emotions/). According to its website (looxidlabs.com/device-2/), the Looxid Labs Development Kit provides a VR headset embedded with miniaturized eye and brain sensors. It uses B EEG channels: Fp1, Fp2, AF7, AF8, AF3, AF4 in international 10-20 system.

To assess a user's state of mind, a computer may be used to analyze the EEG signals produced by the brain of the user. However, the emotional states of a brain are complex, and the brain waves associated with specific emotions seem to change over time. Wei-Long Zheng at Shanghai Jiao Tong University used machine learning to identify the emotional brain states and to repeat it reliably. The machine learning algorithm found a set of patterns that clearly distinguished positive, negative, and neutral emotions that worked for different subjects and for the same subjects over time with an accuracy of about 80 percent. (See Wei-Long Zheng, Jia-Yi Zhu, Bao-Liang Lu, Identifying Stable Patterns over Time for Emotion Recognition from EEG, arxiv.org/abs/1601.02197; see also How One Intelligent Machine Learned to Recognize Human Emotions, MIT Technology Review, Jan. 23, 2016.)

MEG: Magnetoencephalography (MEG) is a functional neuroimaging technique for mapping brain activity by recording magnetic fields produced by electrical currents occurring naturally in the brain, using very sensitive magnetometers. Arrays of SQUIDs (superconducting quantum interference devices) are currently the most common magnetometer, while the SERF (spin exchange relaxation-free) magnetometer is being investigated (Hämäläinen, Matti; Hari, Riitta; Ilmoniemi, Risto J; Knuutila, Jukka; Lounasmaa, Olli V. (1993). “Magnetoencephalography-theory, instrumentation, and applications to noninvasive studies of the working human brain”. Reviews of Modern Physics. 65 (2): 413-497. ISSN 0034-6861. doi:10.1103/RevModPhys.65.413.) It is known that “neuronal activity causes local changes in cerebral blood flow, blood volume, and blood oxygenation” (Dynamic magnetic resonance imaging of human brain activity during primary sensory stimulation. K. K. Kwong, J. W. Belliveau, D. A. Chesler, I. E. Goldberg, R. M. Weisskoff, B. P. Poncelet, D. N. Kennedy, B. E. Happel, M. S. Cohen, and R. Turner). Using “a 122-channel D.C. SQUID magnetometer with a helmet-shaped detector array covering the subject's head” it has been shown that the “system allows simultaneous recording of magnetic activity all over the head.” (122-channel squid instrument for investigating the magnetic signals from the human brain.) A. I. Ahonen, M. S. Hämäläinen, M. J. Kajola, J. E. T. Knuutila, P. P. Leine, O. V. Lounasmaa, L. T. Parkkonen, J. T. Simla, and C. D. Tesche Physica Scripta, Volume 1993, T49A).

In some cases, magnetic fields cancel, and thus the detectable electrical activity may fundamentally differ from the detectable electrical activity obtained via EEG. However, the main types of brain rhythms are detectable by both methods.

See: U.S. Pat. Nos. 5,059,814; 5,118,606; 5,136,687; 5,224,203; 5,303,705; 5,325,862; 5,461,699; 5,522,863; 5,640,493; 5,715,821; 5,719,561; 5,722,418; 5,730,146; 5,736,543; 5,737,485; 5,747,492; 5,791,342; 5,816,247; 6,497,658; 6,510,340; 6,654,729; 6,893,407; 6,950,697; 8,135,957; 8,620,206; 8,644,754; 9,118,775; 9,179,875; 9,642,552; 20030018278; 20030171689; 20080293578; 20070156457; 20070259323; 20080015458; 20080154148; 20080229408; 20100010365; 20100076334; 20100090835; 20120046531; 20120052905; 20130041281; 20150081299; 20150262016. See EP1304073A2; EP1304073A3; WO20000250A1; and WO2001087153A1.

MEGs seek to detect the magnetic dipole emission from an electrical discharge in cells, e.g., neural action potentials. Typical sensors for MEGs are superconducting quantum interference devices (SQUIDs). These currently require cooling to liquid nitrogen or liquid helium temperatures. However, the development of room temperature, or near room temperature superconductors, and miniature cryocoolers, may permit field deployments and portable or mobile detectors. Because MEGs are less influenced by medium conductivity and dielectric properties, and because they inherently detect the magnetic field vector, MEG technology permits volumetric mapping of brain activity and distinction of complementary activity that might suppress detectable EEG signals. MEG technology also supports vector mapping of fields, since magnetic emitters are inherently dipoles, and therefore a larger amount of information is inherently available.

See, U.S. Pat. Nos. 4,862,359; 5,027,817; 5,190,977; 5,230,346; 5,26,315; 5,309,923; 5,325,862; 5,331,970; 5,546,943; 5,568,816; 5,662,10; 5,724,987; 5,797,853; 5,849,040; 5,845,638; 6,042548; 6,080,164; 6,088,611; 6,097,80; 6,144,872; 6,161,031; 6,171,239; 6,249,308; 6,241,686; 6,20,393; 6,309,361; 6,31,205; 6,322515; 6,356,781; 6,370,414; 6,377,833; 6,385,479; 6,390,979; 6,402,689; 6,419,629; 6,466,816; 6,490,472; 6,526,297; 6,527,715; 6,530,884; 6,547,746; 6,551,243; 6,553,252; 6,622036; 6,644,976; 6,648,880; 6,663,571; 6,684,098; 6,697,660; 6,720,564; 6,740,032; 6,743,167; 6,773,490; 6,907,20; 6,947,790; 6,950,698; 6,963,770; 6,963,771; 6,996,261; 7,010,349; 7,011,814; 7,022083; 7,092748; 7,104,947; 7,105,824; 7,120,486; 7,130,673; 7,171,252; 7,177,675; 7,231,245; 7,254,500; 7,23,861; 7,286,871; 7,338,455; 7,346,395; 7,378,056; 7,461,045; 7,489,964; 7,490,085; 7,490,745; 7,510,69; 7,539,528; 7,547,284; 7,565,193; 7,567,693; 7,577,472; 7,613,502; 7,627,370; 7,647,098; 7,653,433; 7,697,979; 7,72755; 7,754,190; 7,756,568; 7,766,827; 7,769,431; 7,778,692; 7,787,937; 7,787,946; 7,794,413; 7,831,305; 7,849,250; 7,856,24; 7,860,552; 7,89,524; 7,904,139; 7,904,144; 7,933,645; 7,962,204; 7,983,741; 7,986,991; 8,000,773; 8,000,793; 8,002,553; 8,014,847; 8,036,434; 8,065,360; 8,069,125; 8,086,296; 8,121,694; 8,190,248; 8,190,248; 8,197,437; 8,224,433; 8,233,682; 8,233,965; 8,236,038; 8,262,714; 8,280,514; 8,295,914; 8,306,607; 8,306,610; 8,313,441; 8,326,433; 8,337,404; 8,346,331; 8,346,342; 8,356,004; 8,358,818; 8,364,271; 8,380,28; 8,380,029; 8,380,314; 8,391,942; 8,391,956; 8,423,125; 8,425,583; 8,429,225; 8,445,851; 8,457,746; 8,467,878; 8,473,024; 8,498,708; 8,509,879; 8,527,035; 8,532756; 8,538,513; 8,543,188; 8,554,325; 8,562,951; 8,571,629; 8,586,932; 8,591,419; 8,606,348; 8,606,356; 8,615,47; 8,626,264; 8,626,301; 8,632,750; 8,644,910; 8,655,817; 8,657,756; 8,666,478; 8,67900; 8,684,926; 8,690,748; 8,696,722; 8,7066,205; 8,706,241; 8,706,510; 8,712512; 8,717,430; 8,725,669; 8,738,395; 8,761,869; 8,761,888; 8,768,022; 8,805,516; 8,814,923; 8,831,731; 8,834,546; 8,838,227; 8,849,392; 8,849,632; 8,85210; 8,855,773; 8,858,441; 8,868,174; 8,888,702; 8,915,741; 8,918,162; 8,938,28; 8,938,290; 8,951,188; 8,951,192; 8,956,277; 8,965,513; 8,977,362; 8,989,836; 8,998,828; 9,005,126; 9,020,576; 9,022,936; 9,026,217; 9,026,218; 9,802,412; 9,033,884; 9,037,224; 9,042,201; 9,050,471; 9,067,052; 9,072,905; 9,084,896; 9,088,410; 9,088,683; 9,082556; 9,095,266; 9,101,276; 9,107,595; 9,116,835; 9,133,024; 9,144,392; 9,149,255; 9,155,50; 9,167,970; 9,167,976; 9,167,977; 9,167,978; 9,171,366; 9,173,609; 9,179,850; 9,179,854; 9,179,850; 9,179,875; 9,192,300; 9,190,637; 9,190,778; 9,204,835; 9,211,077; 9,211,212; 9,213,074; 9,242067; 9,247,890; 9,247,924; 9,248,288; 9,254,097; 9,254,383; 9,268,014; 9,268,015; 9,271,651; 9,271,674; 9,282,930; 9,289,143; 9,302,110; 9,308,372; 9,320,449; 9,322,895; 9,326,742; 9,332,939; 9,336,611; 9,338,227; 9,357,941; 9,367,131; 9,370,308; 9,375,145; 9,375,564; 9,387,320; 9,395,425; 9,492,558; 9,413,038; 9,414,029; 9,436,88; 9,440,064; 9,463,327; 9,470,728; 9,471,978; 9,474,852; 9,486,632; 9,492,313; 9,560,967; 9,579,048; 9,592409; 9,597,493; 9,597,494; 9,615,789; 9,616,166; 9,655,573; 9,655,669; 9,662048; 9,662492; 9,669,185; 9,675,292; 9,682,232; 9,687,187; 9,707,396; 9,713,433; 9,713,444; 20010020127; 2001002800; 20010051774; 20020005784; 20020016552; 20020017994; 20020042563; 20020058867; 20020099273; 20020099295; 20020103428; 20020103429; 20020128638; 20030001098; 20030009096; 20030013981; 20030032870; 20030040660; 20030068605; 20030074032; 20030093004; 20030093005; 20030120140; 20030128801; 20030135128; 20030153810; 20030163027; 20030163028; 20030181821; 20030187358; 20030204135; 20030225335; 20030236458; 20040030585; 20040059241; 2004007233; 20049077960; 20040092809; 20040096395; 20040097802; 20040116798; 20040122787; 20040122790; 20040144925; 20040204656; 20050004488; 20050007091; 20050027284; 20050033122; 20050033154; 20050033379; 20050079474; 20050079636; 20050106713; 20050107654; 20050119547; 20050131311; 20050136002; 20050159670; 20050159671; 20050182456; 20050192514; 20050222638; 20050283053; 20060004422; 20060015034; 20060018525; 20060036152; 20060036153; 20060051814; 20060052706; 20060058683; 20060074290; 20060074298; 20060078183; 20060084858; 20060100526; 20060111644; 20060116556; 20060122481; 20060129324; 20060173510; 2006018866; 20060241373; 20060241382; 20070005115; 20070007454; 20070008172; 20070015985; 20070032737; 20070055145; 20070100251; 20070138886; 20070179534; 20070184507; 20070191704; 20070191727; 20070203401; 20070239058; 20070250138; 20070255135; 20070293760; 20070299370; 20080001600; 20080021332; 20080021340; 20080033297; 20080039698; 20080039737; 20080042067; 20080058664; 20080091118; 20080097197; 20080123927; 20080125669; 20080128626; 20080154126; 20080167571; 20080221441; 20080230702; 20080230705; 20080249430; 20080255948; 20080275340; 20080306365; 20080311548; 20090012387; 20090018407; 20090018431; 20090018462; 20090024050; 20090048507; 20090054788; 20090054800; 20090054958; 20090062676; 20090078875; 20090082828; 20090099627; 20080112017; 20080112273; 20080112277; 20080112278; 20080112278; 20080112280; 20090118622; 20090131995; 20090137923; 20090156907; 20090156955; 20090157323; 20090157481; 20090157482; 20090157625; 20090157662; 20090157751; 20090157813; 20090163777; 20090164131; 20090164132; 20090171164; 20090172540; 20090177050; 20090179642; 20090191131; 20090209845; 20090216091; 2009022042; 20090221928; 20090221930; 20090246138; 20090264785; 20090267758; 20090270694; 20090287271; 20090287272; 20090287273; 20090287274; 20090287467; 2009029280; 20090292713; 20090292724; 20090299168; 20090304582; 20090306531; 20090306534; 20090318773; 20090318794; 2010002378; 20100030073; 20100036233; 20100036453; 20100041962; 20100042011; 20100049276; 20100069738; 20100069777; 20100076274; 20100082506; 20100087718; 20100094154; 20100094155; 20100099975; 20100106043; 20100113958; 20100114193; 20100114237; 20100130868; 20100143256; 20100163027; 20100163028; 20100163035; 20100168525; 20100168528; 20100168602; 20100189318; 20100191095; 20100191124; 20100204748; 20100248275; 20100249573; 20100261993; 20100298735; 20100324441; 20110004115; 20110004412; 20110009777; 20110015515; 20110015538; 20110028858; 2011003482; 20110046491; 20110054345; 20110054562; 20110077503; 20110092800; 20110092882; 20110112394; 20110112426; 20110119212; 20110125048; 20110125238; 20110129129; 2011014452; 20110160543; 20110160607; 20110160608; 20110161011; 20110178358; 20110178441; 20110178442; 20110207988; 20110208094; 20110213200; 20110218405; 20110230738; 20110257517; 20110283962; 20110283968; 20110270074; 20110270914; 20110275927; 20110295143; 20110295166; 20110301448; 20110306845; 20110306846; 20110307029; 20110313288; 20110313487; 20120004561; 20112021394; 20120022343; 20120022884; 20120035765; 20120046531; 201200468971; 20120053448; 20120053483; 20120078327; 20120083700; 20120108998; 20120130228; 20120130228; 20120149042; 20120150545; 20120163688; 20120165898; 20120165904; 20120197163; 20120215114; 2012029507; 20120226091; 20120226185; 20120232327; 20120232433; 20120245493; 2012025328; 20120253434; 20120265267; 20120271148; 20120271151; 20120271376; 20120283502; 20120283604; 20120296241; 20120296253; 20120296568; 20120302867; 20120310107; 20120310298; 20120316793; 20130012804; 20130063434; 20130066350; 20130066391; 20130066394; 20130072780; 2013007962; 20130085678; 20130096441; 20130096454; 21130102897; 20130109996; 20130110616; 20130116561; 20130131755; 20130138177; 20130172716; 20130178693; 20130184728; 20130188854; 20130204085; 2013021238; 20130226281; 20130231580; 20130238063; 20130245422; 20130245424; 20130245486; 20130281506; 20130274586; 20130281878; 20130281890; 20130289386; 20130304153; 20140000630; 20140005518; 20140031703; 20140057232; 20140058241; 20140058292; 20140066763; 2014008115; 20140088377; 20140094718; 20140094720; 20140111335; 20140114207; 20140119621; 20140128763; 20140135642; 20140148657; 20140151563; 20140155952; 20140163328; 20140163368; 20140163408; 20140171748; 20140171757; 20140171818; 20140180088; 20140180092; 20140180093; 20140180094; 20140180095; 20140180096; 20140180097; 20140180098; 20149180100; 20149080112; 20149080113; 20149180176; 20141080177; 20149193336; 20149194726; 20149200414; 20149021593; 20149220649; 20149228702; 20149243614; 20149243652; 20114243714; 20149249360; 20149249445; 20114257073; 20149270438; 20149275807; 20149275851; 20149275891; 20114276013; 20149276014; 20149276187; 20149276702; 20149279746; 20149296646; 20140296655; 20140303425; 20149303486; 20149316248; 2014032384; 201149330268; 20149330394; 20149335489; 20149336489; 20140340084; 20114343397; 20140357962; 2011436470; 20149371573; 20149378830; 20149378941; 20150011866; 20150011877; 20150018665; 20150018905; 20150024356; 20150025498; 20150025422; 20150025610; 20150029087; 20150033245; 2015003325; 20150033259; 20150033262; 20150033266; 20150035959; 20150038812; 20150038822; 20150038869; 20150039066; 20150073237; 20150080753; 20150088120; 20150119650; 20150119689; 20150119698; 20150149520; 2015014152; 20150141773; 20150150473; 20150151142; 20150157266; 20150165239; 20150174418; 20150182417; 20150196800; 20150201879; 20150208994; 20150219732; 2015022370; 20150227722; 20150230744; 20150246238; 20150247921; 20150257700; 20150290420; 20150297106; 20150297893; 20150305799; 20150305800; 20150305801; 20150306349; 20150313549; 20150317796; 20150320591; 20150327813; 20150335281; 20150335294; 20150339363; 20150343242; 20150359431; 20150360039; 20160001065; 20160001096; 20160001098; 20160008620; 201600086322; 2016001528; 2016002065; 2016002067; 20160022168; 20160022206; 20160027342; 20160029946; 20160029965; 20160038049; 20160038559; 20160048659; 20160051161; 20160051622; 20160058354; 20160058392; 2016006682; 20160066838; 20160081613; 20160100768; 20160120480; 20160120864; 20160143541; 20160143574; 20160151018; 20160151620; 20160157820; 20160158553; 2016016609; 20160184599; 20160196393; 20160199241; 20160203597; 20160206380; 20160206871; 20160206877; 20160213276; 20160235324; 20160235980; 20160235983; 20160239966; 20160239968; 20160245670; 20160245766; 20160270723; 20160278687; 20160207118; 20160207436; 20160296746; 20160302720; 20160303397; 20160303402; 20160320210; 20160339243; 20160341684; 20160361534; 20160366482; 20160371721; 2017000161; 20170027539; 20170032090; 20170039706; 20170042474; 20170043167; 20170065349; 20170079530; 20117080320; 20170085855; 2017008672; 20170086763; 20170087367; 20170091410; 2017012493; 20170112427; 20170112446; 2017012577; 20170147578; 20170151435; 20179160360; 20170164861; 20170164862; 20170164893; 20170164894; 20170172527; 20170173262; 20170185714; 20170188862; 20179188866; 20170188860; 20170188869; 20170188932; 20170189691; 20170196501; and 20170202633.
Allen, Philip B., et al. High-temperature superconductivity. Springer Science & Business Media, 2012;
Fausti, Daniele, et al. “Light-induced superconductivity in a stripe-ordered cuprate.” Science 331.8014 (2011):189-191;
Inoue, Mitsuteru, et al. “Investigating the use of magnonic crystals as extremely sensitive magnetic field sensors at room temperature.” Applied Physics Letters 98.13 (2011):132511;
Kaiser, Stefan, et al. “Optically induced coherent transport far above Tc in underdoped YBa2 Cu306+δ.” Physical Review B 89.18 (2014):184518;
Malik, M. A., and B. A. Malik. “High Temperature Superconductivity: Materials, Mechanism and Applications.” Bulgarian J. Physics 41.4 (2014).
Mankowsky, Roman, et al. “Nonlinear lattice dynamics as a basis for enhanced superconductivity in YBa2Cu306. 5.” arXiv preprint arXiv:1405.2288 (2014);
Mcfetridge, Grant. “Room temperature superconductor.” U.S. Pub. App. No. 2002006875.
Mitrano, Matteo, et al. “Possible light-induced superconductivity in K3C60 at high temperature.” Nature 530.7591 (2016): 461-464;
Mourachkine, Andrei. Room-temperature superconductivity. Cambridge Int Science Publishing, 2004;
Narlikar, Anent V., ed. High Temperature Superconductivity 2. Springer Science & Business Media, 2013;
Pickett, Warren E. “Design for a room-temperature superconductor.” J. superconductivity and novel magnetism 19.3 (2008): 291-297;
Sleight, Arthur W. “Room temperature superconductors.” Accounts of chemical research 28.3 (1995):103-108.
Hämäläinen, Matti; Hari, Riitta; Ilmoniemi, Risto J; Knuutila, Jukka; Lounasmaa, Olli V. (1993). “Magnetoencephalography-theory, instrumentation, and applications to noninvasive studies of the working human brain”. Reviews of Modern Physics. 65 (2): 413-497. ISSN 0034-6861. doi:10.1103/RevModPhys.65.413.

EEGs and MEGs can monitor the state of consciousness. For example, states of deep sleep are associated with slower EEG oscillations of larger amplitude. Various signal analysis methods allow for robust identifications of distinct sleep stages, depth of anesthesia, epileptic seizures and connections to detailed cognitive events.

LORETA: Low-resolution brain electromagnetic tomography often referred as LORETA is a functional imaging technology usually using a linearly constrained minimum variance vector beamformer in the time-frequency domain as described in Gross et al., “Dynamic imaging of coherent sources: Studying neural interactions in the human brain”, PNAS 98, 694-699, 2001. It allows to the image (mostly 3D) evoked and induced oscillatory activity in a variable time-frequency range, where time is taken relative to a triggered event. There are three categories of imaging related to the technique used for LORETA. See, wiki.besa.de/index.php?title=Source_Analysis_3 D_Imaging#Multiple_Source_Beamformer_.28MSBF.29. The Multiple Source Beamformer (MSBF) is a tool for imaging brain activity. It is applied in the time-frequency domain and based on single-trial data. Therefore, it can image not only evoked, but also induced activity, which is not visible in time-domain averages of the data. Dynamic Imaging of Coherent Sources (DIES) can find coherence between any two pairs of voxels in the brain or between an external source and brain voxels. DIES requires time-frequency-transformed data and can find coherence for evoked and induced activity. The following imaging methods provides an image of brain activity based on a distributed multiple source model: CLARA is an iterative application of LORETA images, focusing the obtained 3D image in each iteration step. LAURA uses a spatial weighting function that has the form of a local autoregressive function. LORETA has the 3D Laplacian operator implemented as spatial weighting prior. sLORETA is an unweighted minimum norm that is standardized by the resolution matrix. swLORETA is equivalent to sLORETA, except for an additional depth weighting. SSLOFO is an iterative application of standardized minimum norm images with consecutive shrinkage of the source space. A User-defined volume image allows experimenting with the different imaging techniques. It is possible to specify user-defined parameters for the family of distributed source images to create a new imaging technique. If no individual MRI is available, the minimum norm image is displayed on a standard brain surface and computed for standard source locations. If available, an individual brain surface is used to construct the distributed source model and to image the brain activity. Unlike classical LORETA, cortical LORETA is not computed in a 3D volume, but on the cortical surface. Unlike classical CLARA, cortical CLARA is not computed in a 3D volume, but on the cortical surface. The Multiple Source Probe Scan (MSPS) is a tool for the validation of a discrete multiple source model. The Source Sensitivity image displays the sensitivity of a selected source in the current discrete source model and is, therefore, data independent.

See U.S. Pat. Nos. 4,562,540; 4,594,662; 5,650,726; 5,859,533; 6,026,173; 6,182,013; 6,294,917; 6,332,087; 6,393,363; 6,534,86; 6,703,830; 6,791,331; 6,856,830; 6,863,127; 7,030,617; 7,092,748; 7,119,553; 7,170,294; 7,239,731; 7,276,916; 7,206,871; 7,295,019; 7,353,065; 7,363,164; 7,454,243; 7,499,894; 7,648,498; 7,804,441; 7,808,434; 7,841,986; 7,852,087; 7,937,222; 8,000,795; 8,046,076; 8,131,526; 8,174,430; 8,188,749; 8,244,341; 8,263,574; 8,332,191; 8,346,365; 8,362,780; 8,456,166; 8,538,700; 8,565,883; 8,593,154; 8,600,513; 8,706,205; 8,711,655; 8,731,987; 8,756,017; 8,761,438; 8,812,237; 8,829,908; 8,958,882; 8,008,970; 8,035,657; 8,068,087; 8,072,448; 8,091,785; 8,082,895; 8,21,964; 9,133,709; 8,165,472; 8,179,854; 8,320,451; 8,367,738; 9,414,749; 8,414,763; 8414,764; 8,442,088; 8,468,541; 8,513,398; 8,545,225; 8,557,438; 8,562,988; 9,568,635; 8,651,706; 8,675,254; 8,675,255; 8,675,292; 9,713,433; 9,715,032; 2002000080; 20020017905; 20030018277; 20030093004; 20040097802; 20040116798; 20040131998; 20040140811; 20040145370; 20050156602; 20060058856; 20060069058; 20060136135; 20060149160; 20060152227; 20060170424; 20060176062; 20060184058; 20060206108; 20070060974; 20070159185; 20070191727; 20080033513; 20080097235; 20080125830; 20080125831; 20080183072; 20080242976; 2008025816; 20080201667; 20090039889; 20090054801; 20090082688; 20090099783; 20090216146; 20090261832; 20090306534; 20090312663; 20100010366; 20100030097; 20100042011; 20100056276; 20100092934; 20100132448; 20100134113; 20100168053; 20100198519; 2010023120; 20100238763; 20110004115; 20110050232; 20110160607; 20110308789; 20120010493; 20120011927; 20120016430; 20120083690; 20120130641; 20120150257; 20120162002; 2012005448; 20120245474; 20120268272; 20120269385; 20120296569; 20130091941; 20130096408; 20130141103; 20130231709; 20130289385; 20130303934; 20140015852; 20140025133; 20140058528; 20140066739; 20140107519; 20114012073; 20140155740; 20140161352; 20140163328; 20140163893; 201140228702; 20140243714; 20140275944; 20140276012; 20140323899; 20150051663; 20150112409; 20150119689; 20150137817; 20150145519; 20150157235; 20150167459; 20150177413; 20150248615; 20150257648; 20150257649; 2015030108; 20150342472; 20160002523; 20160038049; 20160040514; 20160051161; 20160051622; 20160091448; 20160102500; 20160120436; 20160136427; 20160187524; 20160213276; 2016022080; 20160223703; 20160235983; 20160245952; 20160256109; 20160259085; 20160262623; 20160298449; 20160334534; 20160345856; 20160356911; 20160367812; 20170001016; 20170067323; 20170138132; and 20170151436.

Brain Stimulation: Non-invasive brain stimulation (NIBS) bypasses the correlative approaches of other imaging techniques, making it possible to establish a causal relationship between cognitive processes and the functioning of specific brain areas. NIBS can provide information about where a particular process occurs. NIBS offers the opportunity to study brain mechanisms beyond process localization, providing information about when activity in a given brain region is involved in a cognitive process, and even how it is involved. When using NIBS to explore cognitive processes, it is important to understand not only how NIBS functions but also the functioning of the neural structures themselves. Non-invasive brain stimulation (NIBS) methods, which include transcranial magnetic stimulation (TMS) and transcranial electric stimulation (TES), are used in cognitive neuroscience to induce transient changes in brain activity and thereby alter the behavior of the subject.

The application of NIBS aims at establishing the role of a given cortical area in an ongoing specific motor, perceptual or cognitive process. Physically, NIBS techniques affect neuronal states through different mechanisms. In TMS, a solenoid (coil) is used to deliver a strong and transient magnetic field, or “pulse,” to induce a transitory electric current at the cortical surface beneath the coil. The pulse causes the rapid and above-threshold depolarization of cell membranes affected by the current, followed by the transynaptic depolarization or hyperpolarization of interconnected neurons. Therefore, strong TMS can induce a current that elicits action potentials in neurons, while weak (subthreshold) can modify susceptibility of cells to depolarization. A complex set of coils can deliver a complex 3D excitation field. By contrast, in TES techniques, the stimulation involves the application of weak electrical currents directly to the scalp through a pair of electrodes. As a result, TES induces a subthreshold polarization of cortical neurons that is too weak to generate an action potential. (Superthreshold tES corresponds to electroconvulsive therapy, which is a currently disfavored, but apparently effective treatment for depression). However, by changing the intrinsic neuronal excitability, TES can induce changes in the resting membrane potential and the postsynaptic activity of cortical neurons. This, in turn, can alter the spontaneous firing rate of neurons and modulate their response to afferent signals, leading to changes in synaptic efficacy. The typical application of NIBS involves different types of protocols: TMS can be delivered as a single pulse (spTMS) at a precise time, as pairs of pulses separated by a variable interval, or as a series of stimuli in conventional or patterned protocols of repetitive TMS (rTMS). In tES, different protocols are established by the electrical current used and by its polarity, which can be direct (anodal or cathodal transcranial direct current stimulation: tDCS), alternating at a fix frequency (transcranial alternating current stimulation: tACS), oscillating transcranial direct current stimulation (osc-tDCS), high-definition transcranial direct current stimulation (HD-tDCS), or at random frequencies (transcranial random noise stimulation: tRNS). (Nitsche et al., 2008; Paulus, 2011).

In general, the final effects of NIBS on the central nervous system depend on a lengthy list of parameters (e.g., frequency, temporal characteristics, intensity, geometric configuration of the coil/electrode, current direction), when it is delivered before (off-line) or during (on-line) the task as part of the experimental procedure. In addition, these factors interact with several variables related to the anatomy (e.g., properties of the brain tissue and its location), as well as physiological (e.g., gender and age) and cognitive states of the stimulated area/subject. The entrainment hypothesis, suggests the possibility of inducing a particular oscillation frequency in the brain using an external oscillatory force (e.g., rTMS, but also tACS). The physiological basis of oscillatory cortical activity lies in the timing of the interacting neurons; when groups of neurons synchronize their firing activities, brain rhythms emerge, network oscillations are generated, and the basis for interactions between brain areas may develop. Because of the variety of experimental protocols for brain stimulation, limits on descriptions of the actual protocols employed, and limited controls, consistency of reported studies is lacking, and extrapolability is limited. Thus, while there is some consensus in various aspects of the effects of extra cranial brain stimulation, the results achieved have a degree of uncertainty dependent on details of implementation. On the other hand, within a specific experimental protocol, it is possible to obtain statistically significant and repeatable results. This implies that feedback control might be effective to control implementation of the stimulation for a given purpose; however, prior studies that employ feedback control are lacking.

Changes in the neuronal threshold result from changes in membrane permeability (Liebetanz et al., 2002), which influence the response of the task-related network. The same mechanism of action may be responsible for both TES methods and TMS, i.e., the induction of noise in the system. However, the neural activity induced by TES will be highly influenced by the state of the system because it is a neuromodulatory method (Paulus, 2011), and its effect will depend on the activity of the stimulated area. Therefore, the final result will depend strongly on the task characteristics, the system state and the way in which TES will interact with such a state.

In TMS, the magnetic pulse causes a rapid increase in current flow, which can in some cases cause and above-threshold depolarization of cell membranes affected by the current, triggering an action potential, and leading to the trans-synaptic depolarization or hyperpolarization of connected cortical neurons, depending on their natural response to the firing of the stimulated neuron(s). Therefore, TMS activates a neural population that, depending on several factors, can be congruent (facilitate) or incongruent (inhibit) with task execution. TES induces a polarization of cortical neurons at a subthreshold level that is too weak to evoke an action potential. However, by inducing a polarity shift in the intrinsic neuronal excitability, TES can alter the spontaneous firing rate of neurons and modulate the response to afferent signals. In this sense, TES-induced effects are even more bound to the state of the stimulated area that is determined by the conditions. In short, NIBS leads to a stimulation-induced modulation of the state that can be substantially defined as noise induction. Induced noise will not be just random activity, but will depend on the interaction of many parameters, from the characteristics of the stimulation to the state.

The noise induced by NIBS will be influenced by the state of the neural population of the stimulated area. Although the types and number of neurons “triggered” by NIBS are theoretically random, the induced change in neuronal activity is likely to be correlated with ongoing activity, yet even if we are referring to a non-deterministic process, the noise introduced will not be a totally random element. Because it will be partially determined by the experimental variables, the level of noise that will be introduced by the stimulation and by the context can be estimated, as well as the interaction between the two levels of noise (stimulation and context). Known transcranial stimulation does not permit stimulation with a focused and highly targeted signal to a clearly defined area of the brain to establish a unique brain-behavior relationship; therefore, the known introduced stimulus activity in the brain stimulation is ‘noise.’

Cosmetic neuroscience has emerged as a new field of research. Roy Hamilton, Samuel Messing, and Anjan Chatterjee, “Rethinking the thinking cap—Ethics of neural enhancement using noninvasive brain stimulation.” Neurology, Jan. 11, 2011, vol. 76 no. 2187-193. (www.neurology.org/content/76/2/187.) discuss the use noninvasive brain stimulation techniques such as transcranial magnetic stimulation and transcranial direct current stimulation to enhance neurologic function: cognitive skills, mood, and social cognition.

Electrical brain stimulation (EBS), or focal brain stimulation (FBS), is a form of clinical neurobiology electrotherapy used to stimulate a neuron or neural network in the brain through the direct or indirect excitation of cell membranes using an electric current. See, en.wikipedia.org/wiki/Electrical_brain_stimulation; U.S. Pat. Nos. 7,753,836; 7,948,73; 8,545,378; 9,345,901; 9,610,458; 9,694,178; 20140330337; 20150112403; and 20150119689.

Motor skills can be affected by CNS stimulation.

See. U.S. Pat. Nos. 5,343,871; 5,742,748; 6,057,846; 6,390,979; 6,644,976; 6,656,137; 7,063,535; 7,558,622; 7,618,381; 7,733,224; 7,829,562; 7,863,272; 8,016,597; 8,065,249; 8,069,125; 8,108,036; 8,126,542; 8,150,796; 8,195,593; 8,356,004; 8,449,471; 8,461,988; 8,525,673; 8,525,687; 8,531,291; 8,591,419; 8,606,592; 8,615,479; 8,680,991; 8,682,448; 8,706,510; 8,747,336; 8,750,971; 8,764,651; 8,784,108; 8,858,449; 8,862,236; 8,938,288; 8,962,042; 8,005,648; 8,064,036; 9,107,586; 9,125,780; 9,138,579; 9,149,598; 9,173,582; 9,204,796; 9,211,077; 9,265,450; 9,351,649; 9,358,361; 9,380,976; 9,493,038; 9,418,368; 9,480,541; 9,495,684; 9,545,515; 9,548,691; 9,560,967; 9,577,992; 9,590,886; 20030068605; 2004907033; 20050020483; 20050032027; 20050059689; 2005015326; 20060014753; 20060052386; 20060106326; 20060191543; 20060229164; 20070031790; 20070138886; 20070276270; 20080001735; 20080004904; 20080243005; 2008028780; 20080294919; 20090005654; 20090018407; 20090024050; 20090118593; 20090119154; 20090132275; 20090156907; 20090156955; 20090157323; 20090157481; 20090157482; 20090157625; 20090157660; 20090157751; 20090157813; 20090163777; 20090164131; 20090164132; 20090164302; 20090164491; 20090164493; 20090164450; 20090164503; 20090164549; 20090171164; 20090172549; 200902292; 20090267750; 20090271011; 20090271120; 20090271347; 20090312595; 20090312660; 20090318773; 20090318779; 20090319002; 20100004762; 20100015583; 20100017001; 2010002280; 20100041950; 20100042578; 20100063360; 20100069724; 20100076249; 201100081860; 20100081861; 20100100036; 20100125561; 20100130811; 2010014509; 20100163027; 20100163035; 20100168525; 20100168602; 20100280332; 20110015209; 20110015468; 2011008054; 20110105859; 20110115624; 20110152284; 20110178441; 20110181422; 20110288119; 20120092156; 20120092157; 20120130300; 20120143104; 20120164613; 20120177716; 20120316793; 2012033010; 20130009783; 20130018592; 20130034837; 20130053656; 20130054215; 20130085678; 20130121984; 20130132029; 20130137717; 20130144537; 20130184728; 20130184997; 2013021291; 20130231574; 20130281890; 20130289385; 20130330428; 2014039571; 20149058520; 20149077946; 20149094720; 20149104959; 20149148479; 20149155430; 20114163425; 20140207224; 20140235965; 20149249429; 20150025410; 20150025422; 20150068069; 20150071907; 20150141773; 2015020882; 20150265583; 20150290419; 20150294967; 20150294985; 20150294986; 20150359467; 20150379878; 20160001096; 20160007904; 20160007915; 20160030749; 20160030750; 20160067492; 20160074657; 20160120437; 20160149834; 2016018968; 20160206671; 20160220821; 20160303402; 20160351069; 20160360965; 20170046971; 20170065638; 20170080320; 20170084187; 20170086672; 20170112947; 20170127727; 20170131293; 20170143966; 20170151436; 20170157343; and 20170193831. See:
Abraham, W. C., 2008. Metaplasticity: tuning synapses and networks for plasticity. Nature Reviews Neuroscience 9, 387.
Abrahamyan, A., Clifford, C. W. Arabzadeh, E., Harris, J. A., 2011. Improving visual sensitivity with subthreshold transcranial magnetic stimulation. J. Neuroscience 31, 3290-3294.
Adrian, E D., 1928. The Basis of Sensation. W.W. Norton, New York.
Amassian, V. E., Cracco, R. Q., Maccabee, P. J., Cracco, J. B., Rudell, A., Eberle, L., 1989. Suppression of visual perception by magnetic coil stimulation of human occipital cortex. Electroencephalography and Clin. Neurophysiology 74, 458-462.
Amassian, V. E., Eberle, L., Maccabee, P. J., Cracco, R. Q., 1992. Modelling magnetic coil excitation of human cerebral cortex with a peripheral nerve immersed in a brain-shaped volume conductor: the significance of fiber bending in excitation. Electroencephalography and Clin. Neurophysiology 85, 291-301.
Antal, A., Boros, K., Poreisz, C., Chaieb, L., Terney, D., Paulus, W., 2008. Comparatively weak after-effects of transcranial alternating current stimulation (tACS) on cortical excitability in humans. Brain Stimulation I, 97-105.
Antal, A., Nitsche, M. A., Kruse, W., Kincses, T. Z., Hoffmann, K. P, Paulus, W., 2004. Direct current stimulation over V5 enhances visuomotor coordination by improving motion perception in humans. J. Cognitive Neuroscience 16, 521-527.
Ashbridge, E., Walsh, V., Cowey, A., 1997. Temporal aspects of visual search studied by transcranial magnetic stimulation. Neuropsychologia 35, 1121-1131.
Barker, A. T., Freeston, I. L., Jalinous, R., Jarratt, J. A., 1987. Magnetic stimulation of the human brain and peripheral nervous system: an introduction and the results of an initial clinical evaluation. Neurosurgery 20, 100-109.
Barker, A. T., Jalinous, R., Freeston, I. L., 1985. Non-invasive magnetic stimulation of human motor cortex. Lancet 1, 1106-1107.
Bi, G., Poo, M., 2001. Synaptic modification by correlated activity: Hebb's postulate revisited. Annual Review of Neuroscience 24, 139-166.
Bialek, W., Rieke, F., 1992. Reliability and information transmission in spiking neurons. Trends in Neurosciences 15, 428-434.
Bienenstock, E. L., Cooper, L. N., Munro, P. W., 1982. Theory for the development of neuron selectivity: orientation specificity and binocular interaction in visual cortex. J. Neuroscience 2, 32-48.
Bindman, L. J., Lippold, O. C., Milne, A. R., 1979. Prolonged changes in excitability of pyramidal tract neurones in the cat: a post-synaptic mechanism. J. Physiology 286, 457-477.
Bindman, L. J., Lippold, O. C., Redfearn, J. W., 1962. Long-lasting changes in the level of the electrical activity of the cerebral cortex produced by polarizing currents. Nature 196, 584-585.
Bindman, L. J., Lippold, O. C., Redfearn, J. W., 1964. The action of brief polarizing currents on the cerebral cortex of the rat (1) during current flow and (2) in the production of long-lasting after-effects. J. Physiology 172, 369-382.
Brignani, D., Ruzzoli, M., Mauri, P., Miniussi, C., 2013. Is transcranial alternating current stimulation effective in modulating brain oscillations? PLoS ONE 8, e56589. Buzsaki, G., 2006. Rhythms of the Brain. Oxford University Press, Oxford.
Canolty, R. T., Knight, R. T., 2010. The functional role of cross-frequency coupling. Trends in Cognitive Sciences 14, 506-515.
Carandini, M., Ferster, D., 1997. A tonic hyperpolarization underlying contrast adaptation in cat visual cortex. Science 276, 949-952.
Cattaneo, L., Sandrini, M., Schwarzbach, J., 2010. State-dependent TMS reveals a hierarchical representation of observed acts in the temporal, parietal, and premotor cortices. Cerebral Cortex 20, 2252-2258.
Cattaneo, Z., Rota, F., Vecchi, T., Silvanto, J., 2008. Using state-dependency of trans-cranial magnetic stimulation (TMS) to investigate letter selectivity in the left posterior parietal cortex: a comparison of TMS-priming and TMS-adaptation paradigms. Eur. J. Neuroscience 28, 1924-1929.
Chambers, C. D., Payne, J. M., Stokes, M. G., Mattingley, J. B., 2004. Fast and slow parietal pathways mediate spatial attention. Nature Neuroscience 7, 217-218.
Corthout, E., Uttl, B., Walsh, V., Hallett, M., Cowey, A., 1999. Timing of activity in early visual cortex as revealed by transcranial magnetic stimulation. Neuroreport 10, 2631-2634.
Creutzfeldt, O. D., Fromm, G. H., Kapp, H., 1962. Influence of transcortical d-c currents on cortical neuronal activity. Experimental Neurology 5, 436-452.
Deans, J. K., Powell, A. D., Jefferys, J. G., 2007. Sensitivity of coherent oscillations in rat hippocampus to AC electric fields. J. Physiology 583, 555-565.
Dockery, LA., Hueckel-Weng, R., Birbaumer, N., Plewnia, L, 2009. Enhancement of planning ability by transcranial direct current stimulation. J. Neuroscience 29, 7271-7277.
Ermentrout, G. B., Galan, R. F., Urban, N. N., 2008. Reliability, synchrony and noise. Trends in Neurosciences 31, 428-434.
Epstein, C. M., Rothwell, J. C., 2003. Therapeutic uses of rTMS. Cambridge University Press, Cambridge, pp. 246-263.
Faisal, A. A., Selen, L. P., Wolpert, D. M., 2008. Noise in the nervous system. Nature Reviews Neuroscience 9, 292-303.
Ferbert, A., Caramia, D., Priori, A., Bertolasi, L., Rothwell, J. C., 1992. Cortical projection to erector spinae muscles in man as assessed by focal transcranial magnetic stimulation. Electroencephalography and Clin. Neurophysiology 85, 382-387.
Fertonani, A., Pirulli, C., Miniussi, C., 2011. Random noise stimulation improves neuroplasticity in perceptual learning. J. Neuroscience 31, 15416-15423. Feurra, M., Galli, G., Rossi, S., 2012. Transcranial alternating current stimulation affects decision making. Frontiers in Systems Neuroscience 6, 39.
Guyonneau, R., Vanrullen, R., Thorpe, S. J., 2004. Temporal codes and sparse representations: a key to understanding rapid processing in the visual system. J. Physiology, Paris 98, 487-497.
Hallett, M., 2000. Transcranial magnetic stimulation and the human brain. Nature 406, 147-150.
Harris, I. M., Miniussi, C., 2003. Parietal lobe contribution to mental rotation demonstrated with rTMS. J. Cognitive Neuroscience 15, 315-323.
Harris, J. A., Clifford, C. W., Miniussi, C., 2008. The functional effect of transcranial magnetic stimulation: signal suppression or neural noise generation. J. Cognitive Neuroscience 20, 734-740.
Hebb, D. O., 1949. The Organization of Behavior; A Neuropsychological Theory. Wiley, New York.
Hutcheon, B., Yarom, Y., 2000. Resonance, oscillation and the intrinsic frequency preferences of neurons. Trends in Neurosciences 23, 216-222.
Jacobson, L., Koslowsky, M., Lavidor, M., 2011. tDCS polarity effects in motor and cognitive domains: a meta-analytical review. Experimental Brain Research 216, 1-10.
Joundi, R. A., Jenkinson, N., Brittain, J. S., Aziz, T. Z., Brown, P., 2012. Driving oscillatory activity in the human cortex enhances motor performance. Current Biology 22, 403-407.
Kahn, I., Pascual-Leone, A., Theoret, H., Fregni, F., Clark, D., Wagner, A. D., 2005. Transient disruption of ventrolateral prefrontal cortex during verbal encoding affects subsequent memory performance. J. Neurophysiology 94, 688-698.
Kanai, R., Chaieb, L., Antal, A., Walsh, V., Paulus, W., 2008. Frequency-dependent electrical stimulation of the visual cortex. Current Biology 18, 1839-1843.
Kitajo, K., Doesburg, S. M., Yamanaka, K., Nozaki, D., Ward, L. M., Yamamoto, Y., 2007. Noise-induced large-scale phase synchronization of human-brain activity associated with behavioral stochastic resonance. EPL—Europhysics Letters, 80.
Kitajo, K., Nozaki, D., Ward, L. M., Yamamoto, Y., 2003. Behavioral stochastic resonance within the human brain. Physical Review Letters 90, 218103.
Landi, D., Rossini, P. M., 2010. Cerebral restorative plasticity from normal aging to brain diseases: a never-ending story. Restorative Neurology and Neuroscience 28, 349-366.
Lang, N., Rothkegel, H., Reiber, H., Hasan, A., Sueske, E., Tergau, F., Ehrenreich, H., Wuttke, W., Paulus, W., 2011. Circadian modulation of GABA-mediated cortical inhibition. Cerebral Cortex 21, 2299-2306.
Laycock, R., Crewther, D. P., Fitzgerald, P. B., Crewther, S. G., 2007. Evidence for fast signals and later processing in human V1/V2 and V5/MT+. A TMS study of motion perception. J. Neurophysiology 98, 1253-1262.
Liebetanz, D., Nitsche, M. A., Tergau, F., Paulus, W., 2002. Pharmacological approach to the mechanisms of transcranial DC-stimulation-induced after-effects of human motor cortex excitability. Brain 125, 2238-2247.
Longtin, A., 1997. Autonomous stochastic resonance in bursting neurons. Physical Review E 55, 868-876.
Manenti, R., Capp, S. F., Rossini, P. M., Miniussi, C., 2008. The role of the prefrontal cortex in sentence comprehension: an rTMS study. Cortex 44, 337-344.
Marzi, C. A., Miniussi, C., Maravita, A., Bertolasi, L., Janette, G., Rothwell, J. C., Sanes, J. N., 1998. Transcranial magnetic stimulation selectively impairs interhemispheric transfer of visuo-motor information in humans. Experimental Brain Research 118, 435-438.
Masquelier, T., Thorpe, S. J., 2007. Unsupervised learning of visual features through spike timing dependent plasticity. PLUS Computational Biology 3, e31.
Miniussi, C., Brignani, D., Pellicciari, M. C., 2012a. Combining transcranial electrical stimulation with electroencephalography: a multimodal approach. Clin. EEG and Neuroscience 43, 184-191.
Miniussi, C., Paulus, W., Rossini, P. M., 2012b. Transcranial Brain Stimulation. CRC Press, Boca Raton, FL.
Miniussi, C., Ruzzoli, M., Walsh, V., 2010. The mechanism of transcranial magnetic stimulation in cognition. Cortex 46, 128-130.
Moliadze, V., Zhao, Y., Eysel, U., Funke, K., 2003. Effect of transcranial magnetic stimulation on single-unit activity in the cat primary visual cortex. J. Physiology 553, 665-679.
Moss, F., Ward, L. M., Sannita, W. G., 2004. Stochastic resonance and sensory information processing: a tutorial and review of application. Clin. Neurophysiology 115, 267-281.
Mottaghy, F. M., Gangitano, M., Krause, B. J., Pascual-Leone, A., 2003. Chronometry of parietal and prefrontal activations in verbal working memory revealed by transcranial magnetic stimulation. Neuroimage 18, 565-575.
Nachmias, J., Sansbury, R. V., 1974. Grating contrast: discrimination may be better than detection. Vision Research 14, 1039-1042.
Nitsche, M. A., Cohen, L. G., Wassermann, E. M., Priori, A., Lang, N., Antal, A., Paulus, W., Hummel, F., Boggio, P. S., Fregni, F., Pascual-Leone, A., 2008. Transcranial direct current stimulation: state of the art 2008. Brain Stimulation 1, 206-223.
Nitsche, M. A., Liebetanz, D., Lang, N., Antal, A., Tergau, F., Paulus, W., 2003a. Safety criteria for transcranial direct current stimulation (tDCS) in humans. Clin. Neurophysiology 114, 2220-2222, author reply 2222-2223.
Nitsche, M. A., Niehaus, L., Hoffmann, K. T., Hengst, S., Liebetanz, D., Paulus, W., Meyer, B. U., 2004. MRI study of human brain exposed to weak direct current stimulation of the frontal cortex. Clin. Neurophysiology 115, 2419-2423.
Nitsche, M. A., Nitsche, M. S., Klein, C. C., Tergau, F., Rothwell, J. C., Paulus, W., 2003b. Level of action of cathodal DC polarisation induced inhibition of the human motor cortex. Clin. Neurophysiology 114, 600-604.
Nitsche, M. A., Paulus, W., 2000. Excitability changes induced in the human motor cortex by weak transcranial direct current stimulation. J. Physiology 527 (Pt 3), 633-639.
Nitsche, M. A., Paulus, W., 2011. Transcranial direct current stimulation—update 2011. Restorative Neurology and Neuroscience 29, 463-492.
Nitsche, M. A., Seeber, A., Frommann, K., Klein, C. C., Rochford, C., Nitsche, M. S., Fricke, K., Liebetanz, D., Lang, N., Antal, A., Paulus, W., Tergau, F., 2005. Modulating parameters of excitability during and after transcranial direct current stimulation of the human motor cortex. J. Physiology 568, 291-303.
Pascual-Leone, A., Walsh, V., Rothwell, J., 2000. Transcranial magnetic stimulation in cognitive neuroscience-virtual lesion, chronometry, and functional connectivity. Current Opinion in Neurobiology 10, 232-237.
Pasley, B. N., Allen, E. A., Freeman, R. D., 2009. State-dependent variability of neuronal responses to transcranial magnetic stimulation of the visual cortex. Neuron 62, 291-303.
Paulus, W., 2011. Transcranial electrical stimulation (tES-tDCS; tRNS, tACS) methods. Neuropsychological Rehabilitation 21, 602-617.
Plewnia, C., Rilk, A. J., Soekadar, S. R., Arfeller, C., Huber, H. S., Sauseng, P., Hummel, F., Gerloff, C., 2008. Enhancement of long-range EEG coherence by synchronous bifocal transcranial magnetic stimulation. European J. Neuroscience 27, 1577-1583.
Pogosyan, A., Gaynor, L. D., Eusebio, A., Brown, P., 2009. Boosting cortical activity at Beta-band frequencies slows movement in humans. Current Biology 19, 1637-1641.
Priori, A., Berardelli, A., Rona, S., Accornero, N., Manfredi, M., 1998. Polarization of the human motor cortex through the scalp. Neuroreport 9, 2257-2260.
Redman, T., Datta, A., Peterchev, A. V., 2007. In vitro modulation of endogenous rhythms by AC electric fields: syncing with clinical brain stimulation. J. Physiology 584, 369-370.
Rahnev, D. A., Maniscalco, B., Luber, B., Lau, H., Lisanby, S. H., 2012. Direct injection of noise to the visual cortex decreases accuracy but increases decision confidence. J. Neurophysiology 107, 1556-1563.
Reato, D., Rahman, A., Bikson, M., Parra, L. C., 2010. Low-intensity electrical stimulation affects network dynamics by modulating population rate and spike timing. J. Neuroscience 30, 15067-15079.
Ridding, M. C., Ziemann, U., 2010. Determinants of the induction of cortical plasticity by non-invasive brain stimulation in healthy subjects. J. Physiology 588, 2291-2304.
Rosanova, M., Casali, A., Bellina, V., Resta, F., Mariotti, M., Massimini, M., 2009. Natural frequencies of human corticothalamic circuits. J. Neuroscience 29, 7679-7685.
Rossi, S., Hallett, M., Rossini, P. M., Pascual-Leone, A., Safety of TMS Consensus Group, 2009. Safety, ethical considerations, and application guidelines for the use of transcranial magnetic stimulation in clinical practice and research. Clin. Neurophysiology 120, 2008-2039.
Roth, B. J., 1994. Mechanisms for electrical stimulation of excitable tissue. Critical Reviews in Biomedical Engineering 22, 253-305.
Rothwell, J. L, Day, B. L., Thompson, P. D., Dick, J. P., Marsden, C. D., 1987. Some experiences of techniques for stimulation of the human cerebral motor cortex through the scalp. Neurosurgery 20, 156-163.
Ruohonen, J., 2003. Background physics for magnetic stimulation. Supplements to Clin. Neurophysiology 56, 3-12.
Ruzzoli, M., Abrahamyan, A., Clifford, C. W., Marzi, C. A., Miniussi, C., Harris, J. A., 2011. The effect of TMS on visual motion sensitivity: an increase in neural noise or a decrease in signal strength. J. Neurophysiology 106, 138-143.
Ruzzoli, M., Marzi, C. A., Miniussi, C., 2010. The neural mechanisms of the effects of transcranial magnetic stimulation on perception. J. Neurophysiology 103, 2982-2989.
Sack, A. T., Linden, D. E., 2003. Combining transcranial magnetic stimulation and functional imaging in cognitive brain research: possibilities and limitations. Brain Research: Brain Research Reviews 43, 41-56.
Sandrini, M., Umilta, L, Rusconi, E., 2011. The use of transcranial magnetic stimulation in cognitive neuroscience: a new synthesis of methodological issues. Neuroscience and Biobehavioral Reviews 35, 516-536.
Schutter, D. J., Hortensius, R., 2010. Retinal origin of phosphenes to transcranial alternating current stimulation. Clin. Neurophysiology 121, 1080-1084.
Schwarzkopf, D. S., Silvanto, J., Rees, G., 2011. Stochastic resonance effects reveal the neural mechanisms of transcranial magnetic stimulation. J. Neuro-science 31, 3143-3147.
Schwiedrzik, C. M., 2009. Retina or visual cortex? The site of phosphene induction by transcranial alternating current stimulation. Frontiers in Integrative Neuro-science 3, 6.
Solar, G., Lennie, P., DePriest, D. D., 1989. Contrast adaptation in striate cortex of macaque. Vision Research 29, 747-755.
Seyal, M., Masuoka, L. K., Browne, J. K., 1992. Suppression of cutaneous perception by magnetic pulse stimulation of the human brain. Electroencephalography and Clin. Neurophysiology 85, 397-401.
Siebner, H. R., Lang, N., Rizzo, V., Nitsche, M. A., Paulus, W., Lemon, R. N., Rothwell, 2004. Preconditioning of low-frequency repetitive transcranial magnetic stimulation with transcranial direct current stimulation: evidence for homeostatic plasticity in the human motor cortex. The J. Neuroscience 24, 3379-3385.
Silvanto, J., Muggleton, N., Walsh, V., 2008. State-dependency in brain stimulation studies of perception and cognition. Trends in Cognitive Sciences 12, 447-454.
Silvanto, J., Muggleton, N. G., Cowey, A., Walsh, V., 2007. Neural adaptation reveals state-dependent effects of transcranial magnetic stimulation. Eur. J. Neuroscience 25, 1874-1881.
Solomon, J. A., 2009. The history of dipper functions. Attention, Perception, and Psychophysics 71, 435-443.
Stein, R. B., Gossen, E. R., Jones, K. E., 2005. Neuronal variability: noise or part of the signal? Nature Reviews Neuroscience 8, 389-397.
Terney, D., Chaieb, L., Moliadze, V., Antal, A., Paulus, W., 2008. Increasing human brain excitability by transcranial high-frequency random noise stimulation. J. Neuroscience 28, 14147-14155.
Thut, G., Miniussi, C., 2009. New insights into rhythmic brain activity from TMS-EEG studies. Trends in Cognitive Sciences 13, 182-189.
Thut, G., Miniussi, C., Gross, J., 2012. The functional importance of rhythmic activity in the brain. Current Biology 22, R658-R663.
Thut, G., Schyns, P. G., Gross, J., 2011a. Entrainment of perceptually relevant brain oscillations by non-invasive rhythmic stimulation of the human brain. Front. Psychology 2, 170.
Thut, G., Veniero, D., Romei, V., Miniussi, C., Schyns, P., Gross, J., 2011b. Rhythmic TMS causes local entrainment of natural oscillatory signatures. Current Biology 21, 1178-1185.
Vallar, G., Bolognini, N., 2011. Behavioural facilitation following brain stimula-tion: implications for neurorehabilitation. Neuropsychological Rehabilitation 21, 618-649.
Varela, F., Lachaux, J. P., Rodriguez, E., Martinerie, J., 2011. The brainweb: phase synchronization and large-scale integration. Nature Reviews Neuroscience 2, 229-239.
Veniero, D., Brignani, D., Thut, G., Miniussi, C., 2011. Alpha-generation as basic response-signature to transcranial magnetic stimulation (TMS) targeting the human resting motor cortex: a TMS/EEG co-registration study. Psychophysiology 48, 1381-1389.
Walsh, V., Cowey, A., 2000. Transcranial magnetic stimulation and cognitive neuroscience. Nature Reviews Neuroscience I, 73-79.
Walsh, V., Ellison, A., Battelli, L., Cowey, A., 1998. Task-specific impairments and enhancements induced by magnetic stimulation of human visual area V5. Proceedings: Biological Sciences 285, 537-543.
Walsh, V., Pascual-Leone, A., 2003. Transcranial Magnetic Stimulation: A Neurochronometrics of Mind. MIT Press, Cambridge, MA.
Walsh, V., Rushworth, M., 1999. A primer of magnetic stimulation as a tool for neuropsychology. Neuropsychologia 37, 125-135.
Ward, L. M., Doesburg, S. M., Kitajo, K., MacLean, S. E., Roggeveen, A. B., 2006. Neural synchrony in stochastic resonance, attention, and consciousness. Canadian J. Experimental Psychology 60, 319-326.
Wassermann, E. M., Epstein, C., Ziemann, U., Walsh, V., Paus, T., Lisanby, S., 2008.
Handbook of Transcranial Stimulation. Oxford University Press, Oxford, UK.
Waterston, M. L., Pack, C. C., 2010. Improved discrimination of visual stimuli following repetitive transcranial magnetic stimulation. PLoS ONE 5, e10354.
Wu, S., Amari, S., Nakahara, H., 2002. Population coding and decoding in a neural field: a computational study. Neural Computation 14, 999-1026.
Zaehle, T., Rach, S., Herrmann, C. S., 2010. Transcranial alternating current stimulation enhances individual alpha activity in human EEG. PLoS ONE 5, e13766.

Transcranial Electrical Stimulation (tES): tES (tDCS, tACS, and tRNS) is a set of noninvasive method of cortical stimulation, using weak direct currents to polarize target brain regions. The most used and best-known method is tDCS, as all considerations for the use of tDCS have been extended to the other tES methods. The hypotheses concerning the application of tDCS in cognition are very similar to those of TMS, with the exception that tDCS was never considered a virtual lesion method. tDCS can increase or decrease cortical excitability in the stimulated brain regions and facilitate or inhibit behavior accordingly. tES does not induce action potentials but instead modulates the neuronal response threshold so that it can be defined as subthreshold stimulation.

Michael A. Nitsche, and Armin Kibele. “Noninvasive brain stimulation and neural entrainment enhance athletic performance-a review.” J. Cognitive Enhancement 1.1 (2017): 73-79, discusses that non-invasive brain stimulation (NIBS) bypasses the correlative approaches of other imaging techniques, making it possible to establish a causal relationship between cognitive processes and the functioning of specific brain areas. NIBS can provide information about where a particular process occurs. NIBS offers the opportunity to study brain mechanisms beyond process localization, providing information about when activity in a given brain region is involved in a cognitive process, and even how it is involved. When using NIBS to explore cognitive processes, it is important to understand not only how NIBS functions but also the functioning of the neural structures themselves. Non-invasive brain stimulation (NIBS) methods, which include transcranial magnetic stimulation (TMS) and transcranial electric stimulation (tES), are used in cognitive neuroscience to induce transient changes in brain activity and thereby alter the behavior of the subject. The application of NIBS aims at establishing the role of a given cortical area in an ongoing specific motor, perceptual or cognitive process (Hallett, 2000; Walsh and Cowey, 2000). Physically, NIBS techniques affect neuronal states through different mechanisms. In TMS, a solenoid (coil) is used to deliver a strong and transient magnetic field, or “pulse,” to induce a transitory electric current at the cortical surface beneath the coil. (US 2004078056) The pulse causes the rapid and above-threshold depolarization of cell membranes affected by the current (Barker et al., 1985; 1987), followed by the transynaptic depolarization or hyperpolarization of interconnected neurons. Therefore, TMS induces a current that elicits action potentials in neurons. A complex set of coils can deliver a complex 3D excitation field. By contrast, in tES techniques, the stimulation involves the application of weak electrical currents directly to the scalp through a pair of electrodes (Nitsche and Paulus, 2000; Priori et al., 1998). As a result, tES induces a subthreshold polarization of cortical neurons that is too weak to generate an action potential. However, by changing the intrinsic neuronal excitability, tES can induce changes in the resting membrane potential and the postsynaptic activity of cortical neurons. This, in turn, can alter the spontaneous firing rate of neurons and modulate their response to afferent signals (Bindman et al, 1962,1964,1979; Creutzfeldt et al., 1962), leading to changes in synaptic efficacy. The typical application of NIBS involves different types of protocols: TMS can be delivered as a single pulse (spTMS) at a precise time, as pairs of pulses separated by a variable interval, or as a series of stimuli in conventional or patterned protocols of repetitive TMS (rTMS) (for a complete classification see Rossi et al., 2009). In general, the final effects of NIBS on the central nervous system depend on a lengthy list of parameters (e.g., frequency, temporal characteristics, intensity, geometric configuration of the coil/electrode, current direction), when it is delivered before (off-line) or during (on-line) the task as part of the experimental procedure (e.g., Jacobson et al., 2011; Nitsche and Paulus, 2011; Sandrini et al., 2011). In addition, these factors interact with several variables related to the anatomy (e.g., properties of the brain tissue and its location, Redman et al., 2007), as well as physiological (e.g., gender and age, Landi and Rossini, 2010; Lang et al., 2011; Ridding and Ziemann, 2010) and cognitive (e.g., Miniussi et al., 2010; Silvanto et al., 2008; Walsh et al., 1998) states of the stimulated area/subject.

Transcranial Direct Current Stimulation (tDCS): Cranial electrotherapy stimulation (DES) is a form of non-invasive brain stimulation that applies a small, pulsed electric current across a person's head to treat a variety of conditions such as anxiety, depression and insomnia. See, en.wikipedia.org/wiki/Cranial_electrotherapy_stimulation. Transcranial direct current stimulation (tDCS) is a form of neurostimulation that uses constant, low current delivered to the brain area of interest via electrodes on the scalp. It was originally developed to help patients with brain injuries or psychiatric conditions like major depressive disorder. tDCS appears to have some potential for treating depression. See, en.wikipedia.org/wiki/Transcranial_direct-current_stimulation.

tDCS is being studied for acceleration of learning. The mild electrical shock (usually, a 2-milliamp current) is used to depolarize the neuronal membranes, making the cells more excitable and responsive to inputs. Weisend, Experimental Brain Research, vol 213, p 9 (DARPA) showed that tDCS accelerates the formation of new neural pathways during the time that someone practices a skill. tDCS appears to bring about the flow state. The movements of the subjects become more automatic; they report calm, focused concentration, and their performance improves immediately. (See Adee, Sally, “Zap your brain into the zone: Fast track to pure focus”, New Scientist, No. 2850, Feb. 1, 2012, www.newscientist.com/article/mg21328501-600-zap-your-brain-into-the-zone-fast-track-to-pure-focus/).

U.S. Pat. Nos. 7,859,294; 8,706,241; 8,725,669; 9,037,224; 9,042,201; 9,095,266; 9,248,286; 9,349,178; 9,929,568; 9,693,725; 9,713,433; 20040195512; 20070179534; 20110092882; 20110311021; 20120165696; 20140142654; 20140200432; 20140211593; 20140316243; 20140347265; 20150099946; 20150174418; 20150257700; 20150327813; 20150343242; 20150351655; 20160000354; 20160038049; 20160113569; 20160144175; 20160148371; 20160148372; 20160180042; 20160213276; 20190228702; and 20160235323.

Reinhart, Robert M G. “Disruption and rescue of interareal theta phase coupling and adaptive behavior.” Proceedings of the National Academy of Sciences (2017): provide evidence for a causal relation between interareal theta phase synchronization in frontal cortex and multiple components of adaptive human behavior. Reinhart's results support the idea that the precise timing of rhythmic population activity spatially distributed in frontal cortex conveys information to direct behavior. Given prior work showing that phase synchronization can change spike time-dependent plasticity, together with Reihart's findings showing stimulation effects on neural activity and behavior can outlast a 20-min period of electrical stimulation, it is reasonable to suppose that the externally modulated interareal coupling changed behavior by causing neuroplastic modifications in functional connectivity. Reinhart suggests that we may be able to noninvasively intervene in the temporal coupling of distant rhythmic activity in the human brain to optimize (or impede) the postsynaptic effect of spikes from one area on the other, improving (or impairing) the cross-area communication necessary for cognitive action control and learning. Moreover, these neuroplastic alterations in functional connectivity were induced with a 0° phase, suggesting that inducing synchronization does not require a meticulous accounting of the communication delay between regions such as MFC and IPFC to effectively modify behavior and learning. This conforms to work showing that despite long axonal conduction delays between distant brain areas, theta phase synchronizations at 0° phase lag can occur between these regions and underlie meaningful functions of cognition and action. It is also possible that a third subcortical or posterior region with a nonzero time lag interacted with these two frontal areas to drive changes in goal-directed behavior.
Alexander W H & Brown J W (2011) Medial prefrontal cortex as an action-outcome predictor. Nature Neuroscience 14(10):1338-1344.
Alexander W H & Brown J W (2015) Hierarchical error representation: A computational model of anterior cingulate and dorsolateral prefrontal cortex. Neural Computation 27:2354-2410.
Anguera J A, et al. (2013) Video game training enhances cognitive control in older adults. Nature 501:97-101.
Aron A R, Fletcher P C, Bullmore E T, Sahakian B J, Robbins T W (2003) Stop-signal inhibition disrupted by damage to right inferior frontal gyrus in humans. Nat Neurosci 6:115-116.
Au J, et al. (2015) Improving fluid intelligence with training on working memory: a meta-analysis. Psychonomic Bulletin & Review 22:366-377.
Bellman R, Kalaba R (1959) A mathematical theory of adaptive control processes. Proc Natl Aced Sci USA 45:1288-1290.
Bibbig A, Traub R D, Whittington M A (2002) Long-range synchronization of gamma and beta oscillations and the plasticity of excitatory and inhibitory synapses: A network model. J Neurophysiol 88:1634-1654.
Botvinick M M (2012) Hierarchical reinforcement learning and decision making. Current Opinion in Neurobiology 22(8):956-962.
Botvinick M M, Braver T S, Barch D M, Carter C S, & Cohen J D (2001) Conflict monitoring and cognitive control. Psychological Review 108(3):624-652.
Bryck R L & Fisher P A (2012) Training the brain: practical applications of neural plasticity from the intersection of cognitive neuroscience, developmental psychology, and prevention science. American Psychologist 67:87-100.

Cavanagh J F, Cohen M X, & Allen J J (2009) Prelude to and resolution of an error: EEG phase synchrony reveals cognitive control dynamics during action monitoring. Journal of Neuroscience 29(1):98-105.
Cavanagh J F, Frank M J (2014) Frontal theta as a mechanism for cognitive control. Trends Cogn Sci 18:414-421.
Christie G J, Tata M S (2009) Right frontal cortex generates reward-related theta-band oscillatory activity. Neuroimage 48:415-422.
Cohen M X, Wilmes K, Vijver Iv (2011) Cortical electrophysiological network dynamics of feedback learning. Trends Cogn Sci 15:558-588.
Corbett A, et al. (2015) The effect of an online cognitive training package in healthy older adults: An online randomized controlled trial. J Am Med Dir Assoc 16:990-997.
Dale A M & Sereno M I (1993) Improved localization of cortical activity by combining EEG and MEG with MRI cortical surface reconstruction: A linear approach. Journal of Cognitive Neuroscience 5:162-176.
Dailey J W, Robbins T W (2017) Fractionating impulsivity: Neuropsychiatric implications. Nat Rev Neurosci 18:158-171.
Delorme A & Makeig S (2004) EEGLAB: An open source toolbox for analysis of single-trial EEG dynamics including independent component analysis. Journal of Neuroscience Methods 134(1):9-21.
Diamond A & Lee K (2011) Interventions and programs demonstrated to aid executive function development in children 4-12 years of age. Science 333:959964.
Engel A K, Fries P, Singer W (2001) Dynamic predictions: Oscillations and synchrony in top-down processing. Nat Rev Neurosci 2:704-716.
Fairclough S H & Houston K (2004) A metabolic measure of mental effort. Biological Psychology 66:177-190.
Fell J, Axmacher N (2011) The role of phase synchronization in memory processes. Nat Rev Neurosci 12:105-118.
Fitzgerald K D, et al. (2005) Error-related hyperactivity of the anterior cingulate cortex in obsessive-compulsive disorder. Biol Psychiatry 57:287-294.
Foti D, Weinberg A, Dien J, Hajcak G (2011) Event-related potential activity in the basal ganglia differentiates rewards from nonrewards: Temporospatial principal components analysis and source localization of the feedback negativity. Hum Brain Mapp 32:2207-2216.
Fuchs M, Drenckhahn R, Wischmann H A, & Wagner M (1998) An improved boundary element method for realistic volume-conductor modeling. IEEE Trans Biomed Eng 45(8):980-997.
Gailliot M T & Baumeister R F (2007) The physiology of willpower: linking blood glucose to self-control. Personality and Social Psychology Review 11(4):303-327.
Gandiga P, Hummel F, & Cohen L (2009) Transcranial D C stimulation (tDCS): A tool for double-blind sham-controlled clinical studies in brain stimulation. Clinical Neurophysiology 117(4):845-850.
Gregoriou G G, Gotts Si, Zhou H, Desimone R (2009) High-frequency, long-range coupling between prefrontal and visual cortex during attention. Science 324:1207-1210.
Hillman C H, Erickson K I, & Kramer A F (2008) Be smart, exercise your heart: exercise effects on brain and cognition. Nature Reviews Neuroscience 9(1):5865.
Holroyd C B & Yeung N (2012) Motivation of extended behaviors by anterior cingulate cortex. Trends in Cognitive Sciences 16:122-128.
Inzlicht M, Schmeichel B J, & Macrae C N (2014) Why self-control seems (but may not be) limited. Trends in Cognitive Sciences 18(3):127-133.
Jennings J R & Wood C C (1976) The e-adjustment procedure for repeated measures analyses of variance. Psychophysiology 13:277-278.
Kanai R, Chaieb L, Antal A, Walsh V, & Paulus W (2008) Frequency-dependent electrical stimulation of the visual cortex. Current Biology 18(23):1839-1843.
Kayser J & Tenke C E (2006) Principal components analysis of Laplacian waveforms as a generic method for identifying estimates: II. Adequacy of low density estimates. Clinical Neurophysiology 117:369-380.
Kramer A F & Erickson K I (2007) Capitalizing on cortical plasticity: influence of physical activity on cognition and brain function. Trends in Cognitive Sciences 11:342-348.
Kurland J, Baldwin K, Tauer C (2010) Treatment-induced neuroplasticity following intensive naming therapy in a case of chronic wernicke's aphasia. Aphasiology 24: 737-751.
Lachaux J P, Rodriguez E, Martinerie J, & Varela F J (1999) Measuring phase synchrony in brain signals. Human Brain Mapping 8:194-208.
Lennie P (2003) The cost of cortical computation. Current Biology 13:493-497.
Luft C D B, Nolte G, & Bhattacharya J (2013) High-learners present larger midfrontal theta power and connectivity in response to incorrect performance feedback. Journal of Neuroscience 33(5):2029-2038.
Luft C D B, Nolte G, Bhattacharya J (2013) High-learners present larger mid-frontal theta power and connectivity in response to incorrect performance feedback. J Neurosci 33:2029-2038.
Marco-Pallares J, et al. (2008) Human oscillatory activity associated to reward processing in a gambling task. Neuropsychologia 49:241-248.
Marcora S M, Staiano W, & Manning V (2009) Mental fatigue impairs physical performance in humans. Journal of Applied Physiology 106:857-864.
Miltner W H R, Braun H, & Coles M G H (1997) Event-related brain potentials following incorrect feedback in a time-estimation task: evidence for a “generic” neural system for error detection. Journal of Cognitive Neuroscience 9:788-798.
Noury N, Hipp J F, Siegel M (2019) Physiological processes non-linearly affect electrophysiological recordings during transcranial electric stimulation. Neuroimage 140: 99-109.
Oostenveld R, Fries P, Maris E, & Schoffelen J M (2011) FieldTrip: Open source software for advanced analysis of MEG, EEG, and invasive electrophysiological data. Computational Intelligence and Neuroscience 2011:1-9.
Owen A M, et al. (2010) Putting brain training to the test. Nature 465:775-778.
Pascual-Marqui R D (2002) Standardized low-resolution brain electromagnetic tomography (sLORETA): technical details. Methods & Findings in Experimental & Clinical Pharmacology 24:5-12.
Paulus W (2010) On the difficulties of separating retinal from cortical origins of phosphenes when using transcranial alternating current stimulation (tACS). Clinical Neurophysiology 121:987-991.
Poreisz C, Boros K, Antal A, & Paulus W (2007) Safety aspects of transcranial direct current stimulation concerning healthy subjects and patients. Brain Research Bulletin 72(4-9):208-214.
Raichle M E & Mintun M A (2009) Brain work and brain imaging. Annual Review of Neuroscience 29:449-478.
Reinhart R M G & Woodman G F (2014) Causal control of medial-frontal cortex governs electrophysiological and behavioral indices of performance monitoring and learning. Journal of Neuroscience 34(12):4214-4227.
Reinhart R M G & Woodman G F (2015) Enhancing long-term memory with stimulation tunes visual attention in one trial. Proceedings of the National Academy of Sciences of the USA 112(2):625-630.
Reinhart R M G, Cosman J D, Fukuda K, & Woodman G F (2017) Using transcranial direct-current stimulation (tDCS) to understand cognitive processing. Attention, Perception & Psychophysics 79(1):3-23.
Reinhart R M G, Woodman G F (2014) Oscillatory coupling reveals the dynamic reorganization of large-scale neural networks as cognitive demands change. J Cogn Neurosci 28:175-188.
Reinhart R M G, Xiao W, McClenahan L, & Woodman G F (2016) Electrical stimulation of visual cortex can immediately improve spatial vision. Current Biology 25(14):1867-1872.
Reinhart R M G, Zhu J, Park S, & Woodman G F (2015) Medial-frontal stimulation enhances learning in schizophrenia by restoring prediction-error signaling. Journal of Neuroscience 35(35):12232-12240.
Reinhart R M G, Zhu J, Park S, & Woodman G F (2015) Synchronizing theta oscillations with direct-current stimulation strengthens adaptive control in the human brain. Proceedings of the National Academy of Sciences of the USA 112(30):9448-9453.
Ridderinkhof K R, Ullsperger M, Crone E A, & Nieuwenhuis S (2004) The role of the medial frontal cortex in cognitive control. Science 306:443-447.
Salinas E, Sejnowski T J (2001) Correlated neuronal activity and the flow of neural information. Nat Rev Neurosci 2:539-550.
Schnitzler A, Gross J (2005) Normal and pathological oscillatory communication in the brain. Nat Rev Neurosci 6:285-296.
Schutter D J & Hortensius R (2010) Retinal origin of phosphenes to transcranial alternating current stimulation. Clinical Neurophysiology 121(7):1080-1084.
Shallice T, Gazzaniga M S (2004) The fractionation of supervisory control. The Cognitive Neuroscience (MIT Press, Cambridge, M A), pp 943-956.
Shenhav A, Botvinick M M, & Cohen J D (2013) The expected value of control: An integrative theory of anterior cingulate cortex function. Neuron 79:217-240.
Shenhav A, Cohen J D, & Botvinick M M (2016) Dorsal anterior cingulate cortex and the value of control. Nature Neuroscience 19:1286-1291.
Siegel M, Donner T H, Engel A K (2012) Spectral fingerprints of large-scale neuronal interactions. Nat Rev Neurosci 13:121-134.
Srinivasan R, Winter W R, Ding J, & Nunez P L (2007) EEG and MEG coherence: measures of functional connectivity at distinct spatial scales of neocortical dynamics. Journal of Neuroscience Methods 166(1):41-52.
Tang Y, et al. (2010) Short term mental training induces white-matter changes in the anterior cingulate. Proceedings of the National Academy of Sciences 107:16649-16652.
Tang Y Y, et al. (2009) Central and autonomic nervous system interaction is altered by short term meditation. Proceedings of the National Academy of Sciences 106:8865-8870.
Thrane G, Friborg O, Anke A, Indredavik B (2014) A meta-analysis of constraint-induced movement therapy after stroke. J Rehabil Med 46:833-842.
Uhlhaas P J, Singer W (2006) Neural synchrony in brain disorders: Relevance for cognitive dysfunctions and pathophysiology. Neuron 52:155-168.
Uhlhaas P J, Singer W (2010) Abnormal neural oscillations and synchrony in schizophrenia. Nat Rev Neurosci 11:100-113.
van de Vijver I, Ridderinkhof K R, & Cohen M X (2011) Frontal oscillatory dynamics predict feedback learning and action adjustment. Journal of Cognitive Neuroscience 23:4106-4121.
van Driel J, Ridderinkhof K R, & Cohen M X (2012) Not all errors are alike: Theta and alpha EEG dynamics relate to differences in error-processing dynamics. Journal of Neuroscience 32(47):16795-16806.
van Meel C S, Heslenfeld D J, Oosterlaan J, Sergeant J A (2007) Adaptive control deficits in attention-deficit/hyperactivity disorder (ADHD): The role of error processing. Psychiatry Res 151:211-220.
Varela F, Lachaux J P, Rodriguez E, Martinerie J (2001) The brainweb: Phase synchronization and large-scale integration. Nat Rev Neurosci 2:229-239.
Velligan D I, Ritch J L, Sui D, DiCocco M, Huntzinger C D (2002) Frontal systems behavior scale in schizophrenia: Relationships with psychiatric symptomatology, cognition and adaptive function. Psychiatry Res 113:227-236.
Vicente R, Gollo L L, Mirasso C R, Fischer I, Pipa G (2008) Dynamical relaying can yield zero time lag neuronal synchrony despite long conduction delays. Proc Natl Aced Sci USA 105:17157-17162.
Wagner M, Fuchs M, & Kastner J (2007) SWARM: sLORETA-weighted accurate minimum norm inverse solutions. International Congress Series 1300:185-188.
Wang X J (2010) Neurophysiological and computational principles of cortical rhythms in cognition. Physiol Rev 90:1195-1268.
Wolpert D M, Diedrichsen J, & Flanagan J R (2011) Principles of sensorimotor learning. Nature Reviews Neuroscience 12:739-751.
Xue S, Tang Y Y, Tang R, & Posner M I (2014) Short-term meditation induces changes in brain resting EEG theta networks. Brain & Cognition 87:1-6
Zatorre R I, Fields R D, & Johansen-Berg H (2012) Plasticity in gray and white: neuroimaging changes in brain structure during learning. Nature Neuroscience 15(4):528-536.
See, Daniel Stevenson. “Intro to Transcranial Direct Current Stimulation (tDCS)” (Mar. 26, 2017) (www.slideshare.net/DanielStevenson27/intro-to-transcranial-direct-curent-stimulation-tdcs).

High-Definition-tDCS: High-Definition transcranial Direct Current Stimulation (HD-tDCS) was invented at The City University of New York with the introduction of the 4×1 HD-tDCS montage. The 4×1 HD-tDCS montage allows precise targeting of cortical structures. The region of current flow is circumscribed by the area of the 4× ring, such that decreasing ring radius increases focality. 4×1 HD-tDCS allows for unifocal stimulation, meaning the polarity of the center 1× electrode will determine the direction of neuromodulation under the ring. This is in contrast to conventional tDCS where the need for one anode and one cathode always produces bidirectional modulation (even when an extra-cephalic electrode is used). 4×1 HD-tDCS thus provides the ability not only to select a cortical brain region to target, but to modulate the excitability of that brain region with a designed polarity without having to consider return counter-electrode flow.

Transcranial Alternative Current Stimulation (tACS): Transcranial alternating current stimulation (tACS) is a noninvasive means by which alternating electrical current applied through the skin and skull entrains in a frequency-specific fashion the neural oscillations of the underlying brain. See, en.wikipedia.org/wiki/Transcranial_alternating_ current_stimulation

U.S. Pub. App. No. 20170197081 discloses transdermal electrical stimulation of nerves to modify or induce a cognitive state using transdermal electrical stimulation (TES).

Transcranial alternating current stimulation (tACS) is a noninvasive means by which alternating electrical current applied through the skin and skull entrains in a frequency-specific fashion the neural oscillations of the underlying brain. See, en.wikipedia.org/wiki/Transcranial_alternating_current_stimulation;

U.S. Pat. Nos. 6,804,558; 7,149,773; 7,181,505; 7,278,966; 9,042,201; 9,629,568; 9,713,433; 20010051787; 20020013613; 20020052539; 20020082665; 20050171410; 20140211593; 20140316243; 20150174418; 20150343242; 20160000354; 2016038049; 20160106513; 2016213276; 2016228702; 2016232330; 20160235323; and 20170113056.

Transcranial Random Noise Stimulation (tRNS): Transcranial random noise stimulation (tRNS) is a non-invasive brain stimulation technique and a form of transcranial electrical stimulation (tES). See, en.wikipedia.org/wiki/Transcranial_random_noise_stimulation; U.S. Pat. Nos. 9,198,733; 9,713,433; 20140316243; 20160038049; and 2016213276.

The stimulus may comprise transcranial pulsed current stimulation (tPCS). See:
Shapour Jaberzadeh, Andisheh Bastani, Maryam Loghi, “Anodal transcranial pulsed current stimulation: A novel technique to enhance corticospinal excitability,” DIM. Neurophysiology, Volume 125, Issue 2, February 2014, Pages 344-351, doi.org/10.1016/j.clinph.2013.08.025;
earthpulse.net/tpcs-transcranial-pulsed-current-stimulation/; help.foc.us/article/16-tpcs-transcranial-pulsed-current-stimulation.

Transcranial Magnetic Stimulation: Transcranial magnetic stimulation (TMS) is a method in which a changing magnetic field is used to cause electric current to flow in a small region of the brain via electromagnetic induction. During a TMS procedure, a magnetic field generator, or “coil”, is placed near the head of the person receiving the treatment. The coil is connected to a pulse generator, or stimulator, that delivers a changing electric current to the coil. TMS is used diagnostically to measure the connection between the central nervous system and skeletal muscle to evaluate damage in a wide variety of disease states, including stroke, multiple sclerosis, amyotrophic lateral sclerosis, movement disorders, and motor neuron diseases. Evidence is available suggesting that TMS is useful in treating neuropathic pain, major depressive disorder, and other conditions.

See, en.wikipedia.org/wiki/Transcranial_magnetic_stimulation,

See U.S. Pat. Nos. 4,296,756; 4,367,527; 5,069,218; 5,088,497; 5,359,363; 5,384,588; 5,459,536; 5,711,305; 5,877,801; 5,891,131; 5,954,662; 5,971,923; 6,188,924; 6,259,399; 6,487,441; 6,603,502; 7,714,936; 7,844,324; 7,856,264; 8,221,330; 8,655,817; 8,706,241; 8,725,669; 8,814,115; 9,037,224; 9,042,201; 9,095,266; 9,149,195; 9,248,286; 9,265,450; 9,414,776; 9,445,713; 9,713,433; 20020097332; 20040088732; 20070179534; 20070249949; 20080194981; 20090006001; 20110004412; 2011000712; 20110087127; 20110092882; 20110119212; 20110137371; 20120165696; 20120296569; 20130339043; 20114142654; 20149163320; 20140200432; 2014901593; 20149257047; 20149279746; 20149316243; 20149350369; 20150065803; 20150099946; 20150148617; 20150174418; 20150257700; 20150327813; 20150343242; 20150351655; 20160038049; 20160149306; 20160144175; 20160213276; 20160235323; 20160284082; 20160306942; 20160317077; 20170084175; and 20170113056.

PEMF: Pulsed electromagnetic field (PEMF) when applied to the brain is referred to as Transcranial magnetic stimulation, and has been FDA approved since 2008 for use in people who failed to respond to antidepressants. Weak magnetic stimulation of the brain is often called transcranial pulsed electromagnetic field (tPEMF) therapy. See, en.wikipedia.org/wiki/Pulsed_electromagnetic_field_therapy.

See. U.S. Pat. Nos. 7,280,861; 8,343,027; 8,415,123; 8,430,805; 8,435,166; 8,571,642; 8,657,732; 8,775,340; 8,961,385; 8,968,172; 9,002,477; 9,005,102; 9,278,231; 9,320,913; 9,338,641; 9,387,338; 9,415,233; 9,427,598; 9,433,797; 9,440,089; 9,610,459; 8,630,004; 8,656,096; 20030181791; 20060129022; 20100057655; 20100197993; 2012101544; 20120116149; 20120143285; 20120253101; 20130013339; 20140213843; 20140213844; 2014920726; 20149228620; 20149303425; 20160235983; 20170087367; and 20170165496.

Deep Brain Stimulation (DBS): Deep brain stimulation (DBS) is a neurosurgical procedure involving the implantation of a medical device called a neurostimulator (sometimes referred to as a ‘brain pacemaker’), which sends electrical impulses, through implanted electrodes, to specific targets in the brain (brain nuclei) for the treatment of movement and neuropsychiatric disorders. See, en.wikipedia.org/wiki/Deep_brain_stimulation;

See. U.S. Pat. Nos. 6,539,263; 6,671,555; 6,959,215; 6,990,377; 7,006,872; 7,010,351; 7,024,247; 7,079,977; 7,146,211; 7,146,217, 7,149,572; 7,174,206; 7,184,837; 7,209,787; 7,221,81; 7,231,254; 7,236,830; 7,236,831; 7,239,92; 7,242983; 7,24284; 7,252,090; 7,257,439; 7,267,644; 7,277,750; 7,280,867; 7,282,030; 7,299,096; 7,302,298; 7,305,268; 7,313,442; 7,321,837; 7,324,851; 7,346,382; 7,353,064; 7,493,820; 7,437,196; 7,463,927; 7,483,747; 7,499,752; 7,565,199; 7,565,200; 7,577,481; 7,582,062; 7,594,88; 7,6603,174; 7,66,495; 7,610,096; 7,617,002; 7,620,456; 7,623,928; 7,624,293; 7,629,88; 7,670,838; 7,672730; 7,676,263; 7,680,526; 7,680,549; 7,684,866; 7,684,867; 7,715,919; 7,725,192; 7,729,773; 7,742,820; 7,747,325; 7,747,326; 7,756,584; 7,7689,464; 7,775,993; 7,822,481; 7,831,305; 7,853,322; 7,853,323; 7,853,329; 7,856,264; 7,860,548; 7,894,903; 7,899,545; 7,904,134; 7,908,00; 7,917,206; 7,917,2257,930,035; 7,933,646; 7,945,330; 7,957,797; 7,957,80; 7,976,465; 7,983,762; 7,991,477; 8,000,794; 8,000,795; 8,005,534; 8,027,730; 8,031,076; 8,03222; 8,050,768; 8,055,348; 8,065,012; 8,073,546; 8,082033; 8,09254; 8,121,694; 8,126,567; 8,126,568; 8,135,472; 8,145,295; 8,150,523; 8,150,524; 8,160,680; 8,180,436; 8,180,601; 8,187,181; 8,195,298; 8,195,300; 8,200,349; 8,223,023; 8,229,55; 8,233,990; 8,239,029; 8,244,347; 8,249,718; 8,262714; 8,280,517; 8,290,596; 8,295,934; 8,295,935; 8,301,257; 8,303,636; 8,308,661; 8,315,703; 8,315,710; 8,326,420; 8,326,433; 8,332038; 8,332,041; 8,346,365; 8,364,271; 8,364,272; 8,374,703; 8,378,952; 8,3800,314; 8,388,555; 8,396,565; 8,398,692; 8,491,666; 8,412,335; 8,433,414; 8,437,861; 8,447,392; 8,447,411; 8,456,308; 8,463,374; 8,463,387; 8,467,877; 8,475,506; 8,504,150; 8,506,468; 8,5120; 8,515,549; 8,515,550; 8,538,536; 8,538,543; 8,543,214; 8,554,325; 8,565,883; 8,565,886; 8,574,279; 8,579,786; 8,579,834; 8,583,230; 8,583,252; 8,588,89; 8,588,92; 8,588,933; 8,58,316; 8,594,798; 8,603,790; 8,606,360; 8,606,361; 8,644,945; 8,649,845; 8,655,817; 8,660,642; 8,675,945; 8,676,324; 8,676,330; 8,684,92; 8,690,748; 8,694,087; 8,694,092; 8,696,722; 8,7001,74; 8,706,237; 8,706,241; 8,708,934; 8,716,447; 8,718,777; 8,725,243; 8,725,669; 8,729,049; 8,731,656; 8,734,498; 8,738,136; 8,738,149; 8,751,008; 8,751,011; 8,755,901; 8,758,274; 8,761,88; 8,762,165; 8,768,718; 8,774,923; 8,781,597; 8,788,033; 8,788,044; 8,788,055; 8,792,972; 8,792991; 8,805,518; 8,815,582; 8,821,558; 8,825,166; 8,831,731; 8,834,392; 8,834,546; 8,843,201; 8,843,210; 8,849,497; 8,849,632; 8,855,773; 8,855,775; 8,868,172; 8,868,173; 8,868,201; 8,886,302; 8,892207; 8,900,24; 8,903,486; 8,903,494; 8,906,360; 8,909,345; 8,910,630; 8,914,115; 8,148,118; 8,918,176; 8,910,178; 8,910,813; 8,926,959; 8,929,991; 8,932,562; 8,934,979; 8,936,629; 8,938,290; 8,942,817; 8,945,006; 8,951,201; 8,956,363; 8,958,870; 8,962,58; 8,965,513; 8,965,514; 8,974,365; 8,977,362; 8,983,155; 8,983,620; 8,983,628; 8,983,621; 8,887,178; 8,111,878; 9,113,290; 9,114,823; 9,020,590; 9,020,612; 9,020,789; 9,022,930; 9,026,072; 9,037,224; 9,037,254; 9,037,256; 9,042,201; 9,142,988; 9,043,001; 9,044,188; 9,150,470; 9,050,471; 9,061,153; 9,163,643; 9,072,832; 9,172,870; 9,172,805; 9,079,039; 9,079,949; 9,100,148; 9,114,885; 9,114,896; 9,148,900; 9,009,713; 9,055,266; 9,101,690; 9,011,759; 9,110,766; 9,113,801; 9,126,050; 9,135,490; 9,421,078; 9,167,976; 9,167,977; 9,167,978; 9,173,609; 9,174,055; 9,175,095; 9,179,850; 9,179,875; 9,186,510; 9,187,745; 9,190,563; 9,204,838; 9,781,411; 9,211,417; 9,215,290; 9,220,917; 9,227,056; 9,233,245; 9,233,246; 9,235,685; 9,238,142; 9,238,150; 9,248,20; 9,248,286; 9,248,288; 9,248,296; 9,249,200; 9,249,234; 9,254,383; 9,724,307, 9,259,591; 9,271,674; 9,272,091; 9,272,139; 9,272,153; 9,278,159; 9,284,353; 9,289,143; 9,809,595; 9,209,603; 9,209,609; 9,295,839; 9,302,103; 9,302,119; 9,302,149; 9,302,116; 9,308,372; 9,308,392; 9,309,296; 9,310,985; 9,314,190; 9,320,901; 9,320,914; 9,327,071; 9,333,351; 9,349,588; 9,348,974; 9,352,156; 9,357,949; 9,358,381; 9,358,398; 9,358,44; 9,360,472; 9,364,665; 9,364,679; 9,365,628; 9,375,564; 9,375,571; 9,375,573; 9,381,346; 9,387,320; 9,393,411; 9,393,410; 9,394,347; 9,399,134; 9,399,144; 9,493,001; 9,493,108; 9,908,530; 9,411,935; 9,414,776; 9,415,28; 9,415,222; 9,421,25; 9,740,373; 9,840,379; 9,427,581; 9,427,585; 9,439,150; 9,449,063; 9,449,164; 9,449,070; 9,440,084; 9,452,287; 9,453,212; 9,458,208; 9,463,327; 9,474,903; 9,480,841; 9,480,845; 9,486,632; 9,498,628; 9,501,829; 9,505,817; 9,517,020; 9,522,278; 9,522,288; 9,526,902; 9,526,913; 9,526,914; 9,533,148; 9,533,150; 9,530,951; 9,545,510; 9,561,380; 9,566,426; 9,579,247; 9,586,053; 9,592,004; 9,592,387; 9,592,388; 9,597,493; 9,597,44; 9,597,550; 9,597,504; 9,604,1056; 9,604,106; 9,604,1073; 9,613,184; 9,615,789; 9,622,675; 9,622,701; 9,623,249; 9,623,241; 9,629,548; 9,630,011; 9,636,185; 9,642,552; 9,643,015; 9,643,017; 9,643,19; 9,648,438; 9,649,439; 9,649,501; 9,656,068; 9,656,078; 9,662,502; 9,697,336; 9,706,957; 9,713,433; 9,717,920; 9,724,517; 9,729,252; 20020087201; 20020091419; 20020188330; 20030088274; 20030097159; 20030097161; 20030125786; 20030130706; 20030181955; 20049133110; 20049133118; 20049133120; 20049133248; 20040133390; 20049138516; 20049138517; 20049138510; 20049138536; 20049138580; 20040138581; 20040138647; 20040138711; 20040152950; 20040158119; 20040158298; 20050021105; 20050060001; 20050060007; 20050060008; 20050060008; 20050060010; 20050065427; 20050124848; 20050154425; 20050154426; 20050182389; 20050209512; 20050222522; 20050240253; 20050267011; 20060004422; 20060015153; 20060064138; 20060069415; 20060100671; 20060106274; 20060106430; 20060149337; 20060155348; 20060155495; 2006016120; 20060161384; 20060167370; 20060195155; 20060200206; 2006002090; 20060217781; 2006022442; 20060239482; 20060241710; 20070000372; 20070014454; 20070025608; 20070027486; 20070027498; 20070027498; 20070027500; 20070027501; 20070032834; 20070043401; 20070060974; 20070066915; 20070100278; 20070100389; 20070100392; 20070100390; 20070118197; 20070129768; 20070129774; 20070142874; 20070150026; 20070150029; 20070179534; 20070179558; 20070225774; 20070250119; 20070276441; 20080009772; 20080015459; 20080033503; 20080033508; 20080045775; 20080046012; 20080046035; 20080058773; 20080064934; 20080071150; 20080071326; 20080097553; 20080103547; 20080103548; 20080109050; 20080125829; 20080154331; 20080154332; 20080157980; 20080161700; 20080161879; 20080161880; 20080161881; 2008016282; 20080183097; 20080208285; 2008025112; 20080228238; 20080269812; 20080288843; 20080275526; 20080281381; 20080288018; 20090018462; 20090076567; 20090082828; 20090093862; 20090099627; 20090105785; 20080112273; 20080112277; 20080112278; 20080112279; 20080112280; 20090118786; 20090118787; 20090163982; 20090192556; 20090210018; 2009026288; 20090234418; 20090284788; 20090284954; 20090264955; 20090264956; 20090264957; 20090284958; 20090284967; 20090281594; 20090287035; 20090287271; 20090287272; 20090287273; 20090287274; 20090287467; 20090299126; 20090299435; 20090306491; 20090306741; 20090312808; 20090312817; 20090319000; 20090319001; 20090326604; 20100004500; 20100010383; 20100010388; 20100010391; 20100010392; 20100010571; 20100010572; 20100010573; 20100010574; 20100010575; 20100010576; 20100010577; 20100010578; 20100010579; 20100010580; 20100010584; 20100010585; 20100010587; 20100010588; 20100010588; 20100010590; 20100016783; 20100045467; 20100049276; 20100057158; 20100057160; 20100070001; 20100076525; 20100114237; 20100114272; 20100121415; 20100131030; 20100145427; 20100191305; 20100198090; 20100222845; 20100241020; 20100256592; 20100274106; 20100274141; 20100274147; 20100274305; 20100280334; 20100280335; 20100280500; 20100280571; 20100280574; 20100280579; 20100292802; 20110004270; 20110009928; 2011002970; 20110022981; 20110028798; 20110028798; 20110034812; 20110040356; 20110040546; 20110040547; 20110082522; 20110092882; 20110093033; 20110106206; 20110112590; 20110119212; 20110137371; 20110137381; 20110160796; 20110172554; 20110172562; 201101725864; 20110172567; 20110172738; 20110172743; 20110172927; 20110184487; 20110191275; 20110208012; 20110208284; 20110213222; 20110230701; 20110238130; 20110238136; 20110245734; 20110257501; 20110270348; 20110275927; 20110276107; 20110307030; 20110307079; 20110313288; 20110313487; 20110319726; 20110319975; 20120016430; 20120016432; 20120016435; 20120022340; 20120022611; 20120041498; 20120046531; 20120046715; 20120053508; 20120089205; 20120108998; 20120109020; 20120116244; 20120116475; 20120157963; 20120165696; 20120165898; 20120179071; 20120179228; 20120184801; 20120185020; 20120195860; 20120197322; 20120209346; 20120253421; 20120253428; 20120253442; 20120265287; 20120271148; 20120271183; 20120271188; 20120271374; 20120271375; 20120271376; 20120271380; 20120277833; 20120289868; 20120290058; 20120302912; 20120303087; 20120310050; 20120316630; 20130018435; 20130066392; 20130066394; 20130066395; 20130073022; 20130090706; 20130102918; 20130104066; 20130116578; 20130116748; 20130123568; 20130123684; 20130131746; 20130131753; 20130131755; 20130138176; 20130138177; 20130144353; 20130150921; 20130178913; 20130184781; 20130184792; 20130197401; 2013021183; 20130218232; 2013021881; 20130226261; 20130231708; 20130231716; 2013023172; 20130238048; 20130238050; 20130245466; 20130245486; 20130245711; 20130245712; 20130281758; 20130281811; 20130282075; 20130289385; 20130310908; 20130317474; 20130317568; 20130317580; 20130338526; 20130338738; 20140005743; 20140005744; 20140025133; 20140039577; 20140058288; 20140066796; 20140074060; 20140074178; 20140074180; 20140081071; 20140081347; 20140107397; 20140107398; 20140107728; 20140122378; 20140135642; 20140135886; 20140142854; 20140142868; 20140148872; 20140163627; 2014001894; 20140180358; 20140194720; 20140194728; 20140211593; 20140213842; 20140222013; 20140237073; 20140243613; 20140243926; 20140243934; 20140249396; 20140249445; 20140257047; 20140257437; 20140257438; 20140276185; 20140277282; 20140277286; 20140279746; 20140296646; 20140309614; 20140323924; 20140323946; 20140324118; 20140324138; 20140330334; 20140330335; 20140330345; 20140350634; 20140350636; 20140358024; 20140358198; 20140364721; 20140371515; 20150005680; 20150012057; 20150018698; 2015002540; 2015002542; 20150025610; 2015003278; 20150038822; 20150039066; 20150065831; 20150073505; 20150088224; 20150088220; 20150119688; 20150119889; 20150134931; 20150142082; 20150174496; 20150174410; 20150190636; 20150190637; 20150196246; 20150202447; 20150223721; 20150231395; 20150231397; 20150238693; 20150238765; 20150245781; 20150251016; 20150254413; 20150257700; 20150265207; 20150265830; 20150265836; 20150273201; 20150273223; 20150203379; 20150290453; 20150290454; 20150297893; 20150306391; 20150321000; 20150327813; 2015034305; 20150343242; 20150352363; 20150360026; 20150360039; 20150366482; 20150374983; 20160001065; 20160001096; 20160001090; 20160008600; 20160008632; 20160016014; 20160030666; 20160030749; 20160030750; 20160038049; 20160044841; 20160058358; 20160066789; 20160067494; 20160067496; 20160067526; 20160074661; 20160095546; 20160096025; 20160106997; 20160120437; 2016012114; 2016010116; 2016013642; 20160136430; 20160136443; 20160144175; 20160144186; 20160147964; 2016015162; 20160158553; 20160184596; 20160199662; 20160206380; 20160213276; 20160213314; 2016022082; 20160220850; 20160228204; 20160220649; 20160228702; 20160228705; 20160235323; 20160249846; 20160250473; 20160256690; 20160256691; 20160256693; 20160263380; 20160263393; 20160278870; 20160279410; 20160279417; 20160287436; 20160207869; 20160207889; 20160296746; 20160303322; 20160317077; 20160317824; 20160325111; 20160331970; 20160339243; 20160342762; 20160346542; 20160361549; 20160367800; 2016037525; 20170007820; 20170007828; 20170014625; 20170014630; 20117020161; 20170036024; 20170042474; 20170042713; 20170043167; 20170043178; 20170050046; 20170056642; 20170056663; 20170065348; 20170079573; 20117080234; 20170095670; 20170095676; 20170100591; 20170106193; 20170113046; 20170120043; 20170120052; 20170120054; 20170136230; 20170143966; 20170151433; 20170151435; 20170151436; 20170156622; 20170157410; 20170164895; 20170165481; 20170173326; 20170182285; 20170185741; 20170189685; 20170189686; 20170189687; 20170189680; 20170189689; 20170189700; 2017019700; 20170197086; 20170216595; 20170224990; 20170239486; and 20170239489.

Transcranial Pulse Ultrasound (TPU): Transcranial pulsed ultrasound (TPU) uses low intensity, low frequency ultrasound (LILFU) as a method to stimulate the brain. See, en.wikipedia.org/wiki/Transcranial_pulsed_ultrasound;

U.S. Pat. Nos. 8,591,419; 8,858,440; 8,903,494; 8,921,320; 9,002,458; 9,014,811; 9,036,844; 9,042,201; 9,061,133; 9,233,244; 9,333,334; 9,399,126; 9,403,038; 9,440,070; 9,630,029; 9,669,239; 20120259249; 20120283502; 20120289869; 20130079621; 20130144192; 20130184218; 20140058219; 20140211593; 20140228653; 20140249454; 20140316243; 20150080327; 20150133716; 20150343242; 20160143541; 20160178053; and 20160220850.

Sensory Stimulation: Light, sound or electromagnetic fields may be used to remotely convey a temporal pattern of brainwaves. See:

U.S. Pat. Nos. 5,293,187; 5,422,688; 5,447,166; 5,491,492; 5,546,943; 5,622,168; 5,649,061; 5,720,619; 5,740,812; 5,983,129; 6,050,962; 6,092,058; 6,149,586; 6,325,475; 6,377,833; 6,394,963; 6,428,490; 6,482,165; 6,503,085; 6,520,921; 6,522,906; 6,527,730; 6,556,695; 6,565,518; 6,652,458; 6,652470; 6,701,173; 6,726,624; 6,743,182; 6,746,499; 6,758,813; 6,843,774; 6,896,655; 6,996,261; 7,037,260; 7,070,571; 7,107,090; 7,120,486; 7,212,851; 7,215,994; 7,260,430; 7,269,455; 7,280,870; 7,392,079; 7,407,485; 7,463,142; 7,478,108; 7,488,294; 7,515,054; 7,567,693; 7,647,097; 7,740,592; 7,751,877; 7,831,305; 7,856,264; 7,881,780; 7,970,734; 7,972,278; 7,974,787; 7,991,461; 8,012,107; 8,032,486; 8,033,996; 8,060,194; 8,095,209; 8,209,224; 8,239,030; 8,262,714; 8,320,648; 8,358,818; 8,376,896; 8,380,316; 8,386,312; 8,386,313; 8,839,250; 8,392,253; 8,392,254; 8,392,255; 8,437,844; 8,464,288; 8,475,371; 8,483,816; 8,494,905; 8,517,912; 8,533,042; 8,545,420; 8,560,041; 8,655,428; 8,672,852; 8,682,687; 8,684,742; 8,694,157; 8,706,241; 8,706,510; 8,738,395; 8,753,296; 8,762,202; 8,764,673; 8,768,022; 8,788,030; 8,790,255; 8,790,297; 8,821,376; 8,838,247; 8,864,310; 8,872,641; 8,888,723; 8,915,871; 8,938,288; 8,938,301; 8,942,813; 8,955,010; 8,955,974; 8,958,882; 8,864,290; 8,971,936; 8,883,588; 8,992,230; 8,998,828; 9,004,687; 9,060,671; 9,101,279; 9,135,221; 9,142,145; 9,165,472; 9,173,582; 9,179,855; 9,208,558; 9,051,978; 9,232,984; 9,241,665; 9,242,067; 9,254,098; 9,271,660; 9,275,191; 9,228,927; 9,292,858; 9,292,088; 9,320,450; 9,326,705; 9,330,206; 9,357,941; 9,396,668; 9,398,873; 9,414,780; 9,414,907; 9,424,761; 9,445,738; 9,445,763; 9,451,303; 9,451,89; 9,454,646; 9,468,977; 9,468,541; 9,483,117; 9,492,120; 9,504,420; 9,504,788; 9,526,419; 9,541,383; 9,545,208; 9,545,222; 9,545,225; 9,560,967; 9,560,984; 9,563,741; 9,582,072; 9,596,224; 9,615,746; 9,622,702; 9,622,703; 9,626,756; 9,629,560; 9,642,699; 9,648,030; 9,651,360; 9,655,573; 9,660,694; 9,672,302; 9,672,617; 9,682,322; 9,693,734; 9,694,155; 9,704,205; 9,706,910; 9710,780; RE44408; RE45766; 20020024450; 20020103428; 2002010342; 2002112732; 20020128549; 20030020081; 2003002010; 20030070685; 20030083596; 20030100844; 20030120172; 20030149351; 20030158496; 20030158497; 20030171650; 20049019257; 20049024287; 20040068172; 20049092809; 20041010146; 20049116784; 20049143170; 20049267152; 20050010091; 20050019734; 20050025704; 20050038354; 20050113713; 20050124851; 20050148828; 20050228785; 20050249253; 20050245796; 20050267343; 20050267344; 20050283053; 20060020184; 20060061544; 20060078183; 20060087746; 2006010071; 20060129277; 2006016120; 2006018866; 20060200013; 20060241710; 20060252978; 2006025297; 20070050715; 20070179534; 20070191704; 20070238934; 20070273611; 20070202220; 20070299371; 20080004550; 20080009772; 20080058668; 2080001963; 20080119763; 20080123927; 20080132383; 20080228239; 20080234113; 20080234601; 20080242521; 20080255949; 20090018418; 20090058660; 2009006269; 20090076496; 20090099474; 20090112523; 2009022928; 20090267750; 20090270687; 20090270680; 20090270692; 20090270693; 20090270694; 20090270786; 20090281490; 20090287100; 20090297000; 20090299169; 20090311655; 20090312808; 2009031217; 20090318794; 20090326604; 20100004977; 20100010289; 20100010366; 20100041949; 20100069739; 20100069780; 20100163027; 20100163020; 20100163035; 20100165593; 20100168525; 2010016852; 20100168602; 20100268055; 20100293115; 20110004412; 20110009777; 20110015515; 20110015539; 20110043759; 20110054272; 20110077548; 20110092882; 20110105859; 20110130643; 20110172500; 20110218456; 20110256520; 20110270074; 20110301488; 20110307079; 2012000457; 20112000394; 20120036004; 20120071771; 20120108909; 20120108995; 20120136274; 20120150545; 20120203130; 2012026255; 20120271377; 20120310106; 20130012004; 20130046715; 20130063434; 20130063550; 20130080127; 20130120246; 20130127980; 20130185144; 20130189663; 20130204085; 2013021238; 20130226464; 20130242262; 20130245424; 20130201759; 20130289360; 20130293844; 20130308099; 20130318546; 20149058528; 20149155714; 20149171757; 20140200432; 20149024335; 20149221866; 20149243600; 20149243614; 20140243652; 20149276130; 20149276944; 2014908614, 01420296750; 20140300532; 20149303500; 20149304773; 20149313303; 20114315168; 20149316191; 20140316192; 20140316235; 20149316248; 20149323899; 20149335489; 20149343498; 20149347491; 20149350353; 20149350431; 20149364721; 20149378810; 2015000285; 20150003698; 20150003699; 20150005649; 20150005644; 20150006186; 2015001011; 20150038869; 20150045606; 20150051663; 20150099946; 20150112499; 20150120007; 20150124220; 20150126845; 20150126873; 20150133812; 20150141773; 20150145676; 20150154888; 20150174362; 20150196800; 2015003191; 20150223731; 20150234477; 20150235088; 20150235370; 20150235441; 20150235447; 20150241705; 20150241959; 20150242575; 20150242943; 20150243100; 20150243105; 20150243106; 20150247723; 20150247975; 20150247976; 20150248169; 20150248170; 20150248787; 20150248788; 20150248788; 20150248791; 20150248792; 20150248793; 20150290453; 20150290454; 20150305685; 20150306349; 20150309563; 20150313496; 20150313539; 201503246892; 20150325151; 2015033528; 20150339363; 20150351690; 20150366497; 20150366504; 20150366656; 20150366659; 201503698864; 20150370320; 20160000354; 20160004290; 20160005320; 20160007915; 20160008620; 20160012749; 2016001528; 2016002067; 20160022206; 20160029946; 20160029965; 20160038068; 20160051187; 20160051793; 20160066830; 20160073886; 20160077547; 20160078780; 20160106950; 20160112684; 20160120436; 20160143582; 2016016609; 20160167672; 20160176053; 20160180054; 2016018950; 20160199577; 20160202755; 2016006760; 20160220439; 20160228649; 20160232625; 20160232011; 20160235323; 20160239084; 20160248994; 20160249826; 20160256100; 20160267809; 20160270656; 20160207157; 20160302711; 20160306942; 20160313790; 20160317060; 20160317383; 20160324478; 20160324580; 20160334866; 20160338644; 20160338825; 20160339300; 20160345901; 20160357256; 20160360970; 20160363483; 20170000324; 20170000325; 20170000326; 2017000032; 20170000330; 20170000331; 20170000332; 20170000333; 20170000334; 20170000335; 20170000337; 20170000349; 20170000341; 20170000342; 20170000343; 20170000345; 20170000454; 20170000683; 20170001032; 20170006931; 20170007111; 20170007115; 20170007116; 20170007122; 20170007123; 20170007165; 20170007182; 20170007450; 20170007799; 20170007843; 20170010468; 20170010470; 20170017083; 20170020447; 20170020454; 20170020627; 20170027467; 20170027651; 20170027812; 20170031449; 20170032090; 20170035344; 20170043160; 20170055900; 20170060290; 20170061034; 20170071523; 20170071537; 20170071546; 20170071551; 20170080320; 20170086729; 20170095157; 20170099479; 20170100549; 20170103441; 2017012427; 20170112671; 20170113046; 20170113056; 20170119994; 20170135597; 20170135633; 20170136264; 20170136265; 20170143248; 20170143442; 20170148349; 20170156662; 20170162072; 20170164876; 20170164878; 20170168568; 20170173262; 20170173326; 20170177023; 20170188947; 20170202633; 20170209043; 20170209094; and 20170209737.

Light Stimulation: The functional relevance of brain oscillations in the alpha frequency range (8-13 Hz) has been repeatedly investigated through the use of rhythmic visual stimulation. There are two hypotheses on the origin of steady-state visual evoked potential (SSVEP) measured in EEG during rhythmic stimulation: entrainment of brain oscillations and superposition of event-related responses (ERPs). The entrainment but not the superposition hypothesis justifies rhythmic visual stimulation as a means to manipulate brain oscillations, because superposition assumes a linear summation of single responses, independent from ongoing brain oscillations. Participants stimulated with rhythmic flickering light of different frequencies and intensities, and entrainment was measured by comparing the phase coupling of brain oscillations stimulated by rhythmic visual flicker with the oscillations induced by arrhythmic jittered stimulation, varying the time, stimulation frequency, and intensity conditions. Phase coupling was found to be more pronounced with increasing stimulation intensity as well as at stimulation frequencies closer to each participant's intrinsic frequency. Even in a single sequence of an SSVEP, non-linear features (intermittency of phase locking) was found that contradict the linear summation of single responses, as assumed by the superposition hypothesis. Thus, evidence suggests that visual rhythmic stimulation entrains brain oscillations, validating the approach of rhythmic stimulation as a manipulation of brain oscillations. See, Notbohm A, Kurths J, Herrmann C S, Modification of Brain Oscillations via Rhythmic Light Stimulation Provides Evidence for Entrainment but Not for Superposition of Event-Related Responses, Front Hum Neurosci. 2016 February 3:10:10. doi: 10.3389/fnhum.2016.00010. eCollection 2016.

It is also known that periodic visual stimulation can trigger epileptic seizures.

Cochlear Implant: A cochlear implant is a surgically implanted electronic device that provides a sense of sound to a person who is profoundly deaf or severely hard of hearing in both ears. See, en.wikipedia.org/wiki/Cochlear_implant;

See, U.S. Pat. Nos. 5,999,856; 6,354,299; 6,427,086; 6,430,443; 6,665,562; 6,873,872; 7,359,837; 7,449,806; 7,493,171; 7,610,083; 7,610,100; 7,702,387; 7,747,310; 7,765,088; 7,853,321; 7,890,176; 7,917,199; 7,920,916; 7,957,806; 8,014,870; 8,024,029; 8,065,017; 8,108,033; 8,108,042; 8,140,152; 8,165,687; 8,175,700; 8,195,295; 8,209,010; 8,224,431; 8,315,704; 8,332,024; 8,491,654; 8,433,410; 8,478,417; 8,515,541; 8,538,543; 8,560,041; 8,565,864; 8,574,164; 8,577,464; 8,577,465; 8,577,466; 8,577,467; 8,577,468; 8,577,472; 8,577,478; 8,588,941; 8,594,800; 8,644,946; 8,644,957; 8,652187; 8,676,325; 8,696,724; 8,700,183; 8,718,776; 8,768,446; 8,768,477; 8,788,057; 8,798,728; 8,798,773; 8,812,126; 8,864,806; 8,868,189; 8,929,99; 8,968,376; 8,989,868; 8,996,120; 9,002,471; 9,044,612; 9,061,132; 9,061,151; 9,095,713; 9,135,490; 9,186,503; 9,235,685; 9,242,067; 9,248,290; 9,248,291; 9,259,177; 9,302,083; 9,314,613; 9,327,068; 9,352,145; 9,352,152; 9,358,392; 8,358,393; 8,403,009; 9,409,013; 9,415,215; 9,415,216; 9,421,372; 9,432,777; 9,501,829; 9,526,902; 9,533,144; 9,545,510; 9,550,064; 9,561,380; 9,578,425; 9,592,389; 9,604,067; 9,616,227; 9,643,017; 9,648,493; 9,674,621; 9,682,32; 9,743,197; 9,744,358; 20010014818; 2010029391; 20020099412; 20030114886; 20049073273; 20050149157; 20050182388; 20050182450; 20050182467; 20050182468; 20050182468; 20050187600; 20050192847; 20050209664; 20050209665; 20050209666; 20050220451; 20050240229; 20060064149; 20060094970; 20060094971; 20060094972; 20060095091; 20060095082; 20060161217; 20060173259; 20060178709; 20060195039; 20060206165; 20060235484; 20060235489; 20060247728; 20060282123; 200602876891; 20070038284; 2007004998; 20070156180; 20070198063; 20070213785; 20070244417; 20070255155; 20070255531; 20080049376; 20080149149; 20080161886; 20080208200; 2080235468; 20080249589; 20090163980; 20090163981; 20090243756; 20090259277; 20090270944; 20090280153; 20100030287; 20100100164; 20100198282; 20100217341; 20100231327; 20100241195; 20100268055; 2010026828; 20100318160; 20110004203; 20110060382; 20110166471; 20110295344; 20110295345; 20110295346; 20110295347; 20120035690; 20120116179; 20120116741; 20120150255; 20120245655; 20120262250; 20120265270; 20130165996; 20130197944; 20130235550; 20140032512; 20149009881; 20149200623; 20149249600; 20140275847; 20149330357; 20140350634; 20150018698; 20150045607; 20150051660; 20150065831; 20150066124; 20150080674; 20150320455; 20150374986; 20150374987; 20160067485; 20160243362; 20160261962; 20170056655; 20117087354; 20170087355; 20170087356; 20170113046; 20170117866; 20170135633; and 20170182312.

Vagus Nerve Stimulation: Vagus nerve stimulation (VNS) is a medical treatment that involves delivering electrical impulses to the vagus nerve. It is used as an adjunctive treatment for certain types of intractable epilepsy and treatment-resistant depression. See, en.wikipedia.org/wiki/Vagus_nerve_stimulation;

See, U.S. Pat. Nos. 5,215,086; 5,231,988; 5,299,569; 5,335,657; 5,571,150; 5,928,272; 5,995,868; 6,104,956; 6,167,311; 6,205,358; 6,208,902; 6,248,126; 6,269,270; 6,339,725; 6,341,236; 6,356,788; 6,366,814; 6,418,344; 6,497,698; 6,549,804; 6,556,860; 6,560,486; 6,587,727; 6,591,137; 6,597,954; 6,609,030; 6,622047; 6,665,562; 6,671,556; 6,684,105; 6,708,064; 6,735,475; 6,782,292; 6,788,975; 6,873,872; 6,879,859; 6,882,881; 6,920,357; 6,961,618; 7,003,352; 7,151,961; 7,155,279; 7,167,751; 7,177,678; 7,203,548; 7,209,787; 7,228,167; 7,231,254; 7,242,984; 7,277,758; 7,22,890; 7,313,442; 7,324,851; 7,346,395; 7,366,571; 7,386,347; 7,380,144; 7,403,820; 7,418,290; 7,422,555; 7,444,184; 7,454,245; 7,457,665; 7,463,927; 7,486,869; 7,493,172; 7,499,752; 7,561,918; 7,620,455; 7,623,927; 7,623,928; 7,630,757; 7,634,317; 7,643,881; 7,653,433; 7,657,316; 7,676,263; 7,680,526; 7,684,858; 7,706,871; 7,711,432; 7,734,355; 7,736,382; 7,747,325; 7,747,326; 7,7689,461; 7,783,362; 7,800,601; 7,805,203; 7,840,280; 7,848,803; 7,853,329; 7,853,329; 7,860,548; 7,860,570; 7,865,244; 7,869,867; 7,869,884; 7,869,885; 7,890,185; 7,894,903; 7,899,538; 7,904,134; 7,904,151; 7,904,175; 7,908,008; 7,920,915; 7,925,353; 7,945,316; 7,957,796; 7,962,214; 7,962,288; 7,962,220; 7,974,688; 7,974,693; 7,974,697; 7,974,701; 7,996,079; 8,000,788; 8,027,730; 8,036,745; 8,041,418; 8,041,419; 8,046,076; 8,064,994; 8,060,911; 8,097,926; 8,108,030; 8,121,488; 8,121,538; 8,116,883; 8,150,500; 8,150,524; 8,160,696; 8,172,759; 8,180,601; 8,190,251; 8,190,264; 8,204,603; 8,209,008; 8,209,088; 8,214,035; 8,020,988; 8,224,444; 8,224,451; 8,229,558; 8,239,028; 8,260,426; 8,280,505; 8,306,627; 8,315,708; 8,315,704; 8,326,418; 8,337,404; 8,340,771; 8,346,354; 8,352,031; 8,374,696; 8,374,701; 8,379,852; 8,382,667; 8,401,634; 8,412,334; 8,412,338; 8,417,344; 8,423,155; 8,428,728; 8,452,387; 8,454,555; 8,457,747; 8,467,878; 8,478,428; 8,485,979; 8,489,185; 8,498,698; 8,515,538; 8,536,667; 8,538,523; 8,538,543; 8,548,583; 8,548,594; 8,548,604; 8,560,073; 8,562,536; 8,562,660; 8,565,867; 8,571,643; 8,571,653; 8,588,933; 8,591,419; 8,600,52; 8,603,790; 8,606,360; 8,615,309; 8,630,705; 8,634,922; 8,641,646; 8,644,954; 8,649,871; 8,65,2187; 8,660,666; 8,666,501; 8,676,324; 8,676,330; 8,684,82; 8,694,118; 8,700,163, 8,712,547; 8,716,447; 8,718,779; 8,725,243; 8,738,126; 8,744,562; 8,761,868; 8,762,065; 8,768,471; 8,781,597; 8,815,582; 8,827,912; 8,831,732; 8,843,208; 8,849,408; 8,852,100; 8,855,775; 8,858,440; 8,864,806; 8,868,172; 8,860,177; 8,874,205; 8,874,218; 8,874,227; 8,888,702; 8,914,122; 8,910,178; 8,934,967; 8,942,817; 8,945,006; 8,948,855; 8,965,514; 8,968,376; 8,972,004; 8,972,013; 8,983,155; 8,983,628; 8,983,628; 8,985,119; 8,989,863; 8,989,867; 9,014,804; 9,014,823; 9,020,582; 9,201,598; 9,020,789; 9,026,218; 9,031,655; 9,042,201; 9,042,988; 9,043,001; 9,044,180; 9,050,468; 9,056,195; 9,067,054; 9,067,070; 9,078,840; 9,088,707; 9,009,719; 9,085,303; 9,805,314; 9,810,041; 9,113,801; 9,119,533; 9,135,400; 9,138,580; 9162,051; 9,862,052; 9,174,045; 9,174,066; 9,186,060; 9,186,106; 9,204,838; 9,204,998; 9,220,910; 9,233,246; 9,233,258; 9,235,685; 9,238,150; 9,241,647; 9,242067; 9,242,082; 9,248,286; 9,248,200; 9,249,234; 9,254,383; 9,259,591; 9,285,660; 9,265,661; 9,265,662; 9,285,663; 9,265,931; 9,265,946; 9,272,145; 9,283,394; 9,284,353; 9,288,598; 9,302,109; 9,308,296; 9,314,633; 9,314,635; 9,320,900; 9,328,720; 9,332,893; 9,333,347; 9,338,654; 9,345,886; 9,358,381; 9,358,448; 9,364,674; 9,365,628; 9,375,571; 9,375,573; 9,381,346; 9,394,347; 9,399,133; 9,399,134; 9,402,994; 9,403,000; 9,403,001; 9,403,038; 9,408,022; 9,408,028; 9,415,28; 9,415,222; 9,427,581; 9,440,063; 9,458,208; 9,468,761; 9,474,852; 9,480,845; 9,492656; 9,492678; 9,501,829; 9,504,390; 9,505,817; 9,522,085; 9,522,282; 9,526,902; 9,533,147; 9,533,151; 9,538,951; 9,545,226; 9,545,510; 9,561,380; 9,566,426; 9,578,506; 9,586,047; 9,592,003; 9,592,004; 9,592,409; 9,604,067; 9,604,073; 9,610,442; 9,622,675; 9,623,240; 9,643,017; 9,643,018; 9,656,075; 9,662,068; 9,662,490; 9,675,794; 9,675,808; 9,682,232; 9,682,241; 9,700,256; 9,700,716; 9,700,723; 9,707,390; 9,707,391; 9,717,904; 9,728,252; 9,737,230; 20010003798; 20010029391; 20020013612; 20020072776; 20020072782; 20020099417; 20020099418; 20020151938; 20030023282; 20030045914; 20030083716; 20030114886; 20030181954; 20030195574; 20030236557; 20030236558; 20040015204; 20040015205; 20040073273; 2004013872; 20040153128; 20040172088; 20040172081; 20040172094; 20040193220; 20040243182; 20040260356; 20050027284; 20050033378; 20050043774; 20050049651; 20050137645; 20050149123; 20050149157; 20050154418; 20050154426; 20050165458; 20050182288; 20050182450; 20050182453; 20050182467; 20050182468; 20050182468; 20050187600; 20050192844; 20050192847; 20050197590; 20050197675; 20050197678; 20050208654; 20050208664; 20050208665; 20050208666; 2005026070; 20050216071; 20050251220; 20050287542; 20060009815; 20060047325; 20060052857; 20060064138; 20060064138; 20060064140; 20060079936; 20060111644; 20060129202; 20060142802; 20060155348; 20060167497; 20060173493; 20060173494; 20060173495; 20060195154; 20060206155; 20060212090; 20060212091; 2006007781; 2006022406; 20060259077; 20060282123; 2006029370; 20060293723; 20070005115; 20070021800; 20070043491; 20070060954; 20070060984; 20070066997; 20070067003; 20070067004; 20070093870; 20070100377; 20070100378; 20070100392; 2007012494; 20070150024; 20070150025; 20070162085; 20070173902; 20070198063; 2007003786; 20070233192; 20070233193; 20070255320; 20070255379; 20000021341; 20080027347; 20080027348; 20080027515; 20080033502; 20080039904; 20080065183; 20080077191; 20080086182; 200080091249; 2008012829; 20080149141; 20080147137; 20080154332; 20080161894; 20080167571; 20080183097; 20080269542; 20080269833; 20080269834; 20080269840; 20090018462; 20090036950; 20090054946; 209000088680; 200093493; 20090118780; 20090163982; 20090171405; 20090187230; 20090234419; 20090276011; 20090276012; 20090200153; 20090326605; 20100003656; 20100004705; 20100004717; 20100057159; 20100063563; 2010010607; 20100114190; 20100114192; 20100114193; 201001250; 20100125304; 20100145420; 20100191304; 2010019808; 20100198296; 2010020474; 20100268208; 20100274303; 20100274308; 20100292602; 20110009920; 2011002189; 2011002079; 20110029038; 20110029044; 20110034912; 20110054569; 2011007770; 20110092800; 20110098778; 20110105998; 20110125203; 20110130615; 20110137381; 20110152967; 20110152988; 20110160795; 20110166430; 20110166546; 20110172554; 20110172725; 20110172732; 20110172739; 20110178441; 20110178442; 20110190569; 20110201944; 2011003222; 20110224602; 20110224749; 20110230701; 20110230938; 20110257517; 20110264182; 20110270095; 20110270096; 20110270346; 20110270347; 20110276107; 20110276112; 2011022225; 20110295344; 20110295345; 20110295346; 20110295347; 20110301529; 20110307030; 20110311489; 20110319975; 20120016336; 20120016432; 20120029591; 20120029601; 20120046711; 20120059431; 20120078323; 20120003700; 20120003701; 20120101326; 20120116741; 20120158092; 20120179220; 20120184801; 20120105020; 20120191150; 20120203079; 20120209346; 20120226130; 20120232327; 20120265262; 20120303080; 20120310050; 20120316622; 20120330369; 20130006332; 20130018438; 20130018439; 20130018440; 20130019325; 20130046350; 20130066350; 20130066392; 20130066395; 20130072996; 20130089503; 20130090454; 201300986441; 20130131753; 20130165846; 20130178913; 20130184638; 20130184792; 20130204144; 20130225953; 20130225992; 2013023170; 20130238049; 20130238050; 20130238053; 20130244323; 20130245464; 20130245486; 20130245711; 20130245712; 20130253612; 20130261703; 20130274625; 2013028890; 20130289653; 2013028966; 20130296496; 20130296637; 20130304159; 20130309278; 20130310909; 20130317580; 20130338450; 20149039290; 20114039336; 20149039578; 20149046203; 20149046407; 2014905203; 20140056815; 20114058188; 20140058292; 20149074180; 20114081071; 20114081353; 20149094720; 20149100633; 20149107397; 20149107390; 20114113367; 20149120930; 20149135680; 20149135886; 20149142653; 20149142654; 20149142669; 20140155772; 20140155952; 20149163643; 20140213842; 2014903961; 20140214135; 20149025826; 20140236272; 20149243613; 20149243714; 20149257110; 20140257132; 20149257430; 20149257437; 20149257430; 20114275716; 20149276194; 20140277255; 20149277256; 20149288620; 20140303452; 20149324110; 20114330334; 20114330335; 20114330336; 20140336514; 20149336730; 20149343463; 20149357936; 20149358067; 20149358193; 20114378851; 20150005592; 20150005838; 20150012054; 20150018893; 20150025422; 20150032044; 2015003078; 20150051655; 20150051656; 20150051657; 20150051658; 20150051659; 20150057715; 20150072394; 20150073237; 20150073505; 20150119689; 20150119794; 20150119956; 20150142082; 20150148878; 20150157858; 20150165226; 20150174390; 20150174495; 20150174497; 20150182753; 20150182756; 20150190636; 20150190637; 20150196246; 2015020242; 20150208978; 20150216468; 20150231330; 20150238761; 20150265830; 20150265836; 20150283265; 2015029771; 20150297889; 20150306392; 20150343222; 20150352362; 20150360030; 20150366482; 20150374973; 20150374993; 20160001096; 20160008620; 20160012749; 20160030666; 20160045162; 20160045731; 20160051010; 20160058359; 20160074660; 20160081610; 20160114165; 20160121114; 20160121116; 20160135727; 20160136423; 20160144175; 2016015162; 20160158554; 20160175607; 20160199656; 20160199662; 20160206236; 20160222073; 20160232011; 20160243381; 20160249846; 2016025046; 20160263376; 2016027900; 20160279022; 20160279023; 20160279024; 20160279025; 20160279267; 20160279410; 20160279435; 20160207869; 20160207895; 20160303396; 20160303402; 20160310070; 20160331952; 20160331974; 20160331982; 20160339237; 20160339230; 20160339239; 20160339242; 20160346542; 20160361549; 20160361546; 20160367800; 20160375245; 20170007820; 20170027812; 20170043160; 20170056467; 20170056642; 20170066806; 20170079573; 20170080050; 20170087364; 20170095598; 20170095670; 20170113042; 20170113057; 20170120043; 20170120052; 20170143550; 20170143963; 20170143986; 20170150916; 20170150921; 20170151433; 20170157402; 20170164894; 20170189717; 2017190017; and 20170224994.

Brain Entrainment: Brain entrainment, also referred to as brainwave synchronization and neural entrainment, refers to the capacity of the brain to naturally synchronize its brainwave frequencies with the rhythm of periodic external stimuli, most commonly auditory, visual, or tactile. Brainwave entrainment technologies are used to induce various brain states, such as relaxation or sleep, by creating stimuli that occur at regular, periodic intervals to mimic electrical cycles of the brain during the desired states, thereby “training” the brain to consciously alter states. Recurrent acoustic frequencies, flickering lights, or tactile vibrations are the most common examples of stimuli applied to generate different sensory responses. It is hypothesized that listening to these beats of certain frequencies one can induce a desired state of consciousness that corresponds with specific neural activity. Patterns of neural firing, measured in Hz, correspond with alertness states such as focused attention, deep sleep, etc.

Neural oscillations are rhythmic or repetitive electrochemical activity in the brain and central nervous system. Such oscillations can be characterized by their frequency, amplitude and phase. Neural tissue can generate oscillatory activity driven by mechanisms within individual neurons, as well as by interactions between them. They may also adjust frequency to synchronize with the periodic vibration of external acoustic or visual stimuli. The functional role of neural oscillations is still not fully understood; however, they have been shown to correlate with emotional responses, motor control, and a number of cognitive functions including information transfer, perception, and memory. Specifically, neural oscillations, in particular theta activity, are extensively linked to memory function, and coupling between theta and gamma activity is considered to be vital for memory functions, including episodic memory. Electroencephalography (EEG) has been most widely used in the study of neural activity generated by large groups of neurons, known as neural ensembles, including investigations of the changes that occur in electroencephalographic profiles during cycles of sleep and wakefulness. EEG signals change dramatically during sleep and show a transition from faster frequencies to increasingly slower frequencies, indicating a relationship between the frequency of neural oscillations and cognitive states including awareness and consciousness.

The term ‘entrainment’ has been used to describe a shared tendency of many physical and biological systems to synchronize their periodicity and rhythm through interaction. This tendency has been identified as specifically pertinent to the study of sound and music generally, and acoustic rhythms specifically. The most ubiquitous and familiar examples of neuromotor entrainment to acoustic stimuli is observable in spontaneous foot or finger tapping to the rhythmic beat of a song. Exogenous rhythmic entrainment, which occurs outside the body, has been identified and documented for a variety of human activities, which include the way people adjust the rhythm of their speech patterns to those of the subject with whom they communicate, and the rhythmic unison of an audience clapping. Even among groups of strangers, the rate of breathing, locomotive and subtle expressive motor movements, and rhythmic speech patterns have been observed to synchronize and entrain, in response to an auditory stimulus, such as a piece of music with a consistent rhythm. Furthermore, motor synchronization to repetitive tactile stimuli occurs in animals, including cats and monkeys as well as humans, with accompanying shifts in electroencephalogram (EEG) readings. Examples of endogenous entrainment, which occurs within the body, include the synchronizing of human circadian sleep-wake cycles to the 24-hour cycle of light and dark, and the frequency following response of humans to sounds and music.

Brainwaves, or neural oscillations, share the fundamental constituents with acoustic and optical waves, including frequency, amplitude and periodicity. The synchronous electrical activity of cortical neural ensembles can synchronize in response to external acoustic or optical stimuli and also entrain or synchronize their frequency and phase to that of a specific stimulus. Brainwave entrainment is a colloquialism for such ‘neural entrainment’, which is a term used to denote the way in which the aggregate frequency of oscillations produced by the synchronous electrical activity in ensembles of cortical neurons can adjust to synchronize with the periodic vibration of an external stimuli, such as a sustained acoustic frequency perceived as pitch, a regularly repeating pattern of intermittent sounds, perceived as rhythm, or of a regularly rhythmically intermittent flashing light.

Changes in neural oscillations, demonstrable through electroencephalogram (EEG) measurements, are precipitated by listening to music, which can modulate autonomic arousal ergotropically and trophotropically, increasing and decreasing arousal respectively. Musical auditory stimulation has also been demonstrated to improve immune function, facilitate relaxation, improve mood, and contribute to the alleviation of stress.

The Frequency following response (FFR), also referred to as Frequency Following Potential (FFP), is a specific response to hearing sound and music, by which neural oscillations adjust their frequency to match the rhythm of auditory stimuli. The use of sound with intent to influence cortical brainwave frequency is called auditory driving, by which frequency of neural oscillation is ‘driven’ to entrain with that of the rhythm of a sound source.

See, en.wikipedia.org/wiki/Brainwave_entrainment;

U.S. Pat. Nos. 5,070,399; 5,306,228; 5,409,445; 6,656,137; 7,749,155; 7,819,794; 7,988,613; 8,088,057; 8,167,784; 8,213,670; 8,267,851; 8,298,078; 8,517,909; 8,517,912; 8,579,793; 8,579,795; 8,597,171; 8,636,649; 8,638,950; 8,668,496; 8,852,073; 8,932,218; 8,960,176; 9,330,523; 9,357,941; 9,458,597; 9,480,812; 9,563,273; 9,609,453; 9,640,167; 9,707,372; 2005015326; 20050182287; 20060106434; 20060206174; 20060281543; 20070066493; 20080039677; 20080304691; 20100010289; 20100010844; 20100028841; 20100056854; 20100076253; 20100130812; 20100222649; 20100286747; 20100298624; 201102908706; 20110319482; 20120003615; 20120053394; 20120150545; 20130030241; 20130072292; 20130131537; 20130172663; 20130184516; 20130203019; 20130234823; 20130338730; 20149088341; 20149107491; 20140114242; 20149154647; 20149174277; 20149275741; 20149309484; 20149371516; 20150142082; 2015020301; 2015029628; 20150313496; 20150313949; 20160008560; 20160019434; 20160055842; 20160205489; 20160235980; 20160239084; 20160345901; 20170034630; 20170061760; 20170087330; 20170094385; 20170095157; 20170099713; 20170135597; and 20170149945.
Carter, J., and H. Russell. “A pilot investigation of auditory and visual entrainment of brain wave activity in learning disabled boys.” Texas Researcher 4.1 (1993): 65-75;
Casciaro, Francesco, et al. “Alpha-rhythm stimulation using brain entrainment enhances heart rate variability in subjects with reduced HRV.” World J. Neuroscience 3.04 (2013): 213;
Helfrich, Randolph F., et al. “Entrainment of brain oscillations by transcranial alternating current stimulation.” Current Biology 24.3 (2014): 333-339;
Huang, Tina L., and Christine Charyton. “A comprehensive review of the psychological effects of brainwave entrainment.” Alternative therapies in health and medicine 14.5 (2008): 38;
Joyce, Michael, and Dave Siever. “Audio-visual entrainment program as a treatment for behavior disorders in a school setting.” J. Neurotherapy 4.2 (2000): 9-25;
Keitel, Christian, Cliodhna Duigley, and Philipp Ruhnau. “Stimulus-driven brain oscillations in the alpha range: entrainment of intrinsic rhythms or frequency-following response?” J. Neuroscience 34.31 (2014): 10137-10140;
Lakatos, Peter, et al. “Entrainment of neuronal oscillations as a mechanism of attentional selection.” Science 320.5872 (2008):110-113;
Mori, Toshio, and Shuichi Kai. “Noise-induced entrainment and stochastic resonance in human brain waves.” Physical review letters 88.21(2002): 218101;
Padmanabhan, R., A. J. Hildreth, and D. Laws. “A prospective, randomised, controlled study examining binaural beat audio and pre-operative anxiety in patients undergoing general anaesthesia for day case surgery.” Anaesthesia 60.9 (2005): 874-877;
Schalles, Matt D., and Jaime A. Pineda. “Musical sequence learning and EEG correlates of audiomotor processing.” Behavioural neurology 2015 (2015). www.hindawi.com/journals/bn/2015/638202/
Thaut, Michael H., David A. Peterson, and Gerald C. McIntosh. “Temporal entrainment of cognitive functions.” Annals of the New York Academy of Sciences 1060.1 (2005): 243-254.
Thut, Gregor, Philippe G. Schyns, and Joachim Gross. “Entrainment of perceptually relevant brain oscillations by non-invasive rhythmic stimulation of the human brain.” Frontiers in Psychology 2 (2011);
Trost, Wiebke, et al. “Getting the beat: entrainment of brain activity by musical rhythm and pleasantness.” NeuroImage 103 (2014): 55-64;
Will, Udo, and Eric Berg. “Brain wave synchronization and entrainment to periodic acoustic stimuli.” Neuroscience letters 424.1(2007): 55-60; and
Zhuang, Tianbao, Hong Zhao, and Zheng Tang. “A study of brainwave entrainment based on EEG brain dynamics.” Computer and information science 2.2 (2009): 80.

A baseline correction of event-related time-frequency measure may be made to take pre-event baseline activity into consideration. In general, a baseline period is defined by the average of the values within a time window preceding the time-locking event. There are at least four common methods for baseline correction in time-frequency analysis. The methods include various baseline value normalizations. See,
Canolty R T, Edwards E, Dalal S S, et al. High gamma power is phase-locked to theta oscillations in human neocortex. Science. 2006; 313:1626-1628.
Hoogenboom N, Schoffelen J M, Oostenveld R, Parkes L M, Fries P. Localizing human visual gamma-band activity in frequency, time and space. Neuroimage. 2006; 29:764-773;
Lachaux J P, Rodriguez E, Martinerie J, Varela F J. Measuring phase synchrony in brain signals. Hum Brain Mapp. 1999; 8:194-208,
Le Van Duyen M, Faucher J, Lachaux J, et al. Comparison of Hilbert transform and wavelet methods for the analysis of neuronal synchrony. J Neurosci Methods. 2001; 111:83-98,
Rodriguez E, George N, Lachaux J P, Martinerie J, Renault B, Varela F J. Perception's shadow: long-distance synchronization of human brain activity. Nature. 1999; 397:430-433.,
Spencer K M, Nestor P G, Perlmutter R, et al. Neural synchrony indexes disordered perception and cognition in schizophrenia. Proc Natl Aced Sci USA. 2004; 101:17288-17293;

The question of whether different emotional states are associated with specific patterns of physiological response has long being a subject of neuroscience research See, for example:
James W (1884.) What is an emotion? Mind 9:188-205; Lacey J I, Bateman D E, Vanlehn R (1953) Autonomic response specificity; an experimental study. Psychosom Med 15: 8-21;
Levenson R W, Heider K, Ekman P, Friesen W V (1992) Emotion and Autonomic Nervous-System Activity in the Minangkabau of West Sumatra. J Pers Soc Psychol 62: 972-988.

Some studies have indicated that the physiological correlates of emotions are likely to be found in the central nervous system (CNS). See, for example:
Buck R (1999) The biological affects: A typology. Psychological Review 106: 301-336; Izard C E (2007) Basic Emotions, Natural Kinds, Emotion Schemes, and a New Paradigm. Perspect Psychol Sci 2: 260-280;
Panksepp J (2007) Neurologizing the Psychology of Affects How Appraisal-Based Constructivism and Basic Emotion Theory Can Coexist. Perspect Psychol Sci 2: 281-296.

Electroencephalograms (EEG) and functional Magnetic Resonance Imaging, fMRI have been used to study specific brain activity associated with different emotional states. Mauss and Robinson, in their review paper, have indicated that “emotional state is likely to involve circuits rather than any brain region considered in isolation” (Mauss I B, Robinson M D (2009) Measures of emotion: A review. Cogn Emot 23: 209-237.)

The amplitude, latency from the stimulus, and covariance (in the case of multiple electrode sites) of each component can be examined in connection with a cognitive task (ERP) or with no task (EP). Steady-state visually evoked potentials (SSVEPs) use a continuous sinusoidally-modulated flickering light, typically superimposed in front of a TV monitor displaying a cognitive task. The brain response in a narrow frequency band containing the stimulus frequency is measured. Magnitude, phase, and coherence (in the case of multiple electrode sites) may be related to different parts of the cognitive task. Brain entrainment may be detected through EEG or MEG activity. Brain entrainment may be detected through EEG or MEG activity. See:
Abeln, Vera, et al. “Brainwave entrainment for better sleep and post-sleep state of young elite soccer players-A pilot study.” European J. Sport science 14.5 (2014): 393-402;
Acton, George. “Methods for independent entrainment of visual field zones.” U.S. Pat. No. 9,629,976. 25 Apr. 2017;
Albouy, Philippe, et al. “Selective entrainment of theta oscillations in the dorsal stream causally enhances auditory working memory performance.” Neuron 94.1 (2017): 193-206.
Amengual, J., et al. “P018 Local entrainment and distribution across cerebral networks of natural oscillations elicited in implanted epilepsy patients by intracranial stimulation: Paving the way to develop causal connectomics of the healthy human brain.” Clin. Neurophysiology 128.3 (2017): e18;
Argento, Emanuele, et al. “Augmented Cognition via Brainwave Entrainment in Virtual Reality: An Open, Integrated Brain Augmentation in a Neuroscience System Approach.” Augmented Human Research 2.1(2017): 3;
Bello, Nicholas P. “Altering Cognitive and Brain States Through Cortical Entrainment.” (2014); Costa-Faidella, Jordi, Elyse S. Sussman, and Caries Escera. “Selective entrainment of brain oscillations drives auditory perceptual organization.” NeuroImage (2017);
Börgers, Christoph. “Entrainment by Excitatory Input Pulses.” An Introduction to Modeling Neuronal Dynamics. Springer International Publishing, 2017. 183-192;
Calderone, Daniel J., et al. “Entrainment of neural oscillations as a modifiable substrate of attention.” Trends in cognitive sciences 18.6 (2014): 300-309;
Casciaro, Francesco, et al. “Alpha-rhythm stimulation using brain entrainment enhances heart rate variability in subjects with reduced HRV.” World J. Neuroscience 3.04 (2013): 213;
Chang, Daniel Wonchul. “Method and system for brain entertainment.” U.S. Pat. No. 8,636,840. 28 Jan. 2014;
Colzato, Lorenza S., Amengual, Julia L., et al. “Local entrainment of oscillatory activity induced by direct brain stimulation in humans.” Scientific Reports 7 (2017);
Conte, Elio, et al. “A Fast Fourier Transform analysis of time series data of heart rate variability during alfa-rhythm stimulation in brain entrainment.” NeuroQuantology 11.3 (2013);
Dikker, Suzanne, et al. “Brain-to-brain synchrony tracks real-world dynamic group interactions in the classroom.” Current Biology 27.9 (2017):1375-1380;
Ding, Nai, and Jonathan Z. Simon. “Cortical entrainment to continuous speech: functional roles and interpretations.” Frontiers in human neuroscience 8 (2014);
Doherty, Cormac. “A comparison of alpha brainwave entrainment, with and without musical accompaniment.” (2014);
Falk, Simone, Cosima Lanzilotti, and Daniele Schön. “Tuning neural phase entrainment to speech.” J. Cognitive Neuroscience (2017);
Gao, Junling, et al. “Entrainment of chaotic activities in brain and heart during MBSR mindfulness training.” Neuroscience letters 616 (2016): 218-223;
Gooding-Williams, Gerard, Hongfang Wang, and Klaus Kessler. “THETA-Rhythm Makes the World Go Round: Dissociative Effects of TMS Theta Versus Alpha Entrainment of Right pTPJ on Embodied Perspective Transformations.” Brain Topography (2017):1-4;
Hanslmayr, Simon, Jonas Matuschek, and Marie-Christin Fellner. “Entrainment of prefrontal beta oscillations induces an endogenous echo and impairs memory formation.” Current Biology 24.8 (2014): 904-909;
Heideman, Simone G., Erik S. to Woerd, and Peter Praamstra. “Rhythmic entrainment of slow brain activity preceding leg movements.” Clin. Neurophysiology 126.2 (2015): 348-355;
Helfrich, Randolph F., et al. “Entrainment of brain oscillations by transcranial alternating current stimulation.” Current Biology 24.3 (2014): 333-339;
Henry, Molly J., et al. “Aging affects the balance of neural entrainment and top-down neural modulation in the listening brain.” Nature Communications 8 (2017): ncomms15801;
Horr, Ninja K., Maria Wimber, and Massimiliano Di Luca. “Perceived time and temporal structure: Neural entrainment to isochronous stimulation increases duration estimates.” Neuroimage 132 (2016): 148-156;
Irwin, Rosie. “Entraining Brain Oscillations to Influence Facial Perception.” (2015);
Kalyan, Ritu, and Bipan Kaushal. “Binaural Entrainment and Its Effects on Memory.” (2016);
Keitel, Anne, et al. “Auditory cortical delta-entrainment interacts with oscillatory power in multiple fronto-parietal networks.” Neuroimage 147 (2017): 32-42;
Keitel, Christian, Cliodhna Duigley, and Philipp Ruhnau. “Stimulus-driven brain oscillations in the alpha range: entrainment of intrinsic rhythms or frequency-following response?” J. Neuroscience 34.31 (2014): 10137-10140;
Koelsch, Stefan. “Music-evoked emotions: principles, brain correlates, and implications for therapy.” Annals of the New
York Academy of Sciences1337.1 (2015): 193-201;
Kösem, Anne, et al. “Neural entrainment reflects temporal predictions guiding speech comprehension.” the Eighth Annual Meeting of the Society for the Neurobiology of Language (SNL 2016). 2016;
Lee, Daniel Keewoong, Dongyeup Daniel Synn, and Daniel Chesong Lee. “Intelligent earplug system.” U.S. patent application Ser. No. 15/106,989;
Lefournour, Joseph, Ramaswamy Palaniappan, and Ian V. McLoughlin. “Inter-hemispheric and spectral power analyses of binaural beat effects on the brain.” Matters 2.9 (2016): e201607000001;
Mai, Guangting, James W. Minett, and William S-Y. Wang. “Delta, theta, beta, and gamma brain oscillations index levels of auditory sentence processing.” Neuroimage 133(2016):516-528;
Marconi, Pier Luigi, et al. “The phase amplitude coupling to assess brain network system integration.” Medical Measurements and Applications (MeMeA), 2016 IEEE International Symposium on. IEEE, 2016;
McLaren, Elgin-Skye, and Alissa N. Antle. “Exploring and Evaluating Sound for Helping Children Self-Regulate with a Brain-Computer Application.” Proceedings of the 2017 Conference on Interaction Design and Children. ACM, 2017;
Moisa, Marius, et al. “Brain network mechanisms underlying motor enhancement by transcranial entrainment of gamma oscillations.” J. Neuroscience 38.47 (2016): 12053-12065;
Molinaro, Nicola, et al. “Out-of-synchrony speech entrainment in developmental dyslexia.” Human brain mapping 37.8 (2016): 2767-2783;
Moseley, Ralph. “Immersive brain entrainment in virtual worlds: actualizing meditative states.” Emerging Trends and Advanced Technologies for Computational Intelligence. Springer International Publishing, 2016. 315-346;
Neuling, Toralf, et al. “Friends, not foes: magnetoencephalography as a tool to uncover brain dynamics during transcranial alternating current stimulation.” Neuroimage 118 (2015): 406-413;
Notbohm, Annika, Jurgen Kurths, and Christoph S. Herrmann. “Modification of brain oscillations via rhythmic light stimulation provides evidence for entrainment but not for superposition of event-related responses.” Frontiers in human neuroscience 10 (2016);
Nozaradan, S., et al. “P943: Neural entrainment to musical rhythms in the human auditory cortex, as revealed by intracerebral recordings.” Clin. Neurophysiology 125 (2014): S299;
Palaniappan, Ramaswamy, et al. “Improving the feature stability and classification performance of bimodal brain and heart biometrics.” Advances in Signal Processing and Intelligent Recognition Systems. Springer, Cham, 2016. 175-186;
Palaniappan, Ramaswamy, Somnuk Phon-Amnuaisuk, and Chikkannan Eswaran. “On the binaural brain entrainment indicating lower heart rate variability.” Int. J. Cardiol 190 (2015): 262-263;
Papagiannakis, G., et al. A virtual reality brainwave entrainment method for human augmentation applications. Technical Report, FORTH-ICS/TR-458, 2015;
Park, Hyojin, et al. “Frontal top-down signals increase coupling of auditory low-frequency oscillations to continuous speech in human listeners.” Current Biology 25.12 (2015):1649-1653;
Pérez, Alejandro, Manuel Carreiras, and Jon Andoni Dunabeitia. “Brain-to-brain entrainment: EEG interbrain synchronization while speaking and listening.” Scientific Reports 7 (2017);
Riecke, Lars, Alexander T. Sack, and Charles E. Schroeder. “Endogenous delta/theta sound-brain phase entrainment accelerates the buildup of auditory streaming.” Current Biology 25.24 (2015): 3196-3201;
Spaak, Eelke, Floris P. de Lange, and Ole Jensen. “Local entrainment of alpha oscillations by visual stimuli causes cyclic modulation of perception.” J. Neuroscience 34.10(2014):3536-3544;
Thaut, Michael H. “The discovery of human auditory-motor entrainment and its role in the development of neurologic music therapy.” Progress in brain research 217 (2015): 253-266;
Thaut, Michael H., Gerald C. McIntosh, and Volker Hoemberg. “Neurobiological foundations of neurologic music therapy: rhythmic entrainment and the motor system.” Frontiers in psychology 5 (2014);
Thut, G. “T030 Guiding TMS by EEG/MEG to interact with oscillatory brain activity and associated functions.” Clin. Neurophysiology 128.3 (2017): e9;
Treviño, Guadalupe Villarreal, et al. “The Effect of Audio Visual Entrainment on Pre-Attentive Dysfunctional Processing to Stressful Events in Anxious Individuals.” Open J. Medical Psychology 3.05 (2014): 364;
Trost, Wiebke, et al. “Getting the beat: entrainment of brain activity by musical rhythm and pleasantness.” NeuroImage 103 (2014): 55-64;
Tsai, Shu-Hui, and Yue-Der Lin. “Autonomie feedback with brain entrainment.” Awareness Science and Technology and Obi-Media Computing (iCAST-UMEDIA), 2013 International Joint Conference on. IEEE, 2013;
Vossen, Alexandra, Joachim Gross, and Gregor Thut. “Alpha power increase after transcranial alternating current stimulation at alpha frequency (α-tACS) reflects plastic changes rather than entrainment.” Brain Stimulation 8.3 (2015): 499-508;
Witkowski, Matthias, et al. “Mapping entrained brain oscillations during transcranial alternating current stimulation (tACS).” Neuroimage 140 (2016): 89-98;
Zlotnik, Anatoly, Raphael Nagao, and Istvãn Z. Kiss Jr-Shin Li. “Phase-selective entrainment of nonlinear oscillator ensembles.” Nature Communications 7 (2016).

The entrainment hypothesis (Thut and Miniussi, 2009; Thut et al., 2011a, 2012), suggests the possibility of inducing a particular oscillation frequency in the brain using an external oscillatory force (e.g., rTMS, but also tACS). The physiological basis of oscillatory cortical activity lies in the timing of the interacting neurons; when groups of neurons synchronize their firing activities, brain rhythms emerge, network oscillations are generated, and the basis for interactions between brain areas may develop (Buzsaki, 2006). Because of the variety of experimental protocols for brain stimulation, limits on descriptions of the actual protocols employed, and limited controls, consistency of reported studies is lacking, and extrapolability is limited. Thus, while there is various consensus in various aspects of the effects of extra cranial brain stimulation, the results achieved have a degree of uncertainty dependent on details of implementation. On the other hand, within a specific experimental protocol, it is possible to obtain statistically significant and repeatable results. This implies that feedback control might be effective to control implementation of the stimulation for a given purpose; however, studies that employ feedback control are lacking.

Different cognitive states are associated with different oscillatory patterns in the brain (Buzsàki, 2006; Canolty and Knight, 2010; Varela et al., 2001). Thut et al. (2011b) directly tested the entrainment hypothesis by means of a concurrent EEG-TMS experiment. They first determined the individual source of the parietal-occipital alpha modulation and the individual alpha frequency (magnetoencephalography study). They then applied rTMS at the individual alpha power while recording the EEG activity at rest. The results confirmed the three predictions of the entrainment hypothesis: the induction of a specific frequency after TMS, the enhancement of oscillation during TMS stimulation due to synchronization, and a phase alignment of the induced frequency and the ongoing activity (Thut et al., 2011b).

If associative stimulation is a general principle for human neural plasticity in which the timing and strength of activation are critical factors, it is possible that synchronization within or between areas using an external force to phase/align oscillations can also favor efficient communication and associative plasticity (or alter communication). In this respect associative, cortico-cortical stimulation has been shown to enhance coherence of oscillatory activity between the stimulated areas (Plewnia et al., 2008).

In coherence resonance (Longtin, 1997), the addition of a certain amount of noise in an excitable system results in the most coherent and proficient oscillatory responses. The brain's response to external timing-embedded stimulation can result in a decrease in phase variance and an enhanced alignment (clustering) of the phase components of the ongoing EEG activity (entraining, phase resetting) that can change the signal-to-noise ratio and increase (or decrease) signal efficacy.

If one considers neuron activity within the brain as a set of loosely coupled oscillators, then the various parameters that might be controlled include the size of the region of neurons, frequency of oscillation, resonant frequency or time-constant, oscillator damping, noise, amplitude, coupling to other oscillators, and of course, external influences that may include stimulation and/or power loss. In a human brain, pharmacological intervention may be significant. For example, drugs that alter excitability, such as caffeine, neurotransmitter release and reuptake, nerve conductance, etc. can all influence operation of the neural oscillators. Likewise, sub-threshold external stimulation effects, including DC, AC and magnetic electromagnetic effects, can also influence operation of the neural oscillators.

Phase resetting or shifting can synchronize inputs and favor communication and, eventually, Hebbian plasticity (Hebb, 1949). Thus, rhythmic stimulation may induce a statistically higher degree of coherence in spiking neurons, which facilitates the induction of a specific cognitive process (or hinders that process). Here, the perspective is slightly different (coherence resonance), but the underlining mechanisms are similar to the ones described so far (stochastic resonance), and the additional key factor is the repetition at a specific rhythm of the stimulation.

In the 1970's, the British biophysicist and psychobiologist, C. Maxwell Cade, monitored the brainwave patterns of advanced meditators and 300 of his students. Here he found that the most advanced meditators have a specific brainwave pattern that was different from the rest of his students. He noted that these meditators showed high activity of alpha brainwaves accompanied by beta, theta and even delta waves that were about half the amplitude of the alpha waves. See, Cade “The Awakened Mind: Biofeedback and the Development of Higher States of Awareness” (Dell, 1979). Anna Wise extended Cade's studies, and found that extraordinary achievers which included composers, inventors, artists, athletes, dancers, scientists, mathematicians, CEO's and presidents of large corporations have brainwave patterns differ from average performers, with a specific balance between Beta, Alpha, Theta and Delta brainwaves where Alpha had the strongest amplitude. See, Anna Wise, “The High-Performance Mind: Mastering Brainwaves for Insight, Healing, and Creativity”.

Entrainment is plausible because of the characteristics of the demonstrated EEG responses to a single TMS pulse, which have a spectral composition which resemble the spontaneous oscillations of the stimulated cortex. For example, TMS of the “resting” visual (Rosanova et al., 2009) or motor cortices (Veniero et al., 2011) triggers alpha-waves, the natural frequency at the resting state of both types of cortices. With the entrainment hypothesis, the noise generation framework moves to a more complex and extended level in which noise is synchronized with on-going activity. Nevertheless, the model to explain the outcome will not change, stimulation will interact with the system, and the final result will depend on introducing or modifying the noise level. The entrainment hypothesis makes clear predictions with respect to online repetitive TMS paradigms' frequency engagement as well as the possibility of inducing phase alignment, i.e., a reset of ongoing brain oscillations via external spTMS (Thut et al., 2011a, 2012; Veniero et al., 2011). The entrainment hypothesis is superior to the localization approach in gaining knowledge about how the brain works, rather than where or when a single process occurs. TMS pulses may phase-align the natural, ongoing oscillation of the target cortex. When additional TMS pulses are delivered in synchrony with the phase-aligned oscillation (i.e., at the same frequency), further synchronized phase-alignment will occur, which will bring the oscillation of the target area in resonance with the TMS train. Thus, entrainment may be expected when TMS is frequency-tuned to the underlying brain oscillations (Veniero et al., 2011).

Binaural Beats: Binaural beats are auditory brainstem responses which originate in the superior olivary nucleus of each hemisphere. They result from the interaction of two different auditory impulses, originating in opposite ears, below 1000 Hz and which differ in frequency between one and 30 Hz. For example, if a pure tone of 400 Hz is presented to the right ear and a pure tone of 410 Hz is presented simultaneously to the left ear, an amplitude modulated standing wave of 10 Hz, the difference between the two tones, is experienced as the two wave forms mesh in and out of phase within the superior olivary nuclei. This binaural beat is not heard in the ordinary sense of the word (the human range of hearing is from 20-20,000 Hz). It is perceived as an auditory beat and theoretically can be used to entrain specific neural rhythms through the frequency-following response (FFR)—the tendency for cortical potentials to entrain to or resonate at the frequency of an external stimulus. Thus, it is theoretically possible to utilize a specific binaural-beat frequency as a consciousness management technique to entrain a specific cortical rhythm. The binaural-beat appears to be associated with an electroencephalographic (EEG) frequency-following response in the brain.

Uses of audio with embedded binaural beats that are mixed with music or various pink or background sound are diverse. They range from relaxation, meditation, stress reduction, pain management, improved sleep quality, decrease in sleep requirements, super learning, enhanced creativity and intuition, remote viewing, telepathy, and out-of-body experience and lucid dreaming. Audio embedded with binaural beats is often combined with various meditation techniques, as well as positive affirmations and visualization.

When signals of two different frequencies are presented, one to each ear, the brain detects phase differences between these signals. “Under natural circumstances a detected phase difference would provide directional information. The brain processes this anomalous information differently when these phase differences are heard with stereo headphones or speakers. A perceptual integration of the two signals takes place, producing the sensation of a third “beat” frequency. The difference between the signals waxes and wanes as the two different input frequencies mesh in and out of phase. As a result of these constantly increasing and decreasing differences, an amplitude-modulated standing wave—the binaural beat—is heard. The binaural beat is perceived as a fluctuating rhythm at the frequency of the difference between the two auditory inputs. Evidence suggests that the binaural beats are generated in the brainstem's superior olivary nucleus, the first site of contralateral integration in the auditory system. Studies also suggest that the frequency-following response originates from the inferior colliculus. This activity is conducted to the cortex where it can be recorded by scalp electrodes. Binaural beats can easily be heard at the low frequencies (<30 Hz) that are characteristic of the EEG spectrum.

Synchronized brain waves have long been associated with meditative and hypnagogic states, and audio with embedded binaural beats has the ability to induce and improve such states of consciousness. The reason for this is physiological. Each ear is “hardwired” (so to speak) to both hemispheres of the brain. Each hemisphere has its own olivary nucleus (sound-processing center) which receives signals from each ear. In keeping with this physiological structure, when a binaural beat is perceived there are actually two standing waves of equal amplitude and frequency present, one in each hemisphere. So, there are two separate standing waves entraining portions of each hemisphere to the same frequency. The binaural beats appear to contribute to the hemispheric synchronization evidenced in meditative and hypnagogic states of consciousness. Brain function is also enhanced through the increase of cross-callosal communication between the left and right hemispheres of the brain.
en.wikipedia.org/wiki/Beat_(acoustics) #Binaural_beats.
Atwater, F. H. (2001). Binaural beats and the regulation of arousal levels. Proceedings of the TANS, 11;
Colzato, L. S., Barone, H., Sellaro, R., & Hommel, B. (2017). More attentional focusing through binaural beats: evidence from the global-local task. Psychological research, 81(1), 271-277;
Foster, D. S. (1990). EEG and subjective correlates of alpha frequency binaural beats stimulation combined with alpha biofeedback (Doctoral dissertation, Memphis State University);
Gao, X., Deo, H., Ming, D., Qi, H., Wang, X., Wang, X., B Zhou, P. (2014). Analysis of EEG activity in response to binaural beats with different frequencies. International Journal of Psychophysiology, 94(3), 399-406;
Hink, R. F., Kodera, K., Yamada, O., Kaga, K., & Suzuki, J. (1980). Binaural interaction of a beating frequency-following response. Audiology, 19(1), 36-43;
Kasprzak, C. (2011). Influence of binaural beats on EEG signal. Acta Physica Polonica A, 119(6A), 986-990;
Lane, J. D., Kasian, S. J., Owens, J. E., & Marsh, G. R. (1998). Binaural auditory beats affect vigilance performance and mood. Physiology & behavior, 93(2), 249-252;
Mortazavi, S. M. J., Zahraei-Moghadam, S. M., Masoumi, S., Rafati, A., Haghani, M., Mortazavi, S. A. R., & Lehtabian, M. (2017). Short Term Exposure to Binaural Beats Adversely Affects Learning and Memory in Rats. Journal of Biomedical Physics and Engineering.
Oster, G (October 1973). “Auditory beats in the brain”. Scientific American. 229 (4): 94-102. See:
Padmanabhan, R., Hildreth, A. J., & Laws, D. (2005). A prospective, randomised, controlled study examining binaural beat audio and pre-operative anxiety in patients undergoing general anaesthesia for day case surgery. Anaesthesia, 60(9), 874-877;
Pratt, H., Starr, A., Michalewski, H. J., Dimitrijevic, A., Bleich, N., & Mittelman, N. (2009). Cortical evoked potentials to an auditory illusion: binaural beats. Clinical Neurophysiology, 120(8), 1514-1524;
Pratt, H., Starr, A., Michalewski, H. J., Dimitrijevic, A., Bleich, N., & Mittelman, N. (2010). A comparison of auditory evoked potentials to acoustic beats and to binaural beats. Hearing research, 292(1), 34-44;
Reedijk, S. A., Bolders, A., & Hommel, B. (2013). The impact of binaural beats on creativity. Frontiers in human neuroscience, 7;
Sung, H. C., Lee, W. L., Li, H. M., Lin, C. Y., Wu, Y. Z., Wang, J. J., & Li, T. L. (2017). Familiar Music Listening with Binaural Beats for Older People with Depressive Symptoms in Retirement Homes. Neuropsychiatry, 7(4);

Brain Entrainment Frequency Following Response (or FFR). See, “Stimulating the Brain with Light and Sound,” Transparent Corporation, Neuroprogrammer™ 3, www.transparentcorp.com/products/np/entrainment.php.

Isochronic Tones: Isochronic tones are regular beats of a single tone that are used alongside monaural beats and binaural beats in the process called brainwave entrainment. At its simplest level, an isochronic tone is a tone that is being turned on and off rapidly. They create sharp, distinctive pulses of sound.
www.livingflow.net/isochronic-tones-work/;
Casciaro, F., Laterza, V., Conte, S., Pieralice, M., Federici, A., Todarello, O., . . . & Conte, E. (2013). Alpha-rhythm stimulation using brain entrainment enhances heart rate variability in subjects with reduced HRV. World Journal of Neuroscience, 3(04), 213;
Conte, E., Conte, S., Santacroce, N., Federici, A., Todarello, O., Orsucci, F., . . . & Laterza, V. (2013). A Fast Fourier Transform analysis of time series data of heart rate variability during alfa-rhythm stimulation in brain entrainment. NeuroQuantology, 11(3);
Doherty, C. (2014). A comparison of alpha brainwave entrainment, with and without musical accompaniment;
Huang, T. L., & Charyton, C. (2008). A comprehensive review of the psychological effects of brainwave entrainment. Alternative therapies in health and medicine, 14(5), 38;
Moseley, R. (2015, July). Inducing targeted brain states utilizing merged reality systems. In Science and Information Conference (SAI), 2015 (pp. 657-663). IEEE.
Oster, G. (1973). Auditory beats in the brain. Scientific American, 229(4), 94-102;
Schulze, H. H. (1989). The perception of temporal deviations in isochronic patterns. Attention, Perception, & Psychophysics, 45(4), 291-296;
Trost, W., Fruhholz, S., Schön, D., Labbe, C., Pichon, S., Grandjean, D., & Vuilleumier, P. (2014). Getting the beat: entrainment of brain activity by musical rhythm and pleasantness. NeuroImage, 103, 55-94.

Time-Frequency Analysis: Brian J. Roach and Daniel H. Mathalon, “Event-related EEG time-frequency analysis: an overview of measures and analysis of early gamma band phase locking in schizophrenia. Schizophrenia Bull. USA. 2008; 34:5:907-926., describes a mechanism for EEG time-frequency analysis. Fourier and wavelet transforms (and their inverse) may be performed on EEG signals.

See, U.S. Pat. Nos. 4,497,299; 4,408,616; 4,421,122; 4,493,327; 4,550,736; 4,557,270; 4,579,125; 4,583,190; 4,585,011; 4,610,259; 4,649,482; 4,705,048; 4,736,307; 4,744,029; 4,776,345; 4,792,145; 4,794,533; 4,846,190; 4,862,359; 4,883,067; 4,907,597; 4,924,875; 4,949,058; 5,010,891; 5,020,549; 5,029,082; 5,083,571; 5,092,341; 5,105,354; 5,109,862; 5,289,530; 5,230,344; 5,230,346; 5,233,517; 5,241,967; 5,243,517; 5,269,315; 5,280,791; 5,287,859; 5,309,917; 5,308,923; 5,320,109; 5,339,811; 5,339,826; 5,377,100; 5,406,956; 5,406,957; 5,443,073; 5,447,166; 5,450,117; 5,474,082; 5,555,888; 5,611,350; 5,619,995; 5,632,272; 5,643,325; 5,678,561; 5,685,313; 5,692,517; 5,694,939; 5,699,808; 5,752,521; 5,755,739; 5,771,261; 5,771,897; 5,794,623; 5,795,304; 5,797,840; 5,810,737; 5,813,993; 5,827,195; 5,840,040; 5,846,189; 5,846,208; 5,853,005; 5,871,517; 5,884,626; 5,899,867; 5,916,171; 5,995,868; 6,002,952; 6,011,990; 6,016,444; 6,021,345; 6,032,072; 6,044,292; 6,050,940; 6,052,619; 6,067,462; 6,067,467; 6,070,098; 6,071,246; 6,081,735; 6,097,980; 6,097,981; 6,115,631; 6,117,075; 6,129,681; 6,155,993; 6,157,850; 6,157,857; 6,171,250; 6,195,576; 6,196,972; 6,224,549; 6,236,872; 6,207,328; 6,292,688; 6,293,904; 6,305,943; 6,306,077; 6,308,342; 6,315,736; 6,317,627; 6,325,761; 6,331,164; 6,338,713; 6,343,229; 6,358,201; 6,366,813; 6,370,423; 6,375,614; 6,377,833; 6,385,486; 6,394,963; 6,402,520; 6,475,163; 6,482,165; 6,493,577; 6,496,724; 6,511,424; 6,520,905; 6,520,921; 6,524,249; 6,527,730; 6,529,773; 6,544,170; 6,546,378; 6,547,736; 6,547,746; 6,549,804; 6,556,861; 6,565,510; 6,574,573; 6,594,524; 6,602,202; 6,616,611; 6,622,036; 6,625,485; 6,626,676; 6,650,917; 6,652,470; 6,654,632; 6,658,287; 6,678,548; 6,687,525; 6,699,194; 6,708,398; 6,726,624; 6,731,975; 6,735,467; 6,743,182; 6,745,060; 6,745,156; 6,746,408; 6,751,499; 6,768,920; 6,798,899; 6,801,803; 6,804,661; 6,816,744; 6,818,956; 6,820,420; 6,843,774; 6,865,494; 6,875,174; 6,882,881; 6,886,864; 6,915,241; 6,928,354; 6,931,274; 6,931,275; 6,981,947; 6,985,768; 6,988,056; 6,993,380; 7,011,410; 7,014,613; 7,016,722; 7,037,260; 7,043,293; 7,054,454; 7,089,277; 7,092,748; 7,099,714; 7,104,963; 7,105,824; 7,123,955; 7,120,713; 7,130,691; 7,146,289; 7,150,710; 7,150,715; 7,150,710; 7,163,512; 7,164,941; 7,177,675; 7,190,995; 7,207,948; 7,209,788; 7,215,86; 7,225,013; 7,228,169; 7,228,171; 7,231,245; 7,254,433; 7,254,43; 7,254,500; 7,267,652; 7,269,456; 7,286,871; 7,288,066; 7,297,110; 7,299,089; 7,324,845; 7,328,053; 7,333,619; 7,333,851; 7,343,190; 7,367,948; 7,373,190; 7,376,453; 7,381,185; 7,383,070; 7,392,079; 7,395,292; 7,396,333; 7,392,828; 7,403,814; 7,403,815; 7,418,290; 7,428,247; 7,450,868; 7,454,248; 7,462,151; 7,468,040; 7,469,687; 7,471,971; 7,471,978; 7,488,958; 7,489,648; 7,491,173; 7,496,393; 7,499,741; 7,499,745; 7,509,154; 7,50,161; 7,509,163; 7,510,531; 7,530,955; 7,537,568; 7,538,532; 7,538,533; 7,547,284; 7,558,622; 7,558,903; 7,570,991; 7,572,225; 7,574,007; 7,574,254; 7,593,767; 7,594,122; 7,596,535; 7,603,160; 7,604,621; 7,610,094; 7,623,912; 7,623,928; 7,625,340; 7,630,757; 7,640,055; 7,643,655; 7,647,098; 7,654,948; 7,668,578; 7,668,591; 7,672,717; 7,676,263; 7,678,061; 7,684,856; 7,697,97; 7,702,502; 7,706,871; 7,706,992; 7,711,417; 7,715,910; 7,720,530; 7,727,161; 7,729,753; 7,733,224; 7,734,334; 7,747,325; 7,751,878; 7,754,190; 7,757,690; 7,758,503; 7,764,987; 7,771,364; 7,774,052; 7,774,064; 7,778,693; 7,787,946; 7,794,406; 7,801,592; 7,801,593; 7,803,110; 7,803,118; 7,808,433; 7,811,279; 7,819,812; 7,831,302; 7,853,320; 7,860,561; 7,865,234; 7,865,235; 7,878,965; 7,870,438; 7,887,493; 7,894,890; 7,896,807; 7,895,258; 7,904,144; 7,907,994; 7,909,771; 7,918,779; 7,920,914; 7,930,035; 7,938,782; 7,938,785; 7,941,208; 7,942,824; 7,944,551; 7,962,204; 7,974,696; 7,983,741; 7,983,757; 7,986,991; 7,993,279; 7,996,075; 8,002,553; 8,005,534; 8,005,624; 8,010,347; 8,019,400; 8,019,410; 8,024,032; 8,025,404; 8,032,208; 8,033,996; 8,036,728; 8,036,736; 8,041,136; 8,046,041; 8,046,042; 8,065,011; 8,066,637; 8,066,647; 8,068,904; 8,073,534; 8,075,498; 8,078,953; 8,082,031; 8,086,294; 8,088,283; 8,005,200; 8,103,333; 8,810,036; 8,121,039; 8,114,021; 8,121,673; 8,126,528; 8,128,572; 8,131,354; 8,133,172; 8,137,269; 8,137,270; 8,145,310; 8,152,732; 8,155,736; 8,160,689; 8,172766; 8,177,726; 8,177,727; 8,180,420; 8,180,601; 8,185,207; 8,187,201; 8,190,227; 8,190,249; 8,190,251; 8,197,395; 8,197,437; 8,200,319; 8,204,583; 8,821,035; 8,214,007; 8,224,433; 8,236,005; 8,239,014; 8,241,238; 8,244,340; 8,244,475; 8,248,698; 8,271,077; 8,280,502; 8,280,503; 8,280,514; 8,285,368; 8,290,575; 8,295,914; 8,296,108; 8,298,140; 8,301,232; 8,301,233; 8,306,610; 8,311,622; 8,314,707; 8,315,970; 8,320,648; 8,323,188; 8,323,189; 8,323,204; 8,328,718; 8,332,017; 8,332,024; 8,335,561; 8,337,404; 8,340,752; 8,340,753; 8,343,026; 8,346,342; 8,346,348; 8,352,023; 8,353,837; 8,354,881; 8,356,594; 8,359,800; 8,364,220; 8,364,254; 8,364,255; 8,368,840; 8,374,690; 8,374,703; 8,380,296; 8,382,667; 8,386,244; 8,391,966; 8,396,546; 8,396,557; 8,401,624; 8,401,626; 8,403,848; 8,425,415; 8,420,583; 8,428,696; 8,437,843; 8,437,844; 8,442,620; 8,449,471; 8,452,544; 8,454,555; 8,461,821; 8,463,007; 8,463,348; 8,463,370; 8,465,408; 8,467,877; 8,473,024; 8,473,044; 8,473,306; 8,475,354; 8,475,368; 8,475,387; 8,478,388; 8,478,394; 8,478,402; 8,480,554; 8,484,270; 8,494,829; 8,498,697; 8,500,282; 8,500,636; 8,509,885; 8,508,904; 8,512,221; 8,512,240; 8,515,535; 8,519,053; 8,521,284; 8,525,673; 8,525,687; 8,527,435; 8,531,291; 8,538,512; 8,538,514; 8,538,705; 8,542,900; 8,543,199; 8,543,211; 8,545,416; 8,545,436; 8,554,311; 8,554,325; 8,560,034; 8,560,073; 8,562,525; 8,562,526; 8,562,527; 8,562,951; 8,568,329; 8,571,642; 8,585,568; 8,588,933; 8,591,419; 8,591,490; 8,597,193; 8,600,502; 8,606,351; 8,606,356; 8,606,360; 8,620,419; 8,628,480; 8,630,698; 8,632,485; 8,632,750; 8,641,632; 8,644,914; 8,644,921; 8,647,278; 8,649,866; 8,652,038; 8,655,817; 8,657,756; 8,660,798; 8,666,467; 8,670,603; 8,672,852; 8,680,991; 8,684,900; 8,684,922; 8,684,926; 8,688,208; 8,690,748; 8,693,756; 8,684,087; 8,694,088; 8,694,107; 8,700,137; 8,7001,41; 8,700,142; 8,706,205; 8,706,206; 8,706,207; 8,708,903; 8,712,507; 8,712,513; 8,725,238; 8,725,243; 8,725,311; 8,725,668; 8,727,978; 8,728,001; 8,738,121; 8,744,563; 8,747,313; 8,747,336; 8,750,971; 8,750,974; 8,750,992; 8,755,854; 8,755,856; 8,755,868; 8,755,869; 8,755,871; 8,761,866; 8,761,869; 8,764,651; 8,764,652; 8,764,653; 8,768,447; 8,771,194; 8,775,349; 8,781,193; 8,781,563; 8,781,595; 8,781,597; 8,784,322; 8,786,624; 8,790,255; 8,790,272; 8,792,974; 8,798,735; 8,798,736; 8,80,620; 8,821,498; 8,825,148; 8,825,428; 8,827,917; 8,831,705; 8,838,226; 8,838,227; 8,843,198; 8,843,210; 8,849,390; 8,849,392; 8,849,681; 8,852,100; 8,852,121; 8,855,758; 8,858,44; 8,858,448; 8,862,196; 8,862,221; 8,862,581; 8,868,148; 8,868,163; 8,868,172; 8,868,174; 8,868,175; 8,870,737; 8,880,207; 8,880,576; 8,886,298; 8,888,672; 8,888,673; 8,888,702; 8,888,708; 8,898,037; 8,902,070; 8,903,483; 8,914,100; 8,915,741; 8,915,871; 8,918,162; 8,918,178; 8,922,788; 8,923,958; 8,924,235; 8,932,227; 8,938,301; 8,942,777; 8,948,834; 8,948,860; 8,954,146; 8,958,882; 8,961,386; 8,965,492; 8,968,195; 8,977,362; 8,983,591; 8,983,628; 8,983,628; 8,986,207; 8,989,835; 8,989,836; 8,996,112; 9,008,367; 9,008,754; 9,008,771; 9,014,216; 9,014,453; 9,014,819; 9,015,057; 9,020,576; 9,020,585; 9,020,788; 9,022,936; 9,026,202; 9,028,405; 9,028,412; 9,033,884; 9,037,224; 9,037,225; 9,037,530; 9,042,952; 9,042,958; 9,044,188; 9,055,871; 9,058,473; 9,060,671; 9,060,683; 9,060,695; 9,060,722; 9,060,746; 9,072,482; 9,078,577; 9,084,584; 9,089,310; 9,089,400; 9,095,266; 9,095,288; 9,100,758; 9,107,586; 9,107,595; 9,113,777; 9,113,801; 9,113,830; 9,116,835; 9,119,551; 9,119,583; 9,119,597; 9,119,598; 9,125,574; 9,131,864; 9,135,221; 9,138,183; 9,149,214; 9,149,226; 9,149,255; 9,149,577; 9,155,484; 9,155,487; 9,155,52; 9,165,472; 9,173,582; 9,173,610; 9,179,854; 9,179,876; 9,183,351; RE34015; RE38476; RE38749; RE46189; 20010049480; 20010051774; 20020035338; 20020055675; 20020059158; 20020077536; 20020082513; 20020085174; 20020091318; 20020091335; 20020099295; 20020099306; 20020103512; 20020107454; 20028112732; 20020117176; 20028128544; 20020138013; 20020151771; 20020177882; 20020182574; 20020183644; 20020193670; 20030001098; 20030009078; 20030023183; 20030028121; 20030032888; 20030035301; 20030036688; 20030046018; 20030055355; 20030070685; 20030093004; 20030093129; 20030100844; 20030120172; 20030130708; 20030135128; 20030139681; 20030144601; 20030149678; 20030158466; 20030158496; 20030158587; 20030160622; 20030167018; 20030171658; 20030171685; 20030176804; 20030181821; 20030185408; 20030195429; 2003026654; 20030225341; 20030229291; 20030236458; 20041002635; 20040006285; 20041006376; 20041010203; 2004003928; 20041059203; 20041059241; 20041064120; 20041064066; 20040068164; 20040068198; 20040073098; 20041073129; 20041077967; 20040079372; 20040082862; 2004108287; 20040097802; 20040116784; 20041116791; 20040116798; 20040116825; 20040117098; 20041143170; 20040144925; 20040152995; 20040158300; 20040167418; 20040181162; 20040193068; 20040199482; 20042048636; 20042048637; 2004204865; 20040210146; 20041220494; 20040220782; 20041225179; 2004023010; 20041243017; 20041254493; 20041260168; 20050007091; 20050010116; 20050018858; 20050025704; 20050033154; 20050033174; 20050038354; 20050043774; 20050075568; 20050080348; 20050080828; 20050085744; 20050096517; 20050113713; 20050119586; 20050124848; 20050124863; 20050135102; 20050137494; 20050148893; 20050148894; 20050148895; 20050149123; 20050182456; 20050197590; 20050208517; 20050216071; 20050251055; 20050256385; 20050256418; 20050267362; 20050273017; 20050277813; 20050277912; 20060004298; 20060009704; 20060015034; 20060041201; 20060047187; 20060047216; 20060047324; 20060058590; 20060074334; 20060082727; 20060084877; 20060089541; 20060089548; 20060094968; 20060100530; 2006010271; 20060111644; 20060116556; 20060135880; 20060149144; 20060153396; 20060155206; 20060155207; 20060161071; 20060161075; 20060161218; 20060167370; 20060167722; 20060173364; 20060184058; 20060189880; 20060189882; 20060200016; 20060200034; 20060200035; 20060204532; 20060206033; 2006021760; 20060233390; 20060235315; 20060235324; 20060241562; 20060241718; 20060251303; 20060258896; 20060258950; 20060265022; 20060276695; 20070007454; 20070016095; 20070016264; 2007102673; 200702675; 20070032733; 20070032737; 20070038382; 20070060830; 20070060831; 20070066914; 20070083128; 2007009372; 20070100246; 20070100251; 20070100666; 20070129647; 20070135724; 20070135728; 20070142862; 20070142873; 20070149860; 20070161918; 20070162086; 200701676894; 20070167853; 20070167858; 20070167991; 20070173733; 20070179396; 20070191688; 20070191691; 20070191697; 20070197930; 20070203448; 20070208212; 20070208268; 2007023786; 20070225581; 20070225674; 20070225932; 20070249918; 20070249952; 20070255135; 20070280151; 20070285508; 20070285533; 20070273504; 20070276270; 20070276278; 20070276278; 20070276608; 20070291832; 20080001600; 20080001735; 20080004514; 20080004904; 20080009685; 20080009772; 20080013747; 2008002332; 2008002336; 2008002340; 2008002342; 20080033286; 20080036752; 20080045823; 20080045844; 20080051668; 20080051858; 20080058668; 20080074307; 20080077010; 20080077015; 20000082018; 20080097197; 20080119716; 20080119747; 20080119900; 20080125669; 20080139953; 20080149493; 20080154111; 20080167535; 20080167549; 20080167569; 20080177195; 20080177196; 20080177197; 20080188765; 20080195166; 20080200831; 20080208072; 20080208073; 20080214902; 2008022490; 2008022472; 2008022196; 20080228100; 2008024252; 20080243014; 20080243017; 20080243021; 20080249430; 20080255468; 20080257348; 20080260212; 20080262367; 20080262371; 20080275327; 20080294919; 20080294963; 20080319326; 20080319505; 20090005675; 20090009284; 20090018429; 20090024907; 20090030476; 20090043221; 20090048530; 20090054788; 20090062660; 20090062670; 20090062676; 20090062679; 20090062680; 20090062696; 20090076338; 20090076398; 20090076490; 20090076407; 2080082688; 20000826890; 20090083071; 20090088658; 20090094305; 20090112281; 20090118636; 20090124868; 2009012492; 20090124922; 20090124923; 20090137915; 20090137923; 20090149148; 20090156954; 20090156956; 20090157662; 20090171232; 20090171249; 20090177090; 20090177108; 20090179642; 2009018221; 20090192394; 20090198144; 20090198145; 20090204015; 20090209835; 2000216091; 2009026146; 20090227876; 20090227877; 20090227882; 20090227888; 20090249118; 20090247893; 20090247894; 20090264785; 20090264952; 20090275853; 20090287107; 2009029280; 20090297000; 20090306534; 20090312663; 20090312664; 20090312808; 20090312817; 20090316925; 20090318779; 20090323048; 20090326353; 20100010364; 20100023089; 20100030073; 2010003621; 20100036276; 20100041962; 20100042011; 20100043795; 20100049068; 20100049075; 20100049482; 20100056938; 20100069762; 20100069775; 20100076333; 20100076338; 20100079292; 20100087900; 20100094103; 20100094152; 20100094155; 20100099954; 20100106044; 20100114813; 20100130868; 20100137728; 20100137937; 20100143256; 2010015262; 20100160737; 20100174161; 20100179447; 20100185113; 20100191124; 20100191139; 20100191305; 20100195770; 2010019808; 20100198101; 20100204614; 20100204748; 20100204750; 20100217100; 20100217146; 20100217348; 20100222694; 20100224188; 20100234705; 20100234752; 20100234753; 20100245093; 20100249627; 20100249635; 20100258126; 20100261977; 20100282377; 20100268055; 20100280403; 20100286548; 20100286747; 20100292752; 20100293115; 20100298735; 20100303101; 20100312188; 20100318025; 20100324441; 20100331648; 20100331715; 2011000415; 20110009715; 20110009729; 20110009752; 20110015501; 20110015536; 20110028802; 20110028858; 20110034822; 20110038515; 20110040202; 20110046473; 20110054279; 20110054345; 20110066005; 20110066041; 20110066042; 20110066053; 20110077538; 20110082381; 20110087125; 20110092834; 20110092838; 20110098583; 20110105858; 20110105915; 20110105938; 20110106206; 20110112379; 20110112381; 20110112426; 20110112427; 20110115624; 20110118536; 20110118618; 20110118618; 20110119212; 20110125046; 20110125048; 20110125238; 20110130675; 20110144521; 20110152710; 20110160607; 20110160608; 20110160795; 20110162645; 20110178441; 20110178581; 20110181422; 20110184650; 20110190600; 20110196693; 20110208538; 20110218453; 2011028950; 20110224568; 20110224570; 20110224602; 20110245708; 20110251583; 20110251985; 20110257517; 20110283995; 20110270117; 20110270576; 20110282234; 20110288424; 20110288431; 20110295142; 20110295143; 20110295338; 20110301436; 20110301438; 20110301441; 20110301448; 20110301486; 20110301487; 20110307029; 20110307079; 20110313308; 20110313760; 20110319724; 20120004561; 20120004564; 20120004748; 20120010536; 2012001628; 20120016252; 20120022336; 20120022350; 20120022351; 20120022365; 20120022384; 20120022392; 20120022844; 20120029320; 20120029378; 20120029379; 20120035431; 20120035433; 20120035765; 20120041330; 20120046711; 20120053433; 20120053491; 20120059273; 20120065536; 20120078115; 20112083700; 20112083701; 20120088987; 20120088992; 20112089004; 2012009256; 2012009257; 20120095352; 20120095357; 20120100514; 20120101387; 20120101411; 201201014012; 20120101430; 20120108998; 20120116235; 20120123232; 20120123290; 20120125337; 20120136242; 20120136605; 20120143074; 20120143075; 20120149997; 20120150545; 20120157963; 20120159656; 20120165624; 20120165631; 20120172882; 20120172688; 20120172743; 20120191000; 20120197092; 20120197153; 20120203087; 20120203130; 20120203131; 20120203133; 20120203725; 20120209126; 20120209136; 20120209138; 20120220843; 2012022088; 2012022310; 20120226334; 20120238890; 20120242501; 20120245464; 20120245481; 20120253141; 2012025320; 20120253248; 20120285080; 20120271190; 20120277545; 20120277548; 20120277816; 20120296182; 20120296568; 20120302842; 20120302845; 20120302856; 20120302894; 20112310100; 201203110100; 2012032758; 20120323132; 20120330108; 20130006124; 20130009783; 20130011818; 20130012786; 20130012787; 20130012788; 20130012788; 20130012790; 20130012802; 20130012830; 20130013327; 20130023783; 20130030257; 20130035579; 20130039498; 20130041235; 20130046151; 20130046193; 20130046715; 20130060110; 20130060125; 20130066392; 20130066394; 20130066395; 20130069780; 20130070929; 20130072807; 20130076885; 20130079606; 2013007962; 20130079647; 20130079656; 20130079657; 20130080127; 20130080488; 20130095458; 20130096391; 20130096393; 20130096394; 20130098640; 20130096441; 20130096838; 20130096840; 20130102833; 2013002897; 20130109995; 20130109996; 20130116520; 20130116561; 20130116580; 20130118494; 20130123584; 20130127708; 20130130799; 20130137936; 20130137930; 20130138002; 20130144106; 20130144107; 20130144108; 20130144183; 20130150650; 20130150651; 20130150658; 20130159041; 20130165812; 20130172686; 201301726891; 20130172716; 20130172763; 20130172767; 20130172772; 20130172774; 20130178718; 20130182860; 20130184552; 20130184558; 20130184603; 20130188854; 20130190577; 20130190642; 20130197321; 20130197322; 20130197320; 20130197339; 20130204150; 20130201224; 2013021276; 2013021291; 2013027982; 20130218043; 2013028053; 20130218233; 20130221961; 20130225940; 20130225992; 20130231574; 20130231580; 20130231947; 20130238048; 20130238050; 20130238063; 20130245422; 20130245486; 20130245711; 20130245712; 20130266163; 20130267760; 20130267866; 20130267928; 20130274580; 20130274625; 20130275158; 20130281811; 20130282339; 20130289401; 20130289413; 20130289417; 20130289424; 20130289433; 20130295016; 20130300573; 20130303828; 20130303934; 20130304153; 20130310660; 20130310908; 20130324880; 20130338448; 20130338458; 20130344465; 20130345522; 20130345523; 20140005988; 20140012061; 20140012010; 20140012133; 20140012153; 20140018792; 20140019165; 20140023998; 20140025396; 20140025397; 20140038147; 20140046208; 20140051044; 20140051960; 20140051961; 20140052213; 20140055284; 20140058241; 20140066738; 20140066763; 20140070958; 2014007227; 2014007230; 20140073863; 20140073864; 20140073866; 20140073870; 20140073875; 20140073876; 20140073877; 20140073878; 20140073898; 20140073948; 20140073948; 20140073951; 20140073953; 20140073954; 20140073955; 20140073956; 20140073960; 20140073961; 20140073963; 20140073965; 20140073966; 20140073967; 20140073968; 20140073974; 20140073975; 20140074060; 20140074179; 20140074180; 20140077946; 20140081114; 2014008111; 20140094720; 20140098081; 20140100467; 20140104058; 20140105436; 20140107464; 20140107518; 20140107525; 20140114165; 20140114205; 2140121446; 21140121476; 2140121554; 20140128762; 20114012074; 20140135879; 20140136585; 20140140567; 20140143064; 20140148723; 20140152673; 20140155706; 20140155714; 20140155730; 20140156000; 20140163328; 20140163330; 20140163331; 20140163332; 20140163333; 20140163335; 20140163336; 20140163337; 20140163385; 20140163408; 20140163425; 20140163897; 20140171820; 20140175261; 201401768944; 20140179980; 20140180088; 20140180092; 20140180093; 20140180094; 20140180095; 20140180096; 20140180097; 20140180098; 20140180100; 20140180112; 20140180113; 20140180145; 20140180153; 20140180160; 20140180161; 20114110176; 20140180177; 20140180597; 20140187994; 20140188006; 20140188770; 20140194702; 20140194758; 20140194758; 20140194768; 20140194768; 20140194780; 20140194793; 20140203797; 21140213937; 2014024330; 20140228651; 20140228702; 20140232516; 20140235965; 20140236039; 20140236077; 20140237073; 20140243614; 2014024362; 2014024362; 20140243694; 2014024942; 20140257073; 21401257147; 20140266696; 20140266787; 20140275886; 20140275888; 20140275891; 20140276013; 20140276014; 20140276090; 20140276123; 20140276130; 20140276181; 20140276183; 20140279746; 20140288381; 20140288614; 20140288953; 20140289172; 20140296724; 20140303453; 20140303454; 20140303508; 20140309943; 20140313303; 2014031627; 20140316221; 20140316230; 20140316235; 20140316278; 20140323900; 20140324118; 20140330102; 20140330157; 20140330158; 20140330334; 20140330404; 20140336473; 20140347491; 20140350431; 20140350436; 20140358025; 2014036472; 20140364746; 20140369537; 20140371544; 20140371598; 20140378808; 20140378810; 20140379620; 20150003698; 20150003698; 20150005592; 20150005594; 20150005640; 20150005644; 20150005660; 20150005680; 20150006186; 20150016618; 20150018758; 20150025351; 20150025422; 20150032017; 20150038804; 20150038868; 20150039110; 20150042477; 20150045686; 20150051663; 20150057512; 20150065838; 20150073237; 20150073306; 20150080671; 20150080746; 20150087931; 20150088024; 20150092948; 20150093728; 20150099941; 20150099962; 20150103360; 20150105631; 20150105641; 20150105837; 20150112222; 20150112408; 20150119652; 20150119743; 20150119746; 2015012682; 20150126845; 20150126848; 20150126873; 20150134264; 20150137988; 2015014152; 20150141788; 20150141794; 20150153477; 20150157235; 20150157266; 20150164348; 20150164362; 20150164375; 20150164404; 20150181840; 20150182417; 20150190070; 20150190085; 20150190636; 20150190637; 20150196213; 20150199010; 20150201879; 20150202447; 20150203822; 20150208940; 20150208975; 2015023191; 2015026436; 2015026468; 20150217082; 20150220486; 20150223743; 20150227702; 20150230750; 2015023140; 20150238106; 20150238112; 20150238137; 20150245800; 20150247921; 20150250393; 20150250401; 20150250415; 20150257645; 20150257673; 20150257674; 20150257700; 20150257712; 20150265164; 20150268825; 2015027246; 20150282730; 20150282755; 20150282760; 20150290420; 20150290453; 20150290454; 20150297106; 20150297141; 20150304101; 20150305685; 20150309568; 20150313496; 20150313535; 20150327813; 20150327837; 20150335292; 20150342478; 20150342493; 20150351655; 20150351701; 20150359441; 20150359450; 20150359452; 20150359467; 20150359486; 20150359492; 20150366497; 20150366504; 20150366516; 20150366518; 20150374285; 20150374292; 20150374300; 20150380008; 20160000348; 20160000354; 20160007915; 20160007918; 20160012748; 20160015201; 20160015289; 20160022141; 20160021056; 2016002264; 20160022167; 20160022206; 20160027293; 20160029917; 20160029918; 20160029946; 20160029950; 20160029965; 20160030702; 20160038037; 20160038038; 20160038048; 20160038091; 20160045150; 20160045756; 20160051161; 20160051162; 20160051187; 20160051195; 20160055415; 20160058301; 20160066788; 20160067494; 20160073886; 20160074661; 20160081577; 20160081616; 20160087603; 20160089031; 20160100768; 20160101260; 20160106331; 20160106344; 20160112022; 20160112684; 20160113539; 20160113545; 20160113567; 20160113587; 20160119726; 20160120433; 20160120434; 20160120464; 20160120480; 20160120596; 20160132654; 20160135691; 20160135727; 20160135754; 20160140834; 20160143554; 20160143560; 20160143594; 20160148531; 20160150988; 20160151014; 20160151018; 20160151620; 20160157742; 20160157820; 20160162652; 20160165852; 20160165853; 20160166169; 20160166197; 20160166199; 20160166208; 20160174099; 20160174863; 20160178392; 20160183828; 20160183861; 20160191517; 20160192041; 20160192842; 20160192047; 20160192879; 20160196758; 2016018963; 2016018966; 20160202755; 20160206877; 20160206880; 20160213276; 20160213314; 20160220133; 20160220134; 20160220136; 20160220166; 20160220836; 20160220837; 20160224757; 20160228018; 20160228029; 20160228058; 20160228705; 20160232011; 20160235324; 20160235351; 20160235352; 20160239084; 20160242658; 20160242690; 20160242698; 20160248434; 20160249841; 20160256063; 20160256112; 20160256118; 20160259905; 20160262664; 20160262685; 20160262895; 20160262703; 20160278651; 20160278697; 20160278713; 20160202941; 20160287120; 20160207157; 20160287162; 20160287166; 20160207871; 20160296157; 20160302588; 20160302704; 20160302709; 20160302720; 20160302737; 20160303402; 20160310031; 20160310070; 20160317056; 2016032446; 20160331264; 20160338634; 20160338644; 20160338798; 20160346542; 20160354003; 20160354027; 20160360965; 20160360970; 20160361021; 20160361041; 20160367204; 20160374581; 20160374618; 20170000404; 20170001016; 20170007165; 20170007173; 20170014037; 20170014083; 20170020434; 20170020447; 20170027467; 20170032098; 20170035392; 20170042430; 20170042468; 20170042475; 20170053513; 20170055838; 20170055898; 20170055913; 20170065198; 2017006528; 20170065229; 20170071495; 20170071523; 20170071529; 20170071532; 20170071537; 20170071546; 20170071551; 20170071552; 20170079538; 20170079596; 20170086672; 20170086695; 20170091567; 2017009572; 20170105647; 20170112379; 20170112427; 20170120066; 20170127946; 20170132016; 20170135597; 20170135604; 20170135626; 20170135625; 20170135631; 20170135633; 20170143231; 20170143248; 20170143255; 20170143257; 20170143258; 20170143266; 20170143257; 20170143258; 20170143273; 20170143280; 20170143282; 20170143960; 20170143963; 20170146386; 20170146387; 20170146390; 20170146391; 20170147754; 20170148240; 20170150896; 20170150916; 20170156593; 20170156606; 20170156655; 20170164878; 20170164901; 20170172414; 20170172501; 20170172520; 20170173252; 20170177023; 20170181693; 20170185148; 20170188865; 20170188872; 20170188947; 20170188992; 20170189691; 20170196497; 20170202474; 20170202518; 20170203154; 20170209053; and 20170208083.

There are many approaches to time-frequency decomposition of EEG data, including the short-term Fourier transform (STFT), (Gabor D. Theory of Communication. J. Inst. Electr. Engrs. 1949; 93:429-457) continuous (Daubechies I. Ten Lectures on Wavelets. Philadelphia, Pa: Society for Industrial and Applied Mathematics; 1992:357.21. Combes J M, Grossmann A, Tchamitchian P. Wavelets: Time-Frequency Methods and Phase Space-Proceedings of the International Conference; Dec. 14-18, 1987; Marseille, France) or discrete (Mallat S G. A theory for multiresolution signal decomposition: the wavelet representation. IEEE Trans Pattern Anal Mach Intell.1989; 11:674-693) wavelet transforms, Hilbert transform (Lyons R G. Understanding Digital Signal Processing. 2nd ed. Upper Saddle River, NJ: Prentice Hall PTR; 2004:688), and matching pursuits (Mallat 5, Zhang 7. Matching pursuits with time-frequency dictionaries. IEEE Trans. Signal Proc.1993; 41(12):3397-3415). Prototype analysis systems may be implemented using, for example, MatLab with the Wavelet Toolbox, www.mathworks.com/products/wavelet.html.

See, U.S. Pat. Nos. 6,196,972; 6,338,713; 6,442,421; 6,507,754; 6,524,248; 6,547,736; 6,616,611; 6,816,744; 6,865,494; 6,915,241; 6,936,012; 6,996,261; 7,043,293; 7,054,454; 7,079,771; 7,112,713; 7,146,211; 7,148,572; 7,164,941; 7,208,788; 7,254,431; 7,250,867; 7,282,030; 7,321,837; 7,330,032; 7,333,619; 7,381,185; 7,537,568; 7,559,903; 7,565,193; 7,567,693; 7,604,603; 7,624,293; 7,640,055; 7,715,919; 7,725,174; 7,729,755; 7,751,878; 7,778,693; 7,794,406; 7,797,040; 7,801,592; 7,803,118; 7,803,119; 7,879,043; 7,896,807; 7,899,524; 7,917,206; 7,933,646; 7,937,138; 7,976,465; 8,014,847; 8,033,996; 8,073,534; 8,095,210; 8,137,269; 8,137,270; 8,175,696; 8,177,724; 8,177,726; 8,180,601; 8,187,181; 8,197,437; 8,233,965; 8,236,005; 8,244,341; 8,248,069; 8,249,698; 8,280,514; 8,295,914; 8,326,433; 8,335,664; 8,346,342; 8,355,768; 8,386,312; 8,386,313; 8,392,250; 8,392,253; 8,392,254; 8,392,255; 8,396,542; 8,406,841; 8,406,862; 8,412,655; 8,428,703; 8,428,704; 8,43,374; 8,464,288; 8,475,387; 8,483,815; 8,494,610; 8,494,829; 8,494,905; 8,498,698; 8,509,881; 8,533,042; 8,548,786; 8,571,629; 8,579,786; 8,591,419; 8,606,360; 8,628,480; 8,655,428; 8,666,478; 8,682,422; 8,706,183; 8,706,205; 8,718,747; 8,725,238; 8,738,136; 8,747,382; 8,755,877; 8,761,868; 8,762,202; 8,768,448; 8,781,796; 8,790,255; 8,790,272; 8,821,408; 8,825,149; 8,831,731; 8,843,210; 8,849,392; 8,849,632; 8,855,773; 8,858,440; 8,862,210; 8,862,581; 8,903,479; 8,918,178; 8,934,965; 8,951,190; 8,954,139; 8,955,010; 8,958,868; 8,983,628; 8,983,628; 8,898,835; 9,020,788; 9,028,217; 9,031,644; 9,050,470; 9,060,671; 9,070,492; 9,072,832; 9,072,905; 9,078,584; 9,084,896; 9,095,295; 9,101,276; 9,107,595; 9,116,835; 9,125,574; 9,149,718; 9,155,487; 9,192,309; 9,198,621; 9,204,835; 9,211,417; 9,215,978; 9,232,910; 9,232,984; 9,238,142; 9,242,067; 9,247,911; 9,248,286; 9,254,383; 9,277,871; 9,277,873; 9,282,934; 9,288,603; 9,302,100; 9,307,944; 9,308,372; 9,320,450; 9,336,535; 9,357,941; 9,375,151; 9,375,171; 9,375,571; 9,403,038; 9,415,211; 9,427,581; 9,443,141; 9,451,886; 9,454,646; 9,468,956; 9,462,975; 9,468,541; 9,471,978; 9,480,402; 9,492,084; 9,504,410; 9,522,278; 9,533,113; 9,545,285; 9,560,984; 9,563,740; 9,615,749; 9,616,166; 9,622,672; 9,622,676; 9,622,702; 9,622,703; 9,623,240; 9,636,019; 9,649,036; 9,659,229; 9,668,694; 9,681,814; 9,681,820; 9,682,232; 9,713,428; 20020035338; 20020091318; 20020095098; 20020103428; 20020103428; 20020193670; 20030032888; 20030046018; 20030093129; 20030160622; 20030185408; 2003026654; 20040039288; 20040049484; 20040092808; 20040133118; 20040133120; 20040133390; 20040138536; 20040138580; 20040138711; 20040152958; 20040158118; 20050010091; 20050018858; 20050033174; 20050075568; 20050085744; 20050119547; 20050148893; 20050148894; 20050148895; 20050154290; 20050167588; 20050240087; 20050245796; 20050267343; 20050267344; 20050283053; 20050283090; 20060020184; 20060036152; 20060036153; 20060074290; 20060078183; 20060135879; 20060153396; 20060155495; 20060161384; 20060173364; 20060280013; 2006027816; 20060233390; 20060281980; 20070016095; 20070066915; 20070100278; 20070179395; 20070179734; 20070191704; 20070208668; 20070225932; 20070255122; 20070255135; 20070280151; 20070285508; 20070287896; 20080021345; 20080033508; 20080064934; 20080074307; 20080077015; 2000009118; 20008097197; 20080119716; 28080177196; 20080221401; 2008022441; 20080243014; 20080243017; 20080255948; 20080262367; 20090005667; 20090033333; 20090036791; 20090054801; 20090062676; 20090177144; 20090220425; 20090221930; 20090270758; 20090281448; 20090287271; 20090287272; 20090287273; 20090287467; 20090299168; 20090306534; 20090312846; 20090318794; 20090322331; 20100030073; 2010003621; 20100049276; 20100068751; 20100069738; 20100094152; 2010099975; 20100106041; 20100198090; 20100204604; 20100204748; 20100249638; 20100280372; 20100331976; 2011000415; 20110015515; 20110015538; 20110040713; 20110066041; 20110066042; 20110074396; 20110077538; 20110092834; 20110092838; 20110098583; 20110160543; 20110172725; 20110178441; 20110184305; 20110191350; 2011028950; 20110257518; 20110270074; 20110282230; 20110288431; 20110295143; 20110301441; 20110313268; 20110313487; 20120004518; 20120004561; 2012802394; 20120022343; 20120029378; 20120041279; 20120046535; 20120053473; 20120053476; 20120053478; 20120053479; 20120083708; 20120108918; 20120108997; 20120143038; 20120145152; 20120150545; 20120157804; 20120159656; 20120172882; 20120184828; 20120197153; 20120209138; 20120253261; 20120265287; 20120271151; 20120271376; 20120289868; 20128310105; 2012032758; 20130012804; 20130041235; 20130060125; 20130066392; 20130066395; 20130072775; 20130079621; 20130102897; 20130116520; 20130123607; 20130127708; 20130131438; 20130131461; 20130165804; 20130167360; 20130172716; 20130172772; 20130178733; 20130184597; 20130204122; 20130201238; 20130223708; 20130226261; 20130237874; 20130238048; 20130238050; 20130245416; 20130245424; 2013024548; 20130245486; 20130245711; 20130245712; 20130281490; 20130274562; 20130289364; 20130295016; 20130310422; 20130310908; 20130317380; 20130338518; 20130338803; 20140039279; 20140057232; 2014005821; 20140058528; 20140074179; 20140074180; 20140094710; 20140094720; 2014010752; 20140142854; 20140148657; 20140148716; 20140148728; 20140180153; 20140180160; 20140187901; 20140228702; 20140243647; 20140243714; 20140257120; 20140275807; 20140276130; 20140276187; 20140303454; 20140303508; 20140309614; 20140316217; 20140316248; 20140324118; 20140330334; 20140330335; 20140330336; 20140330404; 20140335488; 20140350634; 20140350864; 20150005646; 20150005660; 20150011907; 20150018665; 20150018698; 20150018702; 20150025422; 20150038868; 20150073294; 20150073306; 20150073505; 20150080671; 20150080695; 20150099962; 2015012680; 20150151142; 20150164431; 20150190070; 20150190636; 20150190637; 2015019623; 20150196248; 2015003191; 2015006439; 20150245800; 20150248470; 20150248615; 20150272652; 20150297106; 20150297893; 20150305686; 20150313490; 20150366482; 20150379370; 20160000348; 20160007899; 20160022167; 2016002216; 20160022207; 20160027423; 20160029965; 20160038042; 20160038043; 20160045120; 20160051812; 20160058304; 20160066838; 20160107309; 20160113587; 20160120420; 20160120432; 20160120437; 20160120457; 20160128596; 20160120597; 20160135754; 20160143594; 20160144175; 20160151620; 20160157742; 20160157820; 20160174863; 20160174907; 20160176053; 20160183881; 20160184029; 20160189873; 20160206380; 2016003261; 20160213317; 20160220850; 20160228028; 20160228702; 20160235324; 20160239966; 20160239960; 20160242645; 20160242665; 2016024266; 20160242690; 20160249841; 20160250355; 20160256063; 2016025605; 20160262664; 20160278653; 20160278713; 20160207117; 20160287162; 2016020716; 20160207869; 20160303402; 20160331264; 20160331307; 20160345895; 20160345911; 20160346542; 20160361041; 20160361546; 20160367186; 20160367198; 20170031449; 20170031441; 20170039706; 20170042444; 20170045601; 20170071521; 20170079500; 20170079509; 20170091410; 20170113046; 20170120041; 20170128015; 20170135594; 20170135626; 20170136240; 20170165020; 20170172446; 20170173326; 20170188870; 20170188905; 20170188916; 20170188922 and 20170196519.

Single instruction, multiple data processors, such as graphic processing units including the nVidia CODA environment or AMD Firepro high-performance computing environment are known, and may be employed for general purpose computing, finding particular application in data matrix transformations.

See, U.S. Pat. Nos. 5,273,038; 5,503,149; 6,249,308; 6,272,370; 6,298,259; 6,370,414; 6,385,479; 6,490,472; 6,556,695; 6,697,660; 6,801,648; 6,907,280; 6,996,261; 7,092,748; 7,254,500; 7,338,455; 7,346,382; 7,490,085; 7,497,828; 7,539,520; 7,565,193; 7,567,693; 7,577,472; 7,597,665; 7,627,370; 7,680,526; 7,729,755; 7,809,434; 7,840,257; 7,860,548; 7,872,235; 7,899,524; 7,904,134; 7,904,131; 7,907,998; 7,983,749; 7,983,741; 8,000,773; 8,014,847; 8,069,125; 8,233,682; 8,233,965; 8,235,907; 8,248,068; 8,356,004; 8,379,952; 8,496,838; 8,423,125; 8,445,851; 8,553,956; 8,586,932; 8,606,349; 8,615,479; 8,644,910; 8,671,100; 8,696,722; 8,712,512; 8,718,747; 8,761,866; 8,781,557; 8,814,923; 8,821,376; 8,834,546; 8,852,103; 8,870,737; 8,936,630; 8,951,188; 8,951,192; 8,958,882; 8,983,155; 9,005,126; 9,020,586; 9,022,936; 9,028,412; 9,033,884; 9,042,950; 9,078,584; 9,101,279; 9,135,490; 9,144,392; 9,114,255; 9,155,521; 9,167,970; 9,179,854; 9,179,850; 9,198,637; 9,204,835; 9,208,550; 9,211,077; 9,213,076; 9,235,685; 9,242,067; 9,247,924; 9,280,014; 9,268,015; 9,271,651; 9,271,674; 9,275,191; 9,292,920; 8,307,925; 9,322,895; 9,326,742; 9,330,206; 9,368,265; 9,395,425; 9,402,558; 9,414,776; 9,436,989; 9,451,883; 9,451,899; 8,468,541; 8,471,978; 9,480,402; 9,480,425; 9,486,160; 9,592388; 9,615,789; 9,626,756; 9,672,302; 9,672,617; 9,682,232; 20020033454; 20020035317; 20020037095; 20020042563; 20020058867; 20020103428; 2002010342; 20030018277; 20030093004; 20030128801; 20040082862; 20049092809; 20040096395; 20049116791; 20049116790; 20049122787; 20049122790; 20049166536; 20040215082; 20050007091; 20050020910; 20050033154; 20050079636; 20050119547; 20050154290; 2005022263; 20050240253; 20050203053; 20060036152; 20060036153; 20060052706; 20060058683; 20060074290; 20060078183; 20060084858; 20060149160; 2006016100; 20060241382; 20060241710; 20070191704; 20070239059; 20080001600; 20080009772; 20080033291; 20080039737; 20080042067; 20008097235; 20080097785; 20080128626; 2080154126; 2008020441; 20080220077; 20080228; 20080230702; 20080230705; 20080249430; 20080262327; 20080275349; 2090012387; 20090018407; 20090022825; 20090024950; 20090062660; 20090078875; 20090118610; 20090156907; 20090156955; 20090157323; 20090157481; 20090157482; 20090157625; 20090157751; 20090157813; 20090163777; 20090164131; 20090164132; 20090171164; 20090172549; 20090179642; 20090209831; 2009020930; 2009024613; 20090299169; 20090304582; 20090306532; 20090306534; 20090312808; 2009031217; 20090318773; 20090318794; 20090322331; 20090326604; 20100021378; 20100036233; 20100041949; 20100042011; 20100049482; 20100069738; 20100069777; 20100082506; 20100113959; 20100249573; 20110015515; 2011001553; 20110020827; 20110077503; 20110118536; 20110125077; 20110125078; 2011012912; 20110160543; 20110161011; 2011017250; 20110172553; 20110178359; 20110190846; 2011028405; 20110224571; 20110230730; 20110257519; 2011023962; 20110263960; 20110270074; 20110288400; 20110301448; 20110306845; 20110306846; 20110313274; 2012000394; 20120022343; 20120035433; 20120053483; 20120163689; 20120165904; 2010215114; 2012009195; 2010219507; 20120245474; 20120253261; 20120253434; 2012028854; 20120310107; 20120316793; 20130012804; 20130060125; 20130063550; 20130085678; 20130096490; 20130110616; 20130116561; 20130123607; 20130131438; 20130131461; 20130178693; 20130178733; 20130184550; 2013021230; 20130221961; 20130245424; 20130274586; 2030289385; 20130289386; 20130303934; 20114058528; 20114066763; 2014911960; 20114151563; 20114155730; 20114163368; 20149171757; 20114180088; 20149180092; 20114180093; 20149180094; 20114180095; 2014180096; 20114180097; 20114180098; 20114180100; 2014918012; 20114180113; 20114180176; 20114180177; 20114184550; 20149193336; 20140200414; 20114243614; 20149257047; 20114275807; 20114303486; 20114315168; 20114316248; 2014932384; 20114335488; 20140343397; 20114343398; 20114343498; 20149364721; 20114378830; 20150011866; 20150038812; 20150051663; 20150099959; 20150112499; 20150119658; 20150119689; 20150148700; 20150150473; 20150196800; 20150200046; 2015029732; 20150223905; 20150227702; 2015024790; 20150248615; 20150253410; 20150289779; 20150290453; 20150290454; 20150313549; 20150317796; 201503246892; 20150366482; 20150375006; 20160005320; 20160027342; 20160029965; 20160051161; 20160051162; 20160055304; 20160058304; 20160058392; 20160066838; 20160103487; 20160120437; 20160120457; 20160143541; 20160157742; 20160184029; 20160196393; 20160228702; 20160231491; 20160239966; 20160239968; 2016026006; 20160267809; 20160270723; 20160302720; 20160303397; 20160317077; 20160345911; 20170027539; 20170039706; 20170045601; 20170061034; 20117085855; 20170091418; 2017012493; 20170113046; 20170120041; 20170160360; 20170164861; 20170169714; 20170172527; and 20170202475.

Statistical analysis may be presented in a form that permits parallelization, which can be efficiently implemented using various parallel processors, a common form of which is a SIMD (single instruction, multiple data) processor, found in typical graphics processors (CPUs).

See, U.S. Pat. Nos. 8,496,890; 8,509,879; 8,542,916; 8,852,103; 8,934,986; 9,022,936; 9,028,412; 9,031,653; 9,033,884; 9,037,530; 9,055,974; 9,149,255; 9,155,521; 9,198,637; 9,247,924; 9,268,014; 9,268,015; 9,367,131; 9,414,780; 9,420,970; 9,430,615; 9,442,525; 9,444,998; 9,445,763; 9,489,956; 9,474,481; 9,489,854; 9,504,420; 9,510,790; 9,519,81; 9,526,906; 9,538,948; 9,585,581; 9,622,672; 9,641,665; 9,652,626; 9,684,335; 9,687,187; 9,693,684; 9,693,724; 9,706,963; 9,712,736; 20090118622; 2010009889; 20110066041; 20110066042; 20110098583; 20110301441; 20120130204; 20120265271; 2012030759; 20130060158; 20130113816; 20130131438; 20130184786; 20114031888; 20114031903; 20140039975; 20114114888; 20149226131; 20149279341; 20149296733; 20114303424; 20149313303; 20114315168; 20149316235; 20149364721; 20114378810; 20150003698; 20150003698; 20150005649; 20150005644; 20150006186; 20150029087; 20150033245; 20150033250; 2015003325; 20150033262; 20150033266; 20150081226; 20150088093; 20150093729; 20150105701; 20150112898; 20150126845; 20150150122; 20150190062; 20150190070; 20150190077; 20150190094; 20150192776; 2015019603; 20150196800; 20150199010; 20150241916; 2015024260; 20150272496; 20150272510; 20150282705; 20150202749; 20150289217; 20150297109; 20150305688; 20150335295; 20150351655; 20150366482; 20160027342; 20160029896; 20160058366; 20160058376; 20160058673; 20160060926; 20160065724; 20160065840; 20160077547; 20160081625; 20160103487; 20160104906; 20160109958; 20160113517; 20160120048; 20160120420; 20160120457; 2016012522; 20160157773; 2016015782; 20160183812; 20160191517; 20160193498; 20160196185; 20160196635; 20160206241; 20160213317; 20160228064; 20160235341; 20160235359; 20160249857; 201602498864; 20160256086; 20160262680; 2016022685; 20160270656; 20160278672; 2016020013; 20160287142; 20160306942; 20160310071; 20160317056; 20160324445; 20160324457; 20160342241; 20160360100; 20160361027; 20160366462; 20160367138; 20160367195; 20160374616; 20160378608; 20160378965; 20170000324; 20170000325; 20170000326; 2017000032; 20170000330; 20170000331; 20170000332; 20170000333; 20170000334; 20170000335; 20170000337; 20170000349; 20170000341; 20170000342; 20170000343; 20170000345; 20170000454; 20170000683; 20170001032; 20170007111; 20170007115; 20170007116; 20170007122; 20170007123; 20170007182; 20170007450; 20170007799; 20170007843; 20170010468; 20170010470; 20170013562; 20170017083; 20170020627; 20170027521; 20170028563; 20170031449; 20170032221; 20170035308; 20170035317; 20170041698; 20170042485; 20170046052; 20170065348; 20117086695; 20117086727; 20170090475; 20170103449; 20170112446; 20170113056; 20170120006; 20170143248; 20170143442; 20170156593; 20170156606; 20170164893; 20170171441; 20170172498; 20170173262; 20170185714; 20170188933; 20170196503; 20170205259; 20170206913; and 2017004786.

Artificial neural networks have been employed to analyze EEG signals.

See, U.S. Pat. No. 9,443,141; 20110218950; 20150248167; 20150248764; 20150248765; 20150310862; 20150331929; 20150338915; 20160026913; 20160062459; 20160085302; 20160125572; 20160247064; 20160274660; 20170053665; 20170099306; 20170173262; and 20170206691.

See also:
Amari, S., Natural gradient works efficiently in learning, Neural Computation 10:251-276, 1998.
Amari S., Cichocki, A. B Yang, H. H., A new learning algorithm for blind signal separation. In: Advances in Neural Information Processing Systems 8, MIT Press, 1996.
Bandettini P A, Wong E C, Hinks R S, Tikofsky R S, Hyde J S, Time course EPI of human brain function during task activation. Magn Reson Med 25:390-7, 1992.
Bell A. J. B Sejnowski T. J. An information-maximization approach to blind separation and blind deconvolution. Neural Comput 7:1129-59, 1995.
Bell, A. J. B Sejnowski, T. J., Learning the higher-order structure of a natural sound, Network: Computation in Neural Systems 7, 1996b.
Bench C J, Frith C D, Grasby P M, Friston K J, Paulesu E, Frackowiak R S, Dolan R J, Investigations of the functional anatomy of attention using the Stroup test. Neuropsychologia 31:907-22, 1993.
Boynton G M, Engel S A, Glover G H, Heeger D J, Linear systems analysis of functional magnetic resonance imaging in human V1. J Neurosci 16:4207-21., 1996.
Bringer, Julien, Hervé Chabanne, and Bruno Kindarji. “Error-tolerant searchable encryption.” In Communications, 2009. ICC'09. IEEE International Conference on, pp. 1-6. IEEE, 2009.
Buckner, R. L., Bandettini, P. A., O'Craven, K M, Savoy, R. L., Petersen, S. E., Raichle, M. E. B Rosen, B. R., Proc Natl Acad Sci USA 93, 14979-93, 1996.
Cardoso, J-F. B Laheld, B., Equivalent adaptive source separation, IEEE Trans. Signal Proc., in press.
Chapman, R. M. B McCrary, J. W., EP component identification and measurement by principal components analysis. Brain Lang. 27, 288-301,1995.
Cichocki A., Unbehauen R., B Rummert E., Robust learning algorithm for blind separation of signals, Electronics Letters 30, 1386-1387, 1994.
Comon P, Independent component analysis, A new concept? Signal Processing 38:11-20,1994.
Cover, T. M. & Thomas, J. A., Elements of Information Theory John Wiley, 1991.
Cox, R. W., AFNI: software for analysis and visualization of functional magnetic resonance neuroimages. Comput Biomed Res 29:192-73,1998.
Cox, R. W. & Hyde J. S. Software tools for analysis and visualization of fMRI data, NMR in Biomedicine, in press.
Dale, A. M. B Sereno, M. I., Improved localization of cortical activity by combining EEG and MEG with MRI cortical surface reconstruction—a linear approach. J. Cogn. Neurosci. 5:162-176, 1993.
Friston K. J., Modes or models: A critique on independent component analysis for fMRI. Trends in Cognitive Sciences, in press.
Friston K. J., Commentary and opinion: II. Statistical parametric mapping: ontology and current issues. J Cereb Blood Flow Metab 15:361-70, 1995.
Friston K. J., Statistical Parametric Mapping and Other Analyses of Functional Imaging Data. In: A. W. Toga, J. C. Mazziotta eds., Brain Mapping, The Methods. San Diego: Academic Press, 1996:393-396, 1995.
Friston K J, Frith C D, Liddle P F, Frackowiak R S, Functional connectivity: the principal-component analysis of large (PET) data sets. J Cereb Blood Flow Metab 13:5-14, 1993.
Friston K J, Holmes A P, Worsley K J, Poline J P, Frith C D, and Frackowiak R. S. J., Statistical Parametric Maps in Functional Imaging: A General Linear Approach, Human Brain Mapping 2:189-210, 1995.
Friston K J, Williams S, Howard R, Frackowiak R S and Turner R, Movement-related effects in fMRI time-series. Magn Reson Med 35:346-55, 1996.
Galambos, R. and S. Makeig, “Dynamic changes in steady-state potentials,” in: Dynamics of Sensory and Cognitive Processing of the Brain, ed. E. Basar Springer, pp. 178-199, 1987.
Galambos, R., S. Makeig, and P. Talmachoff, A 40 Hz auditory potential recorded from the human scalp, Proc Natl Acad Sci USA 78(4):2643-2647,1981.
Gelil, Zvi, Stuart Haber, and Moti Yung. “Cryptographic computation: Secure fault-tolerant protocols and the public-key model.” In Conference on the Theory and Application of Cryptographic Techniques, pp. 135-155. Springer, Berlin, Heidelberg, 1987.
George J S, Aine C J, Mosher J C, Schmidt D M, Ranken D M, Schlitt H A, Wood C C, Lewine J D, Sanders J A, Belliveau J W. Mapping function in the human brain with magnetoencephalography, anatomical magnetic resonance imaging, and functional magnetic resonance imaging. J Clin Neurophysiol 12:406-31, 1995.
Ives, J. R., Warach S, Schmitt F, Edelman R R and Schomer D L. Monitoring the patient's EEG during echo planar MRI, Electroencephalogr Clin Neurophysiol, 87: 417-420, 1993.
Jackson, J. E., A User's Guide to Principal Components. New York: John Wiley & Sons, Inc., 1991.
Jokeit, H. and Makeig, S., Different event-related patterns of gamma-band power in brain waves of fast- and slow-reacting subjects, Proc. Nat. Acad. Sci USA 91:6339-6343, 1994.
Juels, Ari, and Madhu Sudan. “A fuzzy vault scheme.” Designs, Codes and Cryptography 38, no. 2 (2006): 237-257.
Jueptner, M., K. M. Stephan, C. D. Frith, D. J. Brooks, R. S J. Frackowiak & R. E. Passingham, Anatomy of Motor Learning. I. Frontal Cortex and Attention. J. Neurophysiology 77:1313-1324, 1977.
Jung, T-P., Humphries, C., Lee, T-W., Makeig, S., McKeown, M., Iragui, V. and Sejnowski, T. J., “Extended ICA removes artifacts from electroencephalographic recordings,” In: Advances in Neural Information Processing Systems 10: MIT Press, Cambridge, MA, in press.
Jung, T-P., Humphries, C., Lee, T-W., McKeown, M. J., Iragui, V., Makeig, S. & Sejnowski, T. J., Removing electroencephalographic artifacts by blind source separation, submitted-a.
Jung, T-P., S. Makeig, M. Stensmo & T. Sejnowski, Estimating Alertness from the EEG Power Spectrum, IEEE Transactions on Biomedical Engineering, 44(1), 60-69, 1997.
Jung, T-P., Makeig, S., Westerfield, M., Townsend, J., Courchesne, E. and Sejnowski, T. J., Analysis and visualization of single-trial event-related potentials, submitted-b.
Jutten, C. & Herault, J., Blind separation of sources, part I: an adaptive algorithm based on neuromimetic architecture. Signal Processing 24,1-10,1991.
Karhumen, J., Dja, E., Wang, L., Vigario, R. & Joutsenalo, J., A class of neural networks for independent component analysis, IEEE Trans. Neural Networks, in press.
Kwong K. K., Functional magnetic resonance imaging with echo planar imaging. Magn Reson Q 11:1-20, 1995.
Kwong K. K., Belliveau J W, Chesler D A, Goldberg I E, Weisskoff R M, Poncelet B P, Kennedy D N, Happel B E, Cohen M S, Turner R, et al., Dynamic magnetic resonance imaging of human brain activity during primary sensory stimulation. Proc Natl Aced Sci USA 89:5675-9, 1992.
Lee, T.-W., Girolami, M., and Sejnowski, T. J., Independent component analysis using an extended infomax algorithm for mixed Sub-gaussian and Super-gaussian sources, Neural Computation, submitted for publication.
Lewicki, Michael S., and Sejnowski, Terence J., Learning nonlinear overcomplete representations for efficient coding, Eds. M. Kearns, M. Jordan, and S. Salla, Advances in Neural Information Processing Systems 10, in press.
Linsker, R., Local synaptic learning rules suffice to maximise mutual information in a linear network. Neural Computation 4, 691-702, 1992.
Liu A K, Belliveau J W, Dale A M. Spatiotemporal imaging of human brain activity using functional MRI-constrained magnetoencephalography data: Monte Carlo simulations. Proc Natl Aced Sci USA 95:8945-50, 1998
Manoach D S, Schlaug G, Siewert B, Darby D G, Bly B M, Benfield A, Edelman R R, Warach S, Prefrontal cortex fMRI signal changes are correlated with working memory load. Neuroreport 8:545-9, 1997.
McCarthy, G., Luby, M., Gore, J. and Goldman-Rakic, P., Infrequent events transiently activate human prefrontal and parietal cortex as measured by functional MRI. J. Neurophysiology 77: 1630-1634, 1997.
McKeown, M., Makeig, S., Brown, G., Jung, T-P., Kindermann, S., Bell, Iragui, V. and Sejnowski, T. J., Blind separation of functional magnetic resonance imaging (fMRI) data, Human Brain Mapping, 6:160,18, 1998a.
McKeown, M. J., Humphries, C., Achermann, P., Borbely, A. A. and Sejnowski, T. J., A new method for detecting state changes in the EEG: exploratory application to sleep data. J. Sleep Res. 7 suppl. 1: 48-56, 1998b.
McKeown, M. J., Tzyy-Ping Jung, Scott Makeig, Greg Brown, Sandra S. Kindermann, Te-Won Lee and Terrence J. Sejnowski, Spatially independent activity patterns in functional magnetic resonance imaging data during the Stroup color-naming task, Proc. Natl. Acad. Sci USA, 95:803-810, 1998c.
McKeown, M. J. and Sejnowski, T. J., Independent component analysis of fMRI data: examining the assumptions. Human Brain Mapping 6:368-372, 1998d.
Makeig, S. Auditory event-related dynamics of the EEG spectrum and effects of exposure to tones, Electroencephalogr Clin Neurophysiol, 86:283-293, 1993.
Makeig, S. Toolbox for independent component analysis of psychophysiological data, [World Wide Web publication] www.cnl.salk.edu/˜scott/ica.html, 1997.
Makeig, S. and Galambos, R., The CERP: Event-related perturbations in steady-state responses, in: Brain Dynamics Progress and Perspectives, (pp. 375-400), ed. E. Basar and T. H. Bullock, 1989.
Makeig, S. and Inlow, M., Lapses in alertness: coherence of fluctuations in performance and the EEG spectrum, Electroencephalogr clin Neurophysiol, 86:23-35, 1993.
Makeig, S. and Jung, T-P., Changes in alertness are a principal component of variance in the EEG spectrum, NeuroReport 7:213-216, 1995.
Makeig, S. and T-P. Jung, Tonic, phasic, and transient EEG correlates of auditory awareness during drowsiness, Cognitive Brain Research 4:15-25, 1996.
Makeig, S., Bell, A. J., Jung, T-P. and Sejnowski, T. J., “Independent component analysis of electroencephalographic data,” In: D. Touretzky, M. Mazer and M. Hasselmo (Eds). Advances in Neural Information Processing Systems 8:145-151 MIT Press, Cambridge, M A, 1996.
Makeig, S., Jung, T-P, and Sejnowski, T. J., “Using feedforward neural networks to monitor alertness from changes in EEG correlation and coherence,” In: D. Touretzky, M. Mazer & M. Hasselmo (Eds). Advances in Neural Information Processing Systems 8:931-937 MIT Press, Cambridge, MA, 1996.
Makeig, S., T-P. Jung, D. Ghahremani, A. J. Bell & T. J. Sejnowski, Blind separation of auditory event-related brain responses into independent components. Proc. Natl. Acad. Sci. USA, 94:10979-10984, 1997.
Makeig, S., Westerfield, M., Jung, T-P., Covington, J., Townsend, J., Sejnowski, T. J. and Courchesne, E., Independent components of the late positive event-related potential in a visual spatial attention task, submitted.
Mitra P P, Ogawa S, Hu X, Ugurbil K, The nature of spatiotemporal changes in cerebral hemodynamics as manifested in functional magnetic resonance imaging. Magn Reson Med. 37:511-8, 1997.
Nobre A C, Sebestyen G N, Gitelman D R, Mesulam M M, Frackowiak R S, Frith C D, Functional localization of the system for visuospatial attention using positron emission tomography. Brain 120:515-33, 1997.
Nunez, P. L., Electric Fields of the Brain. New York: Oxford, 1981.
Ogawa S, Tank D W, Menon R, Ellermann J M, Kim S G, Merkle H, Ugurbil K, Intrinsic signal changes accompanying sensory stimulation: functional brain mapping with magnetic resonance imaging. Proc Natl Aced Sci USA 89:5951-5, 1992.
Pearlmutter, B. and Parra, L. C. Maximum likelihood blind source separation: a context-sensitive generalization of ICA. In: M. C. Mazer, M. I. Jordan and T. Petsche (Eds.), Advances in Neural Information Processing Systems 9:613-619 MIT Press, Cambridge, MA, 1996.
Sakai K, Hikosaka O, Miyauchi S, Takino R, Sasaki Y, Putz B. Transition of brain activation from frontal to parietal areas in visuomotor sequence learning. J Neurosci 18:1827-40, 1998.
Sahai, Amit, and Brent Waters. “Fuzzy identity-based encryption.” In Annual International Conference on the Theory and Applications of Cryptographic Techniques, pp. 457-473. Springer, Berlin, Heidelberg, 2005.
Scherg, M. & Von Cramon, D., Evoked dipole source potentials of the human auditory cortex. Electroencephalogr. Clin. Neurophysiol. 65:344-601, 1986.
Tallon-Baudry, C., Bertrand, O., Delpuech, C., & Pernier, J., Stimulus Specificity of Phase-Locked and Non-Phase-Locked 40 Hz Visual Responses in Human. J. Neurosci. 16: 4240-4249, 1996.
Thaker, Darshan D., Diana Franklin, John Oliver, Susmit Biswas, Derek Lockhart, Tzvetan Metodi, and Frederic T. Chong. “Characterization of error-tolerant applications when protecting control data.” In Workload Characterization, 2006 IEEE International Symposium on, pp. 142-149. IEEE, 2006.
Tulving E, Markowitsch H J, Craik F E, Habib R, Houle S, Novelty and familiarity activations in PET studies of memory encoding and retrieval. Cereb Cortex 6:71-9, 1996.
Warach, S., J. R. Ives, G. Schaug, M. R. Patel, D. G. Darby, V. Thangaraj, R. R. Edelman and D. L. Schomer, EEG-triggered echo-planar functional MRI in epilepsy, Neurology 47: 89-93, 1996.

Principal Component Analysis: Principal component analysis (PCA) is a statistical procedure that uses an orthogonal transformation to convert a set of observations of possibly correlated variables into a set of values of linearly uncorrelated variables called principal components. If there are n observations with p variables, then the number of distinct principal components is min(n−1,p). This transformation is defined in such a way that the first principal component has the largest possible variance (that is, accounts for as much of the variability in the data as possible), and each succeeding component in turn has the highest variance possible under the constraint that it is orthogonal to the preceding components. The resulting vectors are an uncorrelated orthogonal basis set. PCA is sensitive to the relative scaling of the original variables. PCA is the simplest of the true eigenvector-based multivariate analyses. Often, its operation can be thought of as revealing the internal structure of the data in a way that best explains the variance in the data. If a multivariate dataset is visualized as a set of coordinates in a high-dimensional data space (I axis per variable), PCA can supply the user with a lower-dimensional picture, a projection of this object when viewed from its most informative viewpoint. This is done by using only the first few principal components so that the dimensionality of the transformed data is reduced. PCA is closely related to factor analysis. Factor analysis typically incorporates more domain specific assumptions about the underlying structure and solves eigenvectors of a slightly different matrix. PCA is also related to canonical correlation analysis (NA). NA defines coordinate systems that optimally describe the cross-covariance between two datasets while PCA defines a new orthogonal coordinate system that optimally describes variance in a single dataset. See, en.wikipedia.org/wiki/Principal_component_analysis.

A general model for confirmatory factor analysis is expressed as x=α+Λξ+ε. The covariance matrix is expressed as E[(x−μ)(x−μ)′]=ΛΦΛ′+Θ. If residual covariance matrix Θ=0 and correlation matrix among latent factors Φ=I, then factor analysis is equivalent to principal component analysis and the resulting covariance matrix is simplified to Σ=ΛΛ′. When there are p number of variables and all p components (or factors) are extracted, this covariance matrix can alternatively be expressed into Σ=DΛD′, or Σ=λDAD′, where D=n×p orthogonal matrix of eigenvectors, and Λ=λA, p×p matrix of eigenvalues, where λ is a scalar and A is a diagonal matrix whose elements are proportional to the eigenvalues of Σ. The following three components determine the geometric features of the observed data: λ parameterizes the volume of the observation, D indicates the orientation, and A represents the shaped the observation.

When population heterogeneity is explicitly hypothesized as in model-based cluster analysis, the observed covariance matrix is decomposed into the following general form Σₖ=λₖDₖAₖDₖ^{T},

where λₖ parameterizes the volume of the k^{th} cluster, Dₖ indicates the orientation of that cluster, and Aₖ represents the shaped that cluster. The subscript k indicates that each component (or cluster) can have different volume, shape, and orientation.

Assume a random vector X, taking values in [symbol] ^{m}, has a mean and covariance matrix of μ_{X} and Σ_{X}, respectively. λ₁>λ₂> . . . >λₘ>0 are ordered eigenvalues of Σ_{X}, such that the i-th eigenvalue of Σ_{X} means the i-th largest of them. Similarly, a vector αᵢ is the i-th eigenvector of Σ_{X} when it corresponds to the i-th eigenvalue of Σ_{X}. To derive the form of principal components (PCs), consider the optimization problem of maximizing var[α₁^{T}X]=α₁^{T}Σ_{X}α₁, subject to α₁^{T}α₁=1. The Lagrange multiplier method is used to solve this question.

$$L ( {α}_{1} , {ϕ}_{1} ) = {α}_{1}^{T} {\sum }_{X} {α}_{1} + {ϕ}_{1} ( {α}_{1}^{T} {α}_{1} - 1 )$$

$$\frac{\partial L}{\partial {α}_{1}} = 2 {\sum }_{X} {α}_{1} + 2 ϕ {α}_{1} = 0 ⇒ {\sum }_{X} {α}_{1} = - {ϕ}_{1} {α}_{1} ⇒ var [ {α}_{1}^{T} X ] = - {ϕ}_{1} {α}_{1}^{T} {α}_{1} = - {ϕ}_{1} .$$

Because −ϕ₁ is the eigenvalue of Σ_{X}, with α₁ being the corresponding normalized eigenvector, var[α₁^{T}X] is maximized by choosing α₁ to be the first eigenvector of Σ_{X}. In this case, z₁=α₁^{T}X is named the first PC of X, α₁ is the vector of coefficients for z₁, and var(z₁)=λ₁.

To find the second PC, z₂=α₂^{T}X, we need to maximize var[α₂^{T}X]=α₂^{T}Σ_{X}α₂ subject to z₂ being uncorrelated with z₁. Because, cov(α₁^{T}X, α₂^{T}X)=0 ⇒α₁^{T}Σ_{X}α₂=0⇒α₁^{T}α₂=0, this problem is equivalently set as maximizing α₂^{T}Σ_{X}α₂, subject to α₁^{T}α₂=0 and α₂^{T}α₂=1. We still make use of the Lagrange multiplier method.

$$L ( {α}_{2} , {ϕ}_{1} , {ϕ}_{2} ) = {α}_{2}^{T} {\sum }_{X} {α}_{2} + {ϕ}_{1} {α}_{1}^{T} {α}_{2} + {ϕ}_{2} ( {α}_{2}^{T} {α}_{2} - 1 )$$

$$\frac{\partial L}{\partial {α}_{2}} = 2 {\sum }_{X} {α}_{2} + {ϕ}_{1} {α}_{1} + 2 {ϕ}_{2} {α}_{2} = 0 ⇒$$

$${α}_{1}^{T} ( 2 {\sum }_{X} {α}_{2} + {ϕ}_{1} {α}_{1} + 2 {ϕ}_{2} {α}_{2} ) = 0 ⇒ {ϕ}_{1} = 0 ⇒$$

$${\sum }_{X} {α}_{2} = - {ϕ}_{2} {α}_{2} ⇒ {α}_{2}^{T} {\sum }_{X} {α}_{2} = - {ϕ}_{2} .$$

Because −ϕ₂ is the eigenvalue of Σ_{X}, with α₂ being the corresponding normalized eigenvector, var[α₂^{T}X] is maximized by choosing α₂ to be the second eigenvector of Σ_{X}. In this case, z₂=α₂^{TX} is named the second PC of X, α₂ is the vector of coefficients for z₂, and var(z₂)=λ₂. Continuing in this way, it can be shown that the i-th PC zᵢ=αᵢ^{T}X is constructed by selecting αᵢ to be the i-th eigenvector of Σ_{X}, and has variance of λᵢ. The key result in regards to PCA is that the principal components are the only set of linear functions of original data that are uncorrelated and have orthogonal vectors of coefficients.

For any positive integer p≤m, let B=[β₁, β₂, . . . , βₚ] be an real m×p matrix with orthonormal columns, i.e., βᵢ^{T}βⱼ=δᵢⱼ, and Y=B^{T}X. Then the trace of covariance matrix of Y is maximized by taking B=[α₁, α₂, . . . , αₚ] where αᵢ is the i-th eigenvector of Σ_{X}. Because Σ_{X} is symmetric with all distinct eigenvalues, so {α₁, α₂, . . . , αₘ} is an orthonormal basis with αᵢ being the i-th eigenvector of Σ_{X}, and we can represent the columns of B as

$${β}_{i} = {\sum }_{j = 1}^{m} {c}_{j i} {α}_{j} ,$$

i=1, . . . , p. So we have B=PC, where P=[α₁, . . . , αₘ], C={cᵢⱼ} is an m×p matrix. Then, P^{T}Σ_{X}P=Λ, with Λ being a diagonal matrix whose k-th diagonal element is λₖ, and the covariance matrix of Y is,

Σ_{Y}=B^{T}Σ_{X}B=C^{T}P^{T}Σ_{X}PC=C^{T}ΛC=λ₁c₁c₁^{T}+ . . . +λₘcₘcₘ^{T}

where cᵢ^{T} is the i-th row of C. So,

$$trace ( {\sum }_{Y} ) = {\sum }_{i = 1}^{m} {λ}_{i} trace ( {c}_{i} {c}_{i}^{T} ) = {\sum }_{i = 1}^{m} {λ}_{i} trace ( {c}_{i}^{T} {c}_{i} ) = {\sum }_{i = 1}^{m} {λ}_{i} {c}_{i}^{T} {c}_{i} = {\sum }_{i = 1}^{m} ( {\sum }_{j = 1}^{p} {c}_{ij}^{2} ) {λ}_{i} .$$

Because C^{T}C=B^{T}PP^{T}B=B^{T}B=I, so

$$trace ( {C}^{T} C ) = {\sum }_{i = 1}^{m} {\sum }_{j = 1}^{p} {c}_{i j}^{2} = p ,$$

and the columns of C are orthonormal. By the Gram-Schmidt method, C can expand to D, such that D has its columns as an orthonormal basis of [symbol] ^{m} and contains C as its first p columns. D is square shape, thus being an orthogonal matrix and having its rows as another orthonormal basis of [symbol] ^{m}. One row of C is a part of one row of D, so

$${\sum }_{j = 1}^{p} {c}_{ij}^{2} \leq 1 ,$$

i=1, . . . , m. Considering the constraints

$${\sum }_{j = 1}^{p} {c}_{ij}^{2} \leq 1 , {\sum }_{i = 1}^{m} {\sum }_{j = 1}^{p} {c}_{ij}^{2} = p$$

and the objective

$${\sum }_{i = 1}^{m} ( {\sum }_{j = 1}^{p} {c}_{ij}^{2} ) {λ}_{i} .$$

We derive that trace(Σ_{Y}) is maximized if

$${\sum }_{j = 1}^{p} {c}_{ij}^{2} = 1$$

for i=1, . . . , p, and

$${\sum }_{j = 1}^{p} {c}_{ij}^{2} = 0$$

for i=p+1, . . . , m. When B=[α₁, α₂, . . . , αₚ], straightforward calculation yields that C is an all-zero matrix except cᵢᵢ=1, i=1, . . . , p. This fulfills the maximization condition. Actually, by taking B=[γ₁, γ₂, . . . , γₚ], where {γ₁, γ₂, . . . , γₚ} is any orthonormal basis of the subspace of span{α₁, α₂, . . . , αₚ}, the maximization condition is also satisfied, yielding the same trace of covariance matrix of Y.

Suppose that we wish to approximate the random vector X by its projection onto a subspace spanned by columns of B, where B=[β₁, β₂, . . . , βₚ] is a real m×p matrix with orthonormal columns, i.e., βᵢ^{T}βⱼ=δᵢⱼ. If σᵢ² is the residual variance for each component of X, then

$${\sum }_{i = 1}^{m} {σ}_{i}^{2}$$

is minimized if B=[α₁, α₂, . . . , αₚ], where {α₁, α₂, . . . , αₚ} are the first p eigenvectors of Σ_{X}. In other words, the trace of covariance matrix of X−BB^{T}X is minimized if B=[α₁, α₂, . . . , αₚ]. When E(X)=0, which is a commonly applied preprocessing step in data analysis methods, this property is saying that E∥X−BB^{T}X∥² is minimized if B=[α₁, α₂, . . . , αₚ].

The projection of a random vector X onto a subspace spanned by columns of B is \hat{X}=BB^{T}X Then the residual vector is ε=X−BB^{T}X, which has a covariance matrix

$${\sum }_{ε} = ( I - B {B}^{T} ) {\sum }_{X} ( I - B {B}^{T} ) . Then ,$$

$${{\sum }^{m}}_{i = 1} {σ}_{i}^{2} = trace ( {\sum }_{ε} ) = trace ( {\sum }_{X} - {\sum }_{X} B {B}^{T} - B {B}^{T} {\sum }_{X} + B {B}^{T} {\sum }_{X} B {B}^{T} ) .$$

Also, we know:

trace(Σ_{X}BB^{T})=trace(BB^{T}Σ_{X})=trace(B^{T}Σ_{X}B)

trace(BB^{T}Σ_{X}BB^{T})=trace(B^{T}Σ_{X}BB^{T}B)=trace(B^{T}Σ_{X}B).

The last equation comes from the fact that B has orthonormal columns. So,

$${\sum }_{i = 1}^{m} {σ}_{i}^{2} = trace ( {\sum }_{X} ) - trace ( {B}^{T} {\sum }_{X} B ) .$$

To minimize

$${\sum }_{i = 1}^{m} {σ}_{i}^{2} ,$$

it suffices to maximize trace(B^{T} Σ_{X} B). This can be done by choosing B=[α₁, α₂, . . . , αₚ], where {α₁, α₂, . . . , αₚ} are the first p eigenvectors of Σ_{X}, as above.

See, Pietro Amenta, Luigi D'Ambra, “Generalized Constrained Principal Component Analysis with External Information,” (2000). We assume that data on K sets of explanatory variables and S criterion variables of n statistical units are collected in matrices Xₖ (k=1, . . . , K) and Yₛ (s=1, . . . , S) of orders (n×p₁), . . . , (n×p_{K}) and (n×q₁), . . . , (n×q_{S}) respectively. We suppose, without loss of generality, identity matrices for the metrics of the spaces of variables of Xₖ and Yₛ with Dₙ=diag(1/n), weight matrix of statistical units. We assume, moreover, that Xₖ's and Yₛ's are centered as to the weights Dₙ.

Let X=[X₁| . . . |X_{K}] and Y=[Y₁| . . . |Y_{S}], respectively, be K and S matrices column linked of orders (n×Σₖpₖ) and (n×Σₛqₛ). Let be, also, W_{Y}=YY′ while we denote vₖ the coefficients vector (pₖ, 1) of the linear combination for each Xₖ such that zₖ=Xₖvₖ. Let Cₖ be the matrix of dimension pₖ×m (m≤pₖ), associated to the external information explanatory variables of set k.

Generalized CPCA (GCPCA) (Amenta, D'Ambra, 1999) with external information consists in seeking for K coefficients vectors vₖ (or, in same way, K linear combinations zₖ) subject to the restriction C′ₖzₖ=0 simultaneously, such that:

$$\{ \begin{matrix}max {\sum }_{i = 1}^{K} {\sum }_{j = 1}^{K} 〈 {Y}^{′} {X}_{i} {v}_{i} , {Y}^{′} {X}_{j} {v}_{j} 〉 \\ with the constraints \begin{matrix}{\sum }_{k = 1}^{K} {‖ {X}_{k} {v}_{k} ‖}^{2} = 1 \\ {\sum }_{k = 1}^{K} {C}_{k}^{′} {v}_{k} = 0\end{matrix}\end{matrix} \qquad ( 1 )$$

or, in equivalent way,

$$\{ \begin{matrix}max {v}^{′} ( {A}^{′} A ) v \\ with the constraints \begin{matrix}{v}^{′} Bv = 1 \\ {C}^{′} v = 0\end{matrix}\end{matrix} or$$

$$\{ \begin{matrix}max {f}^{′} {B}^{- 0.5} {A}^{′} {AB}^{- 0.5} f \\ with the constraints \begin{matrix}{f}^{′} f = 1 \\ {C}^{′} v = 0\end{matrix}\end{matrix}$$

where A=Y′X, B=diag(X′₁X₁, . . . , X′_{K}X_{K}), C″=[C′₁| . . . |C′ₖ], v′=(v₁′| . . . |vₖ′) and ƒ=B^{0.5}v, with

$${A}^{′} A = [ \begin{matrix}{X}_{1}^{′} {YY}^{′} {X}_{1} & … & {X}_{1}^{′} {YY}^{′} {X}_{K} \\ ⋮ & ⋱ & ⋮ \\ {X}_{K}^{′} {YY}^{′} {X}_{1} & … & {X}_{k}^{′} {YY}^{′} {X}_{K}\end{matrix} ] .$$

The constrained maximum problem turns out to be an extension of criterion sup_{Σ}_{ₖ}_{∥z}_{ₖ}_{∥}_{²}₌₁ΣᵢΣₖ [symbol] zᵢ, zₖ [symbol] (Sabatier, 1993) with more sets of criterion variables with external information. The solution of this constrained maximum problem leads to solve the eigen-equation

(P_{X}−P_{XB}_{^{−1}}_{C})W_{Y}g=λ_{g}

where g=Xv, P_{X}−P_{XB}_{^{−1}}_{C}=Σₖ₌₁^{K}(P_{X}_{ₖ}−P_{X}_{ₖ}_{(X′}_{ₖ}_{X}_{ₖ}₎_{^{−1}}_{C}_{ₖ}) is the oblique projector operator associated to the direct sum decomposition of [symbol] ⁿ

[symbol]

²=Im(P_{X}−P_{XB}_{^{−1}}_{C})\dot{⊕}Im(P_{C})\dot{⊕}Ker(P_{X})

with X_{X}_{ₖ}=Xₖ(X′ₖXₖ)⁻¹X′ₖ and P_{C}=C(C′B⁻¹C)⁻¹C′B⁻¹, respectively, /and B⁻¹ orthogonal projector operators onto the subspaces spanned by the columns of matrices Xₖ and C. Furthermore, P_{XB}_{^{−1}}_{C}=XB⁻¹(C′B⁻¹C)⁻¹C″B⁻¹X′ is the orthogonal projector operator onto the subspace spanned the columns of the matrix XB⁻¹C. Starting from the relation

(P_{X}_{ₖ}−P_{X}_{ₖ}_{(X′}_{ₖ}_{X}_{ₖ}₎_{^{−1}}_{C}_{ₖ})W_{Y}g=λXₖvₖ

(which is obtained from the expression (I−P_{C})X′W_{Y}g=λBv) the coefficients vectors vₖ and the linear combinations zₖ=Xₖvₖ maximizing (I) can be given by the relations

$${v}_{k} = \frac{1}{λ} {( {X}_{k}^{′} {X}_{k} )}^{- 1} ( I - {P}_{{C}_{k}} ) {X}_{k}^{′} {W}_{Y} Xv and$$

$${z}_{k} = \frac{1}{λ} ( {P}_{{X}_{k}} - {P}_{{{X}_{k} ( {X}_{k}^{′} {X}_{k} )}^{- 1} {C}_{k}} ) {W}_{Y} Xv ,$$

respectively.

The solution eigenvector gran be written, as sum of the linear combinations zₖ: g=ΣₖXₖvₖ. Notice that the eigenvalues associated to the eigen-system are, according to the Sturm theorem, lower or equal than those of GCPCA eigen-system: Σₖ₌₁^{K}P_{X}_{ₖ}W_{Y}g=λg.
Amenta P., D'Ambra L. (1994) Analisi non Simmetrica delle Corrispondenze Multiple con Vincoli Lineari. Atti S.I.S. XXVII Sanremo, Aprile 1994
Amenta P., D'Ambra L. (1996) L'Analisi in Componenti Principali in rapporto ad un sottospazio di riferimento con informazioni esterne, Quaderni del D.M.Q.T.E., Università di Pescara, n. 18.
Amenta P., D'Ambra L. (1999) Generalized Constrained Principal Component Analysis. Atti Riunione Scientifica del Gruppo di Classificazione dell'IFCS su “Classificazione e Analisi dei Dati”, Roma.
D'Ambra L., Lauro N. C. (1982) Analisi in componenti principali in rapporto ad un sottospazio di riferimento, Rivista di Statistica Applicata, n. 1, vol. 15.
D'Ambra L., Sabatier R., Amenta P. (1998) Analisi fattoriale delle matrici a tre vie: sintesi e nuovi approcci, (invited lecture) Atti XXXIX Riunone SIS.
Huon de Kermadec F., Durand J. F., Sabatier R. (1996) Comparaison de méthodes de régression pour l'etude des liens entre données hédoniques, in Third Sensometrics Meeting, E.N.T.I.A.A., Nantes.
Huon de Kermadec F., Durand J. F., Sabatier R. (1997) Comparison between linear and nonlinear PLS methods to explain overall liking from sensory characteristics, Food Quality and Preference, 8, n. 5/6.
Kiers H. A. L. (1991) Hierarchical relations among three way methods Psychometrika, 56.
Kvalheim D. M. (1988) A partial least squares approach to interpretative analysis of multivariate analysis, Chemometrics and Intelligent Laboratory System, 3.
MacFie H. J. H, Thomson D. M. H. (1988) Preference mapping and multidimensional scaling methods, in: Sensory Analysis of Foods. Elsevier Applied Science, London.
Sabatier R. (1993) Critéres et contraintes pour l'ordination simultanée de K tableaux, Biométrie et Environement, Masson, 332.
Schlich P. (1995) Preference mapping: relating consumer preferences to sensory or instrumental measurements, in: Bioflavour: INRA, Dijon.
Wold S., Geladi P., Esbensen K., Ohman J. (1987) Multi-way principal components and PLS-analysis, J. of Chemometrics, vol. 1.

Spatial Principal Component Analysis

Let J(t, i; α, s) be the current density in voxel i, as estimated by LORETA, in condition α at t time-frames after stimulus onset for subject s. Let area:Voxel→ƒBA be a function, which assigns to each voxel i∈Voxel the corresponding fBA b∈fBA. In a first pre-processing step, we calculate for each subject s the value of the current density averaged over each Fba

$$x ( t , b ; α , s ) = \frac{1}{{N}_{b}} {\sum }_{i ∈ b} J ( t , i ; α , s ) \qquad ( 4 )$$

where N_{b} is the number of voxels in the fBA b, in condition α for subject s.

In the second analysis stage, the mean current density x(t, b; α, s) from each fBA b, for every subject s and condition α, was subjected to spatial PCA analysis of the correlation matrix and varimax rotation

In the present study the spatial PCA uses the above-defined fBAs as variables sampled along the time epoch for which EEG has been sampled (0-1000 ms; 512 time-frames), and the inverse solution was estimated. Spatial matrices (each matrix was sized b×t=36×512 elements) for every subject and condition were collected, and subjected to PCA analyses, including the calculation of the covariance matrix; eigenvalue decomposition and varimax rotation, in order to maximize factor loadings. In other words, in the spatial PCA analysis we approximate the mean current density for each subject in each condition as

$$x ( t ; α , s ) \approx {x}_{0} ( α , s ) + {\sum }_{k} {c}_{k} ( t ) {x}_{k} ( α , s ) , \qquad ( 5 )$$

where here x(t; α, s)∈R³⁶ is a vector, which denotes the time-dependent activation of the fBAs, x₀(α, s) is their mean activation, and xₖ(α, s) and cₖ are the principal components and their corresponding coefficients (factor loadings) as computed using the principal component analysis.

See, download.lww.com/wolterskluwer.com/WNR_1_1_2010_03_22_ARZY_1_SDC1.doc.

Nonlinear Dimensionality Reduction: High-dimensional data, meaning data that requires more than two or three dimensions to represent, can be difficult to interpret. One approach to simplification is to assume that the data of interest lie on an embedded non-linear manifold within the higher-dimensional space. If the manifold is of low enough dimension, the data can be visualized in the low-dimensional space. Non-linear methods can be broadly classified into two groups: those that provide a mapping (either from the high-dimensional space to the low-dimensional embedding or vice versa), and those that just give a visualization. In the context of machine learning, mapping methods may be viewed as a preliminary feature extraction step, after which pattern recognition algorithms are applied. Typically, those that just give a visualization are based on proximity data—that is, distance measurements. Related Linear Decomposition Methods include Independent component analysis (IDA), Principal component analysis (PCA) (also called Karhunen-Loève transform—KLT), Singular value decomposition (SVD), and Factor analysis.

The self-organizing map (SOM, also called Kohonen map) and its probabilistic variant generative topographic mapping (GTM) use a point representation in the embedded space to form a latent variable model based on a non-linear mapping from the embedded space to the high-dimensional space. These techniques are related to work on density networks, which also are based around the same probabilistic model.

Principal curves and manifolds give the natural geometric framework for nonlinear dimensionality reduction and extend the geometric interpretation of PCA by explicitly constructing an embedded manifold, and by encoding using standard geometric projection onto the manifold. How to define the “simplicity” of the manifold is problem-dependent, however, it is commonly measured by the intrinsic dimensionality and/or the smoothness of the manifold. Usually, the principal manifold is defined as a solution to an optimization problem. The objective function includes a quality of data approximation and some penalty terms for the bending of the manifold. The popular initial approximations are generated by linear PCA, Kohonen's SOM or autoencoders. The elastic map method provides the expectation-maximization algorithm for principal manifold learning with minimization of quadratic energy functional at the “maximization” step.

An autoencoder is a feed-forward neural network which is trained to approximate the identity function. That is, it is trained to map from a vector of values to the same vector. When used for dimensionality reduction purposes, one of the hidden layers in the network is limited to contain only a small number of network units. Thus, the network must learn to encode the vector into a small number of dimensions and then decode it back into the original space. Thus, the first half of the network is a model which maps from high to low-dimensional space, and the second half maps from low to high-dimensional space. Although the idea of autoencoders is quite old, training of deep autoencoders has only recently become possible through the use of restricted Boltzmann machines and stacked denoising autoencoders. Related to autoencoders is the NeuroScale algorithm, which uses stress functions inspired by multidimensional scaling and Sammon mappings (see below) to learn a non-linear mapping from the high-dimensional to the embedded space. The mappings in NeuroScale are based on radial basis function networks.

Gaussian process latent variable models (GPLVM) are probabilistic dimensionality reduction methods that use Gaussian Processes (GPs) to find a lower dimensional non-linear embedding of high dimensional data. They are an extension of the Probabilistic formulation of PCA. The model is defined probabilistically and the latent variables are then marginalized and parameters are obtained by maximizing the likelihood. Like kernel PCA they use a kernel function to form a nonlinear mapping (in the form of a Gaussian process). However, in the GPLVM the mapping is from the embedded (latent) space to the data space (like density networks and GTM) whereas in kernel PCA it is in the opposite direction. It was originally proposed for visualization of high dimensional data but has been extended to construct a shared manifold model between two observation spaces. GPLVM and its many variants have been proposed specially for human motion modeling, e.g., back constrained GPLVM, GP dynamic model (GPDM), balanced GPDM (B-GPDM) and topologically constrained GPDM. To capture the coupling effect of the pose and gait manifolds in the gait analysis, a multi-layer joint gait-pose manifolds was proposed.

Curvilinear component analysis (CCA) looks for the configuration of points in the output space that preserves original distances as much as possible while focusing on small distances in the output space (conversely to Sammon's mapping which focus on small distances in original space). It should be noticed that CCA, as an iterative learning algorithm, actually starts with focus on large distances (like the Sammon algorithm), then gradually change focus to small distances. The small distance information will overwrite the large distance information, if compromises between the two have to be made. The stress function of CCA is related to a sum of right Bregman divergences. Curvilinear distance analysis (COA) trains a self-organizing neural network to fit the manifold and seeks to preserve geodesic distances in its embedding. It is based on Curvilinear Component Analysis (which extended Sammon's mapping), but uses geodesic distances instead. Diffeomorphic Dimensionality Reduction or Diffeomap learns a smooth diffeomorphic mapping which transports the data onto a lower-dimensional linear subspace. The method solves for a smooth time indexed vector field such that flows along the field which start at the data points will end at a lower-dimensional linear subspace, thereby attempting to preserve pairwise differences under both the forward and inverse mapping.

Perhaps the most widely used algorithm for manifold learning is Kernel principal component analysis (kernel PCA). It is a combination of Principal component analysis and the kernel trick. PCA begins by computing the covariance matrix of the M×n Matrix X. It then projects the data onto the first k eigenvectors of that matrix. By comparison, KPCA begins by computing the covariance matrix of the data after being transformed into a higher-dimensional space. It then projects the transformed data onto the first k eigenvectors of that matrix, just like PCA. It uses the kernel trick to factor away much of the computation, such that the entire process can be performed without actually computing ϕ(x). Of course ϕ must be chosen such that it has a known corresponding kernel.

Laplacian Eigenmaps, (also known as Local Linear Eigenmaps, LLE) are special cases of kernel PCA, performed by constructing a data-dependent kernel matrix. KPCA has an internal model, so it can be used to map points onto its embedding that were not available at training time. Laplacian Eigenmaps uses spectral techniques to perform dimensionality reduction. This technique relies on the basic assumption that the data lies in a low-dimensional manifold in a high-dimensional space. This algorithm cannot embed out of sample points, but techniques based on Reproducing kernel Hilbert space regularization exist for adding this capability. Such techniques can be applied to other nonlinear dimensionality reduction algorithms as well. Traditional techniques like principal component analysis do not consider the intrinsic geometry of the data. Laplacian eigenmaps builds a graph from neighborhood information of the data set. Each data point serves as a node on the graph and connectivity between nodes is governed by the proximity of neighboring points (using e.g., the k-nearest neighbor algorithm). The graph thus generated can be considered as a discrete approximation of the low-dimensional manifold in the high-dimensional space. Minimization of a cost function based on the graph ensures that points close to each other on the manifold are mapped close to each other in the low-dimensional space, preserving local distances. The eigenfunctions of the Laplace-Beltrami operator on the manifold serve as the embedding dimensions, since under mild conditions this operator has a countable spectrum that is a basis for square integrable functions on the manifold (compare to Fourier series on the unit circle manifold). Attempts to place Laplacian eigenmaps on solid theoretical ground have met with some success, as under certain nonrestrictive assumptions, the graph Laplacian matrix has been shown to converge to the Laplace-Beltrami operator as the number of points goes to infinity. In classification applications, low dimension manifolds can be used to model data classes which can be defined from sets of observed instances. Each observed instance can be described by two independent factors termed ‘content’ and ‘style’, where ‘content’ is the invariant factor related to the essence of the class and ‘style’ expresses variations in that class between instances. Unfortunately, Laplacian Eigenmaps may fail to produce a coherent representation of a class of interest when training data consist of instances varying significantly in terms of style. In the case of classes which are represented by multivariate sequences, Structural Laplacian Eigenmaps has been proposed to overcome this issue by adding additional constraints within the Laplacian Eigenmaps neighborhood information graph to better reflect the intrinsic structure of the class. More specifically, the graph is used to encode both the sequential structure of the multivariate sequences and, to minimize stylistic variations, proximity between data points of different sequences or even within a sequence, if it contains repetitions. Using dynamic time warping, proximity is detected by finding correspondences between and within sections of the multivariate sequences that exhibit high similarity.

Like LLE, Hessian LLE is also based on sparse matrix techniques. It tends to yield results of a much higher quality than LLE. Unfortunately, it has a very costly computational complexity, so it is not well-suited for heavily sampled manifolds. It has no internal model. Modified LLE (MLLE) is another LLE variant which uses multiple weights in each neighborhood to address the local weight matrix conditioning problem which leads to distortions in LLE maps. MLLE produces robust projections similar to Hessian LLE, but without the significant additional computational cost.

Manifold alignment takes advantage of the assumption that disparate data sets produced by similar generating processes will share a similar underlying manifold representation. By learning projections from each original space to the shared manifold, correspondences are recovered and knowledge from one domain can be transferred to another. Most manifold alignment techniques consider only two data sets, but the concept extends to arbitrarily many initial data sets. Diffusion maps leverages the relationship between heat diffusion and a random walk (Markov Chain); an analogy is drawn between the diffusion operator on a manifold and a Markov transition matrix operating on functions defined on the graph whose nodes were sampled from the manifold. Relational perspective map is a multidimensional scaling algorithm. The algorithm finds a configuration of data points on a manifold by simulating a multi-particle dynamic system on a closed manifold, where data points are mapped to particles and distances (or dissimilarity) between data points represent a repulsive force. As the manifold gradually grows in size the multi-particle system cools down gradually and converges to a configuration that reflects the distance information of the data points. Local tangent space alignment (LTSA) is based on the intuition that when a manifold is correctly unfolded, all of the tangent hyperplanes to the manifold will become aligned. It begins by computing the k-nearest neighbors of every point. It computes the tangent space at every point by computing the d-first principal components in each local neighborhood. It then optimizes to find an embedding that aligns the tangent spaces. Local Multidimensional Scaling performs multidimensional scaling in local regions, and then uses convex optimization to fit all the pieces together.

Maximum Variance Unfolding was formerly known as Semidefinite Embedding. The intuition for this algorithm is that when a manifold is properly unfolded, the variance over the points is maximized. This algorithm also begins by finding the k-nearest neighbors of every point. It then seeks to solve the problem of maximizing the distance between all non-neighboring points, constrained such that the distances between neighboring points are preserved. Nonlinear PCA (NLPCA) uses backpropagation to train a multi-layer perceptron (MLP) to fit to a manifold. Unlike typical MLP training, which only updates the weights, NLPCA updates both the weights and the inputs. That is, both the weights and inputs are treated as latent values. After training, the latent inputs are a low-dimensional representation of the observed vectors, and the MLP maps from that low-dimensional representation to the high-dimensional observation space. Manifold Sculpting uses graduated optimization to find an embedding. Like other algorithms, it computes the k-nearest neighbors and tries to seek an embedding that preserves relationships in local neighborhoods. It slowly scales variance out of higher dimensions, while simultaneously adjusting points in lower dimensions to preserve those relationships.

Ruffini (2015) discusses Multichannel transcranial current stimulation (tCS) systems that offer the possibility of EEG-guided optimized, non-invasive brain stimulation. A tCS electric field realistic brain model is used to create a forward “lead-field” matrix and, from that, an EEG inverter is employed for cortical mapping. Starting from EEG, 2D cortical surface dipole fields are defined that could produce the observed EEG electrode voltages.

Schestatsky et al. (2017) discuss transcranial direct current stimulation (tDCS), which stimulates through the scalp with a constant electric current that induces shifts in neuronal membrane excitability, resulting in secondary changes in cortical activity. Although tDCS has most of its neuromodulatory effects on the underlying cortex, tDCS effects can also be observed in distant neural networks. Concomitant EEG monitoring of the effects of tDCS can provide valuable information on the mechanisms of tDCS. EEG findings can be an important surrogate marker for the effects of tDCS and thus can be used to optimize its parameters. This combined EEG-tDCS system can also be used for preventive treatment of neurological conditions characterized by abnormal peaks of cortical excitability, such as seizures. Such a system would be the basis of a non-invasive closed-loop device. tDCS and EEG can be used concurrently.
Albert, Jacobo, Sara López-Martin, José Antonio Hinojosa, and Luis Carretié. “Spatiotemporal characterization of response inhibition.” Neuroimage 76 (2013): 272-281.
Arzouan Y, Goldstein A, Faust M. Brainwaves are stethoscopes: ERP correlates of novel metaphor comprehension. Brain Res 2007; 1160: 69-81.
Arzouan Y, Goldstein A, Faust M. Dynamics of hemispheric activity during metaphor comprehension: electrophysiological measures. NeuroImage 2007; 36: 222-231.
Arzy, Shahar, Yossi Arzouan, Esther Adi-Japha, Sorin Solomon, and Olaf Blanke. “The ‘intrinsic’ system in the human cortex and self-projection: a data driven analysis.” Neuroreport 21, no. 8 (2010): 569-574.
Bao, Xuecai, Jinli Wang, and Jianfeng Hu. “Method of individual identification based on electroencephalogram analysis.” In New Trends in Information and Service Science, 2009. NISS'09. International Conference on, pp. 390-393. IEEE, 2009.
Bhattacharya, Joydeep. “Complexity analysis of spontaneous EEG.” Acta neurobiologiae experimentalis 60, no. 4 (2000): 495-502.
Chapman R M, McCrary J W. E P component identification and measurement by principal components analysis. Brain and cognition 1995; 27: 288-310.
Clementz, Brett A., Stefanie K. Barber, and Jacqueline R. Dzau. “Knowledge of stimulus repetition affects the magnitude and spatial distribution of low-frequency event-related brain potentials.” Audiology and Neurotology 7, no. 5 (2002): 303-314.
Dien J, Frishkoff G A, Cerbone A, Tucker D M. Parametric analysis of event-related potentials in semantic comprehension: evidence for parallel brain mechanisms. Brain research 2003; 15: 137-153.
Dien J, Frishkoff G A. Principal components analysis of event-related potential datasets. In: Handy T (ed). Event-Related Potentials: A Methods Handbook. Cambridge, Mass MIT Press; 2004.
Elbert, T. “IIIrd Congress of the Spanish Society of Psychophysiology.” Journal of Psychophysiology 17 (2003): 39-53.
Groppe, David M., Scott Makeig, Marta Kutas, and S. Diego. “Independent component analysis of event-related potentials.” Cognitive science online 6, no. 1 (2008): 1-44.
Have, Mid-Ventrolateral Prefrontal Cortex. “Heschl's Gyrus, Posterior Superior Temporal Gyrus.” J Neurophysiol 97 (2007): 2075-2082.
Hinojosa, J. A., J. Albert, S. López-Martin, and L. Carretié. “Temporospatial analysis of explicit and implicit processing of negative content during word comprehension.” Brain and cognition 87 (2014):109-121.
Jarchi, Delaram, Saeid Sanei, Jose C. Principe, and Bahador Makkiabadi. “A new spatiotemporal filtering method for single-trial estimation of correlated ERP subcomponents.” IEEE Transactions on Biomedical Engineering 58, no. 1 (2011):132-143.
John, Erwin Roy. “A field theory of consciousness.” Consciousness and cognition 10, no. 2 (2001):184-213.
Johnson, Mark H., Michelle de Haan, Andrew Oliver, Warwick Smith, Haralambos Hatzakis, Leslie A. Tucker, and Gergely Csibra. “Recording and analyzing high-density event-related potentials with infants using the Geodesic Sensor Net.” Developmental Neuropsychology 19, no. 3 (2001): 295-323.
Jung, Tzyy-Ping, and Scott Makeig. “Mining Electroencephalographic Data Using Independent Component Analysis.” EEG Journal (2003).
Kashyap, Rajan. “Improved localization of neural sources and dynamical causal modelling of latency-corrected event related brain potentials and applications to face recognition and priming.” (2015).
Klawohn, Julia, Anja Riesel, Rosa Grützmann, Norbert Kathmann, and Tanja Endrass. “Performance monitoring in obsessive-compulsive disorder: A temporo-spatial principal component analysis.” Cognitive, Affective, & Behavioral Neuroscience 14, no. 3 (2014): 983-995.
Lister, Jennifer J., Nathan D. Maxfield, and Gabriel J. Pitt. “Cortical evoked response to gaps in noise: within-channel and across-channel conditions.” Ear and hearing 28, no. 6 (2007): 862.
Maess, Burkhard, Angela D. Friederici, Markus Damian, Antje S. Meyer, and Willem J M Levelt. “Semantic category interference in overt picture naming: Sharpening current density localization by PCA.” Journal of cognitive neuroscience 14, no. 3 (2002): 455-462.
Makeig, Scott, Marissa Westerfield, Jeanne Townsend, Tzyy-Ping Jung, Eric Courchesne, and Terrence J. Sejnowski. “Functionally independent components of early event-related potentials in a visual spatial attention task.” Philosophical Transactions of the Royal Society B: Biological Sciences 354, no. 1387 (1999): 1135-1144.
Matsuda, Izumi, Hiroshi Nittono, Akihisa Hirota, Tokihiro Ogawa, and Noriyoshi Takasawa. “Event-related brain potentials during the standard autonomic-based concealed information test.” International Journal of Psychophysiology 74, no. 1 (2009): 58-68.
Mazaheri, Ali, and Terence W. Picton. “EEG spectral dynamics during discrimination of auditory and visual targets.” Cognitive Brain Research 24, no. 1 (2005): 81-96.
Pirmoradi, Mona, Boutheina Jemel, Anne Gallagher, Julie Tremblay, Fabien D'Hondt, Dang Khoa Nguyen, Renée Béland, and Maryse Lassonde. “Verbal memory and verbal fluency tasks used for language localization and lateralization during magnetoencephalography.” Epilepsy research 119 (2016): 1-9.
Potts G F, Dien J, Hartry-Speiser A L, McDougal L M, Tucker D M. Dense sensor array topography of the event-related potential to task-relevant auditory stimuli. Electroencephalography and clinical neurophysiology 1998; 106: 444-456.
Rosier F, Manzey D. Principal components and varimax-rotated components in event-related potential research: some remarks on their interpretation. Biological psychology 1981; 13: 3-26.
Ruchkin D S, McCalley M G, Glaser E M. Event related potentials and time estimation. Psychophysiology 1977; 14: 451-455.
Schroder, Hans S., James E. Glazer, Ken P. Bennett, Tim P. Moran, and Jason S. Moser. “Suppression of error-preceding brain activity explains exaggerated error monitoring in females with worry.” Biological psychology 122 (2017): 33-41.
Spencer K M, Dien J, Donchin E. Spatiotemporal analysis of the late ERP responses to deviant stimuli. Psychophysiology 2001; 38: 343-358.
Squires K C, Squires N K, Hillyard S A. Decision-related cortical potentials during an auditory signal detection task with cued observation intervals. Journal of experimental psychology 1975; 1: 268-279.
van Boxtel A, Boelhouwer A J, Bos A R. Optimal EMG signal bandwidth and interelectrode distance for the recording of acoustic, electrocutaneous, and photic blink reflexes. Psychophysiology 1998; 35: 690-697.
Veen, Vincent van, and Cameron S. Carter. “The timing of action-monitoring processes in the anterior cingulate cortex.” Journal of cognitive neuroscience 14, no. 4 (2002): 593-602.
Wackermann, Jiri. “Towards a quantitative characterisation of functional states of the brain: from the non-linear methodology to the global linear description.” International Journal of Psychophysiology 34, no. 1(1999): 65-80.

EEG analysis approaches have emerged, in which event-related changes in EEG dynamics in single event-related data records are analyzed. See Allen D. Malony et al., Computational Neuroinformatics for Integrated Electromagnetic Neuroimaging and Analysis, PAR-99-138. Pfurtscheller, reported a method for quantifying the average transient suppression of alpha band (circa 10-Hz) activity following stimulation. Event-related desynchronization (ERD, spectral amplitude decreases), and event-related synchronization (ERS, spectral amplitude increases) are observed in a variety of narrow frequency bands (4-40 Hz) which are systematically dependent on task and cognitive state variables as well as on stimulus parameters. Makeig (1993) reported event-related changes in the full EEG spectrum, yielding a 2-D time/frequency measure he called the event-related spectral perturbation (ERSP). This method avoided problems associated with analysis of a priori narrow frequency bands, since bands of interest for the analysis could be based on significant features of the complete time/frequency transform. Rappelsburger et al. introduced event-related coherence (ERCOH). A wide variety of other signal processing measures have been tested for use on EEG and/or MEG data, including dimensionality measures based on chaos theory and the bispectrum. Use of neural networks has also been proposed for EEG pattern recognition applied to clinical and practical problems, though usually these methods have not been employed with an aim of explicitly modeling the neurodynamics involved. Neurodynamics is the mobilization of the nervous system as an approach to physical treatment. The method relies on influencing pain and other neural physiology via mechanical treatment of neural tissues and the non-neural structures surrounding the nervous system. The body presents the nervous system with a mechanical interface via the musculoskeletal system. With movement, the musculoskeletal system exerts non-uniform stresses and movement in neural tissues, depending on the local anatomical and mechanical characteristics and the pattern of body movement. This activates an array of mechanical and physiological responses in neural tissues. These responses include neural sliding, pressurization, elongation, tension and changes in intraneural microcirculation, axonal transport and impulse traffic.

The availability of and interest in larger and larger numbers of EEG (and MEG) channels led immediately to the question of how to combine data from different channels. Donchin advocated the use of linear factor analysis methods based on principal component analysis (PCA) for this purpose. Temporal PCA assumes that the time course of activation of each derived component is the same in all data conditions. Because this is unreasonable for many data sets, spatial PCA (usually followed by a component rotation procedure such as Varimax or Promax) is of potentially greater interest. To this end, several variants of PCA have been proposed for ERP decomposition.

Bell and Sejnowski published an iterative algorithm based on information theory for decomposing linearly mixed signals into temporally independent by minimizing their mutual information. First approaches to blind source separation minimized third and fourth-order correlations among the observed variables and achieved limited success in simulations. A generalized approach uses a simple neural network algorithm that used joint information maximization or ‘infomax’ as a training criterion. By using a compressive nonlinearity to transform the data and then following the entropy gradient of the resulting mixtures, ten recorded voice and music sound sources were unmixed. A similar approach was used for performing blind deconvolution, and the ‘infomax’ method was used for decomposition of visual scenes.

The first applications of blind decomposition to biomedical time series analysis applied the infomax independent component analysis (ICA) algorithm to decomposition of EEG and event-related potential (ERP) data and reported the use of ICA to monitor alertness. This separated artifacts, and EEG data into constituent components defined by spatial stability and temporal independence. ICA can also be used to remove artifacts from continuous or event-related (single-trial) EEG data prior to averaging. Vigario et al. (1997), using a different ICA algorithm, supported the use of ICA for identifying artifacts in MEG data. Meanwhile, widespread interest in ICA has led to multiple applications to biomedical data as well as to other fields (Jung et al., 2000b). Most relevant to EEG/MEG analysis, ICA is effective in separating functionally independent components of functional magnetic resonance imaging (fMRI) data

Since the publication of the original infomax ICA algorithm, several extensions have been proposed. Incorporation of a ‘natural gradient’ term avoided matrix inversions, greatly speeding the convergence of the algorithm and making it practical for use with personal computers on large data EEG and fMRI data sets. An initial ‘sphering’ step further increased the reliability of convergence of the algorithm. The original algorithm assumed that sources have ‘sparse’ (super-Gaussian) distributions of activation values. This restriction has recently been relaxed in an ‘extended-ICA’ algorithm that allows both super-Gaussian and sub-Gaussian sources to be identified. A number of variant ICA algorithms have appeared in the signal processing literature. In general, these make more specific assumptions about the temporal or spatial structure of the components to be separated, and typically are more computationally intensive than the infomax algorithm.

Since individual electrodes (or magnetic sensors) each record a mixture of brain and non-brain sources, spectral measures are difficult to interpret and compare across scalp channels. For example, an increase in coherence between two electrode signals may reflect the activation of a strong brain source projecting to both electrodes, or the deactivation of a brain generator projecting mainly to one of the electrodes. If independent components of the EEG (or MEG) data can be considered to measure activity within functionally distinct brain networks, however, event-related coherence between independent components may reveal transient, event-related changes in their coupling and decoupling (at one or more EEG/MEG frequencies). ERCOH analysis has been applied to independent EEG components in a selective attention task.

See (each of which is expressly incorporated herein by reference):
en.wikipedia.org/wiki/Beat_(acoustics) #Binaural_beats;
en.wikipedia.org/wiki/Brainwave_entrainment;
www.livingflow.net/isochronic-tones-work/.
“Stimulating the Brain with Light and Sound,” Transparent Corporation, Neuroprogrammer™ 3, www.transparentcorp.com/products/np/entrainment.php.
Abeln, Vera, et al. “Brainwave entrainment for better sleep and post-sleep state of young elite soccer players-A pilot study.” European J. Sport science 14.5 (2014): 393-402.
Acton, George. “Methods for independent entrainment of visual field zones.” U.S. Pat. No. 9,829,978. 25 Apr. 2017.
Adelson, E. H., J. R. Bergen, Spatiotemporal energy models for the perception of motion. J. Opt. Soc. Am. A 2, 284-299 (1985).
Ahmadian, Y., D. B. Rubin, K. D. Miller, Analysis of the stabilized supralinear network. Neural Comput. 25, 1994-2037 (2013).
Albouy, Philippe, et al. “Selective entrainment of theta oscillations in the dorsal stream causally enhances auditory working memory performance.” Neuron 94.1 (2017): 193-206.
Albrecht, D. G., Visual cortex neurons in monkey and cat: Effect of contrast on the spatial and temporal phase transfer functions. Vis. Neurosci. 12, 1191-1210 (1995).
Albright, T. D., G. R. Stoner, Contextual influences on visual processing. Annu. Rev. Neurosci. 25, 339-379 (2002).
Alexander, D. M., T. Ball, A. Schulze-Bonhage, C. van Leeuwen, Large-scale cortical travelling waves predict localized future cortical signals. PLUS Comput. Biol. 15, e1007318 (2019).
Ali, M. M. 2013. “Transcranial alternating current stimulation modulates large-scale cortical network activity by network resonance.” J. Neurosci. 33 (27):11262-75.
Allen, J. J. B., P. M. Keune, M. Schqnenberg, and R. Nusslock. 2018. “Frontal EEG alpha asymmetry and emotion: From neural underpinnings and methodological considerations to psychopathology and social cognition.” Psychophysiology 55, e13028. doi:10.1111/psyp.13028.
Allman, J., F. Miezin, E. McGuinness, Stimulus specific responses from beyond the classical receptive field: Neurophysiological mechanisms for local-global comparisons in visual neurons. Annu. Rev. Neurosci. 8, 407-430 (1985).
Amengual, J., et al. “P018 Local entrainment and distribution across cerebral networks of natural oscillations elicited in implanted epilepsy patients by intracranial stimulation: Paving the way to develop causal connectomics of the healthy human brain.” Clin. Neurophysiology 128.3 (2017): e18.
Angelucci, A., M. Bijanzadeh, L. Nurminen, F. Federer, S. Merlin, P. C. Bressloff, Circuits and mechanisms for surround modulation in visual cortex. Annu. Rev. Neurosci. 40, 425-451(2017).
Annarumma, L., A. D'Atri, V. Alfonsi, and L. De Gennaro. 2018. “The efficacy of transcranial current stimulation techniques to modulate resting-state EEG, to affect vigilance and to promote sleepiness.” Brain Sci., 8:7, 137. doi:10.3390/brainsci8070.
Antal, Andrea, Kla'ra Boros, Csaba Poreisz, Leila Chaieb, Daniella Terney, and Walter Paulus. 2008. “Comparatively weak after-effects of transcranial alternating current stimulation (tACS) on cortical excitability in humans.” Brain Stimul. 1:2, 97-105. doi:10.1016/J.BRS.2007.10.001.
Argento, Emanuele, et al. “Augmented Cognition via Brainwave Entrainment in Virtual Reality: An Open, Integrated Brain Augmentation in a Neuroscience System Approach.” Augmented Human Research 2.1(2017): 3.
Atwater, F. H. (2001). Binaural beats and the regulation of arousal levels. Proceedings of the TANS, 11.
Bair, W., Visual receptive field organization. Curr. Opin. Neurobiol. 15, 459-464 (2005).
Baladron, J., D. Fasoli, O. Faugeras, J. Touboul, Mean-field description and propagation of chaos in networks of Hodgkin-Huxley and FitzHugh-Nagumo neurons. J. Math. Neurosci. 2, 10 (2012).
Barker, A. T. 1985. “Non-invasive magnetic stimulation of human motor cortex.” Lancet 1106-1107.
Bedrosian, Tracy A., Celynn A. Vaughn, Anabel Galan, Ghassan Daye, Zachary M. Weil, and Randy J. Nelson. 2013. “Nocturnal light exposure impairs affective responses in a wavelength-dependent manner.” J. of Neurosci. 33:32, 13081-13087. doi:10.1523/JNEUROSCI.5734-12.2013.
Bello, Nicholas P. “Altering Cognitive and Brain States Through Cortical Entrainment.” (2014); Costa-Faidella, Jordi, Elyse S. Sussman, and Caries Escera. “Selective entrainment of brain oscillations drives auditory perceptual organization.” NeuroImage (2017).
Benucci, A., R. A. Frazor, M. Carandini, Standing waves and traveling waves distinguish two circuits in visual cortex. Neuron 55, 103-117 (2007).
Basle, J., C. A. Schevon, A. D. Mehta, P. Lakatos, R. R. Goodman, G. M. McKhann, R. G. Emerson, and C. E. Schroeder. 2011. “Tuning of the human neocortex to the temporal dynamics of attended events.” The J. of Neurosci. 31:9, 3176-3185. doi:10.1523/JNEUROSCI.4518-10.2011.
Börgers, Christoph. “Entrainment by Excitatory Input Pulses.” An Introduction to Modeling Neuronal Dynamics. Springer International Publishing, 2017. 183-192.
Born, R. T., D. C. Bradley, Structure and function of visual area MT. Annu. Rev. Neurosci. 28, 157-189 (2005).
Brown, Richard. 2006. “What is a brain state?” Philosophical Psychology 19:6, 729-742. doi:10.1080/0951508000923271.
Buck R (1999) The biological affects: A typology. Psychological Review 106: 301-336; Izard C E (2007) Basic Emotions, Natural Kinds, Emotion Schemas, and a New Paradigm. Perspect Psychol Sci 2: 260-280.
Buonomano, D. V., W. Maass, State-dependent computations: Spatiotemporal processing in cortical networks. Nat. Rev. Neurosci. 10, 113-125 (2009).
Calderone, Daniel J., et al. “Entrainment of neural oscillations as a modifiable substrate of attention.” Trends in cognitive sciences 18.6 (2014): 300-309.
Canolty R T, Edwards E, Dalai S S, et al. High gamma power is phase-locked to theta oscillations in human neocortex. Science. 2006; 313:1628-1628.
Carter, J., and H. Russell. “A pilot investigation of auditory and visual entrainment of brain wave activity in learning disabled boys.” Texas Researcher 4.1 (1993): 65-75.
Casciaro, Francesco, et al. “Alpha-rhythm stimulation using brain entrainment enhances heart rate variability in subjects with reduced HRV.” World J. Neuroscience 3.04 (2013): 213.
Cavanaugh, J. R., W. Bair, J. A. Movshon, Nature and interaction of signals from the receptive field center and surround in macaque V I neurons. J. Neurophysiol. 88, 2530-2546 (2002).
Chaieb, L., E. C. Wilpert, T. P. Reber, and J. Fell. 2015. “Auditory beat stimulation and its effects on cognition and mood states.” Front. in Psychiatry 6, 70.
Chalmers, David J. 1995. “Facing up to the problem of consciousness.” J. of Consciousness Studies 2:3, 200-219.
Chang, Daniel Wonchul. “Method and system for brain entertainment.” U.S. Pat. No. 8,636,640. 28 Jan. 2014.
Cline, John. 2011. “Sleep Spindles.” Psychology Today. www.psychologytoday.com/us/blog/sleepless-in-America/201104/sleep-spindles.
Colzato, L. S., Barone, H., Sellaro, R., & Hommel, B. (2017). More attentional focusing through binaural beats: evidence from the global-local task. Psychological research, 81(1), 271-277.
Colzato, Lorenza S., Amengual, Julià L., et al. “Local entrainment of oscillatory activity induced by direct brain stimulation in humans.” Scientific Reports 7 (2017).
Conte, E., Conte, S., Santacroce, N., Federici, A., Todarello, O., Orsucci, F., . . . & Laterza, V. (2013). A Fast Fourier Transform analysis of time series data of heart rate variability during alfa-rhythm stimulation in brain entrainment. NeuroQuantology, 11(3).
Coombes, S., G. J. Lord, M. R. Owen, Waves and bumps in neuronal networks with axodendritic synaptic interactions. Phys. Nonlinear Phenom. 178, 219-241(2003).
Crick, Francis, and Christof Koch. 1990. “Towards a neurobiological theory of consciousness.” Seminars in the Neurosciences. 263-275. opessoa.ffIch.usp.brisites/opessoa.ifich.usp.brifiles/Crick-Koch-1990.pdf.
Crick, Francis, and Christof Koch. 2006. “What are neural correlates of consciousness?” In 23 Problems in Systems Neuroscience, ed. L. van Hemmen and T. J. Sejnowski. (New York: Oxford Univ. Press), 474-490.
Csaba, G., W. Porod, Coupled oscillators for computing: A review and perspective. Appl. Phys. Rev. 7, 011302 (2020).
D'Atri, A., E. De Simoni, M. Gorgoni, M. Ferrara, F. Ferlazzo, P. M. Rossini, and L. De Gennaro. 2016. “Electrical stimulation of the frontal cortex enhances slow-frequency EEG activity and sleepiness.” Neuroscience 324, 119-130. doi:10.1018/j.neuroscience.2016.03.007.
Davis, Z. W., L. Muller, J. Martinez-Trujillo, T. Sejnowski, J. H. Reynolds, Spontaneous travelling cortical waves gate perception in behaving primates. Nature 587, 432-436 (2020).
De Valois, R. L., K. K. De Valois, Spatial Vision (Oxford Univ. Press, 1990).
DeAngelis, G. C., R. D. Freeman, I. Ohzawa, Length and width tuning of neurons in the cat's primary visual cortex. J. Neurophysiol. 71, 347-374 (1994).
Dennis, T. A., and B. Solomon. 2010. “Frontal EEG and emotion regulation: electrocortical activity in response to emotional film clips is associated with reduced mood induction and attention interference effects.” Biological Psychology 85:3 456-484. doi:10.1016/j.biopsycho.2010.09.008.
Dikker, Suzanne, et al. “Brain-to-brain synchrony tracks real-world dynamic group interactions in the classroom.” Current Biology 27.9 (2017):1375-1380.
Ding, Nai, and Jonathan Z. Simon. “Cortical entrainment to continuous speech: functional roles and interpretations.” Frontiers in human neuroscience 8 (2014).
Dobkins, K. R., T. D. Albright, What happens if it changes color when it moves?: The nature of chromatic input to macaque visual area M T. J. Neurosci. 14, 4854-4870 (1994).
Doherty, Cormac. “A comparison of alpha brainwave entrainment, with and without musical accompaniment.” (2014).
Falk, Simone, Cosima Lanzilotti, and Daniele Schon. “Tuning neural phase entrainment to speech.” J. Cognitive Neuroscience (2017).
Field, D. J., A. Hayes, R. F. Hess, Contour integration by the human visual system: Evidence for a local association field. Vision Res. 33, 173-193 (1993).
Fitzpatrick, D., The functional organization of local circuits in visual cortex: Insights from the study of tree shrew striate cortex. Cereb. Cortex 6, 329-341 (1996).
Foster, D. S. (1990). EEG and subjective correlates of alpha frequency binaural beats stimulation combined with alpha biofeedback (Doctoral dissertation, Memphis State University).
Fröhlich, F., K. K. Sellers, and A. L. Curdle. 2015. “Targeting the neurophysiology of cognitive systems with transcranial alternating current stimulation.” Expert Rev. Neurother. 15:2, 145-67.
Fröhlich, Flavin, and David A McCormick. 2010. “Endogenous electric fields may guide neocortical network activity.” Neuron 67:1, 129-143. doi:10.1016/j.neuron.2010.06.005.
Gao, Junling, et al. “Entrainment of chaotic activities in brain and heart during MBSR mindfulness training.” Neuroscience letters 616 (2016): 218-223.
Gao, X., Can, H., Ming, D., Di, H., Wang, X., Wang, X., & Zhou, P. (2014). Analysis of EEG activity in response to binaural beats with different frequencies. International Journal of Psychophysiology, 94(3), 399-406.
Gebodh, Nigel, Laura Vacchi, Devin Adair, Gozde Unal, Alexander Poltorak, Valeria Poltorak, and Marom Bikson. 2019. “Proceedings #11: Replay of endogenous sleep rhythms to produce sleepiness.” Brain Stimul. 12:2, e71-e72. doi:10.1016/j.brs.2018.12.180.
Gebodh, Nigel, Laura Vacchi, Devin Adair, Zeinab Esmaeilpour, Alexander Poltorak, Valeria Poltorak, and Marom Bikson. 2020. “Abstract 77: Modulation of sleepiness and physiology with brain-derived and narrow-band tACS.” Neuromodulation: Technology at the Neural Interface 23, e326. doi:10.1111/ner.13152.
Gencaga, D., Ed. (2014), p. c002, MDPI: Basel, Switzerland, doi:10.3390/ecea-1-c002.
George, M. S.-B. 2000. “A controlled trial of daily left prefrontal cortex TMS for treating depression.” Biol. Psychiatry 48 (10): 962-970.
Gepshtein, Sergei, Ambarish S. Pawar, Sunwoo Kwon, Sergey Savel'ev, and Thomas D. Albright. “Spatially distributed computation in cortical circuits.” Science advances 8, no. 16 (2022): eab15885.
Gervasoni, Damien, Shih-Chieh Lin, Sidarta Ribeiro, Ernesto S. Soares, Janaina Pantoja, and Miguel A. L. Nicolelis. (2004. “Global forebrain dynamics predict rat behavioral states and their transitions.” J. Neurosci. 24:49, 11137-11147. doi:10.1523/JNEUROSCI.3524-04.2004.
Gilbert, C. D., T. N. Wiesel, Columnar specificity of intrinsic horizontal and corticocortical connections in cat visual cortex. J. Neurosci. 9, 2432-2442 (1989).
Gong, P., L van Leeuwen, Distributed dynamical computation in neural circuits with propagating coherent activity patterns. PLUS Comput. Biol. 5, e1000811 (2009).
Gooding-Williams, Gerard, Hongfang Wang, and Klaus Kessler. “THETA-Rhythm Makes the World Go Round: Dissociative Effects of TMS Theta Versus Alpha Entrainment of Right pTPJ on Embodied Perspective Transformations.” Brain Topography (2017):1-4.
Gooley, J. J., J. Lu, T. C. Chou, T. E. Scammell, and C. B. Saper. 2001. “Melanopsin in cells of origin of the retinohypothalamic tract.” Nat Neurosci. 4:12,1165. doi:10.1038/nn768.PMID: 11713469.
Grover, Shrey, John A. Nguyen, and Robert M. G. Reinhart. 2021. “Synchronizing Brain Rhythms to Improve Cognition.” Annual Review of Medicine 72:1, 29-43.
Gulbinaite, R., T. van Viegen, M. Wieling, M. X. Cohen, and R. VanRullen. 2017. “Individual alpha peak frequency predicts 10 Hz flicker effects on selective attention.” J. Neurosci. 37:42, 10173-10184.
Hämäläinen, M., R. Hari, R. J. Ilmoniemi, J. Knuutila, and D. V. Lounasmaa. 1993. “Magnetoencephalography-theory, instrumentation, and applications to noninvasive studies of the working human brain.” Rev. Mod. Phys. 65, 413. doi:10.1103/revmodphys.65.413.
Hanslmayr, S., J. Matuschek, and M. C. Fellner. 2014. “Entrainment of prefrontal beta oscillations induces an endogenous echo and impairs memory formation.” Current Biology 24:8, 904-909. doi:10.1016/j.cub.2014.03.007.
Heideman, Simone G., Erik S. to Woerd, and Peter Praamstra. “Rhythmic entrainment of slow brain activity preceding leg movements.” Clin. Neurophysiology 126.2 (2015): 348-355.
Heitmann, S., G. B. Ermentrout, Direction-selective motion discrimination by traveling waves in visual cortex. PLOS Comput. Biol. 16, e1008184 (2020).
Helfrich, R. F., T. R. Schneider, S. Rach, S. A. Trautmann-Lengsfeld, A. K. Engel, and C. S. Herrmann. 2014. “Entrainment of brain oscillations by transcranial alternating current stimulation.” Current Biology: C B 24:3, 333-339. doi:10.1018/j.cub.2013.12.041.
Henry, Molly J., et al. “Aging affects the balance of neural entrainment and top-down neural modulation in the listening brain.” Nature Communications 8 (2017): ncomms15801.
Herrmann, C. S. 2013. “Transcranial alternating current stimulation: A review of the underlying mechanisms and modulation of cognitive processes.” Front. Hum. Neurosci. 7:279. doi:10.3389/fnhum.2013.00279.
Hess, R. F., A. Hayes, D. J. Field, Contour integration and cortical processing. J. Physiol.-Paris 97, 105-119 (2003).
Hink, R. F., Kodera, K., Yamada, O., Kaga, K., & Suzuki, J. (1980). Binaural interaction of a beating frequency-following response. Audiology, 19(1), 36-43.
Hogendoorn, H., Motion extrapolation in visual processing: Lessons from 25 years of flash-lag debate. J. Neurosci. 40, 5698-5705 (2020).
Holub, R. A., M. Morton-Gibson, Response of visual cortical neurons of the cat to moving sinusoidal gratings: Response-contrast functions and spatiotemporal interactions. J. Neurophysiol. 46, 1244-1259 (1981).
Hoogenboom N, Schoffelen J M, Oostenveld R, Parkes L M, Fries P. Localizing human visual gamma-band activity in frequency, time and space. Neuroimage. 2006; 29:784-773.
Horr, Ninja K., Maria Wimber, and Massimiliano Di Luca. “Perceived time and temporal structure: Neural entrainment to isochronous stimulation increases duration estimates.” Neuroimage 132 (2016): 148-156.
Huang, Tina L., and Christine Charyton. “A comprehensive review of the psychological effects of brainwave entrainment.” Alternative therapies in health and medicine 14.5 (2008): 38.
Hubei, D. H., T. N. Wiesel, Receptive fields and functional architecture of monkey striate cortex. J. Physiol. 195, 215-243 (1968).
Hutchinson, J. M., A. W. Lo, T. Poggio, A nonparametric approach to pricing and hedging derivative securities via learning networks. J. Finance 49, 851-889 (1994).
Irwin, Rosie. “Entraining Brain Oscillations to Influence Facial Perception.” (2015).
Jadi, M. P., T. J. Sejnowski, Regulating cortical oscillations in an inhibition-stabilized network. Proc. IEEE 102, 830-842 (2014).
James W (1884.) What is an emotion? Mind 9:188-205; Lacey J I, Bateman D E, Vanlehn R (1953) Autonomic response specificity; an experimental study. Psychosom Med 15: 8-21.
Jones, J. P., L. A. Palmer, An evaluation of the two-dimensional Gabor filter model of simple receptive fields in cat striate cortex. J. Neurophysiol. 58, 1233-1258 (1987).
Joyce, Michael, and Dave Siever. “Audio-visual entrainment program as a treatment for behavior disorders in a school setting.” J. Neurotherapy 4.2 (2000): 9-25.
Ju, N., Y. Li, F. Liu, H. Jiang, S. L. Macknik, S. Martinez-Conde, S. Tang, Spatiotemporal functional organization of excitatory synaptic inputs onto macaque VI neurons. Nat. Commun. II, 697 (2020).
Kalyan, Ritu, and Bipan Kaushal. “Binaural Entrainment and Its Effects on Memory.” (2016).
Kasprzak, C. (2011). Influence of binaural beats on EEG signal. Acta Physica Polonica A, 119(6A), 986-990.
Keeler, Clyde E. 1923. “Blind mice.” J. Experimental Zoology 51:4, 495-508. doi:10.1002/jez.1400510404.
Keitel, Anne, et al. “Auditory cortical delta-entrainment interacts with oscillatory power in multiple fronto-parietal networks.” Neuroimage 147 (2017): 32-42.
Keitel, Christian, Cliodhna Duigley, and Philipp Ruhnau. “Stimulus-driven brain oscillations in the alpha range: entrainment of intrinsic rhythms or frequency-following response?” J. Neuroscience 34.31 (2014): 10137-10140.
Kirsch, D. L., and F. Nichols. 2013. “Cranial electrotherapy stimulation for treatment of anxiety, depression, and insomnia.” Psychiatric Clinics of North America 36:1, 169-176. doi:10.1016/j.psc.2013.01.006.
Koelsch, Stefan. “Music-evoked emotions: principles, brain correlates, and implications for therapy.” Annals of the New York Academy of Sciences 1337.1 (2015): 193-201.
Kösem, Anne, et al. “Neural entrainment reflects temporal predictions guiding speech comprehension.” the Eighth Annual Meeting of the Society for the Neurobiology of Language (SNL 2016). 2016.
Kovacs, I., B. Julesz, A closed curve is much more than an incomplete one: Effect of closure in figure-ground segmentation. Proc. Natl. Acad. Sci. U.S.A. 90, 7495-7497 (1993).
Kovacs, I., B. Julesz, Perceptual sensitivity maps within globally defined visual shapes. Nature 370, 644-646 (1994).
Kringelbach, Morten L., and Gustavo Deco. 2020. “Brain states and Transitions: Insights from computational neuroscience.” Cell Reports 32:10, 1-11. doi:10.1016/j.celrep.2020.108128.
Krone, Lukas B., Tomoko Yamagata, Cristina Blanco-Duque, Mathilde C. C. Guillaumin, Martin L Kahn, Vincent van der Vinne, Laura E. McKillop, et al. 2021. “A role for the cortex in sleep-wake regulation.” Nature Neuroscience. doi:10.1038/s41593-021-00894-6.
Lachaux J P, Rodriguez E, Martinerie J, Varela F J. Measuring phase synchrony in brain signals. Hum Brain Mapp. 1999; 8:194-208,
Lakatos, P., C. E. Schroeder, D. I. Leitman, and D. C. Javitt. 2013. “Predictive suppression of cortical excitability and its deficit in schizophrenia.” J. Neurosci. 33:28, 11692-11702. doi:10.1523/JNEUROSCI.0010-13.2013.
Lakatos, Peter, et al. “Entrainment of neuronal oscillations as a mechanism of attentional selection.” Science 320.5872 (2008):110-113.
Lane, J. D., Kasian, S. J., Owens, J. E., & Marsh, G. R. (1998). Binaural auditory beats affect vigilance performance and mood. Physiology & behavior, 63(2), 249-252.
Lawson McLean, Aaron. 2019. “Publication trends in transcranial magnetic stimulation: a 30-year panorama.” Brain Stimul. 12:3 619-627. doi:10.1016/j.brs.2019.01.002.
Le Van Ruyan M, Faucher J, Lachaux Li, et al. Comparison of Hilbert transform and wavelet methods for the analysis of neuronal synchrony. J Neurosci Methods. 2001; 111:83-98,
Lee, Daniel Keewoong, Dongyeup Daniel Synn, and Daniel Chesong Lee. “Intelligent earplug system.” U.S. patent application Ser. No. 15/106,989.
Lee, M., L B. Song, G. H. Shin, and S. W. Lee. 2019. “Possible effect of binaural beat combined with autonomous sensory meridian response for inducing sleep.” Front. Hum. Neurosci. 13, 425.
Lefournour, Joseph, Ramaswamy Palaniappan, and Ian V. McLoughlin. “Inter-hemispheric and spectral power analyses of binaural beat effects on the brain.” Matters 2.9 (2016): e201607000001.
Leong, V, and U Goswami. 2014. “Assessment of rhythmic entrainment at multiple timescales in dyslexia: evidence for disruption to syllable timing.” Hearing Research 308, 141-161. doi:10.1016/j.heares.2013.07.015.
Levenson R W, Heider K, Ekman P, Friesen W V (1992) Emotion and Autonomic Nervous-System Activity in the Minangkabau of West Sumatra. J Pers Soc Psychol 62: 972-988.
Levitt, J. B., J. S. Lund, in Cortical Areas (CRC Press, 2002), pp. 145-166.
Li, X., Z.-L. Lu, P. Xu, J. Jin, Y. Zhou, Generating high gray-level resolution monochrome displays with conventional computer graphics cards and color monitors. J. Neurosci. Methods 130, 9-18 (2003).
Lin, A., K. K. Liu, R. P. Bartsch, and P. C. Ivanov. 2020. “Dynamic network interactions among distinct brain rhythms as a hallmark of physiologic state and function.” Communications biology 3:1, 1-11.
Mai, Guangting, James W. Minett, and William S-Y. Wang. “Delta, theta, beta, and gamma brain oscillations index levels of auditory sentence processing.” Neuroimage 133(2016):516-528.
Manahilov, V., Spatiotemporal visual response to suprathreshold stimuli. Vision Res. 35, 227-237 (1995).
Manahilov, V., Triphasic temporal impulse responses and Mach bands in time. Vision Res. 38, 447-458 (1998).
Marconi, Pier Luigi, et al. “The phase amplitude coupling to assess brain network system integration.” Medical Measurements and Applications (MeMeA), 2016 IEEE International Symposium on. IEEE, 2016.
Marr, D., Vision: A Computational Investigation Into the Human Representation and Processing of Visual Information (W. H. Freeman and Company, 1982).
Mathewson, K. E., M. Fabiani, G. Gratton, D. M. Beck, and A. tiaras. 2010. “Rescuing stimuli from invisibility: Inducing a momentary release from visual masking with pre-target entrainment.” Cognition 115:1, 186-191.
McLaren, Elgin-Skye, and Alissa N. Antle. “Exploring and Evaluating Sound for Helping Children Self-Regulate with a Brain-Computer Application.” Proceedings of the 2017 Conference on Interaction Design and Children. ACM, 2017.
McLean, A. L. (2019). Publication trends in transcranial magnetic stimulation: a 30-year panorama. BrainStimul. 12, 619-627. doi: 10.1016/j.brs.2019.01.002
Mejias, J. F., J. D. Murray, H. Kennedy, X.-J. Wang, Feedforward and feedback frequency-dependent interactions in a large-scale laminar network of the primate cortex. Sci. Adv. 2, e1601335 (2016).
Mély, D. A., D. Linsley, T. Serre, Complementary surrounds explain diverse contextual phenomena across visual modalities. Psychol. Rev. 125, 769-784 (2018).
Meyer, T., C. W. E. M. Quaedflieg, K. Weijland, K. Schruers, H. Merckelbach, and T. Smeets. 2018. “Frontal EEG asymmetry during symptom provocation predicts subjective responses to intrusions in survivors with and without PTSD.” Psychophysiology 55:1. doi:10.1111/psyp.12779.
Miller, K. D., Canonical computations of cerebral cortex. Curr. Opin. Neurobiol. 37, 75-84 (2016).
Moisa, Marius, et al. “Brain network mechanisms underlying motor enhancement by transcranial entrainment of gamma oscillations.” J. Neuroscience 38.47 (2016): 12053-12085.
Molinaro, Nicola, et al. “Out-of-synchrony speech entrainment in developmental dyslexia.” Human brain mapping 37.8 (2016): 2767-2783.
Monaco, J. D., K. Rajan, G. M. Hwang, A brain basis of dynamical intelligence for AI and computational neuroscience. arXiv:210507284 [q-bio.NC] (15 May 2021).
Mori, Toshio, and Shuichi Kai. “Noise-induced entrainment and stochastic resonance in human brainwaves.” Physical review letters 88.21(2002): 218101.
Mortazavi, S. M. J., Zahraei-Moghadam, S. M., Masoumi, S., Rafati, A., Haghani, M., Mortazavi, S. A. R., & Lehtabian, M. (2017). Short Term Exposure to Binaural Beats Adversely Affects Learning and Memory in Rats. Journal of Biomedical Physics and Engineering.
Moseley, R. (2015, July). Inducing targeted brain states utilizing merged reality systems. In Science and Information Conference (SAI), 2015 (pp. 657-663). IEEE.
Moseley, Ralph. “Immersive brain entrainment in virtual worlds: actualizing meditative states.” Emerging Trends and Advanced Technologies for Computational Intelligence. Springer International Publishing, 2016. 315-346.
Movshon, J. A., W. T. Newsome, Visual response properties of striate cortical neurons projecting to area MT in macaque monkeys. J. Neurosci. 16, 7733-7741 (1996).
Muller, L., A. Reynaud, F. Chavane, A. Destexhe, The stimulus-evoked population response in visual cortex of awake monkey is a propagating wave. Nat. Commun. 5, 3675 (2014).
Muller, L., F. Chavane, J. Reynolds, T. J. Sejnowski, Cortical travelling waves: Mechanisms and computational principles. Nat. Rev. Neurosci. 19, 255-268 (2018).
Nakano, D., A. Hirose, Recent advances in physical reservoir computing: A review. Neural Netw. 115, 100-123 (2019).
Neuling, Toralf, et al. “Friends, not foes: magnetoencephalography as a tool to uncover brain dynamics during transcranial alternating current stimulation.” Neuroimage 118 (2015): 406-413.
Nichols, J. A., A. R. Nichols, S. M. Smirnakis, N. D. Engineer, M. P. Kilgard, and M. Atzori. 2011. “Vagus nerve stimulation modulates cortical synchrony and excitability through the activation of muscarinic receptors.” Neuroscience 189, 207-14. doi:10.1016/j.neuroscience.2011.05.024.
Nitsche, M. A. 2000. “Excitability changes induced in the human motor cortex by weak transcranial direct current stimulation.” J. Physiology 527:3 633-639.
Notbohm, Annika, Jurgen Kurths, and Christoph S. Herrmann. 2016. “Modification of brain oscillations via rhythmic light stimulation provides evidence for entrainment but not for superposition of event-related responses.” Front. Hum. Neurosci. 10:20. doi:10.3389/fnhum.2016.00010.
Nozaradan, S., et al. “P943: Neural entrainment to musical rhythms in the human auditory cortex, as revealed by intracerebral recordings.” Clin. Neurophysiology 125 (2014): S299.
Nunez, P. L., and R. Srinivasan. 2006. Electric Fields of the Brain: The Neurophysics of EEG (2 ed). Oxford: Oxford Univ. Press.
Nusslock, R., A. J. Shackman, B. W. McMenamin, L. L. Greischar, R. J. Davidson, and M. Kovacs. 2018. “Comorbid anxiety moderates the relationship between depression history and prefrontal EEG asymmetry.” Psychophysiology 55:1.
Oster, G (October 1973). “Auditory beats in the brain”. Scientific American. 229 (4): 94-102.
Uzeki, H., I. M. Finn, E. S. Schaffer, K. D. Miller, D. Ferster, Inhibitory stabilization of the cortical network underlies visual surround suppression. Neuron 62, 578-592 (2009).
Padmanabhan, R., A. J. Hildreth, and D. Laws. “A prospective, randomised, controlled study examining binaural beat audio and pre-operative anxiety in patients undergoing general anaesthesia for day case surgery.” Anaesthesia 60.9 (2005): 874-877.
Palaniappan, Ramaswamy, et al. “Improving the feature stability and classification performance of bimodal brain and heart biometrics.” Advances in Signal Processing and Intelligent Recognition Systems. Springer, Cham, 2016. 175-186.
Palaniappan, Ramaswamy, Somnuk Phon-Amnuaisuk, and Chikkannan Eswaran. “On the binaural brain entrainment indicating lower heart rate variability.” Int. J. Cardiol 190 (2015): 262-263.
Panksepp J (2007) Neurologizing the Psychology of Affects How Appraisal-Based Constructivism and Basic Emotion Theory Can Coexist. Perspect Psychol Sci 2: 281-296.
Papagiannakis, G., et al. A virtual reality brainwave entrainment method for human augmentation applications. Technical Report, FORTH-ICS/TR-458, 2015.
Park, Hyojin, et al. “Frontal top-down signals increase coupling of auditory low-frequency oscillations to continuous speech in human listeners.” Current Biology 25.12 (2015):1649-1653.
Pattadkal, J. J., G. Mato, C. van Vreeswijk, N. J. Priebe, D. Hansel, Emergent orientation selectivity from random networks in mouse visual cortex. Cell Rep. 24, 2042-2050.e6 (2018).
Pawar, A. S., S. Gepshtein, S. Savel'ev, T. D. Albright, Mechanisms of spatiotemporal selectivity in cortical area MT. Neuron 101, 514-527.e2 (2019).
Payne, Russ W. 2021. “The Brain State Identity Theory.” Chap. 7.1 in An Introduction to Philosophy, by Russ W. Payne, 17804. LibreTexts. Accessed Jul. 13, 2021. human.libretexts.org/@go/page/17604.
Pérez, Alejandro, Manuel Carreiras, and Jon Andoni Duñabeitia. “Brain-to-brain entrainment: EEG interbrain synchronization while speaking and listening.” Scientific Reports 7 (2017).
Petersen, C. C., A. Grinvald, B. Sakmann, Spatiotemporal dynamics of sensory responses in layer 2/3 of rat barrel cortex measured in vivo by voltage-sensitive dye imaging combined with whole-cell voltage recordings and neuron reconstructions. J. Neurosci. 23, 1298-1309 (2003).
Polat, U., D. Sagi, Lateral interactions between spatial channels: Suppression and facilitation revealed by lateral masking experiments. Vision Res. 33, 993-999 (1993).
Poltorak, Alexander. 2019. “On the possibility of transplanting mental states.” USF Preprints. doi:10.31219/osf.io/sjqfx.
Pratt, H., Starr, A., Michalewski, H. J., Dimitrijevic, A., Bleich, N., & Mittelman, N. (2009). Cortical evoked potentials to an auditory illusion: binaural beats. Clinical Neurophysiology, 120(8), 1514-1524.
Pratt, H., Starr, A., Michalewski, H. J., Dimitrijevic, A., Bleich, N., & Mittelman, N. (2010). A comparison of auditory evoked potentials to acoustic beats and to binaural beats. Hearing research, 262(1), 34-44.
Priebe, N. J., S. G. Lisberger, J. A. Movshon, Tuning for spatiotemporal frequency and speed in directionally selective neurons of macaque striate cortex. J. Neurosci. 26, 2941-2950 (2006).
Purves, Dale, George J. Augustine, David Fitzpatrick, Lawrence C Katz, Anthony-Samuel LaMantia, James O McNamara, and S Mark Williams. 2001. Neuroscience. 2nd. Sunderland, M A: Sinauer Associates. www.ncbi.nlm.nih.gov/books/NBK10799/.
Reedijk, S. A., Bolders, A., & Hommel, B. (2013). The impact of binaural beats on creativity. Frontiers in human neuroscience, 7.
Rees, G., G. Kreiman, and Christof Koch. 2002. “Neural correlates of consciousness in humans Neuroscience.” Nature Reviews 3:4, 261-270. doi:10.1038/nrn783.
Riecke, Lars, Alexander T. Sack, and Charles E. Schroeder. “Endogenous delta/theta sound-brain phase entrainment accelerates the buildup of auditory streaming.” Current Biology 25.24 (2015): 3196-3201.
Riesen, G., A. M. Norcia, J. L. Gardner, Humans perceive binocular rivalry and fusion in a tristable dynamic state. J. Neurosci. 39, 8527-8537 (2019).
Ringach, D. L., The geometry of masking in neural populations. Nat. Commun. 10, 4879 (2019).
Rinzel, J., D. Terman, X.-J. Wang, B. Ermentrout, Propagating activity patterns in large-scale inhibitory neuronal networks. Science 279, 1351-1355 (1998).
Robinovitch, Louise G. 1911. “Electric analgesia, and electric resuscitation after heart failure under chloroform or electrocution.” Journal of the American Medical Association, LVI (7), 478-481. doi:10.1001/jama.1911.02560070010003.
Robinson, P. A., X. Zhao, K. M. Aquino, J. D. Griffiths, S. Sarkar, G. Mehta-Pandejee, Eigenmodes of brain activity: Neural field theory predictions and comparison with experiment. Neuroimage 142, 79-98 (2016).
Rodriguez E, George N, Lachaux J P, Martinerie J, Renault B, Varela F J. Perception's shadow: long-distance synchronization of human brain activity. Nature. 1999; 397:430-433.,
Rogenmoser, L., N. Zollinger, S. Elmer, and L. Jäncke. 2016. “Independent component processes underlying emotions during natural music listening.” Social Cognitive and Affective Neuroscience 11:9, 1428-1439. doi:10.1093/scan/nsw048.
Romei, Vincenzo, Gregor Thut, and Juha Silvanto. 2016. “Information-based approaches of noninvasive transcranial brain stimulation.” Trends in Neurosciences 39:11, 782-795.
Rosa, M. A., and S. H. Lisanby. 2012. “Somatic treatments for mood disorders.” Neuropsychopharmacology 37 (1): 102-116. doi:10.1038/npp.2011.225.
Rosenberg, O., N. Shoenfeld, A. Langen, M. Kotler, and P. N. Dannon. 2010. “Deep TMS in a resistant major depressive disorder: a brief report.” Depression and Anxiety 27:5, 465-469. doi:10.1002/da.20689.
Rubin, D. B., S. D. Van Hooser, K. D. Miller, The stabilized supralinear network: A unifying circuit motif underlying multi-input integration in sensory cortex. Neuron 85, 402-417 (2015).
Salinas, E., T. J. Sejnowski, Correlated neuronal activity and the flow of neural information. Nat. Rev. Neurosci. 2, 539-550 (2001).
Savel'ev, S., S. Gepshtein, Neural wave interference in inhibition-stabilized networks, in Proceedings of the first International Electronic Conference on Entropy and Its Applications,
Sceniak, M. P., M. J. Hawken, R. Shapley, Contrast-dependent changes in spatial frequency tuning of macaque VI neurons: Effects of a changing receptive field size. J. Neurophysiol. 88, 1363-1373 (2002).
Schalles, Matt D., and Jaime A. Pineda. “Musical sequence learning and EEG correlates of audiomotor processing.” Behavioural neurology 2015 (2015). www.hindawi.com/journals/bn/2015/638202/
Schroeder, C. E., D. A. Wilson, T. Radman, H. Scharfman, and P. Lakatos. 2010. “Dynamics of Active Sensing and perceptual selection.” Current Opinion in Neurobiology 20:2, 172-176. doi:10.1016/j.conb.2010.02.010.
Schulze, H. H. (1989). The perception of temporal deviations in isochronic patterns. Attention, Perception, & Psychophysics, 45(4), 291-296.
Schwartz, O., A. Hsu, P. Dayan, Space and time in visual context. Nat. Rev. Neurosci. 8, 522-535 (2007).
Semedo, J. D., A. Landvakili, C. K. Machens, B. M. Yu, A. Kohn, Cortical areas interact through a communication subspace. Neuron 102, 249-259.e4 (2019).
Senk, J., K. Korvasová, J. Schuecker, E. Hagen, T. Tetzlaff, M. Diesmann, M. Helias, Conditions for wave trains in spiking neural networks. Phys. Rev. Res. 2, 023174 (2020).
Shapley, R., P. Lennie, Spatial frequency analysis in the visual system. Annu. Rev. Neurosci. 8, 547-583 (1985).
Soltész, F., D. Szűcs, V. Leong, S. White, and U. Goswami. 2013. “Differential entrainment of neuroelectric delta oscillations in developmental dyslexia.” PluS One 8:10, e76608. doi:10.1371/journal.pone.0076608.
Song, W.-J., H. Kawaguchi, S. Totoki, Y. Inoue, T. Katura, S. Maeda, S. Inagaki, H. Shirasawa, M. Nishimura, Cortical intrinsic circuits can support activity propagation through an isofrequency strip of the guinea pig primary auditory cortex. Cereb. Cortex 16, 718-729 (2006).
Spaak, Eelke, Floris P. de Lange, and Ole Jensen. “Local entrainment of alpha oscillations by visual stimuli causes cyclic modulation of perception.” J. Neuroscience 34.10(2014):3536-3544.
Spencer K M, Nestor P G, Perlmutter R, et al. Neural synchrony indexes disordered perception and cognition in schizophrenia. Proc Natl Acad Sci USA. 2004; 101:17288-17293.
Steriade, M. 2006. “Grouping of brain rhythms in corticothalamic systems.” Neuroscience 137:4, 1087-1106.
Stettler, D. D., A. Das, J. Bennett, C. D. Gilbert, Lateral connectivity and contextual interactions in macaque primary visual cortex. Neuron 36, 739-750 (2002).
Sung, H. C., Lee, W. L., Li, H. M., Lin, C. Y., Wu, Y. Z., Wang, J. J., & Li, T. L. (2017). Familiar Music Listening with Binaural Beats for Older People with Depressive Symptoms in Retirement Homes. Neuropsychiatry, 7(4).
Tadin, D., J. S. Lappin, L. A. Gilroy, R. Blake, Perceptual consequences of centre-surround antagonism in visual motion processing. Nature 424, 312-315 (2003).
Tadin, D., J. S. Lappin, Optimal size for perceiving motion decreases with contrast. Vision Res. 45, 2059-2064 (2005).
Tadin, D., Suppressive mechanisms in visual motion processing: From perception to intelligence. Vision Res. 115, 58-70 (2015).
Tanaka, G., T. Yamane, J. B. Heroux, R. Nakane, N. Kanazawa, S. Takeda, H. Numata,
Teich, A. F., N. Dian, Learning and adaptation in a recurrent model of VI orientation selectivity. J. Neurophysiol. 89, 2086-2100 (2003).
Thaut, Michael H. “The discovery of human auditory-motor entrainment and its role in the development of neurologic music therapy.” Progress in brain research 217 (2015): 253-266.
Thaut, Michael H., David A. Peterson, and Gerald C. McIntosh. “Temporal entrainment of cognitive functions.” Annals of the New York Academy of Sciences 1060.1 (2005): 243-254.
Thaut, Michael H., Gerald C. McIntosh, and Volker Hoemberg. “Neurobiological foundations of neurologic music therapy: rhythmic entrainment and the motor system.” Frontiers in psychology 5 (2014).
Thomson, J. M., and U. Goswami. 2008. “Rhythmic processing in children with developmental dyslexia: auditory and motor rhythms link to reading and spelling.” Journal of Physiology 102:1-3, 120-129. doi:10.1016/j.jphysparis.2008.03.007.
Thut, G. “T030 Guiding TMS by EEG/MEG to interact with oscillatory brain activity and associated functions.” Clin. Neurophysiology 128.3 (2017): e9.
Thut, Gregor, Philippe G. Schyns, and Joachim Gross. “Entrainment of perceptually relevant brain oscillations by non-invasive rhythmic stimulation of the human brain.” Frontiers in Psychology 2 (2011).
Thut, Gregor, Philippe G. Schyns, and Joachim Gross. 2011. “Entrainment of perceptually relevant brain oscillations by non-invasive rhythmic stimulation of the human brain.” Front. Psychol. 2:170. doi:10.3389/fpsyg.2011.00170.
Trevino, Guadalupe Villarreal, et al. “The Effect of Audio Visual Entrainment on Pre-Attentive Dysfunctional Processing to Stressful Events in Anxious Individuals.” Open J. Medical Psychology 3.05 (2014): 364.
Trost, W., Frühholz, S., Schön, D., Labbé, C., Pichon, S., Grandjean, D., & Vuilleumier, P. (2014). Getting the beat: entrainment of brain activity by musical rhythm and pleasantness. NeuroImage, 103, 55-64.
Tsai, Shu-Hui, and Yue-Der Lin. “Autonomie feedback with brain entrainment.” Awareness Science and Technology and Obi-Media Computing (iCAST-UMEDIA), 2013 International Joint Conference on. IEEE, 2013.
Tsodyks, M. V., W. E. Skaggs, T. J. Sejnowski, B. L. McNaughton, Paradoxical effects of external modulation of inhibitory interneurons. J. Neurosci. 17, 4382-4388 (1997).
Utz, K. S., V. Dimova, K. Oppenlander, and G. Kerkhoff. 2010. “Electrified minds: transcranial direct current stimulation (tDCS) and galvanic vestibular stimulation (GVS) as methods of non-invasive brain stimulation in neuropsychology-a review of current data and future implications.” Neuropsychologia 48:10, 2789. doi:10.1016/j.neuropsychologia.2010.06.002.
Van der Vinne, N., M. A. Vollebregt, M. J. A. M. van Putten, and M. Arns. 2017. “Frontal alpha asymmetry as a diagnostic marker in depression: Fact or fiction? A meta-analysis.” Neuroimage. Clinical 16, 79-87. doi:10.1016/j.nicl.2017.07.006.
Vossen, Alexandra, Joachim Gross, and Gregor Thut. “Alpha power increase after transcranial alternating current stimulation at alpha frequency (α-tACS) reflects plastic changes rather than entrainment.” Brain Stimulation 8.3 (2015): 499-508.
Wang, W., G. Pedretti, V. Milo, R. Carboni, A. Calderoni, N. Ramaswamy, A. S. Spinelli, D. Ielmini, Learning of spatiotemporal patterns in a spiking neural network with resistive switching synapses. Sci. Adv. 4, eaat4752 (2018).
Will, Udo, and Eric Berg. “Brain wave synchronization and entrainment to periodic acoustic stimuli.” Neuroscience letters 424.1(2007): 55-60.
Wilson, H. R., Binocular contrast, stereopsis, and rivalry: Toward a dynamical synthesis. Vision Res. 140, 89-95 (2017).
Wilson, H. R., J. D. Cowan, A mathematical theory of the functional dynamics of cortical and thalamic nervous tissue. Kybernetik 13, 55-80 (1973).
Wilson, H. R., J. D. Cowan, Excitatory and inhibitory interactions in localized populations of model neurons. Biophys. J. 12, 1-24 (1972).
Wilson, H. R., J. R. Bergen, A four mechanism model for threshold spatial vision. Vision Res. 19, 19-32 (1979).
Wilson, H. R., Spikes, Decisions, and Actions: The Dynamical Foundations of Neuroscience (Oxford Univ. Press, 1999).
Witkowski, Matthias, et al. “Mapping entrained brain oscillations during transcranial alternating current stimulation (tACS).” Neuroimage 140 (2016): 89-98.
Xu, W., X. Huang, K. Takagaki, J. Wu, Compression and reflection of visually evoked cortical waves. Neuron 55, 119-129 (2007).
Yu, X., M. Fumoto, Y. Nakatani, T. Sekiyama, H. Kikuchi, Y. Seki, I. Sato-Suzuki, and H. Arita. 2011. “Activation of the anterior prefrontal cortex and serotonergic system is associated with improvements in mood and EEG changes induced by Zen meditation practice.” Int J of Psychophysiology 80:2, 103-111. doi:10.1016/j.ijpsycho.2011.02.004.
Zangeneh-Nejad, F., D. L. Saunas, A. Alù, R. Fleury, Analogue computing with metamaterials. Nat. Rev. Mater. 6, 207-225 (2021).
Lanus, T. P., P. J. Mineault, K. T. Nasiotis, D. Guitton, C. C. Pack, A sensorimotor role for traveling waves in primate visual cortex. Neuron 85, 615-627 (2015).
Zhuang, Tianbao, Hong Zhao, and Zheng Tang. “A study of brainwave entrainment based on EEG brain dynamics.” Computer and information science 2.2 (2009): 80.
Zlotnik, Anatoly, Raphael Nagao, and Istvan Z. Kiss Jr-Shin Li. “Phase-selective entrainment of nonlinear oscillator ensembles.” Nature Communications 7 (2016).

PCT Application, U.S. Patent, and Patent Application Nos. PCT/US18/51513; 62/560,502; Ser. No. 16/134,309, 2019/0082990; 62/568,610; 62/594,452; Ser. No. 16/209,301; 2019/0247662; 62/612,565; Ser. No. 16/237,471; 2019/0201691; Ser. No. 16/237,483; U.S. Pat. No. 11,273,283; 2019/0200888; Ser. No. 16/237,497; 2019/0224441; Ser. No. 17/693,395; PCT/US18/68220; 62/660,839; Ser. No. 16/388,845; 2019/030583; 62/731,674; Ser. No. 16/572,153; 2021/0086078; PCT/US19/51320; 62/852,877; 62/862,656; Ser. No. 16/883,541; 2020/0368491; Ser. No. 17/375,599; 2021/0338973; 2000244956; 2000249265; 221023681; 20210228883; 2000022877; 2000208680; 20210200313; 2000196964; 2000196950; 2000169686; 2000169685; 20210169684; 2000169395; 2000165490; 20210146164; 2020138276; 200138275; 20210138249; 20210137455; 202012005; 20210121697; 200113831; 2020106830; 20210077032; 2000076090; 20210063972; 20210046276; 2000041953; 20210023378; 20210008385; 20200496049; 20200495204; 20200398055; 20200397383; 20200391028; 20200390350; 20200375530; 20200375525; 20200375523; 20200368491; 20200367789; 20200365125; 20200356136; 20200353262; 20200337652; 20200337645; 20200337625; 20200324115; 20200306542; 20200289785; 20200246579; 20200237300; 20200230413; 20200222010; 2020008350; 2020014586; 20200194120; 20200192478; 20200188702; 20200188701; 20200188700; 20200188699; 20200188690; 20200188697; 20200185093; 20200178875; 20200170534; 2020016403; 20200163610; 20200163572; 2020015582; 20200155790; 20200155061; 20200147387; 20200147349; 20200139113; 20200139112; 20200138323; 20200131156; 2020012911; 202001008257; 2020086127; 20200086122; 20200086078; 20200077941; 20200073475; 20200069209; 20200069200; 20200060571; 20200060566; 20200038674; 20200038657; 20200038653; 20200037942; 20200019243; 20200008725; 20200001099; 20200001080; 20190387990; 20190384392; 20190380607; 20190355476; 20190351248; 20190344978; 20190343448; 20190342602; 2019030583; 20190307350; 20190298923; 20190298254; 20190290912; 20190286234; 2019020200; 20190247662; 20190246898; 201902468936; 20190224480; 20190224441; 20190223747; 20190223746; 20190201691; 20190200949; 20190200934; 20190200925; 20190200880; 20190184175; 20190184174; 2019016800; 20190167179; 20190150775; 20190150774; 20190150760; 20190143136; 20190134402; 20190126033; 20190125255; 20190120919; 20190117933; 20190117106; 20190113973; 20190111250; 20190090823; 20190082990; 20190076079; 20190076046; 20190059768; 20190038902; 20190038203; 20190038166; 20190033914; 20190030230; 20190022426; 2019002657; 20180360343; 2018035046; 20180348863; 20180333582; 2018032607; 20180326210; 20180317795; 20180310855; 2018089960; 20180289287; 20180285442; 2018020656; 20180278984; 20180278822; 2018027022; 20180284265; 2018025051; 20180250494; 20180246570; 20180243565; 20180239430; 20180236230; 20180235934; 20180228423; 20180228394; 2018020661; 2018020620; 20180200518; 20180193641; 20180192936; 20180178013; 20180169429; 20180169428; 20180160982; 201801468916; 20180126167; 20180117331; 20180110997; 20180099144; 20180093092; 20180092557; 20180078165; 20180071530; 20180070847; 20180068581; 20180049704; 2018002579; 20170368297; 20170347906; 20170340260; 20170331563; 20170312519; 20170312517; 20170311832; 2017030462; 20170304622; 20170296820; 20170281946; 20170274174; 20170259167; 20170258390; 20170249445; 20170182285; 20170173351; 20170164895; 20170151436; 20170151122; 20170150916; 20170143966; 20170143963; 20170135633; 20170135597; 20170120043; 20170113048; 20170112427; 20170087367; 20170080256; 2017006522; 20170065199; 20170056663; 20170035368; 20170032098; 20160375245; 20160361534; 20160361008; 20160345855; 20160339243; 20160325110; 20160310698; 20160300352; 20160296759; 20160296746; 20160287879; 20160287871; 20160287436; 2016028711; 20160279380; 20160278697; 20160267808; 20160256691; 20160256690; 20160243381; 20160242690; 20160235351; 20160235323; 20160232811; 20160220836; 20160220821; 2016023271; 20160206871; 20160199662; 20160151018; 20160147964; 20160144186; 20160135727; 20160128580; 20160110551; 20160081610; 20160077547; 20160066838; 20160030750; 20160030748; 20160029946; 20160022168; 20160022167; 20160016014; 20160015288; 20160012749; 20160008632; 20160005320; 20160001098; 20160001065; 20150352362; 20150351655; 20150343242; 20150313496; 20150305686; 20150297109; 20150272496; 20150248615; 20150248470; 20150238761; 20150223714; 2015021648; 2015026436; 20150208978; 20150199010; 20150182753; 20150174497; 20150173641; 20150164857; 20150157266; 20150142082; 20150126802; 20150105837; 20150080695; 20150080670; 20150073575; 20150073237; 20150051637; 20150039066; 20150038868; 20150038822; 20150038776; 20150025610; 20150025408; 20150025352; 20150012057; 20150005592; 20140378851; 20114371515; 20140358024; 20114350634; 20114347265; 20140335488; 201140330345; 20140323898; 20140316248; 20114309614; 20140309484; 20149296646; 201140277255; 20114276551; 20149276353; 20114275716; 20114257147; 20140257128; 20149257073; 20140228900; 201140228651; 20140223462; 201492277; 20114023926; 2014021593; 20149194945; 20140180358; 20114164956; 20140155772; 20140148657; 20140135870; 20140135868; 20140135589; 20140081348; 20140081347; 20140058483; 20140058241; 20114052213; 20114046203; 20114039331; 20149031711; 20149023999; 2011491012; 20140010387; 20130345526; 20130338526; 20130338424; 20130296730; 20130261706; 20130260361; 20130245714; 20130245464; 20130245424; 20130225992; 2013008238; 2013021238; 20130204153; 20130204144; 20130197605; 20130177883; 20130144183; 20130131537; 21130102833; 20130096441; 20130072775; 20130046358; 20130012804; 20120296253; 20120277618; 20120277548; 20120271374; 20120271372; 20120271188; 20120271148; 20120265262; 2012221075; 20120203079; 20120191157; 20120172743; 20120150545; 20120150251; 20120116475; 20120116183; 20120108998; 20120108996; 20120083701; 20120083700; 20120080305; 20120078323; 20120071947; 20120071779; 20120071775; 20120071774; 20120059430; 20120053483; 20120046711; 20120022392; 2012002394; 20110319785; 20110307030; 20110298706; 20110270347; 20110270346; 20110270096; 20110270095; 20110270074; 20110251408; 20110248914; 2011023222; 20110207988; 20110201944; 20110190846; 20110172554; 20110166430; 20110162645; 20110160795; 20110159467; 20110137371; 20110125048; 20110118618; 20110112426; 20110061647; 20110054648; 20110028827; 20110015538; 20100324449; 20100292752; 20100292602; 20100274303; 20100224188; 2010020894; 20100198098; 20100191304; 20100185113; 20100168603; 20100145176; 20100137937; 20100125219; 20100121215; 20100114813; 20100113958; 2010010627; 20100106201; 20100106043; 20100100153; 20100100001; 201000876898; 20100069738; 20100056854; 20100042011; 20100028841; 20100016752; 20090318794; 20090318773; 20090234418; 20090227888; 20090112280; 20080112279; 20080112278; 20080112277; 20080112273; 2080099627; 20090076417; 20090069863; 20090062676; 20080286453; 20080269842; 20080269631; 20080289630; 20080234598; 208029917; 20080208074; 20080183097; 20080167571; 20080114417; 20080064934; 20080046026; 20080033508; 20080033502; 20080027515; 20080027348; 20080027347; 2008002514; 20080021341; 20070282388; 20070255677; 20070260286; 20070260147; 20070244417; 2007023786; 20070213785; 20070213784; 20070191704; 20070173902; 20070142874; 20070100392; 20070100278; 20070067008; 20070066915; 20070043491; 20070032737; 20060265022; 20060241562; 2006027781; 20060173522; 20060161384; 20060155495; 20060149337; 20060129324; 20060129204; 20050283053; 20050197590; 20050153268; 20050115561; 20050107838; 20050081847; 20050065427; 20040158118; 20040152958; 20040138711; 20040138647; 20040138581; 20040138580; 20040138536; 20049138518; 20049138517; 20049138516; 20049133390; 20049133248; 20040133121; 20049133119; 20049122335; 20049092808; 20049082984; 20049079372; 20030229291; 20030228371; 20030225349; 20030181955; 20030004429; 20020188330; 20020103429; 20020091335; 20020082513; U.S. Pat. Nos. 11,083,413; 11,083,402; 11,077,305; 11,064,930; 11,051,748; 11,042,982; 11,026,835; 11,026,622; 11,026,616; 11,023,046; 11,006,890; 11,000,685; 10,994,144; 10,993,636; 10,962789; 10,959682; 10,946,196; 10,946,165; 10,945,880; 10,945,879; 10,942,568; 10,940,315; 10,933,241; 10,925,834; 10,910,510; 10,918,324; 10,914,803; 10,905,877; 10,901,509; 10,898,711; 10,898,706; 10,893,822; 10,891,313; 10,874,341; 10,864,376; 10,856,803; 10,856,032; 10,842,932; 10,835,176; 10,827,942; 10,806,717; 10,799,700; 10,799,700; 10,799,667; 10,796,596; 10,779,747; 10,768,665; 10,736,561; 10,729,908; 10,720,076; 10,682,490; 10,675,482; 10,653,883; 10,632,311; 10,625,042; 10,624,875; 10,606,353; 10,580,123; 10,583,299; 10,582,875; 10,576,283; 10,549,09; 10,543,358; 10,538,560; 10,537,703; 10,531,806; 10,531,805; 10,529,450; 10,525,267; 10,507,335; 10,499,843; 10,478,086; 10,471,271; 10,463,271; 10,452,144; 10,449,359; 10,433,753; 10,420,937; 10,413,724; 10,406,364; 10,405,792; 10,405,025; 10,396,905; 10,384,064; 10,371,776; 10,368,802; 10,365,716; 10,363,362; 10,357,654; 10,350,430; 10,349,860; 10,342,986; 10,322,294; 10,321,842; 10,307,324; 10,278,608; 10,272,247; 10,265,528; 10,265,527; 10,265,497; 10,258,291; 10,252,055; 10,238,870; 10,226,629; 10,226,220; 10,209,719; 10,207,111; 10,188,864; 10,180,860; 10,172,567; 10,159,816; 10,159,648; 10,155,121; 10,154,988; 10,154,812; 10,130,766; 10,130,294; 10,120,413; 10,095,837; 10,092,753; 10,087,223; 10,080,895; 10,071,245; 10,071,132; 10,064,912; 10,052,486; 10,052,485; 10,052,452; 10,046,174; 10,046,162; 10,035,027; 10,018,695; 10,009,644; 9,987,45; 9,983,670; 9,974,957; 9,974,956; 9,968,783; 9,955,905; 9,948,004; 9,946,344; 9,943,698; 9,943,688; 9,931,074; 9,889,656; 9,888,302, 9,886,493; 9,078,176; 9,878,155; 9,877,664; 9,855,442; 9,848,300; 9,884,541; 9,824,607; 9,820,663; 9,014,426; 9,808,641; 9,808,207; 9,788,750; 9,788,744; 9,782,590; 9,757,587; 9,750,448; 9,728,252; 9,724,521; 9,724,517; 9,700,723; 9,700,541; 9,700,256; 9,656,075; 9,643,019; 9,622,675; 9,615,788; 9,604,073; 9,597,494; 9,597,493; 9,592,003; 9,579,508; 9,563,950; 9,563,273; 9,554,720; 9,545,226; 9,538,950; 9,533,147; 9,950,760; 9,505,817; 9,504,390; 9,498,628; 9,480,845; 9,480,812; 9,468,761; 9,458,208; 9,451,894; 9,445,730; 9,448,064; 9,415,222; 9,399,144; 9,394,347; 9,387,320; 9,375,573; 9,375,564; 9,365,628; 9,364,163; 9,358,448; 9,357,965; 9,357,941; 9,338,642; 9,332,938; 9,326,720; 9,314,623; 9,308,296; 9,302,111; 9,289,143; 9,284,353; 9,282,930; 9,271,674; 9,265,433; 9,259,591; 9,248,234; 9,248,200; 9,241,647; 9,238,150; 9,224,308; 9,186,106; 9,179,850; 9,173,609; 9,167,978; 9,167,977; 9,167,976; 9,113,801; 9,095,303; 9,079,849; 9,079,038; 9,072,832; 9,072,482; 9,067,070; 9,056,195; 9,050,469; 9,042,990; 9,042,988; 9,042,201; 9,201,585; 9,020,582; 9,014,804; 9,088,863; 8,989,836; 8,965,513; 8,954,152; 8,948,855; 8,938,290; 8,934,965; 8,932,218; 8,918,176; 8,914,115; 8,892,207; 8,888,702; 8,880,167; 8,868,173; 8,868,172; 8,855,775; 8,852,100; 8,849,409; 8,848,390; 8,838,227; 8,834,546; 8,831,732; 8,827,912, 8,825,166; 8,815,582; 8,812,088, 8,788,044; 8,786,624; 8,781,597; 8,771,194; 8,768,471; 8,762,065; 8,761,888; 8,750,010; 8,738,136; 8,725,243; 8,716,447; 8,712,512; 8,706,237; 8,706,181; 8,706,142; 8,696,722; 8,670,830; 8,660,648; 8,649,871; 8,636,649; 8,604,075; 8,603,790; 8,594,798; 8,594,779; 8,576,786; 8,571,643; 8,565,886; 8,562,536; 8,562,524; 8,560,100; 8,560,073; 8,554,325; 8,543,214; 8,543,199; 8,538,512; 8,478,538; 8,478,419; 8,473,068; 8,452,387; 8,447,496; 8,428,733; 8,417,344; 8,396,545; 8,382,667; 8,380,296; 8,374,696; 8,353,837; 8,352,023; 8,337,494; 8,315,970; 8,295,934; 8,267,851; 8,265,743; 8,249,712; 8,244,475; 8,214,035; 8,209,019; 8,209,010; 8,187,181; 8,172,759; 8,150,524; 8,121,646; 8,112,148; 8,086,316; 8,073,545; 8,036,736; 8,027,730; 8,024,032; 8,000,787; 8,976,465; 7,974,697; 7,933,646; 7,930,035; 7,917,222; 7,917,206; 7,904,144; 7,889,525; 7,894,903; 7,892,182; 7,853,328; 7,819,812; 7,811,992; 7,787,946; 7,715,91; 7,711,417; 7,676,263; 7,668,591; 7,630,757; 7,624,293; 7,582,062; 7,555,344; 7,551,951; 7,532,935; 7,498,752; 7,486,986; 7,471,978; 7,373,199; 7,346,391; 7,341,562; 7,321,837; 7,282,030; 7,280,867; 7,288,456; 7,242,983; 7,204,833; 7,177,678; 7,174,206; 7,149,572; 7,146,201; 7,089,271; 7,088,058; 7,076,977; 7,006,872; 7,006,858; 6,983,184; 6,931,275; 6,793,671; 6,671,555; 6,560,486; 6,549,804; 6,547,746; 6,488,617; 6,480,743; 6,463,328; 6,385,486; 6,374,149; 6,330,713; 6,337,997; 6,317,627; 6,304,775; 6,066,163; 6,052,619; 6,042,548; 6,033,073; 6,018,682; 6,016,444; 5,995,868; 5,611,350; 5,450,117; 5,392,788; RE34,015; 4,883,067; 4,705,049; 4,503,863; 4,421,122; 4,408,616; 4,407,299; 4,305,402; 4,201,224; 5,070,399; 5,306,228; 5,409,445; 6,656,137; 7,749,155; 7,819,794; 7,988,613; 8,088,057; 8,167,784; 8,213,671; 8,267,851; 8,290,078; 8,517,909; 8,517,912; 8,576,793; 8,576,795; 8,597,171; 8,636,641; 8,638,950; 8,668,496; 8,852,073; 8,932,218; 8,968,176; 9,330,523; 9,357,941; 9,458,597; 9,480,812; 9,563,273; 9,608,453; 9,649,167; 9,707,372; 20050153288; 20050182287; 20060106434; 20060206174; 20060281543; 20070066413; 20080039677; 200803046891; 20100010288; 20100010844; 20100028841; 20100056854; 20100076253; 20100130812; 20100222641; 20100286747; 20100298624; 20110298706; 20110319482; 20120003615; 20120053394; 20120150545; 20130030241; 20130072292; 20130131537; 20130172663; 20130184516; 20130203018; 20130234823; 20130338738; 20140088341; 20140107401; 20140114242; 20140154647; 20140174277; 20140275741; 20140309484; 20140371516; 20150142082; 20150283019; 20150296288; 20150313496; 20150313949; 20160008568; 20160019434; 20160055842; 20160205488; 20160235980; 20160239084; 20160345901; 20170034638; 20170061760; 20170087330; 20170094385; 20170095157; 20170099713; 20170135597; 20170149945; U.S. Pat. Nos. 5,273,038; 5,503,149; 6,240,308; 6,272,370; 6,298,259; 6,370,414; 6,385,479; 6,490,472; 6,556,695; 6,697,660; 6,800,648; 6,907,280; 6,996,281; 7,092,748; 7,254,500; 7,338,455; 7,346,382; 7,490,085; 7,497,828; 7,535,288; 7,565,193; 7,567,693; 7,577,472; 7,597,665; 7,627,370; 7,680,526; 7,729,755; 7,80,434; 7,840,257; 7,860,548; 7,872,235; 7,899,524; 7,904,134; 7,904,139; 7,907,998; 7,983,740; 7,983,741; 8,000,773; 8,014,847; 8,069,125; 8,233,682; 8,233,965; 8,235,907; 8,248,068; 8,356,004; 8,379,952; 8,406,838; 8,423,125; 8,445,851; 8,553,956; 8,586,932; 8,606,348; 8,615,479; 8,644,910; 8,679,008; 8,696,722; 8,712,512; 8,718,747; 8,761,866; 8,781,557; 8,814,923; 8,821,376; 8,834,546; 8,852,103; 8,870,737; 8,936,630; 8,951,189; 8,951,192; 8,958,882; 8,983,155; 9,005,126; 9,020,586; 9,022,936; 9,028,412; 9,033,884; 9,042,958; 9,078,584; 9,101,279; 9,135,400; 9,144,392; 9,149,255; 9,155,521; 9,167,970; 9,179,854; 9,179,858; 9,198,637; 9,204,838; 9,208,558; 9,211,077; 9,213,076; 9,235,685; 9,242,067; 9,247,924; 9,268,014; 9,268,015; 9,271,651; 9,271,674; 9,275,191; 9,292,920; 9,307,925; 9,322,895; 9,326,742; 9,330,206; 9,368,265; 9,395,425; 9,402,558; 9,414,776; 9,436,989; 9,451,883; 9,451,899; 9,468,541; 9,471,978; 9,480,402; 9,480,425; 9,486,168; 9,592,388; 9,615,788; 9,626,756; 9,672,302; 9,672,617; 9,682,232; 20020033454; 20020035317; 20020037095; 20020042563; 20020058867; 20020103428; 20020103429; 20030018277; 20030093004; 20030128801; 20040082862; 20040092808; 20040096395; 20040116791; 20040116798; 20040122787; 20040122790; 20040166536; 20040215082; 20050007091; 20050020918; 20050033154; 20050079636; 20050119547; 20050154290; 20050222838; 20050240253; 20050283053; 20060036152; 20060036153; 20060052706; 20060058683; 20060074290; 20060078183; 20060084858; 20060149160; 20060161218; 20060241382; 20060241718; 20070191704; 20070239058; 20080001600; 20080009772; 20080033291; 20080039737; 20080042067; 20080097235; 20080097785; 20080128626; 20080154128; 20080221441; 20080228077; 20080228238; 20080230702; 20080230705; 20080249430; 20080282327; 20080275340; 20090012387; 20090018407; 20090022825; 20090024050; 20090062660; 20090078875; 20090118610; 20090156907; 20090156955; 20090157323; 20090157481; 20090157482; 20090157625; 20090157751; 20090157813; 20090163777; 20090164131; 20090164132; 20090171164; 20090172540; 20090179642; 20090209831; 2009022930; 20090246138; 20090299168; 20090304582; 20090306532; 20090306534; 20090312808; 20090312817; 20090318773; 20090318794; 20090322331; 20090326604; 20100021378; 20100036233; 20100041948; 20100042011; 20100049482; 20100069738; 20100069777; 20100082506; 20100113958; 20100249573; 20110015515; 20110015538; 20110028827; 20110077503; 20110118536; 20110125077; 20110125078; 20110129129; 20110160543; 20110161011; 20110172508; 20110172553; 20110178358; 20110190846; 20110218405; 20110224571; 20110230738; 20110257518; 20110283962; 20110283968; 20110270074; 20110288400; 20110301448; 20110306845; 20110306846; 20110313274; 2012021394; 20120022343; 20120035433; 20120053483; 20120163688; 20120165904; 20120215114; 2012029195; 2012219507; 20120245474; 20120253261; 20120253434; 2012028854; 20120310107; 20120316793; 20130012804; 20130060125; 20130063550; 20130085678; 2013009640; 20130110616; 20130116561; 20130123607; 20130131438; 20130131461; 20130178693; 20130178733; 20130184558; 2013021238; 20130221961; 20130245424; 20130274586; 20130289385; 20130289386; 20130303934; 20140058528; 20140066763; 20140119621; 20140151563; 20140155730; 20140163368; 20140171757; 20140180088; 20140180092; 20140180093; 20140180094; 20140180095; 20140180096; 20140180097; 20140180098; 20140180100; 20140180112; 20140180113; 2011400176; 20140180177; 20140184550; 20140193336; 20140200414; 20140243614; 20140257047; 20140275807; 20140303486; 20140315168; 20140316248; 20140323848; 20140335488; 20140343397; 20140343398; 2014034340; 2014036472; 20140378830; 20150011866; 20150038812; 20150051663; 20150099958; 20150112408; 20150119658; 20150119688; 20150148700; 20150150473; 20150196800; 20150200046; 20150219732; 20150223905; 20150227702; 2015024792; 20150248615; 20150253410; 20150289779; 20150290453; 20150290454; 20150313540; 20150317796; 201503246892; 20150366482; 20150375006; 20160005320; 20160027342; 20160029965; 20160051161; 20160051162; 20160055304; 20160058304; 20160058392; 20160066838; 20160103487; 20160120437; 20160120457; 20160143541; 20160157742; 20160184029; 20160196393; 20160228702; 20160231401; 20160239966; 20160239968; 20160260216; 20160287808; 20160270723; 20160302720; 20160303397; 20160317077; 20160345911; 20170027539; 20170039706; 20170045601; 20170061034; 20170085855; 20170091410; 2017012493; 20170113046; 20170120041; 20170160360; 20170164861; 20170169714; 20170172527; 20170202475; U.S. Pat. Nos. 6,196,972; 6,338,713; 6,442,421; 6,507,754; 6,524,249; 6,547,736; 6,616,611; 6,816,744; 6,865,494; 6,915,241; 6,936,012; 6,996,261; 7,043,293; 7,054,454; 7,079,977; 7,128,713; 7,146,21; 7,149,572; 7,164,941; 7,201,788; 7,254,439; 7,280,867; 7,282,030; 7,321,837; 7,330,032; 7,333,619; 7,381,185; 7,537,560; 7,558,903; 7,565,193; 7,567,693; 7,604,603; 7,624,293; 7,640,055; 7,715,919; 7,725,174; 7,729,755; 7,751,878; 7,778,693; 7,794,496; 7,797,041; 7,801,592; 7,803,11; 7,803,119; 7,879,043; 7,896,807; 7,899,524; 7,917,206; 7,933,646; 7,937,138; 7,976,465; 8,014,847; 8,033,996; 8,073,534; 8,095,210; 8,137,268; 8,137,270; 8,175,696; 8,177,724; 8,177,726; 8,180,601; 8,187,181; 8,197,437; 8,233,965; 8,236,005; 8,244,341; 8,248,068; 8,248,698; 8,280,514; 8,295,914; 8,326,433; 8,335,664; 8,346,342; 8,355,768; 8,386,312; 8,386,313; 8,392,250; 8,392,253; 8,392,254; 8,392,255; 8,396,542; 8,496,841; 8,406,862; 8,412,655; 8,428,703; 8,428,794; 8,463,374; 8,464,288; 8,475,387; 8,483,815; 8,494,610; 8,494,829; 8,494,905; 8,498,699; 8,509,881; 8,533,042; 8,548,786; 8,571,629; 8,579,786; 8,591,419; 8,606,360; 8,628,480; 8,655,428; 8,666,478; 8,682,422; 8,706,183; 8,706,205; 8,718,747; 8,725,238; 8,738,136; 8,747,382; 8,755,877; 8,761,869; 8,762,202; 8,768,449; 8,781,796; 8,790,255; 8,790,272; 8,821,40; 8,825,149; 8,831,731; 8,843,210; 8,848,392; 8,848,632; 8,855,773; 8,858,449; 8,862,210; 8,862,581; 8,903,479; 8,918,178; 8,934,965; 8,951,190; 8,954,139; 8,955,010; 8,958,868; 8,983,628; 8,983,629; 8,989,835; 9,020,788; 9,026,217; 9,031,644; 9,050,470; 9,060,671; 9,070,492; 9,072,832; 9,072,905; 9,078,584; 9,084,896; 9,085,295; 9,101,276; 9,107,595; 9,116,835; 9,125,574; 9,14,9719; 9,155,487; 9,192,309; 9,918,621; 9,204,835; 9,210,417; 9,215,978; 9,232,910; 9,232,984; 9,238,142; 9,242,067; 9,247,911; 9,248,286; 9,254,383; 9,277,871; 9,277,873; 9,282,934; 9,288,603; 9,302,110; 9,307,944; 9,308,372; 9,320,450; 9,336,535; 9,357,941; 9,375,151; 9,375,171; 9,375,571; 9,413,030; 9,415,219; 9,427,581; 9,443,141; 9,451,886; 9,454,646; 9,468,956; 9,462,975; 9,468,541; 9,471,978; 9,480,402; 9,492,084; 9,504,410; 9,522,278; 9,533,113; 9,545,285; 9,560,984; 9,563,741; 9,615,749; 9,616,166; 9,622,672; 9,622,676; 9,622,702; 9,622,703; 9,623,241; 9,636,019; 9,649,036; 9,659,228; 9,668,694; 9,681,814; 9,681,820; 9,682,232; 9,713,420; 20020035338; 20020091318; 20020095098; 20020103420; 20020103429; 20020193670; 20030032888; 20030046018; 20030093129; 20030160622; 2003018540; 2003026654; 20040039288; 20041049484; 20041092808; 20041133118; 20041133120; 20041133390; 20041138536; 20041138580; 20040138711; 20041152958; 20041158118; 20050010091; 20050018858; 20050033174; 20050075568; 20050085744; 20050119547; 20050148893; 20050148894; 20050148895; 20050154290; 20050167588; 20050241087; 20050245796; 20050267343; 20050267344; 20050283053; 20050283090; 20060020184; 20060036152; 20060036153; 20060074290; 20060078183; 20060135878; 20060153396; 20060155495; 20060161384; 20060173364; 20060210013; 2006027816; 20060233390; 20060281980; 20070016095; 20070066915; 20070100278; 20070179395; 20070179734; 20070191704; 20070201668; 20070225932; 20070255122; 20070255135; 20070260151; 20070265508; 20070287896; 20080021345; 20080033508; 20080064934; 20080074307; 20080077015; 2000009111; 20008097197; 20080119716; 20080177196; 20080221411; 2008022441; 20080243014; 20080243017; 20080255948; 20080262367; 20090005667; 20090033333; 20090036791; 20090054801; 20090062676; 20090177144; 20090220425; 20090221930; 20090270758; 20090281448; 20090287271; 20090287272; 20090287273; 20090287467; 20090299168; 20090306534; 20090312646; 20090318794; 20090322331; 20100030073; 2010003621; 20100049276; 20100068751; 20100069738; 20100094152; 20100099975; 20100106041; 20100198090; 20100204604; 20100204748; 20100249638; 20100280372; 20100331976; 2011000415; 20110015515; 20110015538; 20110041713; 20110066041; 20110066042; 20110074396; 20110077538; 20110092834; 20110092838; 20110098583; 20110160543; 20110172725; 20110178441; 20110184305; 20110191350; 20110218950; 20110257518; 20110270074; 20110282230; 20110288431; 20110295143; 20110301441; 20110313268; 20110313487; 20120004518; 20120004561; 2012021394; 20120022343; 20120029378; 20120041279; 20120046535; 20120053473; 20120053476; 20120053478; 20120053479; 20112083708; 2012011108908; 20120108997; 20120143038; 20120145152; 20120150545; 20120157804; 20120159656; 20120172682; 20120184826; 20120197153; 20120209138; 20120253261; 20120265267; 20120271151; 20120271376; 20120289868; 20120310105; 2012032758; 20130012804; 20130041235; 20130060125; 20130066392; 20130066395; 20130072775; 20130079621; 21130102897; 20130116520; 20130123607; 20130127708; 20130131438; 20130131461; 20130165804; 20130167360; 20130172716; 20130172772; 20130178733; 20130184597; 20130204122; 2013021238; 20130223708; 20130226261; 20130237874; 20130238048; 20130238050; 20130245416; 20130245424; 2013024548; 20130245486; 20130245711; 20130245712; 20130261490; 20130274562; 20130289364; 20130295016; 20130310422; 20130310908; 20130317380; 20130338510; 20130338803; 12014039279; 20149057232; 201490582; 2014905852; 20149074179; 20149074180; 20149094710; 2014909472; 20114910750; 20114142654; 20149148657; 20149148716; 20149148726; 20149180153; 20149180160; 20149187901; 201149220702; 2014243647; 20149243714; 20114257120; 20149275807; 20149276130; 20149276187; 20140303454; 20140303500; 20114309614; 2011403167; 20140316248; 20140324110; 20140330334; 20140330335; 20140330336; 20140330404; 20140335489; 20140350634; 20140350864; 20150005646; 20150005660; 20150011907; 20150018665; 20150018699; 20150018702; 20150025422; 20150038869; 20150073294; 20150073306; 20150073505; 20150080671; 20150080695; 20150099962; 201501268; 20150151142; 20150164431; 20150190071; 20150190636; 20150190637; 20150196213; 20150196248; 2015003191; 2015006439; 20150245800; 20150248471; 20150248615; 2050272652; 20150297106; 20150297893; 20150305686; 20150313498; 20150366482; 20150379370; 20160000348; 20160007899; 20160022167; 2016002060; 20160022207; 20160027423; 20160029965; 20160038042; 20160038043; 2016004512; 20160051812; 20160058304; 20160066830; 20160107308; 20160113587; 20160120420; 20160120432; 20160120437; 20160120457; 20160128596; 20160128597; 20160135754; 20160143594; 20160144175; 20160151628; 20160157742; 20160157828; 20160174863; 20160174907; 20160176053; 20160183881; 20160184029; 20160189873; 20160206380; 2016003261; 20160213317; 20160220850; 2016022028; 20160228702; 20160235324; 20160239966; 20160239960; 20160242645; 20160242665; 2016024266; 20160242690; 20160249841; 20160250355; 20160256063; 20160256105; 20160262664; 20160278653; 20160278713; 20160207117; 20160287162; 2016020716; 20160207869; 20160303402; 20160331264; 20160331307; 20160345895; 20160345911; 20160346542; 20160361041; 20160361546; 20160367186; 20160367198; 20170031449; 20170031441; 20170039706; 20170042444; 20170045601; 20170071521; 20170079580; 20170079589; 20170091418; 20170113046; 20170120041; 20170128015; 20170135594; 20170135626; 20170136249; 20170165021; 20170172446; 20170173326; 20170188870; 20170188905; 20170188916; 20170188922; and 20170196519.

### SUMMARY OF THE INVENTION

According to the present invention, sensory or transcranial stimulation is employed to induce a desired brain state by replicating its neural correlates.

Brain states, which correlate with specific motor, cognitive, and emotional states, may be monitored with noninvasive techniques such as electroencephalography (EEG) and magnetoencephalography (MEG) that measure macroscopic cortical activity manifested as oscillatory network dynamics. These rhythmic cortical signatures provide insight into the neuronal activity used to identify pathological cortical function in numerous neurological and psychiatric conditions. Sensory and transcranial stimulation, entraining the brain with specific brain rhythms, can effectively induce desired brain states (such as state of sleep or state of attention) correlated with such cortical rhythms. A desired brain state may be induced by replicating these neural correlates through stimulation.

Brain waves are recorded from a “donor” using EEG/MEG to extract cortical signatures of the brain state, which are then processed and used to entrain the brain of a “recipient” via sensory or transcranial stimulation. These cortical signatures are then inverted and used to entrain the brain of a “recipient” via sensory or transcranial stimulation. Brain states may thus be transferred between people by acquiring an associated cortical signature from a donor, which, following processing, is applied to a recipient through sensory or transcranial stimulation. This technique provides an effective neuromodulation approach to the noninvasive, non-pharmacological treatment of a variety of psychiatric and neurological disorders for which current treatments are mostly limited to pharmacotherapeutic interventions.

The model presumes that detectable cortical brainwaves have a pattern which is modulated by a low-dimensionality modulation vector. The brainwaves are coupled to sensory neural pathways. Further, the brainwaves act as if generated by an oscillator that has various natural frequencies with biological significance, and which can rapidly transition between these states.

It is therefore an object to provide a method of enhancing memory, comprising: providing a substreshold transcranial brain stimulation to a subject; determining a range of endogenous frequencies of brain oscillation of the subject; after the substreshold transcranial brain stimulation, entraining brainwaves of the subject with a first sensory stimulation pattern having frequencies within the range of endogenous frequencies of brain oscillation of the subject; after entraining brainwaves of the subject with a first sensory stimulation pattern, entraining brainwaves of the subject with a second sensory stimulation pattern having a distinct range of frequencies from the range of endogenous frequencies of brain oscillation of the subject, the second sensory stimulation pattern comprising a pattern derived from a human brainwave pattern corresponding to a cognitive task; and engaging the subject in the cognitive task concurrent with exposure of the subject to the second sensory stimulation pattern.

It is therefore an object to provide a method of enhancing memory, comprising: providing a substreshold transcranial brain stimulation to a subject; determining a range of endogenous frequencies of brain oscillation of the subject after the substreshold transcranial brain stimulation; after the substreshold transcranial brain stimulation, entraining brainwaves of the subject with a first sensory stimulation pattern having frequencies within the range of endogenous frequencies of brain oscillation of the subject; after entraining brainwaves of the subject with the first sensory stimulation pattern, entraining brainwaves of the subject with a second sensory stimulation pattern having a distinct range of frequencies from the range of endogenous frequencies of brain oscillation of the subject, the second sensory stimulation pattern comprising a pattern derived from a human brainwave pattern corresponding to a cognitive task; and engaging the subject in the cognitive task concurrent with exposure of the subject to the second sensory stimulation pattern.

It is also an object to provide a method of brain entrainment, comprising: selecting at least one donor having a respective brain state; recording brain electrical or magnetic activities of the brain of the at least one donor in the respective brain state; processing the recorded electrical or magnetic activities to extract a dominant frequency of the at least one donor's brain in the respective brain state; detecting electrical or magnetic activities of the brain of a subject to determine the endogenous dominant frequency of the subject's brainwaves; stimulating the subject's brain with at least one external stimulus having a frequency corresponding to the endogenous dominant frequency of the subject's brainwaves to establish an Arnold tongue; and after establishing the Arnold tongue, changing a frequency of the external stimulus from the endogenous dominant frequency to the dominant frequency of the at least one donor's brain in the respective brain state.

It is a further object to provide a system for enhancing memory, comprising: a stimulator configured to provide a substreshold transcranial brain stimulation to a subject; a user interface configured to engage the subject in a cognitive task; at least one processor configured to: determine a range of endogenous frequencies of brain oscillation of the subject after the substreshold transcranial brain stimulation; after the substreshold transcranial brain stimulation, entrain brainwaves of the subject with a first sensory stimulation pattern having frequencies within the range of endogenous frequencies of brain oscillation of the subject; after entrainment of brainwaves of the subject with the first sensory stimulation pattern, entrain brainwaves of the subject with a second sensory stimulation pattern having a distinct range of frequencies from the range of endogenous frequencies of brain oscillation of the subject, the second sensory stimulation pattern comprising a pattern derived from a human brainwave pattern corresponding to a cognitive task; and engage the subject through the user interface in the cognitive task concurrent with exposure of the subject to the second sensory stimulation pattern.

The respective brain state brain state may be a sleeping state, a waking state, a relaxation state, a state of mental focus, a state of mental flow, a state of altered consciousness, a meditative state, and an emotional state.

The at least one donor may be of the same biological sex as the subject. The at least one donor may have an age within 5 years of the subject's age. The steps of processing and detecting may be performed on the recordings of cortical activity of the plurality of donors.

The step of stimulating the subject may comprise subjecting the subject to at least one of a light, a sound, a tactile vibration, a transcranial electric stimulation, a transcranial direct current stimulation (tDCS), a transcranial alternating current stimulation (tACS), and a transcranial magnetic stimulation.

The step of processing the recorded electrical or magnetic activities to extract a dominant frequency of the at least one donor's brain in the respective brain state may comprise performing a statistical pattern classification, a principal component analysis, a multilinear principal component analysis, an independent component analysis, a clustering, a linear discriminant analysis, a factor analysis, a quadratic discriminant analysis, a multinomial logistic regression, or a EEG multifractal analysis.

The step of processing the recorded electrical or magnetic activities to extract a dominant frequency of the at least one donor's brain in the respective brain state may be performed by a support vector machine, a maximum entropy Markov model, a recurrent neural network, or a deep neural network.

It is a further object to provide a method of brain entrainment for transplanting a desired brain state, comprising: extracting a dominant brainwave frequency of at least one donor in the desired brain state; detecting an endogenous dominant brainwave frequency of a subject desirous of achieving the desired brain state; stimulating the subject's brain with at least one external stimulus having a frequency corresponding to the endogenous dominant brainwave frequency of the subject to lock phase and establish an Arnold tongue; and after establishing the Arnold tongue, incrementally changing the frequency of the external stimulus from the endogenous dominant frequency to the dominant brainwave frequency of said at least one donor's brain, thereby assisting the subject in achieving the desired brain state.

It is also an object to provide 14. A system for transplanting a desired brain state through brain entrainment, comprising: at least one memory configured to store an extracted dominant brainwave frequency of at least one donor in the desired brain state; and at least one automated processor configured to: detect an endogenous dominant brainwave frequency of a subject desirous of achieving the desired brain state; stimulate the subject's brain with at least one external stimulus having a frequency corresponding to the endogenous dominant brainwave frequency of the subject to lock phase and establish an Arnold tongue; and after establishing the Arnold tongue, incrementally change the frequency of the external stimulus from the endogenous dominant frequency to the dominant brainwave frequency of said at least one donor's brain, to thereby assist the subject in achieving the desired brain state.

The desired brain state may be a sleeping state.

The respective brain state may be one of a waking state, a relaxation state, a state of hyper-focus, a state of flow, a state of altered consciousness, a meditative state, and an emotional state.

The at least one donor may be a plurality of donors, the method further comprising a step of: computing an average dominant frequency of brainwaves of said plurality of donors, wherein said plurality of donors are of the same biological sex and having an age within 5 years of the subject's age.

The stimulation of the subject may comprise stimulating the subject with at least one of a visual, an auditory, a tactile, a transcranial electric stimulation, a transcranial direct current stimulation (tDCS), a transcranial alternating current stimulation (tACS), and a transcranial magnetic stimulation.

The extraction of the dominant frequency may comprise performing one of a statistical pattern classification, a principal component analysis, a multilinear principal component analysis, a support vector machine analysis, an independent component analysis, a linear discriminant analysis, a quadratic discriminant analysis, a maximum entropy Markov model analysis, a multinomial logistic regression, a clustering, a factor analysis, or a EEG multifractal analysis.

The extraction of the dominant frequency over time may comprise using a recurrent neural network or a deep neural network.

The at least one external stimulus may have a frequency defined using a recurrent neural network or a deep neural network.

The external stimulus may comprise at least one of binaural beats and isochronic tones. The at least one external stimulus may comprise a visual stimulus selected from at least one of a red light and a near-infrared light.

The at least one donor may be a plurality of donors, wherein the extracted dominant brainwave frequency of at least one donor in the desired brain state comprises a computed average dominant frequency of brainwaves of said plurality of donors, said plurality of donors being of the same biological sex and having an age within 5 years of the subject's age.

The system may further comprise a stimulator configured to stimulate the subject's with at least one of a visual, an auditory, a tactile, a transcranial electric stimulation, a transcranial direct current stimulation (tDCS), a transcranial alternating current stimulation (tACS), and a transcranial magnetic stimulation.

The system may further comprise an automated processor configured to extract the dominant frequency through one of a statistical pattern classification, a principal component analysis, a multilinear principal component analysis, a support vector machine analysis, an independent component analysis, a linear discriminant analysis, a quadratic discriminant analysis, a maximum entropy Markov model analysis, a multinomial logistic regression, a clustering, a factor analysis, and a EEG multifractal analysis.

The system may further comprise a recurrent neural network or a deep neural network, configured to control the frequency of the external stimulus.

It is also an object to provide a method of enhancing memory, comprising: providing a transcranial brain stimulation to a subject; determining a range of endogenous frequencies of brain oscillation of the subject after the transcranial brain stimulation; after the transcranial brain stimulation, entraining brainwaves of the subject with a first sensory stimulation pattern having frequencies within the determined range of endogenous frequencies of brain oscillation of the subject; after entraining brainwaves of the subject with the first sensory stimulation pattern, entraining brainwaves of the subject with a second sensory stimulation pattern having a distinct range of frequencies from the range of endogenous frequencies of brain oscillation of the subject, the second sensory stimulation pattern comprising a temporal pattern derived from a human brainwave pattern corresponding to a cognitive task; and engaging the subject in the cognitive task concurrent with exposure of the subject to the second sensory stimulation pattern. The second sensory stimulation pattern may be derived from the human brainwave pattern corresponding to a cognitive task through a deconvolution operation.

It is a further object to provide a system for enhancing memory, comprising: a stimulator configured to provide a transcranial brain stimulation to a subject; a user interface configured to engage the subject in a cognitive task; at least one processor configured to: entrain brainwaves of the subject with a first sensory stimulation pattern having frequencies within a range of endogenous frequencies of brain oscillation of the subject; entrain brainwaves of the subject with a second sensory stimulation pattern having a distinct range of frequencies from the range of endogenous frequencies of brain oscillation of the subject, the second sensory stimulation pattern comprising a temporal pattern derived from a human brainwave pattern corresponding to a cognitive task; control a sequence of the first sensory stimulation pattern and the second sensory stimulation pattern; and engage the subject through the user interface in the cognitive task concurrent with exposure of the subject to the second sensory stimulation pattern. The at least one processor may be further configured to deconvolve the human brainwave pattern corresponding to the cognitive task.

It is a still further object to provide an apparatus for brain entrainment, comprising: a sleeping mask having an internal compartment for housing electronics; a motherboard having a memory module, a video controller, an audio controller, a wireless receiver, and a power connector, said motherboard positioned inside the sleeping mask with the power connector protruding outside the mask for connecting to a power cable; a first printed circuit board (PCB) containing a plurality of light-emitting diodes (LED) capable of emitting light in at least one of a red and a near-infrared portion of the spectrum, and controlled by the video controller, the first PCB position on the inner surface of the sleeping mask opposite a first eye; a second printed circuit board (PCB) containing a plurality of light-emitting diodes (LED) capable of emitting light in at least one of a red and a near-infrared portion of the spectrum, and controlled by the video controller, the second PCB position on the inner surface of the sleeping mask opposite a second eye, wherein light emitted by the LEDs on the first PCB and LEDs on the second PCB are synchronized with each other and light emitted by LEDs is modulated by a waveform having a frequency corresponding to a frequency characteristic of a desired brain state being extracted from brainwaves of at least one donor in the desired brain state; two earphones capable of emitting one of binaural beats and isochronic tones, said earphones controlled by the audio controller modulating the waveform on one of a frequency of sound and amplitude of sound, wherein binaural beats has the waveform frequency modulated on the frequency difference in each audio channel and the isochronic tones have the waveform frequency modulated on sound amplitude; and a battery contained in a compartment inside the sleeping mask; wherein the apparatus being configured to stimulate a subject wearing the sleeping mask to achieve brain entrainment via Arnold tongue in order to achieve the desired brain state.

### BRIEF DESCRIPTION OF THE DRAWING

The FIGURE is an illustration of a system for transplantation of brain states. Electroencephalography (EEG) is used to register a cortical signature associated with a distinct brain state from a donor. This signal is digitally processed and inverted before being applied to a recipient via transcranial electric stimulation (TES).

### DETAILED DESCRIPTION DF THE PREFERRED EMBODIMENTS

Brain States

Brain states refer to the synthesis of endogenous activity in part shaped by previous experience and current sensory input to create an overall state accessible to objective measurement. A brain state is sometimes described as a snapshot in time of the central nervous system (CNS). However, such a static picture taken in a particular moment in time does not reflect temporal patterns that are critical for describing the brain state. Brain states are expressed as patterns of active neurons, active synapses, and neural oscillations. When speaking of brain states, measurable patterns of neural activity and the ability to actively manipulate these spatiotemporal patterns in order to alter behavior are of concern. Some define brain states as patterns of synchronous neural firing (Brown, 2006). Brain states are interpreted here as macroscopic patterns of electrical activity in the brain that repeatedly occur as a function of endogenous neuronal dynamics and their response to sensory input. The neural correlates of brain states are rhythmic activity patterns resulting from neuronal oscillations. These rhythmic activity patterns represent measurable cortical signatures.

The concept of brain states has also been referred to as “mental states”. (Poltorak 2019) There is poor consensus about what mental states are and the nature of their relationship with the brain. According to identity theory, mental states are identical with brain states (Payne 2021). Others disagree (Chalmers 1995). Herein, the phrase “brain states” is employed to encompass the field, which concerns brain states as defined by measurable patterns of neural activity.

Brain states include conscious and unconscious states. Conscious states include inter alia the state of focus, the state of flow, and various emotional, motor, and cognitive states as well as altered states of consciousness. Unconscious states include various stages of sleep and the state of general anesthesia (Gervasoni et al., 2004).

An example of a state that is not a pure brain state is a state of relaxation, which is predominantly a state of the peripheral nervous system (PNS), not the central nervous system (CNS). The PNS regulates the relaxation state, which includes relaxation of the skeletal muscles regulated by the somatic nervous system, relaxation of smooth muscles as well as vasodilation, the slowing of the heartbeat and respiration, etc., all regulated by the autonomous nervous system (ANS). To induce this state, one must stimulate the vagus nerve, not the brain. Indeed, it is the excitation of the parasympathetic nervous system that causes the state of relaxation.

Sleep states are of particular interest, because each stage of sleep is (at least partially) defined by prominent rhythmic network activity in specific frequency bands (Steriade, 2006). The first non-REM stage of sleep, N1, is referred to as relaxed wakefulness. During this transitional stage the alpha waves (8-13 Hz) are replaced with the theta waves (5-7 Hz). The second non-REM stage of sleep, N2, is characterized by the appearance of sleep spindles (short bursts of high-frequency neural activity further subdivided into fast spindles of 11-13 Hz and slow spindles of 13-15 Hz (Cline, 2011)) and K-complexes (single long delta waves that last for only a second). The third non-REM stage of sleep, N3, is deep sleep, during which theta waves are replaced with delta waves (4 Hz and below). (See also (Lin et al., 2020).) Previously, N3 was subdivided into N3 and N4. For a summary of the four-stage sleep classification, see for example (Purves et al., 2001). It is worth pointing out that classification of sleep stages is not based exclusively on the detection of a single, dominant frequency but rather accounts for a series of criteria about frequency, amplitude, waveform shape, the occurrence of transient synchronized events (such as spindles and K-complexes), auxiliary signals about muscle tone and eye movements, and so forth.

The complexity of neural activity suggests that the reduction of brain states to an oscillatory signal of a single frequency can be expected to fail to capture the full complexity of brain states. Indeed, the conventional focus on individual oscillations and frequency bands as signatures of brain states is limiting. Nevertheless, the research literature reveals an almost exclusive focus on single-frequency stimulation waveforms for the modulation of brain states with noninvasive brain stimulation (such as the use of a sine wave in transcranial alternating current stimulation, tACS). Taking into account the rich temporal structure of brain activity beyond a single (dominant) frequency in the spectrum should enable more effective brain entrainment through stimulation. Thus, a naturalistic modulation waveform based on cortical signatures extracted from endogenous brain waves of a sleeping subject contains all attendant rhythms, which dynamically change in their prominence as the subject moves through various stages of the healthy sleep cycle. Using such a naturalistic, multifrequency dynamic waveform for stimulation is expected to be more efficacious than using a single static frequency, typically 5 Hz, that is traditionally used to induce sleep. Thus, neuromodulation with such naturalistic waveforms may prove beneficial in treating insomnia and other sleep disorders (Gebodh et al., 2019; Gebodh et al., MO).

Brain Waves as Neural Correlates of Brain States

Oscillating neurons produce emergent meso- and macroscale rhythmic electric signals often referred to as brain rhythms or brain waves, which can be detected and recorded using noninvasive techniques. Brain waves can be considered the neural correlates of brain states.

To be sure, brain waves are not identical with brain states and may not contain all information encoded in brain states. Measurements of brain waves, of course, are subject to all the limitations (such as noise or signal averaging across vast numbers of neurons) of the instruments being utilized, and loss of information is inevitable. However, a bilateral correlation between brain waves and brain states is posited to exist.

There is no doubt that brain states cause (via neuronal oscillations) brain waves. The question is, do brain waves cause brain states? Specific spatiotemporal patterns of neural activity in the brain that are correlated with particular brain states can also cause these brain states. So, if brain state A causes brain waves X (characterized by their frequencies and spatial distribution), replicating the same brain waves X (at least in the same person) results in brain state A. Therefore, the causal nature of the relationship can be experimentally investigated. This is more likely to hold true for brain states that are predominantly cortical states, because cortical brain states are more likely to be the product of cortical activity patterns such that, when a specific cortical activity is induced through stimulation, the brain switches to the corresponding state.

A growing number of studies have recently shown that modulating brain rhythms causes changes in cognitive performance, indicative of a causal role of brain oscillations in brain states (Grover et al., 2021; Romei et al., 2016; Frohlich et al., 2015). Sensory entrainment was shown to result in behavioral (perceptual) changes (Mathewson et al., HID). Therefore, a causal relationship that can be experimentally investigated is posited to exist. This is more likely to hold true for brain states that are predominantly cortical states, because cortical brain states are more likely to be the product of cortical activity patterns such that, when a specific cortical activity is induced through stimulation, the brain switches to the corresponding state.

On the other hand, this conjecture is less likely to hold for brain states that involve deep brain structures and specialized nuclei or the brain stem. For example, it can be imagined that certain spatiotemporal patterns are indicative of an underlying brain state not accessible with noninvasive measurements of brain activity. Imposing the corresponding activity patterns may fail to induce the corresponding brain state, since controlling (e.g., stimulating) inaccessible subcortical brain areas (or, more broadly, physiological processes) would be necessary. The state of sleep, for example, is regulated by the hypothalamus (including suprachiasmatic nuclei (SCN) responsible for circadian rhythms or the ventrolateral preoptic nucleus (VLPO)), brain stem, thalamus, pineal gland, basal forebrain, and amygdala. There is little reason to expect that the cortical signatures of the sleeping donor would entrain subcortical structures (such as the SEN or VLPO) of the recipient. Nevertheless, recent research confirms that cortical structures play a role in regulating sleep homeostasis and global sleep-wake dynamics (Krone et al., 2021), leaving room for the possibility that the state of sleep may be induced by entrainment.

This distinction results from the fact that neither recording nor stimulation techniques have access to all physiological processes that (at least in theory) contribute to a brain state.

Functional neuroimaging such as EEG or MEG can capture the neuronal activity of localized brain regions, which correlate with distinct cognitive or behavioral states (brain states). EEG recordings have demonstrated, for example, that the pattern of brain activity changes during meditative acts, and frontal cortex EEG activity has been associated with emotion induction and regulation (Yu et al., 2011; Dennis and Solomon, MD). EEG recordings reflect ionic fluctuations resulting from neuronal communication in the cortex arising from dendritic depolarizations (Nunez and Srinivasan, 2006). Alternatively, MEG measurements reflect intracellular ionic fluctuations resulting from action potentials (Hamalainen et al., 1993). In both cases, output measures correlate with localized cortical activity. EEG signals are not easy to localize. However, the overall macroscopic EEG signal reflects smaller scale endogenous rhythmic processes.

EEG or MEG recordings can be used to extract cortical signatures. This can be done using statistical techniques such as principal components analysis (PCA), independent components analysis (ICA), spectral analysis and similar techniques, or machine-learning pattern recognition.

Deconvolution of Brain States

As noted above, the stimulation pattern is defined based on brain states, e.g., EEG signals, but the stimulation needs to be “inverted” in order to have the desired effect. This is based on the fact that the EEG signals are produced by waves of depolarization of neurons in the brain, in a cyclic pattern. The EEG measures the convoluted pattern at the scalp. With a large number of signals, and knowledge of skull and brain anatomy, inferences may be drawn on the location and state of the population of neurons that generate components of the signal. For example, using statistical separation techniques such as principal component analysis and related techniques, independent sources may be inferred from complex superpositions. However, because neurons are linked, and share blood supply, cerebrospinal fluid, etc., the independence of sources is incomplete. Further, the superpositions of sources may be non-linear, further complicating the analysis. However, various techniques are known for addressing each type of deviation from the presumptions of independent sources, normal distribution statistics, and linearity.

The essential process for inversion is to determine what kind of stimulus leads to a desired brain wave pattern. To the extent that both desired and undesired waves are generated from a single stimulus, it is possible to suppress or cancel, or otherwise distinguish desired and undesired waves.

The system is generally considered underactuated, with, for example, two eyes and/or two ears available for sensory stimulation, and feasible skin or proprioceptive stimulation on the order of <16 stimuli, while the EEG may have 64 or 128 electrodes, and the brain model may have hundreds of relevant sources or voxels. See:
Abutaleb, A., Aya Fawzy, and Khaled Sayed. “Blind deconvolution of EEG signals using the stochastic calculus.” In 2012 Cairo International Biomedical Engineering Conference (CIBEC), pp. 175-178. IEEE, 2012.
Edlinger, Gtinter, Paul Wach, and Gert Pfurtscheller. “On the realization of an analytic high-resolution EEG.” IEEE transactions on biomedical engineering 45, no. 6 (1998): 736-745.
Ehinger, Benedikt V., and Olaf Dimigen. “Unfold: an integrated toolbox for overlap correction, non-linear modeling, and regression-based EEG analysis.” PeerJ 7 (2019): e7838.
Grandchamp, Romain, Claire Braboszcz, Scott Makeig, and Arnaud Delorme. “Stability of ICA decomposition across within-subject EEG datasets.” In 2012 Annual International Conference of the IEEE Engineering in Medicine and Biology Society, pp. 6735-6739. IEEE, 2012.
Huang, Lu, and Hong WANG. “Blind separation of EEG based on blind deconvolution.” Journal of Northeastern University (Natural Science) 37, no. 8 (2016): 1100.
Kazemipour, Abbas, Ji Liu, Krystyna Solarana, Daniel A. Nagode, Patrick D. Kanold, Min Wu, and Behtash Babadi. “Fast and stable signal deconvolution via compressible state-space models.” IEEE Transactions on Biomedical Engineering no. 1 (2017): 74-86.
Naït-Ali, A. M., D. Adam, and J-F. Motsch. “The brainstem auditory evoked potentials estimation using a bayesian deconvolution method.” In Proceedings of 18th Annual International Conference of the IEEE Engineering in Medicine and Biology Society, vol. 4, pp. 1516-1517. IEEE, 1996.
Nunez, Paul L. “Spatial analysis of EEG.” In Images of the Twenty-First Century. Proceedings of the Annual International Engineering in Medicine and Biology Society, pp. 691-692. IEEE, 1989.
Panwar, Sharaj, Paul Rad, John Duarles, and Yufei Huang. “Generating EEG signals of an RSVP experiment by a class conditioned wasserstein generative adversarial network.” In 2019 IEEE International Conference on Systems, Man and Cybernetics (SMC), pp. 1304-1310. IEEE, 2019.
Penny, W., N. Trujillo-Barreto, and E. Aubert. “Spatia-temporal models for EEG.” Friston et al. (Eds.), Statistical Parametric Mapping: Models for Brain Imaging. Elsevier (2006).
Sanei, S., and A. R. Leyman. “EEG brain map reconstruction using blind source separation.” In Proceedings of the 11th IEEE Signal Processing Workshop on Statistical Signal Processing (Cat. No. DITH8563), pp. 233-236. IEEE, 2001.
Satherley, Brenda Lee. “Zero-based ensemble deconvolution and EEG spectral topography.” (1994).
Sjontoft, Erik. “A deconvolution procedure for use in extracting information in average evoked response EEG signals.” IEEE Transactions on Biomedical Engineering 4 (1980): 227-230.
Trujillo-Barreto, Nelson J., Eduardo Aubert-Vázquez, and William D. Penny. “Bayesian M/EEG source reconstruction with spatia-temporal priors.” Neuroimage 39, no. 1 (2008): 318-335.
Wen, Tingxi, and Zhongnan Zhang. “Deep convolution neural network and autoencoders-based unsupervised feature learning of EEG signals.” IEEE Access B (2018): 25399-25410.

Neuromodulation

These cortical signatures may be inverted in order to stimulate cortical activity. Endogenous electric fields (i.e., brain waves) directly entrain neuronal network activity (Frohlich and McCormick, 2010). This fundamental discovery enables the design of low-amplitude electromagnetic brain stimulation modalities to modulate and enhance oscillatory network dynamics. Indeed, transcranial electric stimulation (TES) (Annarumma et al., 2018), including transcranial alternating current stimulation (tACS) (Antal et al., 2008) and transcranial direct current stimulation (tDCS) (Nitsche, 2000; see also Utz et al., 2010), are used to electrically stimulate cortical activity, whereas transcranial magnetic stimulation (TMS) (Barker, 1985; Lawson McLean, 2019) uses a spatially targeted magnetic field to achieve a similar endpoint of controlling electric activity in brain networks. Typical brain stimulation methods use constant stimulation as a waveform (e.g., tDCS) or a synthetic waveform, which may be a step function modulated on a direct current, such as “electrosleep” (Robinovitch, 1911), a sinusoid modulated on an oscillatory direct current (osc-tDCS) (D'Atri et al., 2016), or a single fixed frequency of alternating current (tACS) (Rosa and Lisanby, 2012; Herrmann, 2013). (Helfrich et al., 2014) utilized simultaneous tACS stimulation combined with EEG recordings to show that, when tACS was applied to the parieto-occipital lobe of the brain, alpha oscillations increased and became synchronized with the entrainment frequency. Similarly, brain entrainment can be achieved by stimulating the brain via sensory pathways using visual or auditory stimuli (Notbohm et al., 2016).

Whereas TES and TMS modulate neuronal activity, entrainment using sensory pathways can also change the brain state. Indeed, the endogenous circadian rhythm responsible for the transition from the sleeping state to the waking state is primarily entrained by light (Bedrosian et al., 2013).

Endogenous circadian rhythms are entrained-both in terms of the firing of action potentials and gene expression-to the diurnal cycle. Although such entrainment happens on a distinctly slower timescale than the neuronal oscillations discussed here so far, this serves as an example of how sensory input alters rhythmic processes in the brain. Rhythmic brain activity is a reflection of rhythmic processes in our environment (from the slow timescales of the day-night cycle to the fast oscillations in auditory input) across an extremely wide band of frequencies.

It is noteworthy that suprachiasmatic nuclei (SEN) owe their ability to sense the cycle of light and darkness to the retinohypothalamic tract (RHT), connecting the intrinsically photosensitive retinal ganglion cells (ipRGC) in the retina to SCN via the optic nerve (Cooley et al., 2001). The RHT provides a natural pathway to use photobiomodulation for stimulating the hypothalamus and, through it, other structures of the brain.

The brain is very efficient at processing light and sound stimuli. Therefore, stimulation with light (photobiomodulation) or sound (isochronic tones or binaural beats) holds significant promise for entraining a brain state. These natural stimuli have a greater likelihood of reproducing naturalistic brain rhythms than single frequency stimulation waveforms do. (See, for example, Pérez et al., 2017).

Sensory stimulation is particularly promising since it uses established neuronal circuits for the brain to respond to external input and avoids the pitfalls inherent in recent debates about the amount of energy actually delivered to the brain by transcranial current stimulation. However, it is also conceivable that certain structural and functional brain networks are configured to be robust to perturbations caused by sensory input. These circuits may be preferentially modulated by electromagnetic stimulation.

These techniques may be used to transplant a desirable brain state from one subject (the “donor”), who is in the desired brain state, to another subject (the “recipient,” who is either another person or the same subject at another time), who wishes to attain this brain state. This may be achieved by recording and subsequently inducing desired brain states. Thus, EEG or MEG may be used to record the brain state of the first subject (the donor), from which cortical signatures may be extracted and inverted to create modulation waveforms. Such waveforms may then be modulated on various physical signals, such as direct or alternating current, magnetic field, light, sound, or vibration and applied via TES (tDCS, osc-tDCS, tACS), TMS, or sensory (visual, auditory, or tactile) stimulation to entrain the brain of the recipient with the cortical signatures of the donor, thereby inducing the cognitive-behavioral state of the donor within the recipient.

This technique (transplanting brain state) was tested in the domain of sleep (Gebodh et al., 2019; Gebodh et al., 2020). In these studies, TES (tACS modulated with endogenous sleep-derived waveform, “tESD”) was used. For the reasons explained above, light and sound appear to be more promising modalities for inducing the desired brain state than direct brain stimulation with TES would be.

Generally, the notion of “transplanting” brain states, including sleep, attention, and learning as well as emotional valence is of particular interest. Attention states in the brain primarily result from the cognitive control process of the selective direction of information processing resources to behaviorally relevant stimuli and from active suppression of the detection and processing of irrelevant stimuli (Gulbinaite et al., 2017). Thus, functionally, attention serves as both a filter, eliminating less relevant stimuli from conscious perception, and an amplifier, increasing the salience of behaviorally relevant stimuli. This cognitive state is associated with specific neuronal oscillations (Schroeder et al., 2010), which may be captured by EEG or MEG. The neural oscillations associated with attention have been shown to be disrupted in a number of conditions, including epilepsy (Besle et al., 2011), dyslexia (Thomson and Goswami, 2008; Leong and Goswami, 2014; Soltész et al., 2013), and schizophrenia (Lakatos et al., 2013). Therefore, acquiring a brain wave signature during states of attention in a healthy “donor” may prove valuable when applied to a recipient exhibiting attention deficits associated with disrupted or otherwise irregular cortical oscillations.

Stimulating the brain using a waveform with a fixed frequency that significantly differs from the frequency of the endogenous brain waves (regular or irregular), as frequently done in TES and TMS studies, may cause interference and is unlikely to change the rhythm of endogenous brain waves. In contrast, brain entrainment should optimally start where the brain is, not where it is desired to be.

The Arnold tongue is a theoretical framework for entrainment, essentially suggesting that it is easier to entrain oscillations closer to endogenous frequencies within a given subject. Thus, neuromodulation with a dynamic waveform used for entrainment should start at the current frequency of the endogenous brain waves since such close matching of stimulation and endogenous frequency is required for phase locking as indicated by the Arnold tongue (Ali, 2013) for entrainment of neural oscillations. Once entrainment is achieved at this initial frequency, the frequency of stimulation can be gradually changed to move the endogenous rhythm toward the desired frequency in order to achieve successful entrainment at the desired target frequency (Notbohm et al., 2016; Thut et al., 2011). This approach also avoids interference issues.

Previous research shows that memory functions are acutely sensitive to neural entrainment and may be disrupted via TMS (Hanslmayr et al., 2014), indicating the possibility of an inverse, positive entrainment of these oscillations.

Similarly, emotional arousal and valence are correlated with distinct cortical signatures observable through EEG (Allen et al., 2018). Previous data indicate that happiness resulting from musical experience, for instance, is associated with increased theta frequency oscillations in the left frontal cortical hemisphere (Rogenmoser et al., 2016). Cortical oscillations associated with negative affect conversely correlate with decreased theta frequency oscillations in this same region. Notably, aberrant cortical oscillations have been observed in a range of affective disorders, including major depression (Van der Vinne et al., 2017). Indeed, the left frontal hemisphere exhibits disrupted cortical rhythms in patients diagnosed with major depression when compared with healthy controls (Nusslock et al., 2018). Similar data have highlighted cortical asymmetry of frontal lobe oscillations in post-traumatic stress disorder (PTSD) (Meyer et al., 2018). Simple cortical entrainment via binaural beat stimulation has already proven adequate for inducing specific emotional states (Chaieb et al., 2015). More directly, cranial electrotherapy has been demonstrated as an effective treatment for depression, anxiety, and certain forms of insomnia (Kirsch and Nichols, 2013). In fact, certain forms of depression may respond better to transcranial approaches, such as TMS, as has been demonstrated in early data on patients with treatment-resistant major depression (George, 2000; Rosenberg et al., 2010).

Transplanting Brain States

Thus, the “transplanting” (transferring) of brain states by replicating neural correlates of the donor's state in a recipient (who may be a different person or the same person at a different time) is founded on two primary principles. First, cortical signatures found in brain waves are neural correlates of brain states. A large body of literature has identified distinct, measurable cortical signatures associated with specific brain states, ranging from those defining the sleep/wake cycle to those underlying emotional experiences. Second, TES, TMS, and sensory stimulation by light and sound have been repeatedly demonstrated as efficacious, safe means by which cortical rhythms may be entrained with a high degree of location-specificity, with sensory stimulation using light or sound holding particular promise for brain entrainment. Third, brain waves resulting from brain entrainment causally induce the desired brain state associated with the cortical signatures that are encoded in the modulation waveforms used for entrainment. Thus, a bidirectional causal relationship is apparent between brain states and cortical signatures found in brain waves.

To be sure, brains differ. While the donor and the recipient may on occasion be the same person, when the donor and the recipient are not the same individual, they can be expected to differ in skull structure, brain size, and brain morphology. Moreover, the phase lag between oscillations and stimulus can differ across individuals during neural entrainment. The same is true for the optimal timing of tACS when it is applied to modulate entrainment. Although it is possible to adjust the waveform based on the specifics of the recipient brain obtained by fMRI and using computational models of the brain, this problem can be sidestepped entirely by using sensory stimulation that does not act on the brain itself but rather acts on the sensory organs and allows the brain to assimilate the signals it receives from these sensory organs on its own terms. This is yet another reason why sensory stimulation using visual and/or auditory pathways seems highly preferable to using transcranial electric (TES) or magnetic (TMS) stimulation.

The complexity of identifying and transplanting brain states should not be underestimated. The brain may be investigated on microscopic neurochemical levels or as a complex and widely distributed network. It is not immediately obvious which is the correct level to be acquiring and transplanting patterns that would affect changes in behavior (that is, mental states). For example, the macroscopic representations measured by EEG may be sufficient to transplant generic physiological states, such as a state associated with a specific sleep stage. However, more refined states, such as specific cognitive states, may require a higher spatial resolution to be fully captured and transplanted by non-invasive methods. Cortical signatures and representation patterns may be carefully investigated before transplanting or replicating subjective contents of cognitive processes.

It is also possible that cortical activity signals as measured by EEG do not fully capture certain brain states. For example, REM sleep can be distinguished from waking activity by the absence of muscle tone in major muscle groups as determined by electromyography (EMG). Therefore, additional physiological signals can be used to capture brain states more fully. In particular, capturing the status of the autonomic nervous system, such as, for example, the dynamic balance of sympathetic and parasympathetic activity reflected in measures such as heart rate variability, may turn out to be necessary for high-fidelity identification of brain states. These signals are also open to noninvasive modulation, such as through the stimulation of the vagus nerve.

Combining vagus nerve stimulation (VNS) with brain entrainment is promising. It has been shown that VNS significantly increased and decorrelated spontaneous activity and suppressed entrainment at 6-8 Hz (Nichols et al., 2011).

Finally, it is worth discussing the difference between online and offline effects of stimulation. The current brain stimulation literature suggests that cortical states can be effectively enhanced during stimulation, what are referred to as “online” effects. Transplanting brain states focuses on augmenting, restoring, and inducing brain states with stimulation, and is not dependent on a full understanding of the mechanism by which more long-lasting effects occur in brain networks after the conclusion of stimulation. Brief perturbations may be sufficient to switch to another state either with or without continued stimulation. The main mechanisms are based on the fundamental property of neuronal oscillations to respond to (weak) perturbations through entrainment.

### CONCLUSION

Together these findings provide the basis for the transplant (transfer) of brain states and provide the means by which a cortical signature may be obtained via EEG or EMG associated with the desired brain state of a “donor” that may, in turn, be processed, inverted or deconvoluted, and subsequently applied to a “recipient”—who may be another person, or the same person at another time-to induce this state through cortical rhythm entrainment using preferably sensory stimuli, such as light or sound, a combination thereof, or, possibly, TES or TMS. Using cortical signatures acquired from a donor, rather than a fixed-frequency synthetic waveform stimulation as is currently typical for TES techniques, offers the distinct advantage of replicating multiphasic, multifrequency, temporally dynamic, naturalistic signals, which is more likely to modulate neuronal network activity effectively (Frohlich and McCormick, 2010) and, more important, induce naturalistic brain states due to the additional information contained in the complete spectrum of macroscale brain signals. Therefore, this technique provides a novel and effective neuromodulation approach to the noninvasive, non-pharmacological treatment of a variety of psychiatric and neurological disorders for which current treatments are mostly limited to pharmacotherapeutic interventions.

What is claimed is:

1. A method of brain entrainment for transplanting a desired brain state, comprising:
extracting a dominant brainwave frequency of at least one donor in the desired brain state;
detecting an endogenous dominant brainwave frequency of a subject desirous of achieving the desired brain state;
stimulating the subject's brain with at least one external stimulus having a frequency corresponding to the endogenous dominant brainwave frequency of the subject to lock phase and establish an Arnold tongue; and
after establishing the Arnold tongue, incrementally changing the frequency of the external stimulus from the endogenous dominant frequency to the dominant brainwave frequency of said at least one donor's brain, thereby assisting the subject in achieving the desired brain state.

2. The method of claim 1, wherein the desired brain state is a sleeping state.

3. The method of claim 1, wherein the respective brain state is one of a waking state, a relaxation state, a state of hyper-focus, a state of flow, a state of altered consciousness, a meditative state, and an emotional state.

4. The method of claim 1, wherein said at least one donor is a plurality of donors, the method further comprising a step of:
computing an average dominant frequency of brainwaves of said plurality of donors,
wherein said plurality of donors are of the same biological sex and having an age within 5 years of the subject's age.

5. The method of claim 1, wherein the step of stimulating the subject comprises stimulating the subject with at least one of a visual, an auditory, a tactile, a transcranial electric stimulation, a transcranial direct current stimulation (tDCS), a transcranial alternating current stimulation (tACS), and a transcranial magnetic stimulation.

6. The method of claim 1, wherein the step of extracting the dominant frequency comprises performing one of a statistical pattern classification, a principal component analysis, a multilinear principal component analysis, a support vector machine analysis, an independent component analysis, a linear discriminant analysis, a quadratic discriminant analysis, a maximum entropy Markov model analysis, or a multinomial logistic regression.

7. The method of claim 1, wherein the step of extracting the dominant frequency over time comprises performing a clustering.

8. The method of claim 1, wherein the step of extracting the dominant frequency over time comprises performing a factor analysis.

9. The method of claim 1, wherein the step of extracting the dominant frequency over time comprises performing a EEG multifractal analysis.

10. The method of claim 1, wherein the step of extracting the dominant frequency over time comprises using a recurrent neural network or a deep neural network.

11. The method of claim 1, wherein the at least one external stimulus has a frequency defined using a recurrent neural network or a deep neural network.

12. The method of claim 1, wherein the external stimulus comprises at least one of binaural beats and isochronic tones.

13. The method of claim 1, wherein the at least one external stimulus comprises a visual stimulus selected from at least one of a red light and a near-infrared light.

14. A system for transplanting a desired brain state through brain entrainment, comprising:
at least one memory configured to store an extracted dominant brainwave frequency of at least one donor in the desired brain state;
at least one automated processor configured to:
detect an endogenous dominant brainwave frequency of a subject desirous of achieving the desired brain state;
stimulate the subject's brain with at least one external stimulus having a frequency corresponding to the endogenous dominant brainwave frequency of the subject to lock phase and establish an Arnold tongue; and
after establishing the Arnold tongue, incrementally change the frequency of the external stimulus from the endogenous dominant frequency to the dominant brainwave frequency of said at least one donor's brain,
to thereby assist the subject in achieving the desired brain state.

15. The system of claim 14, wherein said at least one donor is a plurality of donors, wherein the extracted dominant brainwave frequency of at least one donor in the desired brain state comprises a computed average dominant frequency of brainwaves of said plurality of donors, said plurality of donors being of the same biological sex and having an age within 5 years of the subject's age.

16. The system of claim 14, further comprising a stimulator configured to stimulate the subject's with at least one of a visual, an auditory, a tactile, a transcranial electric stimulation, a transcranial direct current stimulation (tDCS), a transcranial alternating current stimulation (tACS), and a transcranial magnetic stimulation.

17. The system of claim 14, further comprising an automated processor configured to extract the dominant frequency through one of a statistical pattern classification, a principal component analysis, a multilinear principal component analysis, a support vector machine analysis, an independent component analysis, a linear discriminant analysis, a quadratic discriminant analysis, a maximum entropy Markov model analysis, a multinomial logistic regression, a clustering, a factor analysis, and a EEG multifractal analysis.

18. The system of claim 14, further comprising a recurrent neural network or a deep neural network, configured to control the frequency of the external stimulus.

19. The system of claim 14, wherein the external stimulus comprises at least one of binaural beats, isochronic tones, a red light, and a near-infrared light.

20. A method of enhancing memory, comprising:
providing a transcranial brain stimulation to a subject;
determining a range of endogenous frequencies of brain oscillation of the subject after the transcranial brain stimulation;
after the transcranial brain stimulation, entraining brainwaves of the subject with a first sensory stimulation pattern having frequencies within the determined range of endogenous frequencies of brain oscillation of the subject;
after entraining brainwaves of the subject with the first sensory stimulation pattern, entraining brainwaves of the subject with a second sensory stimulation pattern having a distinct range of frequencies from the range of endogenous frequencies of brain oscillation of the subject, the second sensory stimulation pattern comprising a temporal pattern derived from a human brainwave pattern corresponding to a cognitive task; and
engaging the subject in the cognitive task concurrent with exposure of the subject to the second sensory stimulation pattern.

21. The method according to claim 20, wherein the second sensory stimulation pattern is derived from the human brainwave pattern corresponding to a cognitive task through a deconvolution operation.

22. A system for enhancing memory, comprising:
a stimulator configured to provide a transcranial brain stimulation to a subject;
a user interface configured to engage the subject in a cognitive task;
at least one processor configured to:
entrain brainwaves of the subject with a first sensory stimulation pattern having frequencies within a range of endogenous frequencies of brain oscillation of the subject;
entrain brainwaves of the subject with a second sensory stimulation pattern having a distinct range of frequencies from the range of endogenous frequencies of brain oscillation of the subject, the second sensory stimulation pattern comprising a temporal pattern derived from a human brainwave pattern corresponding to a cognitive task;
control a sequence of the first sensory stimulation pattern and the second sensory stimulation pattern; and
engage the subject through the user interface in the cognitive task concurrent with exposure of the subject to the second sensory stimulation pattern.

23. The system according to claim 19, wherein the at least one processor is further configured to deconvolve the human brainwave pattern corresponding to the cognitive task.

24. An apparatus for brain entrainment, comprising:
a sleeping mask having an internal compartment for housing electronics;
a motherboard having a memory module, a video controller, an audio controller, a wireless receiver, and a power connector, said motherboard positioned inside the sleeping mask with the power connector protruding outside the mask for connecting to a power cable;
a first printed circuit board (PCB) containing a plurality of light-emitting diodes (LED) capable of emitting light in at least one of a red and a near-infrared portion of the spectrum, and controlled by the video controller, the first PCB position on the inner surface of the sleeping mask opposite a first eye;
a second printed circuit board (PCB) containing a plurality of light-emitting diodes (LED) capable of emitting light in at least one of a red and a near-infrared portion of the spectrum, and controlled by the video controller, the second PCB position on the inner surface of the sleeping mask opposite a second eye, wherein light emitted by the LEDs on the first PCB and LEDs on the second PCB are synchronized with each other and light emitted by LEDs is modulated by a waveform having a frequency corresponding to a frequency characteristic of a desired brain state being extracted from brainwaves of at least one donor in the desired brain state;
two earphones capable of emitting one of binaural beats and isochronic tones, said earphones controlled by the audio controller modulating the waveform on one of a frequency of sound and amplitude of sound, wherein binaural beats has the waveform frequency modulated on the frequency difference in each audio channel and the isochronic tones have the waveform frequency modulated on sound amplitude;
a battery contained in a compartment inside the sleeping mask;
wherein the apparatus being configured to stimulate a subject wearing the sleeping mask to achieve brain entrainment via Arnold tongue in order to achieve the desired brain state.
